(19) United States
(12) Patent Application Publication
(10) Pub. No.: US 2024/0117392 A1
(43) Pub. Date: Apr. 11, 2024

(54) ENGINEERED BACTERIA AND METHODS OF PRODUCING TRIACYLGLYCERIDES

(71) Applicant: PRESIDENT AND FELLOWS OF HARVARD COLLEGE, Cambridge, MA (US)

(72) Inventors: Shannon Noel NANGLE, Cambridge, MA (US); Marika ZIESACK, Cambridge, MA (US)

(73) Assignee: PRESIDENT AND FELLOWS OF HARVARD COLLEGE, Cambridge, MA (US)

(21) Appl. No.: 18/274,757

(22) PCT Filed: Feb. 9, 2022
(86) PCT No.: PCT/US2022/015793

§ 371 Date: Jul. 28, 2023

Related U.S. Application Data
(60) Provisional application No. 63/165,941, filed on Mar. 25, 2021.
(60) Provisional application No. 63/147,496, filed on Feb. 9, 2021.

(51) Int. Cl. C12P 7/6458; C12N 1/20; C12N 9/10; C12N 9/12; C12N 9/16

(52) U.S. Cl. CPC C12P 7/6458; C12N 1/205; C12N 9/1029; C12N 9/1205; C12N 9/16; C12N 2500/05; C12N 2500/34; C12N 2500/35; C12Y 203/01015; C12Y 203/0102; C12Y 207/01107; C12Y 301/03004

(57) ABSTRACT

The technology described herein is directed to engineered chemoautotrophic bacteria and methods of producing triacylglycerides. Also described herein are systems or bioreactors comprising said engineered bacteria.

### CROSS-REFERENCE TO RELATED APPLICATIONS

This application claims benefit under 35 U.S.C. § 119(e) of U.S. Provisional Application No. 63/147,496 filed Feb. 9, 2021, and U.S. Provisional Application No. 63/165,941 filed Mar. 25, 2021, the contents of each of which are incorporated herein by reference in their entireties.

### GOVERNMENT SUPPORT

This invention was made with Government support under DE-AR0001509 awarded by the Department of Energy (USDOE). The Government has certain rights in the invention.

### SEQUENCE LISTING

The instant application contains a Sequence Listing which has been submitted in ASCII format via EFS-Web and is hereby incorporated by reference in its entirety. Said ASCII copy, created on Feb. 9, 2022, is named 002806-099270WOPT_SL.txt and is 336,121 bytes in size.

### TECHNICAL FIELD

The technology described herein relates to engineered bacteria and methods of producing triacylglycerides.

### BACKGROUND

A sustainable future relies, in part, on minimizing the usage of petrochemicals and reducing greenhouse gas (GHG) emissions. One way to accomplish this goal is through increasing the usage of sustainable bioproducts from engineered microorganisms, i.e., microbial bioproduction. Traditional microbial bioproduction utilizes carbohydrate-based feedstocks, but some of the cheapest and most sustainable feedstocks are gases (e.g., CO, CO₂, H₂, CH₄) from various point sources (e.g., steel mills, ethanol production plants, steam reforming plants, biogas). Compared to traditional bioproduction, gas fermentation represents a more cost-effective method that uses land more efficiently and has a smaller carbon footprint.

C. necator H16 (formerly known as Ralstonia eutropha H16) is an attractive species for industrial gas fermentation. It is a facultative chemolithotrophic bacterium that derives its energy from H₂ and carbon from CO₂, is genetically tractable, can be cultured with inexpensive minimal media components, is non-pathogenic, has a high-flux carbon storage pathway, and fixes the majority of fed CO₂ into biomass. However, many previous C. necator bioproduction methods have relied upon carbohydrate-based feedstocks (see e.g., U.S. Pat. No. 7,622,277; EP Patent 2,935,599; Green et al. Biomacromolecules. 2002 January-February, 3(1):208-13; Brigham et al. Deletion of Glyoxylate Shunt Pathway Genes Results in a 3-Hydroxybutyrate Overproducing Strain of Ralstonia eutropha. 2015 Synthetic Biology: Engineering, Evolution & Design. Poster Abstract 17: p. 32; the content of each of which is incorporated by reference in its entirety). There is a need to expand from this work by engineering C. necator to produce a large diversity of products using gas fermentation in order to promote the sustainable development of industrial bioproduction.

The area of animal-free replacements for lipids remains largely untapped. Milk fats are largely responsible for texture, flavor, energy content, and the solubility of some vitamins in dairy products. The possibility of biomanufacturing such animal-free milk fats to provide an alternative option for milk, butter, cheese, creams, ice cream, and meat represents a critical part of the solution for utilizing synthetic biology to lessen the environmental impacts of addressing humanity's increasing food production demands. Worldwide, cattle farming produces 11% of all greenhouse gas emissions and the dairy industry emits 3% annually. Current dairy alternatives are currently limited by the ability of plant fats to confer the same properties as dairy fats. Milk lipids are in a large part responsible for the taste and texture of dairy products, especially in the case of cheese and butter. Currently, there are no commercially available animal-free replacements for these fats, and plant-based options lack the physical properties for many applications as well as introduce unwanted flavors. There is thus a great need for engineered non-plant organisms that can produce such milk fats, such as triacylglycerides (TAGs), which are the major class of fats in dairy.

### SUMMARY

The technology described herein is directed to engineered chemoautotrophic bacteria and methods of using them to produce triacylglycerides (TAGs). Herein, C. necator is shown to bridge the gap between cheap feedstocks and versatile bioproduction. The methods and compositions described herein permit the production of tailored polymers using C. necator, something not achieved by prior applications. The engineered bacteria and methods described herein can reduce greenhouse gas (GHG) emissions, e.g., when industrially scaled.

Accordingly, in one aspect described herein is an engineered Cupriavidus necator bacterium, comprising: (a) at least one exogenous copy of at least one functional acyltransferase gene; and/or (b) at least one exogenous copy of at least one functional phosphatidic acid (PA) phosphatase gene.

In one aspect, described herein is an engineered Cupriavidus necator bacterium, comprising: (a) at least one exogenous copy of at least one functional acyltransferase gene encoding an acyltransferase enzyme that catalyzes transesterification of the sn3 OH group of a diacylglycerol with a fatty acid; and/or (b) at least one exogenous copy of at least one functional phosphatidic acid (PA) phosphatase gene.

In some embodiments of any of the aspects, the acyltransferase gene encodes for an acyltransferase enzyme that catalyzes transesterification of the sn3 OH group, the sn2 OH group, or the sn1 OH group of a triacylglycerol (TAG) precursor with a fatty acid

In some embodiments of any of the aspects, the acyltransferase gene encodes an acyltransferase enzyme that catalyzes transesterification of the sn3 OH group of a diacylglycerol with a fatty acid.

In some embodiments of any of the aspects, the acyltransferase gene is a functional diglyceride acyltransferase (DGAT) gene, a functional wax synthase (WS) gene, or a hybrid thereof.

In some embodiments of any of the aspects, the functional DGAT gene is heterologous.

In some embodiments of any of the aspects, the functional heterologous DGAT gene comprises a Acinetobacter baylyi DGAT gene, a Thermomonospora curvata DGAT gene, a Theobroma cacao DGAT gene, or a Rhodococcus opacus DGAT gene.

In some embodiments of any of the aspects, the acyltransferase gene encodes an acyltransferase enzyme that catalyzes transesterification of the sn2 OH group of a lysophosphatidic acid with a fatty acid.

In some embodiments of any of the aspects, the acyltransferase gene is a functional lysophosphatidic acid acyltransferase (LPAT) gene.

In some embodiments of any of the aspects, the functional LPAT gene is heterologous.

In some embodiments of any of the aspects, the functional heterologous LPAT gene comprises a Theobroma cacao LPAT gene.

In some embodiments of any of the aspects, the acyltransferase gene encodes an acyltransferase enzyme that catalyzes transesterification of the sn1 OH group of a glyceraldehyde-3-phosphate with a fatty acid.

In some embodiments of any of the aspects, the acyltransferase gene is a functional glycerol-3-phosphate acyltransferase (GPAT) gene.

In some embodiments of any of the aspects, the functional GPAT gene is heterologous.

In some embodiments of any of the aspects, the functional heterologous GPAT gene comprises a Durio zibethinus GPAT gene, Gossypium arboreum GPAT gene, Hibiscus syriacus GPAT gene, or a Theobroma cacao GPAT gene.

In some embodiments of any of the aspects, the fatty acid is esterified with acyl carrier protein (ACP) or with acetyl-CoA.

In some embodiments of any of the aspects, the functional phosphatidic acid (PA) phosphatase gene encodes a phosphatidic acid (PA) phosphatase enzyme that catalyzes dephosphorylation at the sn3 position of phosphatidic acid (PA).

In some embodiments of any of the aspects, the phosphatidic acid (PA) phosphatase gene is a functional phosphatidate phosphatase (PAP) gene.

In some embodiments of any of the aspects, the functional PAP gene is heterologous.

In some embodiments of any of the aspects, the functional heterologous PAP gene comprises a Rhodococcus opacus PAP gene, or a Rhodococcus jostii PAP gene.

In some embodiments of any of the aspects, the engineered bacteria further comprises: at least one exogenous copy of at least one functional thioesterase (TE) gene.

In some embodiments of any of the aspects, the functional thioesterase gene is heterologous.

In some embodiments of any of the aspects, the functional heterologous thioesterase gene is selected from the group consisting of: a Marvinbryantia formatexigens TE gene, a Cuphea palustris FatB1 gene, a Cuphea palustris FatB2 gene, a Cuphea palustris FatB2-FatB1 hybrid gene, a Arachis hypogaea FatB2-1 gene, a Mangifera indica FatA gene, a Morella rubra FatA gene, a Pistacia vera FatA gene, a Theobroma cacao FatA gene, a Theobroma cacao FatB gene (e.g., FatB1, FatB2, FatB3, BatB4, FatB5, or FatB6), or a Limosilactobacillus reuteri TE gene.

In some embodiments of any of the aspects, the engineered bacteria further comprises: (i) at least one endogenous polyhydroxyalkanoate (PHA) synthase gene comprising at least one engineered inactivating modification; or (ii) at least one exogenous inhibitor of an endogenous polyhydroxyalkanoate (PHA) synthase gene or gene product.

In some embodiments of any of the aspects, the engineered inactivating modification of the endogenous PHA synthase comprises one or more of i) deletion of the entire coding sequence, ii) deletion of the promoter of the gene, iii) a frameshift mutation, iv) a nonsense mutation (i.e., a premature termination codon), v) a point mutation, vi) a deletion, vii) or an insertion.

In some embodiments of any of the aspects, the endogenous PHA synthase comprises phaC.

In some embodiments of any of the aspects, the engineered bacteria further comprises: (i) at least one endogenous diacylglycerol kinase gene comprising at least one engineered inactivating modification; or (ii) at least one exogenous inhibitor of an endogenous diacylglycerol kinase gene or gene product.

In some embodiments of any of the aspects, the engineered inactivating modification of the endogenous diacylglycerol kinase comprises one or more of i) deletion of the entire coding sequence, ii) deletion of the promoter of the gene, iii) a frameshift mutation, iv) a nonsense mutation (i.e., a premature termination codon), v) a point mutation, vi) a deletion, vii) or an insertion.

In some embodiments of any of the aspects, the endogenous diacylglycerol kinase comprises dgkA.

In some embodiments of any of the aspects, the engineered bacteria further comprises: (i) at least one endogenous beta-oxidation gene comprising at least one engineered inactivating modification; or (ii) at least one exogenous inhibitor of an endogenous beta-oxidation gene or gene product.

In some embodiments of any of the aspects, the engineered inactivating modification of the endogenous beta-oxidation gene comprises one or more of i) deletion of the entire coding sequence, ii) deletion of the promoter of the gene, iii) a frameshift mutation, iv) a nonsense mutation (i.e., a premature termination codon), v) a point mutation, vi) a deletion, vii) or an insertion.

In some embodiments of any of the aspects, the endogenous beta-oxidation gene comprises FadE or FadB.

In some embodiments of any of the aspects, said engineered bacteria is a chemoautotroph.

In some embodiments of any of the aspects, said engineered bacteria uses CO₂ as its sole carbon source, and/or said engineered bacteria uses H₂ as its sole energy source.

In some embodiments of any of the aspects, said engineered bacteria uses fructose as its sole carbon source.

In some embodiments of any of the aspects, said engineered bacteria uses glycerol as its sole carbon source.

In some embodiments of any of the aspects, said engineered bacteria produces triacylglycerides.

In some embodiments of any of the aspects, said engineered bacteria produces animal triacylglycerides.

In some embodiments of any of the aspects, said engineered bacteria produces milk fats.

In one aspect, described herein is a method of producing triacylglycerides (TAGs), comprising: (a) culturing an engineered bacterium as described herein in a culture medium comprising CO₂ and/or H₂; and (b) isolating, collecting, or concentrating TAGs from said engineered bacterium or from the culture medium of said engineered bacterium.

In some embodiments of any of the aspects, the culture medium comprises CO₂ as the sole carbon source, and/or the culture medium comprises H₂ as the sole energy source.

In some embodiments of any of the aspects, the total TAG isolated comprises at least 50% TAGs comprising C4-C18 R-group fatty acids.

In some embodiments of any of the aspects, the total TAG isolated comprises at least 50% TAGs comprising C4-C8 R-group fatty acids.

In some embodiments of any of the aspects, the total TAG isolated comprises at least 50% TAGs comprising C16 R-group fatty acids.

In one aspect, described herein is a method of producing triacylglycerides (TAGs), comprising: (a) culturing an engineered bacterium as described herein in a culture medium comprising fructose and/or H₂; and (b) isolating, collecting, or concentrating TAGs from said engineered bacterium or from the culture medium of said engineered bacterium.

In some embodiments of any of the aspects, the culture medium comprises fructose as the sole carbon source, and/or the culture medium comprises H₂ as the sole energy source.

In some embodiments of any of the aspects, the total TAG isolated comprises at least 50% TAGs comprising C4-C18 R-group fatty acids.

In some embodiments of any of the aspects, the total TAG isolated comprises at least 50% TAGs comprising C4-C8 R-group fatty acids.

In some embodiments of any of the aspects, the total TAG isolated comprises at least 50% TAGs comprising C16 R-group fatty acids.

In one aspect, described herein is a method of producing triacylglycerides (TAGs), comprising: (a) culturing an engineered bacterium as described herein in a culture medium comprising glycerol and/or H₂; and (b) isolating, collecting, or concentrating TAGs from said engineered bacterium or from the culture medium of said engineered bacterium.

In some embodiments of any of the aspects, the culture medium comprises glycerol as the sole carbon source, and/or the culture medium comprises H₂ as the sole energy source.

In some embodiments of any of the aspects, the total TAG isolated comprises at least 50% TAGs comprising C4-C18 R-group fatty acids.

In some embodiments of any of the aspects, the total TAG isolated comprises at least 50% TAGs comprising C4-C8 R-group fatty acids.

In some embodiments of any of the aspects, the total TAG isolated comprises at least 50% TAGs comprising C16 R-group fatty acids.

In one aspect, described herein is a system comprising: (a) a reactor chamber with a solution contained therein, wherein the solution comprises hydrogen (H₂) and a carbon source; and (b) an engineered bacterium as described herein in the solution.

In some embodiments of any of the aspects, the system further comprises a pair of electrodes in contact with the solution that split water to form the hydrogen.

In some embodiments of any of the aspects, the carbon source is carbon dioxide (CO₂), fructose, and/or glycerol.

In some embodiments of any of the aspects, the system further comprises an isolated gas volume above a surface of the solution within a head space of a reactor chamber.

In some embodiments of any of the aspects, the isolated gas volume comprises primarily carbon dioxide.

In some embodiments of any of the aspects, the system further comprises a power source comprising a renewable source of energy.

In some embodiments of any of the aspects, the renewable source of energy comprises a solar cell, wind turbine, generator, battery, or grid power.

### BRIEF DESCRIPTION OF THE DRAWINGS

FIG. 1A-1B is a series of schematics showing the composition of milk and biosynthesis of triacylglycerides. FIG. 1A is a schematic showing an exemplary composition of milk. TAGs form the majority (e.g., ˜98%) of total fats. The insert shows a general reaction formula for the production of TAGs from glycerol and fatty acids. FIG. 1B is a schematic showing biosynthesis of TAGs. The metabolic pathways were tuned by engineering key biosynthesis enzymes: e.g., thioesterases (TE), diglyceride acyltransferases (DGAT), and phosphatidate phosphatase (PAP).

FIG. 2A-2B is a series of schematics showing C. necator strains expressing engineered TAG biosynthesis pathways. FIG. 2A is a bar graph showing a normalized Nile Red fluorescence assay. FIG. 2B is a bar graph showing raw fluorescence and optical density from the Nile Red assay in FIG. 2A. Abbreviations: R. opacus PAP and A. baylyi DGAT (RoAb; “Strain 1”); R. jostii PAP and A. baylyi DGAT (RjAb; “Strain 2”); R. opacus PAP and T. curvata DGAT (RoTc; “Strain 3”); R. jostii PAP and T. curvata DGAT (RjTc; “Strain 4”); R. opacus PAP, T. curvata DGAT and Chimera 4 TE (Ch4RoTc; “Strain 5”); and R. opacus PAP, T. curvata (DGAT), and M. formatexigens (TE) (MfRoTc; “Strain 6”); all strains are on the ΔphaC C. necator background.

FIG. 3 is a bar graph showing the fatty acid profile in lipids of ΔphaC C. necator or strain 1 (R. opacus PAP and A. baylyi DGAT (RoAb) in ΔphaC C. necator) in 4 L or 10 L conditions. “C14” indicates acids that are 14 carbons long (e.g., myristic acid). “C16” indicates fatty acids that are 16 carbons long (e.g., palmitic acid). “C16:1” indicates fatty acids that are 16 carbons long with 1 unsaturated double bond (e.g., palmitoleic acid). RoAb has a higher fatty acid content and an altered distribution compared to ΔphaC. An overall increase in fatty acids and a change in fatty acid composition indicates TAG production.

FIG. 4A is a schematic representation of a reactor. FIG. 4B is a schematic representation of the production of one or more products within the reactor of FIG. 4A (indicated by dashed circle in FIG. 4A). Adapted from US 2018/0265898 A1.

FIG. 5 is a schematic of a triglyceride molecule showing the Sn positions and the numerical and alphabetical nomenclatures of fatty acids.

FIG. 6 is a schematic showing an exemplary TAG engineering strategy.

FIG. 7A-7B is a series of images showing PCR verification of engineered bacteria. “phaC” denotes Cupriavidus necator H16 ΔphaC1. “H16” denotes wild-type Cupriavidus necator H16. The strain designations for 873, 875, 878, 881, 884, and 887 are shown in Table 6. All PCRs were done using cells. FIG. 7A used a primer set that amplifies constructs on the plasmid (e.g., pBadT), showing inclusion of the plasmid (and associated added enzymes) in the engineered strains. FIG. 7B used a primer set that amplifies the genomic phaC1 region, showing phaC1 knockout in the engineered strains (lower bands).

FIG. 8 is an image showing thin layer chromatography (TLC) for TAG visualization. Note that the engineered 873 strain with induction (e.g., arabinose) shows produced of TAG tri-14, while no detectable TAGs were produced in the engineered 873 strain without induction.

FIG. 9 is an image showing high performance liquid chromatography data (HPLC). See e.g., Table 6 for strain designations of 873 and 881.

FIG. 10 is an image showing high performance gas chromatography-mass spectroscopy data (GC-MS) from strain 873.

### DETAILED DESCRIPTION

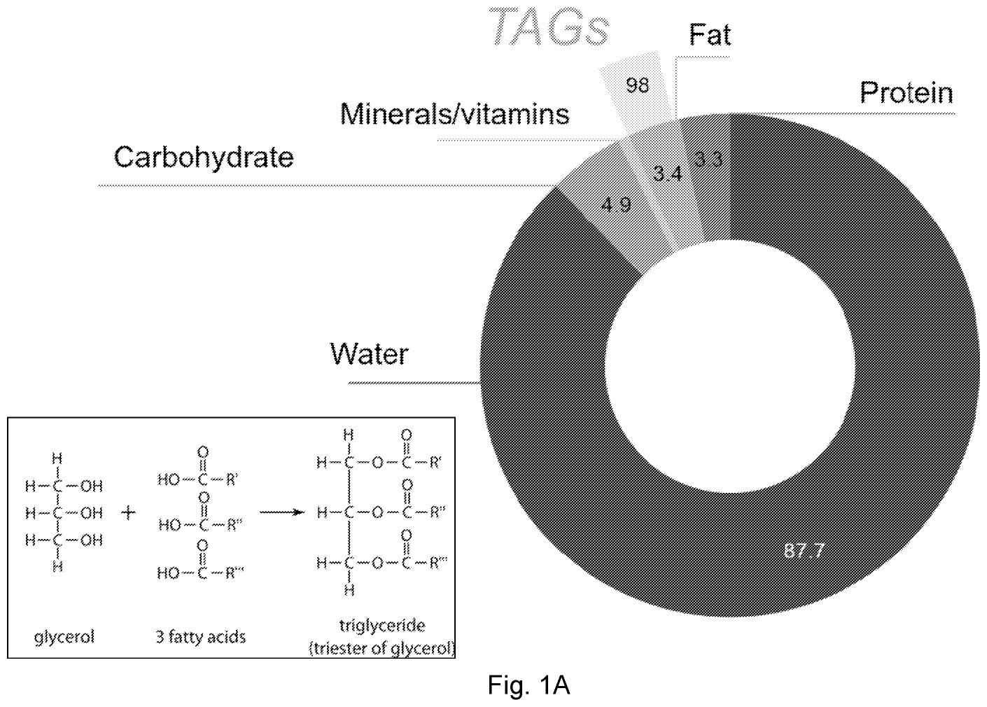

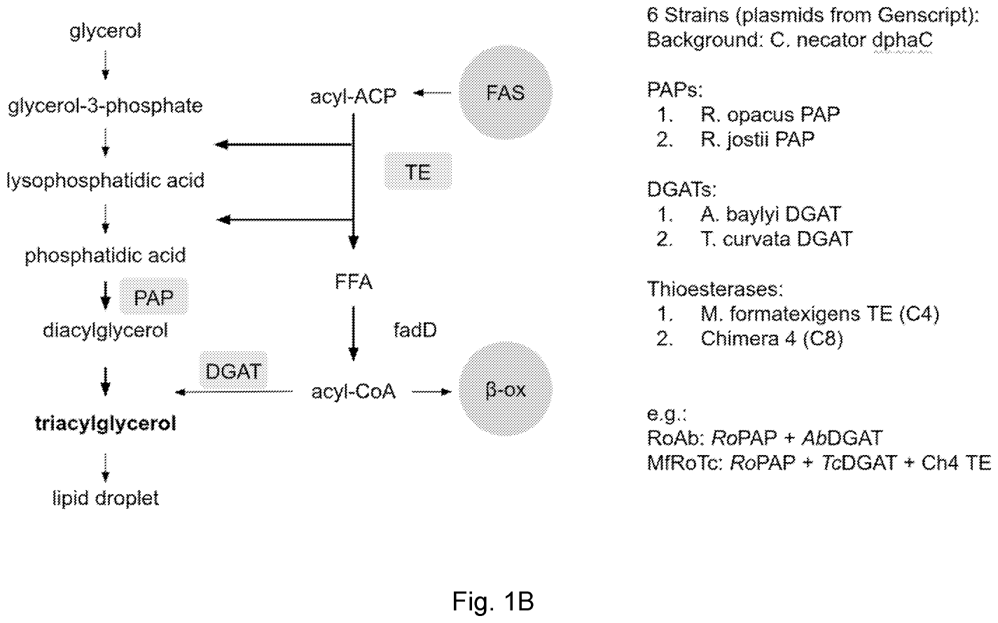

Embodiments of the technology described herein are directed to engineered bacteria and methods of producing triacylglycerides (TAG). The methods and compositions described herein permit the production of triacylglycerides using C. necator. In one aspect, described herein are engineered bacteria and corresponding methods, compositions, and systems for the production of tailored animal triacylglycerides. Formula I below shows the general formula for a triacylglyceride (see e.g., FIG. 1A). TAGs can also be referred to interchangeably as triglyceride (TG) or triacylglycerol (TAG).

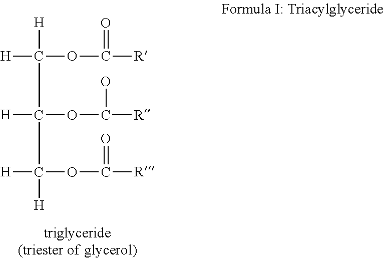

As shown herein, coupling recent advancements in genetic engineering of microbes and gas-driven fermentation provides a path towards sustainable commodity chemical production. C. necator H16 is a suitable species primarily because it effectively utilizes H₂ and CO₂ and is genetically tractable. Demonstrated herein is the versatility of this organism in lithotrophic (e.g., using C02 as a carbon source) or heterotrophic conditions (e.g., using glycerol as a carbon source), for example the production of triacylglycerides.

Described herein are engineered bacteria that can be used to sustainably produce triacylglycerides. In some embodiments of any of the aspects, the engineered bacterium is a chemoautotroph. In some embodiments of any of the aspects, the engineered bacterium can grow under chemoautotrophic (i.e., lithotrophic) conditions. As used herein, the term “chemoautotroph” refers to an organism that uses inorganic energy sources to synthesize organic compounds from carbon dioxide. The term “chemolithotroph” can be used interchangeably with chemoautotroph. Chemoautotrophs stand in contrast to heterotrophs. As used herein, the term “heterotroph” refers to an organism that derives its nutritional requirements from complex organic substances (e.g., sugars).

In some embodiments of any of the aspects, the engineered bacterium is a chemolithotroph. As used herein, the term “chemolithotroph” refers to an organism that is able to use inorganic reduced compounds (e.g., hydrogen, nitrite, iron, sulfur) as a source of energy (e.g., as electron donors). The chemolithotrophy process is accomplished through oxidation of inorganic compounds and ATP synthesis. The majority of chemolithotrophs are able to fix carbon dioxide (CO₂) through the Calvin cycle, a metabolic pathway in which carbon enters as CO₂ and leaves as glucose (see e.g., Kuenen, G. (2009). “Oxidation of Inorganic Compounds by Chemolithotrophs”. In Lengeler, J.; Drews, G.; Schlegel, H. (eds.). Biology of the Prokaryotes. John Wiley & Sons. p. 242. ISBN 9781444313307). The chemolithotroph group of organisms includes sulfur oxidizers, nitrifying bacteria, iron oxidizers, and hydrogen oxidizers. The term “chemolithotrophy” refers to a cell's acquisition of energy from the oxidation of inorganic compounds, also known as electron donors. This form of metabolism is known to occur only in prokaryotes. See e.g., Table 1 for non-limiting examples of chemolithotrophic bacteria and archaea.

TABLE 1
Chemolithotrophic bacteria and archaea

| | Non-Limiting | Source of | Respiration |
|---|---|---|---|
| | Examples of | energy and | electron |
| Bacteria | Chemolithotrophs | electrons | acceptor |
| Iron | Acidithiobacillus | Fe²⁺ (ferrous iron) → | O₂ (oxygen) → |
| bacteria | ferrooxidans | Fe³⁺ (ferric iron) + | H₂O (water) |
| | | e⁻ | |
| Nitrosifying | Nitrosomonas | NH₃ (ammonia) → | O₂ (oxygen) → |
| bacteria | | NO₂⁻ (nitrite) + | H₂O (water) |
| | | e⁻ | |
| Nitrifying | Nitrobacter | NO₂⁻ (nitrite) → | O₂ (oxygen) → |
| bacteria | | NO₃⁻ (nitrate) + | H₂O (water) |
| | | e⁻ | |
| Chemotrophic | Halothiobacillaceae | S²⁻ (sulfide) → | O₂ (oxygen) → |
| purple sulfur | | S⁰ (sulfur) + e⁻ | H₂O (water) |
| bacteria | | | |
| Sulfur-oxidizing | Chemotrophic Rhodobacteraceae | S⁰ (sulfur) → | O₂ (oxygen) → |
| bacteria | and Thiotrichaceae | SO₄²⁻ (sulfate) + | H₂O (water) |
| | | e⁻ | |
| Aerobic hydrogen | Cupriavidus necator; | H₂ (hydrogen) → H₂O | O₂ (oxygen) → |
| bacteria | Cupriavidus metallidurans | (water) + e⁻ | H₂O (water |
| Anammox | Planctomycetes | NH₄⁺ (ammonium) → | N₂ (nitrogen) + |
| bacteria | | NO₂⁻ (nitrite) | H₂O (water) |
| Thiobacillus | Thiobacillus | S⁰ (sulfur) → | NO₃⁻ (nitrate) |
| denitrificans | denitrificans | SO₄²⁻ (sulfate) + | |
| | | e⁻ | |
| Sulfate-reducing | Desulfovibrio | H₂ (hydrogen) → H₂O | Sulfate |
| bacteria: Hydrogen | paquesii | (water) + e⁻ | (SO₄²⁻) |
| bacteria | | | |
| Sulfate-reducing | Desulfotignum | PO₃³⁻ (phosphite) → | Sulfate |
| bacteria: Phosphite | phosphitoxidans | PO₄³⁻ (phosphate) + | (SO₄²⁻) |
| bacteria | | e⁻ | |
| Methanogens | Archaea | H₂ (hydrogen) → H₂O | CO₂ (carbon |
| | | (water) + e⁻ | dioxide) |
| Carboxydotrophic | Carboxydothermus | carbon monoxide | H₂O (water) → |
| bacteria | hydrogenoformans | (CO) → carbon dioxide | H₂ (hydrogen) |
| | | (CO₂) + e⁻ | |

In some embodiments of any of the aspects, the engineered bacteria is a chemolithotroph belonging to a classification selected from the group consisting of Acidithiobacillus, Alcaligenes, Carboxydothermus, Cupriavidus, Desulfotignum, Desulfovibrio, Halothiobacillaceae, Hydrogenomonas, Nitrobacter, Nitrosomonas, Planctomycetes, Ralstonia, Rhodobacteraceae, Thiobacillus, Thiotrichaceae, and Wautersia. In some embodiments of any of the aspects, the engineered organism is a methanogenic archaea (e.g., belonging to the genera Methanosarcina or Methanothrix). In some embodiments of any of the aspects, the engineered bacteria is selected from the group consisting of Acidithiobacillus ferrooxidans, Carboxydothermus hydrogenoformans, Cupriavidus metallidurans, Cupriavidus necator, Desulfotignum phosphitoxidans, Desulfovibrio paquesii, and Thiobacillus denitrificans. In some embodiments of any of the aspects, the engineered bacteria is further engineered to be chemolithotrophic. In some embodiments of any of the aspects, the engineered bacterium is aerobic and uses O₂ as its respiration electron acceptor. In some embodiments of any of the aspects, the engineered bacteria can be a heterotroph or a chemolithotroph, e.g., depending on environmental conditions.

In some embodiments of any of the aspects, the engineered bacteria uses CO₂ as its sole carbon source or H₂ as its sole energy source. In some embodiments of any of the aspects, the engineered bacteria uses CO₂ as its sole carbon source and H₂ as its sole energy source. In some embodiments of any of the aspects, the engineered bacteria uses H₂ as its sole energy source. In some embodiments of any of the aspects, the engineered bacteria uses CO₂ as its sole carbon source.

In some embodiments of any of the aspects, the engineered bacteria is engineered from a bacteria that uses CO₂ as its sole carbon source or H₂ as its sole energy source. In some embodiments of any of the aspects, the engineered bacteria is engineered from a bacteria that uses CO₂ as its sole carbon source and H₂ as its sole energy source. In some embodiments of any of the aspects, the engineered bacteria is engineered from a bacteria that uses H₂ as its sole energy source. In some embodiments of any of the aspects, the engineered bacteria is engineered from a bacteria that uses CO₂ as its sole carbon source.

In some embodiments of any of the aspects, the engineered bacteria obtains at least 90%, at least 95%, at least 98%, at least 99% or more of its carbon from CO₂. In some embodiments of any of the aspects, the engineered bacteria obtains at least 90%, at least 95%, at least 98%, at least 99% or more of its energy from H₂. In some embodiments of any of the aspects, the engineered bacteria obtains at least 90%, at least 95%, at least 98%, at least 99% or more of its carbon from CO₂ and at least 90%, at least 95%, at least 98%, at least 99% or more of its energy from H₂.

As used herein, the term “carbon source” refers to the molecules used by an organism as the source of carbon for building its biomass; a carbon source can be an organic compound or an inorganic compound. “Source” denotes an environmental source. In some embodiments of any of the aspects, the engineered bacteria fixes carbon dioxide (CO₂) through the Calvin cycle, a metabolic pathway in which carbon enters as CO₂ and leaves as glucose. As used herein, the term “sole carbon source” denotes that the engineered bacteria uses only the indicated carbon source (e.g., CO₂) and no other carbon sources. For example, “sole carbon source” is intended to mean where the suitable conditions comprise a culture media containing a carbon source such that, as a fraction of the total carbon atoms in the media, the specific carbon source (e.g., CO₂), respectively, represent about 100% of the total carbon atoms in the media. In some embodiments, the sole carbon source of the engineered bacteria is inorganic carbon, including but not limited to carbon dioxide (CO₂) and bicarbonate (HCO₃⁻). In some embodiments of any of the aspects, the sole carbon source is atmospheric CO₂.

In some embodiments of any of the aspects, the engineered bacteria uses CO₂ as its major carbon source, meaning at least 50% of its carbon atoms are obtained from CO₂. As a non-limiting example, the engineered bacteria obtains at least 50%, at least 60%, at least 70%, at least 80%, at least 90%, at least 95%, or at least 99% of its carbon atoms from CO₂.

In some embodiments of any of the aspects, the engineered bacteria does not use organic carbon as a carbon source. Non-limiting example of organic carbon sources include fatty acids, gluconate, acetate, fructose, decanoate; see e.g., Jiang et al. Int J Mol Sci. 2016 July; 17(7): 1157).

In some embodiments of any of the aspects, the engineered bacteria uses a simple organic carbon source as its sole carbon source. Non-limiting examples of simple organic carbon sources include: glucose, glycerol, gluconate, acetate, fructose, or decanoate. In some embodiments of any of the aspects, the engineered bacteria uses fructose as its sole carbon source. In some embodiments of any of the aspects, the engineered bacteria uses fructose and CO₂ as its carbon sources. In some embodiments of any of the aspects, the engineered bacteria is engineered from a bacteria that uses fructose as its sole carbon source. In some embodiments of any of the aspects, the engineered bacteria obtains at least 90%, at least 95%, at least 98%, at least 99% or more of its carbon from fructose. In some embodiments of any of the aspects, the engineered bacteria uses fructose as its major carbon source, meaning at least 50% of its carbon atoms are obtained from fructose. As a non-limiting example, the engineered bacteria obtains at least 50%, at least 60%, at least 70%, at least 80%, at least 90%, at least 95%, or at least 99% of its carbon atoms from fructose.

In some embodiments of any of the aspects, the engineered bacteria uses glycerol as its sole carbon source. In some embodiments of any of the aspects, the engineered bacteria uses glycerol and CO₂ as its carbon sources. In some embodiments of any of the aspects, the engineered bacteria is engineered from a bacteria that uses glycerol as its sole carbon source. In some embodiments of any of the aspects, the engineered bacteria obtains at least 90%, at least 95%, at least 98%, at least 99% or more of its carbon from glycerol. In some embodiments of any of the aspects, the engineered bacteria uses glycerol as its major carbon source, meaning at least 50% of its carbon atoms are obtained from glycerol. As a non-limiting example, the engineered bacteria obtains at least 50%, at least 60%, at least 70%, at least 80%, at least 90%, at least 95%, or at least 99% of its carbon atoms from glycerol.

In some embodiments of any of the aspects, the engineered bacteria uses H₂ as its sole energy source. As used herein, the term “energy source” refers to molecules that contribute electrons and contribute to the process of ATP synthesis. As described here, the engineered bacterium can be a chemolithotroph, i.e., an organism that is able to use inorganic reduced compounds (e.g., hydrogen, nitrite, iron, sulfur) as a source of energy (e.g., as electron donors). As used herein, the term “sole energy source” denotes that the engineered bacteria uses only the indicated energy source (e.g., H₂) and no other energy sources. In some embodiments of any of the aspects, the sole energy source is atmospheric H₂.

In some embodiments of any of the aspects, the engineered bacteria uses H₂ as its major energy source, meaning at least 50% of its donated electrons (e.g., used for ATP synthesis) are obtained from H₂. As a non-limiting example, the engineered bacteria obtains at least 50%, at least 60%, at least 70%, at least 80%, at least 90%, at least 95%, or at least 99% of its donated electrons from H₂.

Bacteria used in the systems and methods disclosed herein may be selected so that the bacteria both oxidize hydrogen as well as consume carbon dioxide. Accordingly, in some embodiments, the bacteria may include an enzyme capable of metabolizing hydrogen as an energy source such as with hydrogenase enzymes. Additionally, the bacteria may include one or more enzymes capable of performing carbon fixation such as Ribulose-1,5-bisphosphate carboxylase/oxygenase (RuBisCO). One possible class of bacteria that may be used in the systems and methods described herein to produce a product include, but are not limited to, chemolithoautotrophs. Additionally, appropriate chemolithoautotrophs may include any one or more of Ralstonia eutropha (R. eutropha) as well as Alcaligenes paradoxs I 360 bacteria, Alcaligenes paradoxs 12/X bacteria, Nocardia opaca bacteria, Nocardia autotrophica bacteria, Paracoccus denitrificans bacteria, Pseudomonas facilis bacteria, Arthrobacter species 1IX bacteria, Xanthobacter autotrophicus bacteria, Azospirillum lipferum bacteria, Derxia gummosa bacteria, Rhizobium japonicum bacteria, Microcyclus aquaticus bacteria, Microcyclus ebruneus bacteria, Renobacter vacuolatum bacteria, and any other appropriate bacteria.

In some embodiments of any of the aspects, the engineered bacteria belongs to the Cupriavidus genus. The Cupriavidus genus of bacteria includes the former genus Wautersia. Cupriavidus bacteria are characterized as Gram-negative, motile, rod-shaped organisms with oxidative metabolism. Cupriavidus bacteria possess peritrichous flagella, are obligate aerobic organisms, and are chemoorganotrophic or chemolithotrophic. In some embodiments of any of the aspects, the engineered bacteria is selected from the group consisting of Cupriavidus alkaliphilus, Cupriavidus basilensis, Cupriavidus campinensis, Cupriavidus gilardii, Cupriavidus laharis, Cupriavidus metallidurans, Cupriavidus necator, Cupriavidus nantongensis, Cupriavidus numazuensis, Cupriavidus oxalaticus, Cupriavidus pampae, Cupriavidus pauculus, Cupriavidus pinatubonensis, Cupriavidus plantarum, Cupriavidus respiraculi, Cupriavidus taiwanensis, and Cupriavidus yeoncheonensis.

In some embodiments of any of the aspects, the engineered bacterium is Cupriavidus necator. Cupriavidus necator can also be referred to as Ralstonia eutropha, Hydrogenomonas eutrophus, Alcaligenes eutropha, or Wautersia eutropha. In some embodiments of any of the aspects, the engineered bacterium is Cupriavidus necator strain H16. In some embodiments of any of the aspects, the engineered bacterium is Cupriavidus necator strain N-1.

Members of the species and genera described herein can be identified genetically and/or phenotypically. By way of non-limiting example, the engineered bacterium as described herein comprises a 16S rDNA sequence at least 97% identical to a 16S rDNA sequence present in a reference strain operational taxonomic unit for Cupriavidus necator. In some embodiments of any of the aspects, the engineered bacterium as described herein comprises a 16S rDNA that comprises SEQ ID NO: 1 or SEQ ID NO: 2 or a sequences that is at least 95%, at least 96%, at least 97%, at least 98%, or at least 99% identical to the sequence of SEQ ID NO: 1 or SEQ ID NO: 2. In some embodiments of any of the aspects, the bacterium as described herein is engineered from Cupriavidus necator (e.g., strain H16 or strain N-1).

Cupriavidus necator strain N-1 16S ribosomal RNA,
partial sequence, NCBI Reference Sequence:
NR_028766.1 1356 nucleotides (nt)
SEQ ID NO: 1
TTAGATTGAACGCTGGCGGCATGCCTTACACATGCAAGTCGAACGGCAGC
ACGGGCTTCGGCCTGGTGGCGAGTGGCGAACGGGTGAGTAATACATCGGA
ACGTGCCCTGTAGTGGGGGATAACTAGTCGAAAGATTAGCTAATACCGCA
TACGACCTGAGGGTGAAAGCGGGGGACCGCAAGGCCTCGCGCTACAGGAG
CGGCCGATGTCTGATTAGCTAGTTGGTGGGGTAAAAGCCTACCAAGGCGA
CGATCAGTAGCTGGTCTGAGAGGACGATCAGCCACACTGGGACTGAGACA
CGGCCCAGACTCCTACGGGAGGCAGCAGTGGGGAATTTTGGACAATGGGG
GCAACCCTGATCCAGCAATGCCGCGTGTGTGAAGAAGGCCTTCGGGTTGT
AAAGCACTTTTGTCCGGAAAGAAATGGCTCTGGTTAATACCCGGGGTCGA
TGACGGTACCGGAAGAATAAGCACCGGCTAACTACGTGCCAGCAGCCGCG
GTAATACGTAGGGTGCGAGCGTTAATCGGAATTACTGGGCGTAAAGCGTG
CGCAGGCGGTTTTGTAAGACAGGCGTGAAATCCCCGAGCTCAACTTGGGA
ATGGCGCTTGTGACTGCAAGGCTAGAGTATGTCAGAGGGGGGTAGAATTC
CACGTGTAGCAGTGAAATGCGTAGAGATGTGGAGGAATACCGATGGCGAA
GGCAGCCCCCTGGGACGTCACTGACGCTCATGCACGAAAGCGTGGGGAGC
AAACAGGATTAGATACCCTGGTAGTCCACGCCCTAAACGATGTCAACTAG
TTGTTGGGGATTCATTTCTTCAGTAACGTAGCTAACGCGTGAAGTTGACC
GCCTGGGGAGTACGGTCGCAAGATTAAAACTCAAAGGAATTGACGGGGAC
CCGCACAAGCGGTGGATGATGTGGATTAATTCGATGCAACGCGAAAAACC
TTACCTACCCTTGACATGCCACTAACGAAGCAGAGATGCATTAGGTGCCC
GAAAGGGAAAGTGGACACAGGTGCTGCATGGCTGTCGTCAGCTCGTGTCG
TGAGATGTTGGGTTAAGTCCCGCAACGAGCGCAACCCTTGTCTCTAGTTG
CTACGAAAGGGCACTCTAGAGAGACTGCCGGTGACAAACCGGAGGAAGGT
GGGGATGACGTCAAGTCCTCATGGCCCTTATGGGTAGGGCTTCACACGTC
ATACAATGGTGCGTACAGAGGGTTGCCAACCCGCGAGGGGGAGCTAATCC
CAGAAAACGCATCGTAGTCCGGATCGTAGTCTGCAACTCGACTACGTGAA
GCTGGAATCGCTAGTAATCGCGGATCAGCATGCCGCGGTGAATACGTTCC
CGGTCT,
Cupriavidus necator strain H16 16S ribosomal RNA,
1537 nt
SEQ ID NO: 2
AGATTGAACTGAAGAGTTTGATCCTGGCTCAGATTGAACGCTGGCGGCAT
GCCTTACACATGCAAGTCGAACGGCAGCACGGGCTTCGGCCTGGTGGCGA
GTGGCGAACGGGTGAGTAATACATCGGAACGTGCCCTGTAGTGGGGGATA
ACTAGTCGAAAGATTAGCTAATACCGCATACGACCTGAGGGTGAAAGCGG
GGGACCGCAAGGCCTCGCGCTACAGGAGCGGCCGATGTCTGATTAGCTAG
TTGGTGGGGTAAAAGCCTACCAAGGCGACGATCAGTAGCTGGTCTGAGAG
GACGATCAGCCACACTGGGACTGAGACACGGCCCAGACTCCTACGGGAGG
CAGCAGTGGGGAATTTTGGACAATGGGGGCAACCCTGATCCAGCAATGCC
GCGTGTGTGAAGAAGGCCTTCGGGTTGTAAAGCACTTTTGTCCGGAAAGA
AATGGCTCTGGTTAATACCCGGGGTCGATGACGGTACCGGAAGAATAAGC
ACCGGCTAACTACGTGCCAGCAGCCGCGGTAATACGTAGGGTGCGAGCGT
TAATCGGAATTACTGGGCGTAAAGCGTGCGCAGGCGGTTTTGTAAGACAG
GCGTGAAATCCCCGAGCTCAACTTGGGAATGGCGCTTGTGACTGCAAGGC
TAGAGTATGTCAGAGGGGGAAGAATTCCACGTGTAGCAGTGAAATGCGTA
GAGATGTGGAGGAATACCGATGGCGAAGGCAGCCCCCTGGGACGTCACTG
ACGCTCATGCACGAAAGCGTGGGGAGCAAACAGGATTAGATACCCTGGTA
GTCCACGCCCTAAACGATGTCAACTAGTTGTTGGGGATTCATTTCTTCAG
TAACGTAGCTAACGCGTGAAGTTGACCGCCTGGGGAGTACGGTCGCAAGA
TTAAAACTCAAAGGAATTGACGGGGACCCGCACAAGCGGTGGATGATGTG
GATTAATTCGATGCAACGCGAAAAACCTTACCTACCCTTGACATGCCACT
AACGAAGCAGAGATGCATTAGGTGCCCGAAAGGGAAAGTGGACACAGGTG
CTGCATGGCTGTCGTCAGCTCGTGTCGTGAGATGTTGGGTTAAGTCCCGC
AACGAGCGCAACCCTTGTCTCTAGTTGCTACGAAAGGGCACTCTAGAGAG
ACTGCCGGTGACAAACCGGAGGAAGGTGGGGATGACGTCAAGTCCTCATG
GCCCTTATGGGTAGGGCTTCACACGTCATACAATGGTGCGTACAGAGGGT
TGCCAACCCGCGAGGGGGAGCTAATCCCAGAAAACGCATCGTAGTCCGGA
TCGTAGTCTGCAACTCGACTACGTGAAGCTGGAATCGCTAGTAATCGCGG
ATCAGCATGCCGCGGTGAATACGTTCCCGGGTCTTGTACACACCGCCCGT
CACACCATGGGAGTGGGTTTTGCCAGAAGTAGTTAGCCTAACCGCAAGGA
GGGCGATTACCACGGCAGGGTTCATGACTGGGGTGAAGTCGTAACAAGGT
AGCCGTATCGGAAGGTGCGGCTGGATCACCTCCTTTC.

In some embodiments of any of the aspects, the engineered bacterium comprises at least one engineered inactivating modification of at least one endogenous gene. In some embodiments of any of the aspects, an engineered inactivating modification of an endogenous gene comprises one or more of: i) deletion of the entire coding sequence, ii) deletion of the promoter of the gene, iii) a frameshift mutation, iv) a nonsense mutation (i.e., a premature termination codon), v) a point mutation, vi) a deletion, vii) or an insertion. Non-limiting examples of inactivating modifications include a mutation that decreases gene or polypeptide expression, a mutation that decreases gene or polypeptide transport, a mutation that decreases gene or polypeptide activity, a mutation in the active site of an enzyme that decreases enzymatic activity, or a mutation that decreases the stability of a nucleic acid or polypeptide. Examples of loss-of-function mutations for each gene can be clear to a person of ordinary skill (e.g., a premature stop codon, a frameshift mutation); they can be measurable by an assay of nucleic acid or protein function, activity, expression, transport, and/or stability; or they can be known in the art.

In some embodiments of any of the aspects, an inactivating modification of an endogenous gene can be engineered in a bacterium using an integration vector (e.g., pT18mobsacB). In some embodiments of any of the aspects, the engineering of an inactivating modification of an endogenous gene in a bacterium further comprises conjugation methods and/or counterselection methods. In some embodiments of any of the aspects, the introduction of an integration vector comprising an endogenous gene comprising an inactivating modification causes the endogenous gene to be replaced with the endogenous gene comprising an inactivating modification.

In some embodiments of any of the aspects, the engineered bacterium comprises at least one overexpressed gene. In some embodiments of any of the aspects, the overexpressed gene is endogenous. In some embodiments of any of the aspects, the overexpressed gene is exogenous. In some embodiments of any of the aspects, the overexpressed gene is heterologous. In some embodiments of any of the aspects, a gene can be overexpressed using an expression vector (e.g., pBAD, pCR2.1).

In some embodiments of any of the aspects, the engineered bacterium comprises at least one exogenous copy of a functional gene. As a non-limiting example, the engineered bacterium can comprise 1, 2, 3, 4, or at least 5 exogenous copies of a functional gene. As used herein, the term “functional” refers to a form of a molecule which possesses either the native biological activity of the naturally existing molecule of its type, or any specific desired activity, for example as judged by its ability to bind to ligand molecules. In some embodiments of any of the aspects, a functional molecule can comprise at least 50%, at least 60%, at least 70%, at least 75%, at least 80%, at least 85%, at least 90%, at least 95%, at least 96%, at least 97%, at least 98%, at least 99%, or at least 99% of the activity of the wild-type molecule, e.g., in its native organism.

In some embodiments of any of the aspects, a functional gene as described herein is exogenous. In some embodiments of any of the aspects, a functional gene as described herein is ectopic. In some embodiments of any of the aspects, a functional gene as described herein is not endogenous.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism, in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

In some embodiments of any of the aspects, the engineered bacterium comprises at least one functional heterologous gene. As used herein, the term “heterologous” refers to that which is not endogenous to, or naturally occurring in, a referenced sequence, molecule (including e.g., a protein), virus, cell, tissue, or organism. For example, a heterologous sequence of the present disclosure can be derived from a different species, or from the same species but substantially modified from an original form. Also for example, a nucleic acid sequence that is not normally expressed in a virus or a cell is a heterologous nucleic acid sequence. The term “heterologous” can refer to DNA, RNA, or protein that does not occur naturally as part of the organism in which it is present or which is found in a location or locations in the genome that differ from that in which it occurs in nature. It is DNA, RNA, or protein that is not endogenous to the virus or cell and has been artificially introduced into the virus or cell.

In some embodiments of any of the aspects, at least one exogenous copy of a functional gene can be engineered into a bacterium using an expression vector (e.g., pBadT). In some embodiments of any of the aspects, the expression vector (e.g., pBadT) is translocated from a donor bacterium (e.g., MFDpir) into the engineered bacterium under conditions that promote conjugation.

In some embodiments of any of the aspects, at least one exogenous or heterologous gene as described herein can comprise a detectable label, including but not limited to c-Myc, HA, VSV-G, HSV, FLAG, V5, HIS, or biotin. Detectable labels can also include, but are not limited to, radioisotopes, bioluminescent compounds, chromophores, antibodies, chemiluminescent compounds, fluorescent compounds, metal chelates, and enzymes.

In some embodiments of any of the aspects, the engineered bacterium further comprises a selectable marker. Non-limiting examples of selectable markers include a positive selection marker; a negative selection marker; a positive and negative selection marker; resistance to at least one of ampicillin, kanamycin, triclosan, and/or chloramphenicol; or an auxotrophy marker. In some embodiments of any of the aspects, the selectable marker is selected from the group consisting of beta-lactamase, Neo gene (e.g., Kanamycin resistance cassette) from Tn5, mutant FabI gene, and an auxotrophic mutation.

Described herein are bacteria engineered for the production of TAGs (e.g., animal TAGs or milk fats). In one aspect, described herein is an engineered (e.g., Cupriavidus necator) bacterium, comprising at least one of the following: (a) at least one exogenous copy of at least one functional acyltransferase gene; and/or (b) at least one exogenous copy of at least one functional phosphatidic acid (PA) phosphatase gene. In some embodiments of any of the aspects, the acyltransferase gene encodes for an acyltransferase enzyme that catalyzes transesterification of the sn3 OH group, the sn2 OH group, or the sn1 OH group of a TAG precursor (e.g., diacylglycerol, lysophosphatidic acid, or glyceraldehyde-3-phosphate) with a fatty acid. In some embodiments of any of the aspects, the acyltransferase gene encodes an acyltransferase enzyme that catalyzes transesterification of the sn3 OH group of a diacylglycerol with a fatty acid. In some embodiments of any of the aspects, the acyltransferase gene is a functional diglyceride acyltransferase (DGAT) gene, a functional wax synthase (WS) gene, a hybrid of a DGAT and a WS, a functional lysophosphatidic acid acyltransferase (LPAT) gene, or a functional glycerol-3-phosphate acyltransferase (GPAT) gene. In one aspect, described herein is an engineered (e.g., Cupriavidus necator) bacterium, comprising at least one of the following: (a) at least one exogenous copy of at least one functional thioesterase (TE) gene; (b) at least one exogenous copy of at least one functional diglyceride acyltransferase (DGAT) gene; and/or (c) at least one exogenous copy of at least one phosphatidate phosphatases (PAP) gene. In some embodiments of any of the aspects, the engineered bacterium further comprises: (i) at least one endogenous polyhydroxyalkanoate (PHA) synthase gene comprising at least one engineered inactivating modification; or (ii) at least one exogenous inhibitor of an endogenous polyhydroxyalkanoate (PHA) synthase gene or gene product. In some embodiments of any of the aspects, the engineered bacterium is selected from Table 3.

TABLE 3
Exemplary engineered TAG bacteria (“X” indicates
inclusion in the engineered TAG bacteria)

| Exogenous functionsl gene(s) | | | Inactivated or inhibited endogenous gene(s) | | |
|---|---|---|---|---|---|
| acyltransferase (e.g., | | | | beta- | diacylglycerol |
| DGAT, LPAT, and/or | | | | oxidation | kinase (e.g., |
| GPAT, see e.g., Table 7) | PAP | TE | PHA | (e.g., fadE) | dgkA) |
| X | | | | | |
| | X | | | | |
| X | X | | | | |
| | | X | | | |
| X | | X | | | |
| | X | X | | | |
| X | X | X | | | |
| | | | X | | |
| X | | | X | | |
| | X | | X | | |
| X | X | | X | | |
| | | X | X | | |
| X | | X | X | | |
| | X | X | X | | |
| X | X | X | X | | |
| | | | | X | |
| X | | | | X | |
| | X | | | X | |
| X | X | | | X | |
| | | X | | X | |
| X | | X | | X | |
| | X | X | | X | |
| X | X | X | | X | |
| | | | X | X | |
| X | | | X | X | |
| | X | | X | X | |
| X | X | | X | X | |
| | | X | X | X | |
| X | | X | X | X | |
| | X | X | X | X | |
| X | X | X | X | X | |
| | | | | | X |
| X | | | | | X |
| | X | | | | X |
| X | X | | | | X |
| | | X | | | X |
| X | | X | | | X |
| | X | X | | | X |
| X | X | X | | | X |
| | | | X | | X |
| X | | | X | | X |
| | X | | X | | X |
| X | X | | X | | X |
| | | X | X | | X |
| X | | X | X | | X |
| | X | X | X | | X |
| X | X | X | X | | X |
| | | | | X | X |
| X | | | | X | X |
| | X | | | X | X |
| X | X | | | X | X |
| | | X | | X | X |
| X | | X | | X | X |
| | X | X | | X | X |
| X | X | X | | X | X |
| | | | X | X | X |
| X | | | X | X | X |
| | X | | X | X | X |
| X | X | | X | X | X |
| | | X | X | X | X |
| X | | X | X | X | X |
| | X | X | X | X | X |
| X | X | X | X | X | X |

In some embodiments of any of the aspects, the engineered bacterium is a chemoautotroph. In some embodiments of any of the aspects, the engineered bacterium uses CO₂ as its sole carbon source, and/or said engineered bacteria uses H₂ as its sole energy source. In some embodiments of any of the aspects, the engineered bacterium is Cupriavidus necator.

In some embodiments of any of the aspects, the engineered bacterium produces TAGs (e.g., C16 TAGs). In some embodiments of any of the aspects, the TAGs are produced and/or isolated using methods as described further herein.

In some embodiments of any of the aspects, the engineered bacterium comprises one or more of the following: (a)(i) at least one endogenous polyhydroxyalkanoate (PHA) synthase gene comprising at least one engineered inactivating modification or (a)(ii) at least one exogenous inhibitor of an endogenous polyhydroxyalkanoate (PHA) synthase gene or gene product (e.g., mRNA, protein). In some embodiments of any of the aspects, the engineered bacterium comprises (a)(i) at least one endogenous polyhydroxyalkanoate (PHA) synthase gene comprising at least one engineered inactivating modification. In some embodiments of any of the aspects, the engineered bacterium comprises (a)(ii) at least one exogenous inhibitor of an endogenous polyhydroxyalkanoate (PHA) synthase gene or gene product (e.g., mRNA, protein)

In some embodiments of any of the aspects, the engineered bacterium comprises an engineered inactivating modification of an endogenous polyhydroxyalkanoate (PHA) synthase gene. In some embodiments of any of the aspects, the endogenous PHA synthase comprises phaC. PhaC is a class I poly(R)-hydroxyalkanoic acid synthase, and is the key enzyme in the polymerization of polyhydroxyalkanoates (PHAs). PhaC catalyzes the polymerization of 3-R-hydroxyalkyl CoA thioester to form PHAs with concomitant release of CoA. In some embodiments of any of the aspects, the endogenous PHA synthase comprises Cupriavidus necator phaC.

In some embodiments of any of the aspects, the engineered bacterium comprises an engineered inactivating modification of the endogenous Cupriavidus necator phaC gene. In some embodiments of any of the aspects, the nucleic acid sequence of the endogenous Cupriavidus necator phaC gene comprises SEQ ID NO: 3 or a sequence that is at least 80%, at least 85%, at least 90%, at least 91%, at least 92%, at least 93%, at least 94%, at least 95%, at least 96%, at least 97%, at least 98%, or at least 99% identical to the sequence of SEQ ID NO: 3 that maintains the same functions as SEQ ID NO: 3 (e.g., PHA synthase).

Cupriavidus necator N-1 chromosome 1, REGION:
1478083-1479852 GenBank: CP002877.1, 1770 bp DNA
SEQ ID NO: 3
ATGGCGACCGGCAAAGGCGCGGCAGCTTCCACTCAGGAAGGCAAGTCCCA
ACCATTCAAGTTCACGCCGGGGCCATTCGATCCAGCCACATGGCTGGAAT
GGTCCCGCCAGTGGCAGGGCACTGAAGGCAACGGCCACGCGGCCGCGTCC
GGCATTCCGGGCCTGGATGCGCTGGCAGGCGTCAAGATCGAGCCGGCGCA
GCTGGGTGATATCCAGCAGCGTTACATGAAGGACTTCTCAGCCCTGTGGC
AGGCCATGGCCGAGGGCAAGGCCGAGGCCACCGGGCCGCTGCACGACCGG
CGCTTCGCCGGCGACGCGTGGCGCACCAACCTGCCATACCGCTTCGCTGC
CGCGTTCTACCTGCTCAATGCGCGCGCCTTGACCGAGCTGGCCGATGCTG
TTGAGGCCGATGCCAAGACGCGCCAGCGCATCCGCTTTGCGATCTCGCAA
TGGGTCGATGCGATGTCGCCCGCCAACTTCCTCGCCACGAATCCCGAGGC
GCAGCGCCTGCTGATCGAGTCGGGCGGCGAATCGCTGCGTGCCGGCGTGC
GCAACATGATGGAAGACCTGACGCGCGGCAAGATCTCGCAGACCGACGAG
AGCGCGTTTGAGGTCGGCCGCAATGTCGCGGTGAGCGAAGGCGCCGTAGT
CTTCGAGAACGAATACTTCCAGCTGTTGCAGTACAAGCCGCTGACCGACA
AGGTGCATGCGCGCCCGCTGCTGATGGTGCCGCCGTGCATCAACAAGTAC
TACATCCTGGACCTGCAGCCGGAGAGCTCGCTGGTGCGTCATGTGGTGGA
GCAGGGGCATACGGTGTTCCTGGTGTCGTGGCGCAATCCGGACGCCAGCA
TGGCTGGCAGCACCTGGGACGACTACATCGAGCACGCGGCCATCCGCGCC
ATCGAAGTCGCGCGCGACATCAGCGGCCAGGACAAGATCAACGTGCTCGG
CTTCTGCGTGGGCGGCACCATTGTGTCGACTGCGCTGGCGGTGATGGCCG
CGCGCGGCCAGCACCCGGCTGCCAGCGTCACGCTGCTGACCACGCTGCTG
GACTTTGCCGACACCGGCATCCTCGACGTCTTTGTCGACGAGGGCCATGT
GCAGCTGCGCGAGGCCACGCTGGGCGGCGCCGCCGGCGCGCCGTGCGCGC
TGCTGCGCGGCCTTGAGCTGGCCAATACCTTCTCGTTCCTGCGCCCGAAC
GACCTGGTGTGGAACTACGTGGTCGACAACTACCTGAAGGGCAACACGCC
GGTGCCGTTCGACCTGCTGTTCTGGAACGGCGACGCCACCAACCTGCCGG
GGCCTTGGTACTGCTGGTACCTGCGCCACACCTACCTGCAGGACGAGCTC
AAGGTGCCGGGCAAGCTGACTGTGTGCGGCGTGCCCGTGGACCTGGCCAG
CATCGACGTGCCGACCTACATCTACGGCTCGCGCGAAGACCATATCGTGC
CATGGACCGCGGCCTATGCCTCGACCGCGCTGCTGGCGAACAAGCTGCGC
TTCGTGCTGGGTGCGTCGGGCCATATCGCCGGTGTGATCAACCCGCCGGC
CAAGAACAAGCGCAGCCACTGGACCAACGATGCGCTGCCGGAGTCGCCGC
AGCAATGGCTGGCTGGCGCCACCGAGCATCACGGCAGCTGGTGGCCGGAC
TGGACCGCATGGCTGGCAGGCCAGGCCGGCGCGAAACGTGCCGCGCCCGC
CAACTACGGCAATGCGCGCTATCCCGCGATCGAACCCGCGCCTGGGCGAT
ACGTCAAAGCCAAGGCATGA

In some embodiments of any of the aspects, the amino acid sequence encoded by the endogenous Cupriavidus necator phaC gene comprises SEQ ID NO: 4 or an amino acid sequence that is at least 80%, at least 85%, at least 90%, at least 91%, at least 92%, at least 93%, at least 94%, at least 95%, at least 96%, at least 97%, at least 98%, or at least 99% identical to the sequence of SEQ ID NO: 4 that maintains the same functions as SEQ ID NO: 4 (e.g., PHA synthase).

class I poly(R)-hydroxyalkanoic acid synthase
[Cupriavidus necator], NCBI Reference Sequence:
WP_013956451.1, 589 aa
SEQ ID NO: 4
MATGKGAAASTQEGKSQPFKFTPGPFDPATWLEWSRQWQGTEGNGHAAAS
GIPGLDALAGVKIEPAQLGDIQQRYMKDFSALWQAMAEGKAEATGPLHDR
RFAGDAWRTNLPYRFAAAFYLLNARALTELADAVEADAKTRQRIRFAISQ
WVDAMSPANFLATNPEAQRLLIESGGESLRAGVRNMMEDLTRGKISQTDE
SAFEVGRNVAVSEGAVVFENEYFQLLQYKPLTDKVHARPLLMVPPCINKY
YILDLQPESSLVRHVVEQGHTVFLVSWRNPDASMAGSTWDDYIEHAAIRA
IEVARDISGQDKINVLGFCVGGTIVSTALAVMAARGQHPAASVTLLTTLL
DFADTGILDVFVDEGHVQLREATLGGAAGAPCALLRGLELANTFSFLRPN
DLVWNYVVDNYLKGNTPVPFDLLFWNGDATNLPGPWYCWYLRHTYLQDEL
KVPGKLTVCGVPVDLASIDVPTYIYGSREDHIVPWTAAYASTALLANKLR
FVLGASGHIAGVINPPAKNKRSHWTNDALPESPQQWLAGATEHHGSWWPD
WTAWLAGQAGAKRAAPANYGNARYPAIEPAPGRYVKAKA

In some embodiments of any of the aspects, the engineered inactivating modification of an endogenous polyhydroxyalkanoate (PHA) synthase gene comprises a point mutation. Non-limiting examples of inactivating point mutations of C. necator phaC (see e.g., SEQ ID NO: 4) include non-conservative substitutions of residues T323, C438, Y445, L446, or E267 (e.g., T323I, T323S, C438G, Y445F, L446K, or E267K). Additional non-limiting examples of point mutations of C. necator phaC (see e.g., SEQ ID NO: 4) include C319S, C459S, S260A, S260T, S5461, E267K, T323S, T323I, C438G, Y445F, L446K, W425A, D480N, H508Q, S35P, S80P, A154V, L231P, D306A, L358P, A391T, T393A, V470M, N519S, S546G, and A565E. In some embodiments of any of the aspects, the engineered inactivating modification of an endogenous polyhydroxyalkanoate (PHA) synthase gene comprises a deletion. Non-limiting examples include deletions of regions D281-D290, A372-C382, E578-A589 and/or V585-A589 of C. necator phaC (see e.g., SEQ ID NO: 4). See e.g., Rehm et al., Molecular characterization of the poly(3 hydroxybutyrate) (PHB) synthase from Ralstonia eutropha: in vitro evolution, site-specific mutagenesis and development of a PHB synthase protein model, Biochimica et Biophysica Acta 1594 (2002) 178-190, the content of which is incorporated herein by reference in its entirety. In some embodiments of any of the aspects, the engineered inactivating modification of an endogenous polyhydroxyalkanoate (PHA) synthase gene comprises a deletion of the entire coding sequence (e.g., a knockout of the endogenous phaC gene, denoted herein as ΔphaC).

In some embodiments of any of the aspects, the engineered bacterium comprises an engineered inactivating modification of an endogenous gene involved in the PHA synthesis pathway. In some embodiments of any of the aspects, the endogenous gene involved in the PHA synthesis pathway comprises phaA, phaB, and/or phaC (e.g., a Class I PHA synthase operon). In some embodiments of any of the aspects, the PHA synthesis pathway comprises Cupriavidus necator phaA, Cupriavidus necator phaB, and/or Cupriavidus necator phaC.

PhaA is an acetyl-CoA acetyltransferase that catalyzes the condensation of two acetyl-coA units to form acetoacetyl-CoA. PhaA is involved in the biosynthesis of PHAs (e.g., polyhydroxybutyrate (PHB)). PhaA also catalyzes the reverse reaction, i.e. the cleavage of acetoacetyl-CoA, and is therefore also involved in the reutilization of PHB.

In some embodiments of any of the aspects, the engineered bacterium comprises an engineered inactivating modification of the endogenous Cupriavidus necator phaA gene. In some embodiments of any of the aspects, the nucleic acid sequence of the endogenous Cupriavidus necator phaA gene comprises SEQ ID NO: 5 or a sequence that is at least 80%, at least 85%, at least 90%, at least 91%, at least 92%, at least 93%, at least 94%, at least 95%, at least 96%, at least 97%, at least 98%, or at least 99% identical to the sequence of SEQ ID NO: 5 that maintains the same functions as SEQ ID NO: 5 (e.g., acetyl-CoA acetyltransferase).

Cupriavidus necator phaA acetyl-CoA acetyl-
transferase, Cupriavidus necator H16 chromosome
1, complete sequence, GenBank: CP039287.1, REGION:
1557857-1559035, 1179 bp
SEQ ID NO: 5
ATGACTGACGTTGTCATCGTATCCGCCGCCCGCACCGCGGTCGGCAAGTT
TGGCGGCTCGCTGGCCAAGATCCCGGCACCGGAACTGGGTGCCGTGGTCA
TCAAGGCCGCGCTGGAGCGCGCCGGCGTCAAGCCGGAGCAGGTGAGCGAA
GTCATCATGGGCCAGGTGCTGACCGCCGGTTCGGGCCAGAACCCCGCACG
CCAGGCCGCGATCAAGGCCGGCCTGCCGGCGATGGTGCCGGCCATGACCA
TCAACAAGGTGTGCGGCTCGGGCCTGAAGGCCGTGATGCTGGCCGCCAAC
GCGATCATGGCGGGCGACGCCGAGATCGTGGTGGCCGGCGGCCAGGAAAA
CATGAGCGCCGCCCCGCACGTGCTGCCGGGCTCGCGCGATGGTTTCCGCA
TGGGCGATGCCAAGCTGGTCGACACCATGATCGTCGACGGCCTGTGGGAC
GTGTACAACCAGTACCACATGGGCATCACCGCCGAGAACGTGGCCAAGGA
ATACGGCATCACACGCGAGGCGCAGGATGAGTTCGCCGTCGGCTCGCAGA
ACAAGGCCGAAGCCGCGCAGAAGGCCGGCAAGTTTGACGAAGAGATCGTC
CCGGTGCTGATCCCGCAGCGCAAGGGCGACCCGGTGGCCTTCAAGACCGA
CGAGTTCGTGCGCCAGGGCGCCACGCTGGACAGCATGTCCGGCCTCAAGC
CCGCCTTCGACAAGGCCGGCACGGTGACCGCGGCCAACGCCTCGGGCCTG
AACGACGGCGCCGCCGCGGTGGTGGTGATGTCGGCGGCCAAGGCCAAGGA
ACTGGGCCTGACCCCGCTGGCCACGATCAAGAGCTATGCCAACGCCGGTG
TCGATCCCAAGGTGATGGGCATGGGCCCGGTGCCGGCCTCCAAGCGCGCC
CTGTCGCGCGCCGAGTGGACCCCGCAAGACCTGGACCTGATGGAGATCAA
CGAGGCCTTTGCCGCGCAGGCGCTGGCGGTGCACCAGCAGATGGGCTGGG
ACACCTCCAAGGTCAATGTGAACGGCGGCGCCATCGCCATCGGCCACCCG
ATCGGCGCGTCGGGCTGCCGTATCCTGGTGACGCTGCTGCACGAGATGAA
GCGCCGTGACGCGAAGAAGGGCCTGGCCTCGCTGTGCATCGGCGGCGGCA
TGGGCGTGGCGCTGGCAGTCGAGCGCAAA

In some embodiments of any of the aspects, the amino acid sequence encoded by the endogenous Cupriavidus necator phaA gene comprises SEQ ID NO: 6 or an amino acid sequence that is at least 80%, at least 85%, at least 90%, at least 91%, at least 92%, at least 93%, at least 94%, at least 95%, at least 96%, at least 97%, at least 98%, or at least 99% identical to the sequence of SEQ ID NO: 6 that maintains the same functions as SEQ ID NO: 6 (e.g., PHA synthase).

phaA, acetyl-CoA C-acetyltransferase
[Cupriavidus], NCBI Reference Sequence:
WP_010810132.1, 393 aa
SEQ ID NO: 6
MTDVVIVSAARTAVGKFGGSLAKIPAPELGAVVIKAALERAGVKPEQVSE
VIMGQVLTAGSGQNPARQAAIKAGLPAMVPAMTINKVCGSGLKAVMLAAN
AIMAGDAEIVVAGGQENMSAAPHVLPGSRDGFRMGDAKLVDTMIVDGLWD
VYNQYHMGITAENVAKEYGITREAQDEFAVGSQNKAEAAQKAGKFDEEIV
PVLIPQRKGDPVAFKTDEFVRQGATLDSMSGLKPAFDKAGTVTAANASGL
NDGAAAVVVMSAAKAKELGLTPLATIKSYANAGVDPKVMGMGPVPASKRA
LSRAEWTPQDLDLMEINEAFAAQALAVHQQMGWDTSKVNVNGGAIAIGHP
IGASGCRILVTLLHEMKRRDAKKGLASLCIGGGMGVALAVERK,

In some embodiments of any of the aspects, the engineered inactivating modification of an endogenous gene involved in the PHA synthesis pathway comprises a deletion of the entire coding sequence (e.g., a knockout of the endogenous phaA gene, denoted herein as ΔphaA).

In some embodiments of any of the aspects, the engineered bacterium comprises an engineered inactivating modification of the endogenous Cupriavidus necator phaB gene. PhaB is an acetoacetyl-CoA reductase that catalyzes the chiral reduction of acetoacetyl-CoA to (R)-3-hydroxybutyryl-CoA. PhaB is involved in the biosynthesis of PHAs (e.g., polyhydroxybutyrate (PHB)). PhaB can also be referred to as phbB. In some embodiments of any of the aspects, the nucleic acid sequence of the endogenous Cupriavidus necator phaB gene comprises SEQ ID NO: 7 or a sequence that is at least 80%, at least 85%, at least 90%, at least 91%, at least 92%, at least 93%, at least 94%, at least 95%, at least 96%, at least 97%, at least 98%, or at least 99% identical to the sequence of SEQ ID NO: 7 that maintains the same functions as SEQ ID NO: 7 (e.g., acetoacetyl-CoA reductase).

Cupriavidus necator strain A-04 acetoacetyl-CoA
reductase (phbB) gene, complete cds, GenBank:
FJ897462.1, 741 bp
SEQ ID NO: 7
ATGACTCAGCGCATTGCGTATGTGACCGGCGGCATGGGTGGTATCGGAAC
CGCCATTTGCCAGCGGCTGGCCAAGGATGGCTTTCGTGTGGTGGCCGGTT
GCGGCCCCAACTCGCCGCGCCGCGAAAAGTGGCTGGAGCAGCAGAAGGCC
CTGGGCTTCGATTTCATTGCCTCGGAAGGCAATGTGGCTGACTGGGACTC
GACCAAGACCGCATTCGACAAGGTCAAGTCCGAGGTCGGCGAGGTTGATG
TGCTGATCAACAACGCCGGTATCACCCGCGACGTGGTGTTCCGCAAGATG
ACCCGCGCCGACTGGGATGCGGTGATCGACACCAACCTGACCTCGCTGTT
CAACGTCACCAAGCAGGTGATCGACGGCATGGCCGACCGTGGCTGGGGCC
GCATCGTCAACATCTCGTCGGTGAACGGGCAGAAGGGCCAGTTCGGCCAG
ACCAACTACTCCACCGCCAAGGCCGGCCTGCATGGCTTCACCATGGCACT
GGCGCAGGAAGTGGCGACCAAGGGCGTGACCGTCAACACGGTCTCTCCGG
GCTATATCGCCACCGACATGGTCAAGGCGATCCGCCAGGACGTGCTCGAC
AAGATCGTCGCGACGATCCCGGTCAAGCGCCTGGGCCTGCCGGAAGAGAT
CGCCTCGATCTGCGCCTGGTTGTCGTCGGAGGAGTCCGGTTTCTCGACCG
GCGCCGACTTCTCGCTCAACGGCGGCCTGCATATGGGCTGA,

In some embodiments of any of the aspects, the amino acid sequence encoded by the endogenous Cupriavidus necator phaC gene comprises SEQ ID NO: 8 or an amino acid sequence that is at least 80%, at least 85%, at least 90%, at least 91%, at least 92%, at least 93%, at least 94%, at least 95%, at least 96%, at least 97%, at least 98%, or at least 99% identical to the sequence of SEQ ID NO: 8 that maintains the same functions as SEQ ID NO: 8 (e.g., e.g., acetoacetyl-CoA reductase).

phaB 3-ketoacyl-ACP reductase [Cupriavidus], NCBI
Reference Sequence: WP_010810131.1, 246 aa
SEQ ID NO: 8
MTQRIAYVTGGMGGIGTAICQRLAKDGFRVVAGCGPNSPRREKWLEQQKA
LGFDFIASEGNVADWDSTKTAFDKVKSEVGEVDVLINNAGITRDVVFRKM
TRADWDAVIDTNLTSLFNVTKQVIDGMADRGWGRIVNISSVNGQKGQFGQ
TNYSTAKAGLHGFTMALAQEVATKGVTVNTVSPGYIATDMVKAIRQDVLD
KIVATIPVKRLGLPEEIASICAWLSSEESGFSTGADFSLNGGLHMG

In some embodiments of any of the aspects, the engineered inactivating modification of an endogenous gene involved in the PHA synthesis pathway comprises a deletion of the entire coding sequence (e.g., a knockout of the endogenous phaB gene, denoted herein as AphaB).

In some embodiments of any of the aspects, the engineered bacterium comprises an engineered inactivating modification of an endogenous phaA (e.g., SEQ ID NOs: 5, 6), an engineered inactivating modification of an endogenous phaB (e.g., SEQ ID NOs: 7, 8), or an engineered inactivating modification of an endogenous phaC (e.g., SEQ ID NOs: 3, 4). In some embodiments of any of the aspects, the engineered bacterium comprises an engineered inactivating modification of an endogenous phaA (e.g., SEQ ID NOs: 5, 6). In some embodiments of any of the aspects, the engineered bacterium comprises an engineered inactivating modification of an endogenous phaB (e.g., SEQ ID NOs: 7, 8). In some embodiments of any of the aspects, the engineered bacterium comprises an engineered inactivating modification of an endogenous phaC (e.g., SEQ ID NOs: 3, 4).

In some embodiments of any of the aspects, the engineered bacterium comprises an engineered inactivating modification of an endogenous phaA (e.g., SEQ ID NOs: 5, 6), and an engineered inactivating modification of an endogenous phaB (e.g., SEQ ID NOs: 7, 8). In some embodiments of any of the aspects, the engineered bacterium comprises an engineered inactivating modification of an endogenous phaA (e.g., SEQ ID NOs: 5, 6) and an engineered inactivating modification of an endogenous phaC (e.g., SEQ ID NOs: 3, 4). In some embodiments of any of the aspects, the engineered bacterium comprises an engineered inactivating modification of an endogenous phaB (e.g., SEQ ID NOs: 7, 8) and an engineered inactivating modification of an endogenous phaC (e.g., SEQ ID NOs: 3, 4). In some embodiments of any of the aspects, the engineered bacterium comprises an engineered inactivating modification of an endogenous phaA (e.g., SEQ ID NOs: 5, 6), an engineered inactivating modification of an endogenous phaB (e.g., SEQ ID NOs: 7, 8), and an engineered inactivating modification of an endogenous phaC (e.g., SEQ ID NOs: 3, 4).

In some embodiments of any of the aspects, an organism can comprise alternative groups of genes involved in the PHA synthesis pathway. As an example, the Class II PHA synthase operon (e.g., in Pseudomonas oleovorans) comprises phaC1, phaZ, phaC2, and phaD. As another example, the Class III PHA synthase operon (e.g., in Allochromatium vinosum) comprises phaC, phaE, phaA, ORF4, phaP, and phaB. As such, an engineered bacterium can comprise an engineered inactivating modification and/or an inhibitor of at least one endogenous gene involved in the PHA synthesis pathway (e.g., phaC1, phaZ, phaC2, phaD, phaC, phaE, phaA, ORF4, phaP, and/or phaB).

In some embodiments of any of the aspects, the engineered bacterium comprises an inhibitor of an endogenous PHA synthase gene. Non-limiting examples of PHA synthase (e.g., PhaC, Enzyme Commission (E.C.) 2.3.1) inhibitors include carbadethia CoA analogs, sT-CH₂-CoA, sTet-CH₂-CoA, and sT-aldehyde. See e.g., Zhang et al., Chembiochem. 2015 Jan. 2; 16(1): 156-166, the contents of which are incorporated herein in be reference in their entireties. In some embodiments of any of the aspects, the engineered bacterium comprises an inhibitor of at least one endogenous gene involved in the PHA synthesis pathway. Non-limiting examples of such inhibitors include an inhibitory RNA (e.g., siRNA, miRNA) against a gene involved in PHA synthesis (e.g., a PHA synthase, PhaC, PhaB, PhaA), a small molecule inhibitor of a gene involved in PHA synthesis (e.g., a PHA synthase, PhaC, PhaB, PhaA), and the like.

In some embodiments of any of the aspects, the engineered bacterium comprises at least one exogenous copy of at least one functional thioesterase gene. In some embodiments of any of the aspects, the engineered bacterium does not comprise a functional endogenous thioesterase gene. Thioesterases are enzymes which belong to the esterase family. Esterases, in turn, are one type of the several hydrolases known. Thioesterases exhibit Esterase activity (e.g., splitting of an ester into acid and alcohol, in the presence of water) specifically at a thiol group. Thioesterases or thiolester hydrolases are identified as members of E.C.3.1.2.

Thioesterases (TEs) can determine the chain length of substrate fatty acids, for example in the synthesis of PHAs. As such, TEs can modulate polymer length and ratio or components of the PHA. In some embodiments of any of the aspects, the functional thioesterase gene preferentially produces or leads to the production of TAGs comprising a specific type of fatty acid R group (e.g., C16), as described herein. As such, the functional thioesterase gene can be selected from any thioesterase gene from any species that preferentially produces or leads to the production of TAGs comprising a specific type of fatty acid R group (e.g., C16). In some embodiments of any of the aspects, the functional thioesterase is an Acyl-Acyl Carrier Protein (acyl-ACP) Thioesterase. In some embodiments of any of the aspects, the functional thioesterase gene is heterologous. In some embodiments of any of the aspects, a thioesterase polypeptide as described herein is truncated to remove an organelle targeting sequence(s); in some embodiments, such a targeting sequence can contribute to poor expression of the thioesterase polypeptide, e.g., in the engineered bacteria described herein.

In some embodiments of any of the aspects, the functional heterologous thioesterase is from a plant species (e.g., Cuphea palustris, Arachis hypogaea, Mangifera indica, Morella rubra, Pistacia vera, or Theobroma cacao). In some embodiments of any of the aspects, the functional heterologous thioesterase gene comprises a Cuphea thioesterase. In some embodiments of any of the aspects, the functional heterologous thioesterase gene comprises a Arachis thioesterase. In some embodiments of any of the aspects, the functional heterologous thioesterase gene comprises a Mangifera thioesterase. In some embodiments of any of the aspects, the functional heterologous thioesterase gene comprises a Morella thioesterase. In some embodiments of any of the aspects, the functional heterologous thioesterase gene comprises a Pistacia thioesterase. In some embodiments of any of the aspects, the functional heterologous thioesterase gene comprises a Theobroma thioesterase. In some embodiments of any of the aspects, the functional heterologous thioesterase gene comprises a Cuphea palustris FatB1 gene (i.e., CpFatB1), a Cuphea palustris FatB2 gene (i.e., CpFatB2), a Cuphea palustris FatB2-FatB1 hybrid gene (i.e., CpFatB2-CpFatB1), a Arachis hypogaea FatB2-1 gene, a Mangifera indica FatA gene, a Morella rubra FatA gene, a Pistacia vera FatA gene, a Theobroma cacao FatA gene, a Theobroma cacao FatB gene (e.g., FatB1, FatB2, FatB3, BatB4, FatB5, or FatB6), or a Limosilactobacillus reuteri TE gene.

In some embodiments of any of the aspects, the engineered bacterium comprises at least one exogenous copy of at least one functional thioesterase gene comprising one of SEQ ID NOs: 9-11, SEQ ID NOs: 99-121 or a nucleic acid sequence that is at least 80%, at least 85%, at least 90%, at least 91%, at least 92%, at least 93%, at least 94%, at least 95%, at least 96%, at least 97%, at least 98%, or at least 99% identical to the sequence of at least one of SEQ ID NOs: 9-11 or SEQ ID NOs: 99-121, that maintains the same functions as at least one of SEQ ID NOs: 9-11 or SEQ ID NOs: 99-121 (e.g., thioesterase).

Cuphea palustris FatB1, GenBank: U38188.1, 1236 bp, complete
CDS
SEQ ID NO: 9
ATGGTGGCTGCTGCAGCAAGTTCTGCATGCTTCCCTGTTCCATCCCCAGGAGCCTCCCCT
AAACCTGGGAAGTTAGGCAACTGGTCATCGAGTTTGAGCCCTTCCTTGAAGCCCAAGTCA
ATCCCCAATGGCGGATTTCAGGTTAAGGCAAATGCCAGTGCGCATCCTAAGGCTAACGG
TTCTGCAGTAACTCTAAAGTCTGGCAGCCTCAACACTCAGGAGGACACTTTGTCGTCGTC
CCCTCCTCCCCGGGCTTTTTTTAACCAGTTGCCTGATTGGAGTATGCTTCTGACTGCAATC
ACAACCGTCTTCGTGGCACCAGAGAAGCGGTGGACTATGTTTGATAGGAAATCTAAGAG
GCCTAACATGCTCATGGACTCGTTTGGGTTGGAGAGAGTTGTTCAGGATGGGCTCGTGTT
CAGACAGAGTTTTTCGATTAGGTCTTATGAAATATGCGCTGATCGAACAGCCTCTATAGA
GACGGTGATGAACCACGTCCAGGAAACATCACTCAATCAATGTAAGAGTATAGGTCTTC
TCGATGACGGCTTTGGTCGTAGTCCTGAGATGTGTAAAAGGGACCTCATTTGGGTGGTTA
CAAGAATGAAGATAATGGTGAATCGCTATCCAACTTGGGGCGATACTATCGAGGTCAGT
ACCTGGCTCTCTCAATCGGGGAAAATCGGTATGGGTCGCGATTGGCTAATAAGTGATTGC
AACACAGGAGAAATTCTTGTAAGAGCAACGAGTGTGTATGCCATGATGAATCAAAAGAC
GAGAAGATTCTCAAAACTCCCACACGAGGTTCGCCAGGAATTTGCGCCTCATTTTCTGGA
CTCTCCTCCTGCCATTGAAGACAACGACGGTAAATTGCAGAAGTTTGATGTGAAGACTGG
TGATTCCATTCGCAAGGGTCTAACTCCGGGGTGGTATGACTTGGATGTCAATCAGCACGT
AAGCAACGTGAAGTACATTGGGTGGATTCTCGAGAGTATGCCAACAGAAGTTTTGGAGA
CTCAGGAGCTATGTTCTCTCACCCTTGAATATAGGCGGGAATGCGGAAGGGACAGTGTG
CTGGAGTCCGTGACCTCTATGGATCCCTCAAAAGTTGGAGACCGGTTTCAGTACCGGCAC
CTTCTGCGGCTTGAGGATGGGGCTGATATCATGAAGGGAAGAACTGAGTGGCGGCCGAA
GAATGCAGGAACTAACGGGGCGATATCAACAGGAAAGACTTGA
Cuphea palustris FatB2, complete CDS, GenBank: U38189.1,
1408 bp
SEQ ID NO: 10
CCACGCGTCCGCTGAGTTTGCTGGTTACCATTTTCCCTGCGAACAAACATGGTGGCTGCC
GCAGCAAGTGCTGCATTCTTCTCCGTCGCAACCCCGCGAACAAACATTTCGCCATCGAGC
TTGAGCGTCCCCTTCAAGCCCAAATCAAACCACAATGGTGGCTTTCAGGTTAAGGCAAAC
GCCAGTGCCCATCCTAAGGCTAACGGTTCTGCAGTAAGTCTAAAGTCTGGCAGCCTCGAG
ACTCAGGAGGACAAAACTTCATCGTCGTCCCCTCCTCCTCGGACTTTCATTAACCAGTTG
CCCGTCTGGAGTATGCTTCTGTCTGCAGTCACGACTGTCTTCGGGGTGGCTGAGAAGCAG
TGGCCAATGCTTGACCGGAAATCTAAGAGGCCCGACATGCTTGTGGAACCGCTTGGGGTT
GACAGGATTGTTTATGATGGGGTTAGTTTCAGACAGAGTTTTTCGATTAGATCTTACGAA
ATAGGCGCTGATCGAACAGCCTCGATAGAGACCCTGATGAACATGTTCCAGGAAACATC
TCTTAATCATTGTAAGATTATCGGTCTTCTCAATGACGGCTTTGGTCGAACTCCTGAGATG
TGTAAGAGGGACCTCATTTGGGTGGTCACGAAAATGCAGATCGAGGTGAATCGCTATCC
TACTTGGGGTGATACTATAGAGGTCAATACTTGGGTCTCAGCGTCGGGGAAACACGGTAT
GGGTCGAGATTGGCTGATAAGTGATTGCCATACAGGAGAAATTCTTATAAGAGCAACGA
GCGTGTGGGCTATGATGAATCAAAAGACGAGAAGATTGTCGAAAATTCCATATGAGGTT
CGACAGGAGATAGAGCCTCAGTTTGTGGACTCTGCTCCTGTCATTGTAGACGATCGAAAA
TTTCACAAGCTTGATTTGAAGACCGGTGATTCCATTTGCAATGGTCTAACTCCAAGGTGG
ACTGACTTGGATGTCAATCAGCACGTTAACAATGTGAAATACATCGGGTGGATTCTCCAG
AGTGTTCCCACAGAAGTTTTCGAGACGCAGGAGCTATGTGGCCTCACCCTTGAGTATAGG
CGAGAATGCGGAAGGGACAGTGTGCTGGAGTCCGTGACCGCTATGGATCCATCAAAAGA
GGGAGACCGGTCTCTTTACCAGCACCTTCTCCGACTCGAGGACGGGGCTGATATCGTCAA
GGGGAGAACCGAGTGGCGGCCGAAGAATGCAGGAGCCAAGGGAGCAATATTAACCGGA
AAGACCTCAAATGGAAACTCTATATCTTAGAAGGAGGAAGGGACCTTTCCGAGTTGTGT
GTTTATTTGCTTTGCTTTGATTCACTCCATTGTATAATAATACTACGGTCAGCCGTCTTTGT
ATTTGCTAAGACAAATAGCACAGTCATTAAGTT
Engineered chimera of C. palustris FatB1 (aa 1-218) and FatB2
(aa 219-316) thioesterase—Chimera 4 (981 bp)
SEQ ID NO: 11
ATGCTGCTGACCGCCATCACGACCGTGTTCGTGGCCCCGGAGAAGCGCTGGACCATGTTC
GACCGCAAGTCGAAGCGCCCGAACATGCTGATGGACTCGTTCGGCCTGGAGCGCGTGGT
GCAGGACGGCCTGGTGTTCCGCCAGTCGTTCTCGATCCGCTCGTACGAGATCTGCGCCGA
CCGCACCGCCTCGATCGAGACCGTGATGAACCACGTGCAGGAGACCTCGCTGAACCAGT
GCAAGTCGATCGGCCTGCTGGACGACGGCTTCGGCCGTTCGCCGGAGATGTGCAAGCGC
GACCTGATCTGGGTGGTGACCCGCATGAAGATCATGGTGAACCGCTACCCGACCTGGGG
CGACACCATCGAGGTGAGCACCTGGCTGTCGCAGTCGGGCAAGATCGGCATGGGCCGCG
ATTGGCTGATCTCGGACTGCAACACCGGCGAGATCCTGGTGCGCGCCACCTCGGTGTACG
CCATGATGAACCAGAAGACCCGCCGCTTCTCGAAGCTGCCGCACGAGGTGCGCCAGGAG
TTCGCCCCGCACTTCCTGGATTCACCACCGGCCATCGAGGACAATGACGGCAAGCTGCAG
AAGTTCGACGTGAAGACCGGCGACTCGATCCGCAAGGGCCTGACCCCGGGCTGGTACGA
CCTGGACGTGAACCAGCACGTGAACAACGTGAAGTACATCGGCTGGATCCTGCAGTCGG
TGCCGACCGAGGTGTTCGAGACCCAGGAGCTGTGCGGCCTGACCCTGGAGTATCGCCGC
GAATGCGGCCGCGATTCGGTGCTGGAATCGGTGACCGCCATGGACCCGTCGAAGGAGGG
CGATCGCTCGCTGTACCAGCACCTGCTGCGCCTGGAAGATGGCGCCGACATCGTGAAGG
GCCGCACCGAATGGCGCCCGAAGAATGCAGGCGCAAAGGGCGCCATTCTGACCGGCAAG
ACCTCAGGCGGCCACCACCACCACCATCATTGA
Arachis hypogaea Acyl-[acyl-carrier-protein] hydrolase
(AhFatB2-1) codon-optimized, 1245 nt
SEQ ID NO: 99
ATGGTGGCCACCGCCGCCACCTCGTCGTTCTTCCCGGTGACCTCGCGCACCGGCGGCGAG
GGCGGCGGCGGCATCCCGGCCTCGCTGGGCGGCGGCCTGAAGCAGAACCACCGCTCGTC
GTCGGTGAAGGCCAACGCCCACGCCCCGTCGAAGATCAACGGCACCGCCACCAAGGTGC
CGAAGTCGATGGAGTCGATGAAGCTGGAGTCGTCGTCGACCACCGGCGCCAACGCCCCG
CGCACCTTCATCAACCAGATCCCGGACTGGTCGATGCTGCTGGCCGCCATCACCACCGCC
TTCCTGGCCGCCGAGAAGCAGTGGATGATGATCGACTGGAAGCCGAAGCGCTCGGACGT
GCTGTCGGACCCGTTCGGCATCGGCCGCATCGTGCAGGACGGCCTGGCCTTCCGCCAGAA
CTTCTCGATCCGCTCGTACGAGATCGGCGCCGACAAGACCGCCTCGATCGAGACCCTGAT
GAACCACCTGCAGGAGACCGCCCTGAACCACGTGAAGACCGCCGGCCTGCTGGGCGACG
GCTTCGGCTCGACCCCGGAGATGTGCAAGAAGAACCTGATCTGGGTGGTGACCCGCATG
CAGGTGGAGGTGGACCGCTACCCGACCTGGGGCGACGTGGTGCAGGTGGACACCTGGGT
GTCGGCCTCGGGCAAGAACGGCATGCGCCGCGACTGGATCATCCGCGACGCCAACACCG
GCGAGATCCTGACCCGCGCCTCGTCGATCTGGGTGATGATGAACAAGGTGACCCGCCGC
CTGTCGAAGATCCCGGAGGAGGTGCGCCAGGAGATCGCCTCGTACTTCGTGGACTCGCC
GCCGGTGGTGGAGGAGGACAACCGCAAGCTGTCGAAGCTGGACGACACCGCCGACCAC
ATCCGCCGCGGCCTGTCGCCGCGCTGGTCGGACCTGGACGTGAACCAGCACGTGAACAA
CGTGAAGTACATCGGCTGGCTGCTGGAGTCGGCCCCGCAGGCCATCCTGGAGTCGCACG
AGCTGCGCGCCATGACCCTGGAGTACCGCCGCGAGTGCGGCAAGGACTCGGTGCTGGAC
TCGCTGACCGACGTGTCGGGCGCCGACATCGGCAACCTGGCCGGCGGCGGCTCGCTGGA
GTGCAAGCACCTGCTGCGCCTGGAGGACGGCGGCGAGATCGTGCGCGGCCGCACCGAGT
GGCGCCCGAAGCCGGTGAACAACTTCGGCGCCATGAACCAGGTGTTCCCGGCCGAGAAC
TAA,
Arachis hypogaea palmitoyl-acyl carrier protein thioesterase,
chloroplastic (AhFatB2-1), NCBI Reference Sequence:
XM_025825221.1, 1245 nt
SEQ ID NO: 100
ATGGTTGCTACTGCTGCTACGTCGTCGTTTTTCCCTGTGACGTCACGAACCGGTGGAGAA
GGAGGAGGAGGAATCCCTGCCAGCCTCGGCGGAGGGCTCAAACAAAATCACAGGTCTTC
AAGTGTTAAGGCCAATGCGCATGCTCCTTCAAAGATCAACGGAACCGCCACAAAGGTTC
CAAAATCCATGGAGAGCATGAAGCTGGAATCCTCGTCGACGACGGGGGCTAATGCGCCG
AGGACTTTCATTAACCAGATTCCGGATTGGAGCATGCTGCTGGCCGCCATCACGACAGCC
TTCCTTGCGGCGGAGAAGCAGTGGATGATGATCGATTGGAAGCCGAAGCGATCCGATGT
GCTATCTGATCCATTTGGTATTGGGAGGATTGTGCAGGATGGGCTTGCTTTCAGGCAAAA
TTTCTCCATTCGATCTTACGAGATAGGCGCCGATAAGACCGCGTCTATAGAGACGCTAAT
GAATCATTTGCAGGAAACTGCACTTAATCATGTTAAGACTGCTGGGCTTCTTGGTGATGG
CTTTGGTTCGACGCCGGAAATGTGTAAGAAGAACCTGATATGGGTTGTGACTCGGATGCA
AGTTGAAGTTGATCGTTACCCAACATGGGGAGATGTAGTTCAAGTTGACACTTGGGTTTC
TGCATCAGGGAAAAATGGTATGCGTCGTGATTGGATCATACGTGATGCCAATACGGGTG
AAATCTTGACAAGAGCCTCCAGTATTTGGGTCATGATGAATAAAGTGACAAGGAGACTA
TCCAAAATTCCAGAAGAAGTCAGGCAAGAGATTGCGTCGTATTTTGTGGATTCTCCTCCA
GTTGTCGAAGAGGATAACAGAAAACTGTCTAAACTTGATGATACTGCAGATCATATTCGT
CGTGGTCTAAGTCCTAGATGGAGTGATCTAGATGTTAATCAGCATGTTAACAATGTGAAG
TACATTGGCTGGCTTCTGGAGAGTGCTCCACAGGCAATCTTGGAGAGTCATGAGCTGCGG
GCCATGACTCTGGAGTACAGGAGGGAATGTGGCAAGGACAGTGTGCTGGATTCCCTAAC
TGATGTCTCTGGTGCTGATATCGGGAACTTAGCTGGCGGCGGATCTCTCGAGTGCAAACA
CTTGCTTAGGCTTGAAGATGGTGGTGAGATTGTGAGGGGTAGGACTGAATGGAGGCCCA
AGCCTGTGAACAACTTTGGTGCTATGAATCAGGTTTTTCCAGCAGAAAACTGA,
Arachis hypogaea Acyl-[acyl-carrier-protein] hydrolase (AhFatB2-
1) truncated codon-optimized (corresponds to nt 187-1245 of
SEQ ID NO: 99), 1059 nt
SEQ ID NO: 101
ATGGAGTCGATGAAGCTGGAGTCGTCGTCGACCACCGGCGCCAACGCCCCGCGCACCTT
CATCAACCAGATCCCGGACTGGTCGATGCTGCTGGCCGCCATCACCACCGCCTTCCTGGC
CGCCGAGAAGCAGTGGATGATGATCGACTGGAAGCCGAAGCGCTCGGACGTGCTGTCGG
ACCCGTTCGGCATCGGCCGCATCGTGCAGGACGGCCTGGCCTTCCGCCAGAACTTCTCGA
TCCGCTCGTACGAGATCGGCGCCGACAAGACCGCCTCGATCGAGACCCTGATGAACCAC
CTGCAGGAGACCGCCCTGAACCACGTGAAGACCGCCGGCCTGCTGGGCGACGGCTTCGG
CTCGACCCCGGAGATGTGCAAGAAGAACCTGATCTGGGTGGTGACCCGCATGCAGGTGG
AGGTGGACCGCTACCCGACCTGGGGCGACGTGGTGCAGGTGGACACCTGGGTGTCGGCC
TCGGGCAAGAACGGCATGCGCCGCGACTGGATCATCCGCGACGCCAACACCGGCGAGAT
CCTGACCCGCGCCTCGTCGATCTGGGTGATGATGAACAAGGTGACCCGCCGCCTGTCGAA
GATCCCGGAGGAGGTGCGCCAGGAGATCGCCTCGTACTTCGTGGACTCGCCGCCGGTGG
TGGAGGAGGACAACCGCAAGCTGTCGAAGCTGGACGACACCGCCGACCACATCCGCCGC
GGCCTGTCGCCGCGCTGGTCGGACCTGGACGTGAACCAGCACGTGAACAACGTGAAGTA
CATCGGCTGGCTGCTGGAGTCGGCCCCGCAGGCCATCCTGGAGTCGCACGAGCTGCGCG
CCATGACCCTGGAGTACCGCCGCGAGTGCGGCAAGGACTCGGTGCTGGACTCGCTGACC
GACGTGTCGGGCGCCGACATCGGCAACCTGGCCGGCGGCGGCTCGCTGGAGTGCAAGCA
CCTGCTGCGCCTGGAGGACGGCGGCGAGATCGTGCGCGGCCGCACCGAGTGGCGCCCGA
AGCCGGTGAACAACTTCGGCGCCATGAACCAGGTGTTCCCGGCCGAGAACTAA,
Arachis hypogaea palmitoyl-acyl carrier protein thioesterase,
chloroplastic (AhFatB2-1) truncated (corresponds to nt 187-
1245 of SEQ ID NO: 100), 1059 nt
SEQ ID NO: 102
ATGGAGAGCATGAAGCTGGAATCCTCGTCGACGACGGGGGCTAATGCGCCGAGGACTTT
CATTAACCAGATTCCGGATTGGAGCATGCTGCTGGCCGCCATCACGACAGCCTTCCTTGC
GGCGGAGAAGCAGTGGATGATGATCGATTGGAAGCCGAAGCGATCCGATGTGCTATCTG
ATCCATTTGGTATTGGGAGGATTGTGCAGGATGGGCTTGCTTTCAGGCAAAATTTCTCCA
TTCGATCTTACGAGATAGGCGCCGATAAGACCGCGTCTATAGAGACGCTAATGAATCATT
TGCAGGAAACTGCACTTAATCATGTTAAGACTGCTGGGCTTCTTGGTGATGGCTTTGGTT
CGACGCCGGAAATGTGTAAGAAGAACCTGATATGGGTTGTGACTCGGATGCAAGTTGAA
GTTGATCGTTACCCAACATGGGGAGATGTAGTTCAAGTTGACACTTGGGTTTCTGCATCA
GGGAAAAATGGTATGCGTCGTGATTGGATCATACGTGATGCCAATACGGGTGAAATCTT
GACAAGAGCCTCCAGTATTTGGGTCATGATGAATAAAGTGACAAGGAGACTATCCAAAA
TTCCAGAAGAAGTCAGGCAAGAGATTGCGTCGTATTTTGTGGATTCTCCTCCAGTTGTCG
AAGAGGATAACAGAAAACTGTCTAAACTTGATGATACTGCAGATCATATTCGTCGTGGTC
TAAGTCCTAGATGGAGTGATCTAGATGTTAATCAGCATGTTAACAATGTGAAGTACATTG
GCTGGCTTCTGGAGAGTGCTCCACAGGCAATCTTGGAGAGTCATGAGCTGCGGGCCATG
ACTCTGGAGTACAGGAGGGAATGTGGCAAGGACAGTGTGCTGGATTCCCTAACTGATGT
CTCTGGTGCTGATATCGGGAACTTAGCTGGCGGCGGATCTCTCGAGTGCAAACACTTGCT
TAGGCTTGAAGATGGTGGTGAGATTGTGAGGGGTAGGACTGAATGGAGGCCCAAGCCTG
TGAACAACTTTGGTGCTATGAATCAGGTTTTTCCAGCAGAAAACTGA,
Mangifera indica palmitoyl-acyl carrier protein thioesterase,
chloroplastic-like (MiFatA), NCBI Reference Sequence:
XM_044638751.1 region 13-1161, 1149 nt
SEQ ID NO: 103
ATGACATCCGTGGCATGTAAGATCATCCTTTCCAGAGAATTATTCAAGGAAGAGAAGAA
GATTAAGCCCATGGCGACGGCCAAAGTGGGGCTTTGTTCATCAGGGAATTTGATAAGAC
GGAAACATGGAAGGCATTTGTTGATAGCAAGTGCCAGTAATCCAAATGGTCTAGACATG
ATGAAAGGAAAAAAGGTGAACGGAATTCACCATAACGAAGAGACTCATCATCAGCTGCT
ACTTAAACAAAGGGTTTCTAAGGCACCCCTTCATGCATGTTTGCTTGGAAGGTTTGTAGG
TGATAGGTTTATGTATAGACAAACCTTCATTATAAGATCATATGAAATTGGACCAGATAA
AACTGCCACTATGGAAACACTCTTGAATCTCCTTCAGGAGACTGCTCTGAATCATGTAAC
GGGCTCGGGACTAGCTGGGAACGGGTTTGGCGCTACCCGAGAGATGAGTCTTCGAAAAC
TCATCTGGGTCGTCACTCGCATCAACATTCAAGTGCAGAGATATAGCTGCTGGGGAGATG
TTGTGGAGATAGATACTTGGGTTGATTCTTCAGGAAAGAATGCCATGCGCAGGGACTGG
ATTATCCGAGATTATCATACCCAGGAAATAATAACAAGGGCAACAAGCACCTGGGTGAC
CATGAACAGAGAAACAAGGAGGTTATCAAAGATTCCTGAACAAGTAAAGCAAGAAGTG
TTTCCGTTTTACCTAGACAGAGTTGCAATTGCAAAAGAACAAAATGATGTTGGGAAAATT
GACAAGCTAACTGATGAAACTGCAGAGAGAATTCGATCTGGTTTAGCTCCAAGATGGAA
TGACATGGATGCCAATCAGCATGTAAACAATGTCAAATACATTGGATGGATTTTAGAGA
GTGTTCCAATACATGTATTAAAAGATTACAATATGACAAGCATGACCCTGGAGTATCGAC
GTGAGTGTCGCCAATCAAATTTGTTGGAGTCCTTGACAAGCTCAACAGCCAGCGTCACTG
GAGACCCCAACAATAATTCCAACAATCGCATTGCAGACTTGAAATACACACATCTACTTC
GAATGCAAGCTGATAAGGCTGAGATAGTCCGTGCCAGATCAGAATGGCAATCCAAACAA
ATAACACAAGTCATCACATAG,
Morella rubra Palmitoyl-acyl carrier protein thioesterase,
chloroplastic (MrFatA), GenBank: CM025852.1 region: complement
(27992667-27995211), CDS join (1-555, 852-985, 1369-1482,
1554-1731, 1875-1943, 2294-2545), 1302 nt
SEQ ID NO: 104
ATGTTGGAAACTTTTATTTTTTGTCTCCTCATCAGGCAACGTGAGTTCGAAGCTACATCGG
CCACCTTATATTTTCAAACCTATATTTTGCTCGGTTCTACAATTTCCCGCGGTATAGTATA
TCTCTCTGTTCAATCAGCGATGACAGTGATGGCTGCACTGAGTAACGCTCGACTGTATTT
TGGAGGGGATTTTTGCAGGGGAGATAAGAAGAATATGGCCATGGCGAGGTTGGGTTATT
ATCCTTCATTGAATTGTGCAATCAAGCCGAAGCAGCCAAGTTTATTAGTGATAGCAAGTG
CTAGTACTCCTCCGAGCATATATACCATCAACGGAAAGAAGGTGAATGGAATAAACTTC
GGGGAGGCTCCATTTAGGAGTAACAAGTATAGCGATTCAGCAAAAGAAAGCTGTGTTGA
TGCACCTCTTCATGCAGTTTTGCTTGGAAGGTTCGTGGAGGACCAGTTTGTTTATAGACA
GACATTCATTATCAGGTCTTACGAGATTGGACCAGACAAAACTGCTACCATGGAAACACT
GATGAATCTCCTTCAGGAGACAGCTCTCAATCATGTGACAAGCTCTGGCCTTGCTGGGAA
TGGCTTTGGTGCCACCCGCGAGATGAGCCTACGGAAACTTATTTGGGTTGTCACACGGGT
CCACATTCAAGTACAGAGATATAGCTGCTGGGGAGATGTTGTTGAGATTGATACTTGGGT
GGATGCCGAGGGGAAAAATGGGATGCGCAGGGACTGGATAATCCGGGATTACCACACA
CAAGAGATCATTACCAGGGCCACAAGTACTTGGGTTATCATGAATCAAGAAACACGACG
GTTGTCTAAGATCCCAGAACAAGTGAGAGAGGAAGTGGTACCGTTCTACCTGGACAGAC
TTGCAGTTTCCGCAGAAATGAATGATAGTGAAAAAATTGACAAGCTCACTGACGAAACC
GCAGAAAGAATTCGATCAGGATTAGCCCCAAGATGGAGCGACATGGATGCCAATCAGCA
CGTGAACAACGTGAAATATATTGGATGGATCCTGGAGAGCGTGCCCATAAACGTGCTTG
GAAATTATGATCTTACGAGCATGACTCTGGAGTACCGCCGTGAGTGCCGGCAATCAAATT
TGCTGGAGTCTTTGACAAGTTCAACAGCAAATGCAACTGAAGCCTCGTATAACTCCTTAC
ATCGATCTAAACCAGACTTGGAATTCACACACCTGCTTCGCATGCTAGCAGACAAGGCA
GAGATAGTACGGGCAAGAACACAGTGGCATTCCAAACAAAAACGAAACTGA,
Pistacia vera palmitoyl-acyl carrier protein thioesterase,
chloroplastic-like (PvFatA), NCBI Reference Sequence:
XM_031391868.1 region 92-1252, 1161 nt
SEQ ID NO: 105
ATGATATCCGTGGCACGAACAAGTTATGTAAGATTATCCTTTCCAGAGAATTTATTCAAG
GAAGAGAAGGAGATCGTACCCATGGCCATGGCCAAGGTCGGGTTTTGTTGCTCGTTGAA
TTTGATCCGACCAAAACATGGAAGGCTTTTGGTGATAGCAAGTGCTAGTAATGCGAAGA
GCCTGGACATCATGAATGGAAAAAAGGTGAATGGAATTCACGTTAATGAAGAGACTCGT
CATCAGCGGCTACTTAATCAAAGAGTTGCTGACGCACCCCTCCATGCATGTTTGCTTGGA
AAATTTGTAGAGGATAGGTTTTTGTATAGACAAACCTTCATTGTCAGGTCATATGAAATT
GGACCAGATAAAACTGCCACTATGGAAACACTCTTGAATCTCCTTCAGGAGACAGCTCTG
AATCATGTAACGAGCTCGGGACTTGCTGGGAACGGGTTTGGTGCTACCCGAGAGATGAG
TGTTAGAAAACTCATCTGGGTCGTCACTCGCATCAACATTCAAGTACAGAGATATAGCTG
CTGGGGAGATGTTGTTGAGATAGATACTTGGGTTGATGCAGCAGGAAAGAATGCGATGC
GCCGGGACTGGATTATCCGAGATTATCGTACCCAAGAGATAATAACAAGAGCAACAAGC
ACCTGGGTGATCATGAACAGAGAAACAAGAAGATTATCAAAGATTCCTGAACAAGTAAG
GCAAGAAGTGTTACCATTTTACCTAGGTAGAGTTGCAATTGCAAAAGAACAAAATGATG
TTGGGAAAATTGACAAGCTTACTGATGAAACTGCAGAGAGAATTCGGTCTGGTTTAGCTC
CAAGATGGAATGACATGGATGCCAATCAGCATGTAAATAATGTCAAATACATAGGATGG
ATTTTGGAGAGTGTGCCAATACACGTCTTAAAAGATTACAATCTGACAAGCATGACCCTG
GAGTATCGACGTGAATGTCGCCAATCAAATTTGCTAGAGTCCTTGACAAGTTCAACAGCC
AGTGTCACTGGAGACCCCAACAATAATTCCAATAATCGCATTGCAGACTTGGAATACAC
ACATCTACTTCGTATGCAAGCTGATAAAGCTGAGATAGTCCGAGCCAGATCAGAATGGC
AGTCCAAACAAATAACACAAGCCATCACTTGA,
Theobroma cacao oleoyl-acyl carrier protein thioesterase 1,
chloroplastic (TcFATA) codon-optimized, 1128 nt
SEQ ID NO: 106
ATGCTGAAGCTGTCGTCGTGCAACGTGACCGACCAGCGCCAGGCCCTGGCCCAGTGCCG
CTTCCTGGCCCCGCCGGCCCCGTTCTCGTTCCGCTGGCGCACCCCGGTGGTGGTGTCGTG
CTCGCCGTCGTCGCGCCCGAACCTGTCGCCGCTGCAGGTGGTGCTGTCGGGCCAGCAGCA
GGCCGGCATGGAGCTGGTGGAGTCGGGCTCGGGCTCGCTGGCCGACCGCCTGCGCCTGG
GCTCGCTGACCGAGGACGGCCTGTCGTACAAGGAGAAGTTCATCGTGCGCTGCTACGAG
GTGGGCATCAACAAGACCGCCACCGTGGAGACCATCGCCAACCTGCTGCAGGAGGTGGG
CTGCAACCACGCCCAGTCGGTGGGCTACTCGACCGACGGCTTCGCCACCACCCGCACCAT
GCGCAAGCTGCACCTGATCTGGGTGACCGCCCGCATGCACATCGAGATCTACAAGTACC
CGGCCTGGTCGGACGTGATCGAGATCGAGACCTGGTGCCAGTCGGAGGGCCGCATCGGC
ACCCGCCGCGACTGGATCCTGAAGGACTTCGGCACCGGCGAGGTGATCGGCCGCGCCAC
CTCGAAGTGGGTGATGATGAACCAGGACACCCGCCGCCTGCAGAAGGTGTCGGACGACG
TGCGCGAGGAGTACCTGGTGTTCTGCCCGCGCGAGCTGCGCCTGGCCTTCCCGGAGGAG
AACAACAACTCGCTGAAGAAGATCGCCAAGCTGGACGACTCGTTCCAGTACTCGCGCCT
GGGCCTGATGCCGCGCCGCGCCGACCTGGACATGAACCAGCACGTGAACAACGTGACCT
ACATCGGCTGGGTGCTGGAGTCGATGCCGCAGGAGATCATCGACACCCACGAGCTGCAG
ACCATCACCCTGGACTACCGCCGCGAGTGCCAGCAGGACGACGTGGTGGACTCGCTGAC
CTCGCCGGAGCAGGTGGAGGGCACCGAGAAGGTGTCGGCCATCCACGGCACCAACGGCT
CGGCCGCCGCCCGCGAGGACAAGCAGGACTGCCGCCAGTTCCTGCACCTGCTGCGCCTG
TCGTCGGACGGCCAGGAGATCAACCGCGGCCGCACCGAGTGGCGCAAGAAGCCGGCCCG
CTAA,
Theobroma cacao oleoyl-acyl carrier protein thioesterase 1,
chloroplastic (TcFATA), NCBI Reference Sequence: XM_007049650.2,
1128 nt
SEQ ID NO: 107
ATGTTGAAGCTTTCTTCCTGCAATGTGACGGACCAAAGACAGGCCTTGGCCCAATGCAGA
TTCCTCGCTCCGCCTGCTCCGTTCTCCTTTCGCTGGCGTACCCCAGTCGTCGTCTCCTGCTC
TCCTTCCAGCAGACCTAACCTGTCGCCTCTTCAAGTTGTCCTGTCTGGCCAGCAGCAAGC
TGGGATGGAGCTGGTCGAATCCGGGTCGGGGAGTTTGGCTGACCGGCTCCGGTTGGGTA
GCTTGACGGAAGATGGTTTGTCCTATAAGGAGAAGTTTATTGTGAGGTGTTATGAGGTGG
GAATTAACAAAACTGCCACTGTTGAAACCATTGCCAATCTCTTGCAGGAGGTTGGATGTA
ACCATGCTCAAAGTGTTGGATATTCCACAGATGGGTTTGCTACCACTCGCACCATGAGAA
AATTGCATCTCATTTGGGTAACTGCACGCATGCACATTGAAATATACAAATACCCTGCTT
GGAGTGATGTGATTGAAATAGAGACATGGTGCCAAAGTGAGGGAAGAATTGGAACCAG
AAGGGACTGGATTCTTAAGGACTTTGGAACTGGTGAAGTTATTGGAAGAGCTACTAGCA
AGTGGGTGATGATGAACCAGGACACTAGGCGGCTACAGAAAGTCAGTGATGATGTCAGG
GAAGAATATTTAGTCTTCTGTCCACGAGAACTCAGATTAGCATTTCCAGAGGAGAACAAT
AATAGTTTGAAGAAAATTGCCAAATTAGATGACTCTTTTCAGTATTCCAGGCTAGGGCTT
ATGCCAAGAAGAGCTGATCTGGACATGAACCAGCATGTCAATAATGTCACCTACATTGG
ATGGGTTCTGGAGAGCATGCCTCAAGAGATCATTGACACCCATGAACTGCAAACTATCA
CGTTAGATTACAGACGGGAATGCCAACAGGATGACGTGGTGGATTCACTTACCAGTCCA
GAACAAGTGGAGGGTACCGAAAAAGTTTCAGCGATTCACGGAACAAATGGGTCTGCAGC
TGCAAGAGAAGATAAGCAGGACTGCCGTCAGTTTTTGCATCTGTTGAGATTGTCTAGTGA
TGGACAGGAAATAAATCGAGGCCGTACTGAGTGGAGAAAGAAACCTGCGAGATGA,
Theobroma cacao oleoyl-acyl carrier protein thioesterase 1,
chloroplastic (TcFATA) truncated codon-optimized (corresponds
to nt 244-1128 of SEQ ID NO: 106), 888 nt
SEQ ID NO: 108
ATGCTGACCGAGGACGGCCTGTCGTACAAGGAGAAGTTCATCGTGCGCTGCTACGAGGT
GGGCATCAACAAGACCGCCACCGTGGAGACCATCGCCAACCTGCTGCAGGAGGTGGGCT
GCAACCACGCCCAGTCGGTGGGCTACTCGACCGACGGCTTCGCCACCACCCGCACCATG
CGCAAGCTGCACCTGATCTGGGTGACCGCCCGCATGCACATCGAGATCTACAAGTACCC
GGCCTGGTCGGACGTGATCGAGATCGAGACCTGGTGCCAGTCGGAGGGCCGCATCGGCA
CCCGCCGCGACTGGATCCTGAAGGACTTCGGCACCGGCGAGGTGATCGGCCGCGCCACC
TCGAAGTGGGTGATGATGAACCAGGACACCCGCCGCCTGCAGAAGGTGTCGGACGACGT
GCGCGAGGAGTACCTGGTGTTCTGCCCGCGCGAGCTGCGCCTGGCCTTCCCGGAGGAGA
ACAACAACTCGCTGAAGAAGATCGCCAAGCTGGACGACTCGTTCCAGTACTCGCGCCTG
GGCCTGATGCCGCGCCGCGCCGACCTGGACATGAACCAGCACGTGAACAACGTGACCTA
CATCGGCTGGGTGCTGGAGTCGATGCCGCAGGAGATCATCGACACCCACGAGCTGCAGA
CCATCACCCTGGACTACCGCCGCGAGTGCCAGCAGGACGACGTGGTGGACTCGCTGACC
TCGCCGGAGCAGGTGGAGGGCACCGAGAAGGTGTCGGCCATCCACGGCACCAACGGCTC
GGCCGCCGCCCGCGAGGACAAGCAGGACTGCCGCCAGTTCCTGCACCTGCTGCGCCTGT
CGTCGGACGGCCAGGAGATCAACCGCGGCCGCACCGAGTGGCGCAAGAAGCCGGCCCG
CTAA,
Theobroma cacao oleoyl-acyl carrier protein thioesterase 1,
chloroplastic (TcFATA) truncated (corresponds to nt 244-1128
of SEQ ID NO: 107), 888 nt
SEQ ID NO: 109
ATGTTGACGGAAGATGGTTTGTCCTATAAGGAGAAGTTTATTGTGAGGTGTTATGAGGTG
GGAATTAACAAAACTGCCACTGTTGAAACCATTGCCAATCTCTTGCAGGAGGTTGGATGT
AACCATGCTCAAAGTGTTGGATATTCCACAGATGGGTTTGCTACCACTCGCACCATGAGA
AAATTGCATCTCATTTGGGTAACTGCACGCATGCACATTGAAATATACAAATACCCTGCT
TGGAGTGATGTGATTGAAATAGAGACATGGTGCCAAAGTGAGGGAAGAATTGGAACCAG
AAGGGACTGGATTCTTAAGGACTTTGGAACTGGTGAAGTTATTGGAAGAGCTACTAGCA
AGTGGGTGATGATGAACCAGGACACTAGGCGGCTACAGAAAGTCAGTGATGATGTCAGG
GAAGAATATTTAGTCTTCTGTCCACGAGAACTCAGATTAGCATTTCCAGAGGAGAACAAT
AATAGTTTGAAGAAAATTGCCAAATTAGATGACTCTTTTCAGTATTCCAGGCTAGGGCTT
ATGCCAAGAAGAGCTGATCTGGACATGAACCAGCATGTCAATAATGTCACCTACATTGG
ATGGGTTCTGGAGAGCATGCCTCAAGAGATCATTGACACCCATGAACTGCAAACTATCA
CGTTAGATTACAGACGGGAATGCCAACAGGATGACGTGGTGGATTCACTTACCAGTCCA
GAACAAGTGGAGGGTACCGAAAAAGTTTCAGCGATTCACGGAACAAATGGGTCTGCAGC
TGCAAGAGAAGATAAGCAGGACTGCCGTCAGTTTTTGCATCTGTTGAGATTGTCTAGTGA
TGGACAGGAAATAAATCGAGGCCGTACTGAGTGGAGAAAGAAACCTGCGAGATGA,
Theobroma cacao palmitoyl-acyl carrier protein thioesterase,
chloroplastic (TcFatB1), NCBI Reference Sequence:
XM_007044056.2, 1188 nt
SEQ ID NO: 110
ATGGCTACTTTCTCTTGTCCAATCTCCTTCCCTTTCAGATGTTCTTTTAACGGTAATCATAA
CCATAACCACAGAGTCGATATCAAAATCAATGGAACTCATACAGGGCCCATTAAGCTCG
ACACATTGAATGAAATAGCTGCAGTTGTAAAAGCTGATTCCACTCCTCTTGCTAATGTCC
ACGAAAACGGGTACATATGCCAAGAGAAGATTCGGCAGAGGATTCCAACGCAGAAGCA
GCTGGTTGATCCTTACCGTCAAGGGCTTATCATTGAAAGGGGAGTTGGCTATAGACAGAC
TGTTGTCATCCGCTCCTATGAAGTTGGCCCTGATAAAACTGCTACCCTGGAGAGCCTCCT
TAATCTTTTCCAGGAAACAGCACTAAATCATGTCTGGATGTCTGGACTTCTGAGCAATGG
ATTTGGAGCCACACATGGAATGATGAGGAACAATCTCATATGGGTCGTCTCAAGAATGC
ACGTCCAAGTGCATCACTATCCCATATGGGGAGAGGTAGTGGAAATCGACACATGGGTT
GGAGCATCAGGGAAGAATGGGATGAGGCGAGACTGGCTAATTCGGAGTCAAGCCACCG
GCATCACCTACGCACGTGCAACCAGCACTTGGGTAATGATGAACGAGCAAACAAGGCGC
CTCTCAAAGATGCCGGAGGAAGTGAGGGGTGAAATCTCTCCTTGGTTTATTGAGAAGCA
AGCAATCAAAGAAGATGCTCCCGAGAAAATTGTCAAGTTGGACGACAAAGCAAAATATG
TGAACTCTGACTTGAAGCCAAAGAGGAGTGATTTGGACATGAACCACCATGTAAACAAT
GTCAAGTATGTACGATGGATGCTTGAGACAATTCCTGACAAATTTTTGGAGTCTCACCAG
CTATCTAGTATTGTACTAGAATATAGAAGGGAATGTGGGAGTTCGGATAAAGTTCAATCA
CTTTGCCAACCAGATGAAGATAGAATTTTAGCAAATGGACTGGAACAAAGTCTACTTGA
AAATATAGTTTTGGCATCGGGAATCATGCTAGGAAATGGACACCTAGGCTCCCTGGGTAT
GAAGACATATGGATTTACTCATCTCCTCCAAATCAAAGGGGACAGTCAAAATGACGAGA
TAGTCAGAGGGAGGACCAGATGGAAGAAAAAGCAATCTACCACGCCGTATTCCACTTAA,
Theobroma cacao palmitoyl-acyl carrier protein thioesterase,
chloroplastic (TcFatB1) truncated (corresponds to nt 280-1188
of SEQ ID NO: 110), 912 nt
SEQ ID NO: 111
ATGGGAGTTGGCTATAGACAGACTGTTGTCATCCGCTCCTATGAAGTTGGCCCTGATAAA
ACTGCTACCCTGGAGAGCCTCCTTAATCTTTTCCAGGAAACAGCACTAAATCATGTCTGG
ATGTCTGGACTTCTGAGCAATGGATTTGGAGCCACACATGGAATGATGAGGAACAATCT
CATATGGGTCGTCTCAAGAATGCACGTCCAAGTGCATCACTATCCCATATGGGGAGAGGT
AGTGGAAATCGACACATGGGTTGGAGCATCAGGGAAGAATGGGATGAGGCGAGACTGG
CTAATTCGGAGTCAAGCCACCGGCATCACCTACGCACGTGCAACCAGCACTTGGGTAAT
GATGAACGAGCAAACAAGGCGCCTCTCAAAGATGCCGGAGGAAGTGAGGGGTGAAATC
TCTCCTTGGTTTATTGAGAAGCAAGCAATCAAAGAAGATGCTCCCGAGAAAATTGTCAA
GTTGGACGACAAAGCAAAATATGTGAACTCTGACTTGAAGCCAAAGAGGAGTGATTTGG
ACATGAACCACCATGTAAACAATGTCAAGTATGTACGATGGATGCTTGAGACAATTCCTG
ACAAATTTTTGGAGTCTCACCAGCTATCTAGTATTGTACTAGAATATAGAAGGGAATGTG
GGAGTTCGGATAAAGTTCAATCACTTTGCCAACCAGATGAAGATAGAATTTTAGCAAAT
GGACTGGAACAAAGTCTACTTGAAAATATAGTTTTGGCATCGGGAATCATGCTAGGAAA
TGGACACCTAGGCTCCCTGGGTATGAAGACATATGGATTTACTCATCTCCTCCAAATCAA
AGGGGACAGTCAAAATGACGAGATAGTCAGAGGGAGGACCAGATGGAAGAAAAAGCAA
TCTACCACGCCGTATTCCACTTAA,
Theobroma cacao palmitoyl-acyl carrier protein thioesterase,
chloroplastic isoform X1 (TcFatB2), NCBI Reference Sequence:
XM_018116899.1, 1398 nt
SEQ ID NO: 112
ATGCACCAATGCCACTCGAATGCTCCCCTATTTGCGTGCAAGCTAACGACTGAGGTTTCT
CGATTTCTCCCCCACCCATTGGAAGTGGTTGAGCTGCAAATTGCATCTGCTCCTCTAGAT
GCTGGAGCTAGGGAGCTAGGGACTGCCATCTGTTTCTTTTTCCGAACTCGAAAGTTTGCT
AGCTTGGCTCTTTTTTTTTGTTCGCGGGTGAGCAAATGCGAGGCACTTACCATGGCATCA
ATGGCCAAAGCAAGCAATGTAACTTCATTATTTCTAGGGGGTGTATGCAAGGAAGAGAA
AACGAAGAATGTAGCCATGGCGAAGTTGGGTTTTTATTCTTCATGGAACTTGATCAAACC
GAAACGGAAAGGCCTTTTGCTAATTGCAAGTGCTAAAAATCCTCATAATCTGGACATGAT
CAACGGGAAAAAAGTAAATGGAATTTTTGTTGGTGAAGCTCCATATACGGGAAAGAAGA
GCACTGTGTTGATAAAAGAACACGTCCCTTATAAACAAGCCCACGCGGCGAGTTTGGTTG
GAAGGTTTGTGGAGGATAGGCATGTCTACAGGCAGACCTTCATCATCAGGTCTTATGAAA
CTGGACCGGACAAAACTGCCACCATGGAAACGGTTATGAATCTCCTTCAGGAAACAGCT
TTGAATCATGTAAGGAGCTCTGGTCTTGCTGGGAATGGCTTTGGGGCTACCCGTGAGATG
AGCCTTAGGAAACTCATATGGGTCGTCACCCGCATCCACGTTCAAGTGGAGAGATATAG
CTGCTGGGGAGATGTTGTGGAGATTGATACTTGGGTTGATGCAGCAGGAAAGAATGCAA
TGCGTAGGGACTGGATAATCAGAGACTACAATACACAGGAGATCATAACAAGAGCAACA
AGCACATGGGTGATTATGAACCACGAAACACGAAGATTAACCAAGATTCCTGAACAAGT
TAGGCAAGAAGTGATTCCATTCTACCTAAACAGGATTGCAATCGCTGAAGAGAAGAACG
ATATCGGAAAGATTGATAAGCTTACTGATGAAAACGCAGAAAGAATACGGTCTGGCTTA
GCTCCAAGATGGAGCGACATGGATGCCAATCAGCATGTAAACAATGTTAAATACATCGG
ATGGATTTTGGAGAGTGTGCCAATGGACGTCCTGGAAGAGTATCGTCTGACGAGCATGA
CCCTGGAGTATCGACGTGAATGCCGGAAATCCAATTTGCTAGAGTCCTTGACGAGTTCAA
CAGCAAATGTGACAGAAGATTCCAACAACAATTCTAATAATCGCAAGGCAGACTTGGAA
TACACACATCTGCTTCGCATGCAAGACGACGTGGCAGAGATAGTCCGAGCTAGATCAGA
ATGGCAATCCAAGGACAAACACAGCTGGTGA,
Theobroma cacao palmitoyl-acyl carrier protein thioesterase,
chloroplastic isoform X1 (TcFatB2) truncated (corresponds to
nt 547-1398 of SEQ ID NO: 112), 855 nt
SEQ ID NO: 113
ATGGTGGAGGATAGGCATGTCTACAGGCAGACCTTCATCATCAGGTCTTATGAAACTGG
ACCGGACAAAACTGCCACCATGGAAACGGTTATGAATCTCCTTCAGGAAACAGCTTTGA
ATCATGTAAGGAGCTCTGGTCTTGCTGGGAATGGCTTTGGGGCTACCCGTGAGATGAGCC
TTAGGAAACTCATATGGGTCGTCACCCGCATCCACGTTCAAGTGGAGAGATATAGCTGCT
GGGGAGATGTTGTGGAGATTGATACTTGGGTTGATGCAGCAGGAAAGAATGCAATGCGT
AGGGACTGGATAATCAGAGACTACAATACACAGGAGATCATAACAAGAGCAACAAGCA
CATGGGTGATTATGAACCACGAAACACGAAGATTAACCAAGATTCCTGAACAAGTTAGG
CAAGAAGTGATTCCATTCTACCTAAACAGGATTGCAATCGCTGAAGAGAAGAACGATAT
CGGAAAGATTGATAAGCTTACTGATGAAAACGCAGAAAGAATACGGTCTGGCTTAGCTC
CAAGATGGAGCGACATGGATGCCAATCAGCATGTAAACAATGTTAAATACATCGGATGG
ATTTTGGAGAGTGTGCCAATGGACGTCCTGGAAGAGTATCGTCTGACGAGCATGACCCTG
GAGTATCGACGTGAATGCCGGAAATCCAATTTGCTAGAGTCCTTGACGAGTTCAACAGC
AAATGTGACAGAAGATTCCAACAACAATTCTAATAATCGCAAGGCAGACTTGGAATACA
CACATCTGCTTCGCATGCAAGACGACGTGGCAGAGATAGTCCGAGCTAGATCAGAATGG
CAATCCAAGGACAAACACAGCTGGTGA,
Theobroma cacao palmitoyl-acyl carrier protein thioesterase,
chloroplastic isoform X2, (TcFatB3), NCBI Reference Sequence:
XM_018116900.1, 1167 nt
SEQ ID NO: 114
ATGGCATCAATGGCCAAAGCAAGCAATGTAACTTCATTATTTCTAGGGGGTGTATGCAAG
GAAGAGAAAACGAAGAATGTAGCCATGGCGAAGTTGGGTTTTTATTCTTCATGGAACTT
GATCAAACCGAAACGGAAAGGCCTTTTGCTAATTGCAAGTGCTAAAAATCCTCATAATCT
GGACATGATCAACGGGAAAAAAGTAAATGGAATTTTTGTTGGTGAAGCTCCATATACGG
GAAAGAAGAGCACTGTGTTGATAAAAGAACACGTCCCTTATAAACAAGCCCACGCGGCG
AGTTTGGTTGGAAGGTTTGTGGAGGATAGGCATGTCTACAGGCAGACCTTCATCATCAGG
TCTTATGAAACTGGACCGGACAAAACTGCCACCATGGAAACGGTTATGAATCTCCTTCAG
GAAACAGCTTTGAATCATGTAAGGAGCTCTGGTCTTGCTGGGAATGGCTTTGGGGCTACC
CGTGAGATGAGCCTTAGGAAACTCATATGGGTCGTCACCCGCATCCACGTTCAAGTGGA
GAGATATAGCTGCTGGGGAGATGTTGTGGAGATTGATACTTGGGTTGATGCAGCAGGAA
AGAATGCAATGCGTAGGGACTGGATAATCAGAGACTACAATACACAGGAGATCATAACA
AGAGCAACAAGCACATGGGTGATTATGAACCACGAAACACGAAGATTAACCAAGATTCC
TGAACAAGTTAGGCAAGAAGTGATTCCATTCTACCTAAACAGGATTGCAATCGCTGAAG
AGAAGAACGATATCGGAAAGATTGATAAGCTTACTGATGAAAACGCAGAAAGAATACG
GTCTGGCTTAGCTCCAAGATGGAGCGACATGGATGCCAATCAGCATGTAAACAATGTTA
AATACATCGGATGGATTTTGGAGAGTGTGCCAATGGACGTCCTGGAAGAGTATCGTCTG
ACGAGCATGACCCTGGAGTATCGACGTGAATGCCGGAAATCCAATTTGCTAGAGTCCTTG
ACGAGTTCAACAGCAAATGTGACAGAAGATTCCAACAACAATTCTAATAATCGCAAGGC
AGACTTGGAATACACACATCTGCTTCGCATGCAAGACGACGTGGCAGAGATAGTCCGAG
CTAGATCAGAATGGCAATCCAAGGACAAACACAGCTGGTGA,
Theobroma cacao palmitoyl-acyl carrier protein thioesterase,
chloroplastic isoform X2, (TcFatB3) truncated (corresponds to
nt 316-1167 of SEQ ID NO: 114), 855 nt
SEQ ID NO: 115
ATGGTGGAGGATAGGCATGTCTACAGGCAGACCTTCATCATCAGGTCTTATGAAACTGG
ACCGGACAAAACTGCCACCATGGAAACGGTTATGAATCTCCTTCAGGAAACAGCTTTGA
ATCATGTAAGGAGCTCTGGTCTTGCTGGGAATGGCTTTGGGGCTACCCGTGAGATGAGCC
TTAGGAAACTCATATGGGTCGTCACCCGCATCCACGTTCAAGTGGAGAGATATAGCTGCT
GGGGAGATGTTGTGGAGATTGATACTTGGGTTGATGCAGCAGGAAAGAATGCAATGCGT
AGGGACTGGATAATCAGAGACTACAATACACAGGAGATCATAACAAGAGCAACAAGCA
CATGGGTGATTATGAACCACGAAACACGAAGATTAACCAAGATTCCTGAACAAGTTAGG
CAAGAAGTGATTCCATTCTACCTAAACAGGATTGCAATCGCTGAAGAGAAGAACGATAT
CGGAAAGATTGATAAGCTTACTGATGAAAACGCAGAAAGAATACGGTCTGGCTTAGCTC
CAAGATGGAGCGACATGGATGCCAATCAGCATGTAAACAATGTTAAATACATCGGATGG
ATTTTGGAGAGTGTGCCAATGGACGTCCTGGAAGAGTATCGTCTGACGAGCATGACCCTG
GAGTATCGACGTGAATGCCGGAAATCCAATTTGCTAGAGTCCTTGACGAGTTCAACAGC
AAATGTGACAGAAGATTCCAACAACAATTCTAATAATCGCAAGGCAGACTTGGAATACA
CACATCTGCTTCGCATGCAAGACGACGTGGCAGAGATAGTCCGAGCTAGATCAGAATGG
CAATCCAAGGACAAACACAGCTGGTGA,
Theobroma cacao palmitoyl-acyl carrier protein thioesterase,
chloroplastic isoform X3 (TcFatB4), NCBI Reference Sequence:
XM_018116901.1, 1158 nt
SEQ ID NO: 116
ATGCACCAATGCCACTCGAATGCTCCCCTATTTGCGTGCAAGCTAACGACTGAGGTTTCT
CGATTTCTCCCCCACCCATTGGAAGTGGTTGAGCTGCAAATTGCATCTGCTCCTCTAGAT
GCTGGAGCTAGGGAGCTAGGGACTGCCATCTGTTTCTTTTTCCGAACTCGAAAGTTTGCT
AGCTTGGCTCTTTTTTTTTGTTCGCGGGTGAGCAAATGCGAGGCACTTACCATGGCATCA
ATGGCCAAAGCAAGCAATGTAACTTCATTATTTCTAGGGGGTGTATGCAAGGAAGAGAA
AACGAAGAATGTAGCCATGGCGAAGTTGGGTTTTTATTCTTCATGGAACTTGATCAAACC
GAAACGGAAAGGCCTTTTGCTAATTGCAAGTGCTAAAAATCCTCATAATCTGGACATGAT
CAACGGGAAAAAAGTAAATGGAATTTTTGTTGGTGAAGCTCCATATACGGGAAAGAAGA
GCACTGTGTTGATAAAAGAACACGTCCCTTATAAACAAGCCCACGCGGCGAGTTTGGTTG
GAAGGTTTGTGGAGGATAGGCATGTCTACAGGCAGACCTTCATCATCAGGTCTTATGAAA
CTGGACCGGACAAAACTGCCACCATGGAAACGGTTATGAATCTCCTTCAGGAAACAGCT
TTGAATCATGTAAGGAGCTCTGGTCTTGCTGGGAATGGCTTTGGGGCTACCCGTGAGATG
AGCCTTAGGAAACTCATATGGGTCGTCACCCGCATCCACGTTCAAGTGGAGAGATATAG
CTGCTGGGGAGATGTTGTGGAGATTGATACTTGGGTTGATGCAGCAGGAAAGAATGCAA
TGCGTAGGGACTGGATAATCAGAGACTACAATACACAGGAGATCATAACAAGAGCAACA
AGCACATGGGTGATTATGAACCACGAAACACGAAGATTAACCAAGATTCCTGAACAAGT
TAGGCAAGAAGTGATTCCATTCTACCTAAACAGGATTGCAATCGCTGAAGAGAAGAACG
ATATCGGAAAGATTGATAAGCTTACTGATGAAAACGCAGAAAGAATACGGTCTGGCTTA
GCTCCAAGATGGAGCGACATGGATGCCAATCAGCATGTAAACAATGTTAAATACATCGG
ATGGATTTTGGAGGCATTCACCAACTAA,
Theobroma cacao palmitoyl-acyl carrier protein thioesterase,
chloroplastic isoform X3 (TcFatB4) truncated (corresponds to nt
547-1158 of SEQ ID NO: 116), 615 nt
SEQ ID NO: 117
ATGGTGGAGGATAGGCATGTCTACAGGCAGACCTTCATCATCAGGTCTTATGAAACTGG
ACCGGACAAAACTGCCACCATGGAAACGGTTATGAATCTCCTTCAGGAAACAGCTTTGA
ATCATGTAAGGAGCTCTGGTCTTGCTGGGAATGGCTTTGGGGCTACCCGTGAGATGAGCC
TTAGGAAACTCATATGGGTCGTCACCCGCATCCACGTTCAAGTGGAGAGATATAGCTGCT
GGGGAGATGTTGTGGAGATTGATACTTGGGTTGATGCAGCAGGAAAGAATGCAATGCGT
AGGGACTGGATAATCAGAGACTACAATACACAGGAGATCATAACAAGAGCAACAAGCA
CATGGGTGATTATGAACCACGAAACACGAAGATTAACCAAGATTCCTGAACAAGTTAGG
CAAGAAGTGATTCCATTCTACCTAAACAGGATTGCAATCGCTGAAGAGAAGAACGATAT
CGGAAAGATTGATAAGCTTACTGATGAAAACGCAGAAAGAATACGGTCTGGCTTAGCTC
CAAGATGGAGCGACATGGATGCCAATCAGCATGTAAACAATGTTAAATACATCGGATGG
ATTTTGGAGGCATTCACCAACTAA,
Theobroma cacao palmitoyl-acyl carrier protein thioesterase,
chloroplastic, (TcFatB5), NCBI Reference Sequence:
XM_007023975.2, 1131 nt
SEQ ID NO: 118
ATGGCAGCATCTTCAAACATTATGACATCGAAGTTCTTCATGGCGACCTCTCCCTCATCCT
GGAATTCAACGAATAAATCAAAGATTTGCTTGCAACAAATCGATAGAAGTTCAAATACG
AATGGAAAGATGGTTAAGTTTACTCGAGACAGTAGTTTGAAGGTCAAATTGCAGGCTCA
AGCACTGCTTACTAATGACAGTAGAGCAACATCAATGATAGAAAGCCTGAAGGATGAGG
AAATGATGACTTCACCACCCGCAACAATGGAACACCTGACAACGGAAGGAAGATTGATA
AATGACGGACTGGTTTTCCAGCAGAATTTTTCCATTAGGTCATTCGAGATAGACTCTGAG
TACAAAGTTTCAGCAAGGGCTATAATGAATTATTTGCAGGAGTCATCACTTAACCATAGA
AAGAAGATGGGGATGTCAAGCGATTCCCTTGTAGGTGTAACGCCAGAGATGATTAAAAG
GGACTTGCTATGGATATTCCGTGGCATGTGTATTGAGGTGGATCGCTATCCTTCTTGGGCT
GATGTTGTCCAGATATACCACCGGATCTATACATCAGGAAGGACTGGTTTGCGTTTGGAA
TGGATTGTCAATGACAGCAAGACAGGCGAAACTCTAGTTCGAGCATCATGCTTAGCTGTG
ATGATGAATAAGAAAACAAGAAAAACATGCAAGTTTCCGGAGGAAGTCAAACAGGAGC
TAAAGCCTTATCTTACGACAGACGCTGAGCCTCTCTTCGAAGCTGACAAAATTTTATGTC
CCCAAGTTGGGAAAATGGATAACATCCGAACCGGATTGACTTCCTGTTGGCATGACCTGG
ATTTCAATTACCATGTAAACAATGCAAAGTATCTTGACTGGATTTTGGAGGGTACTCCTA
CCTCTCTTATACATAGCCATGAGCTTTCTAGAGTAAGTCTCCAGTACCGAAAGGAGTGCT
TGAAAGATGATGTGATTCAATCTTTATCCAGAGTTGTTACCAAGGAAACTGGTCTCTCAA
CGAACAATCAAGAAATTGAATTAGAACACGTCCTCCGTCTTGAGAGTGGACCGGAACTT
GCAGGGGCAAGGACCGCTTGGAGGCCAAAATCTATCTGCCGACAAACTATAAATTAA
Theobroma cacao palmitoyl-acyl carrier protein thioesterase,
chloroplastic, (TcFatB5) truncated (corresponds to nt 292-1131
of SEQ ID NO: 118), 843 nt
SEQ ID NO: 119
ATGTTGATAAATGACGGACTGGTTTTCCAGCAGAATTTTTCCATTAGGTCATTCGAGATA
GACTCTGAGTACAAAGTTTCAGCAAGGGCTATAATGAATTATTTGCAGGAGTCATCACTT
AACCATAGAAAGAAGATGGGGATGTCAAGCGATTCCCTTGTAGGTGTAACGCCAGAGAT
GATTAAAAGGGACTTGCTATGGATATTCCGTGGCATGTGTATTGAGGTGGATCGCTATCC
TTCTTGGGCTGATGTTGTCCAGATATACCACCGGATCTATACATCAGGAAGGACTGGTTT
GCGTTTGGAATGGATTGTCAATGACAGCAAGACAGGCGAAACTCTAGTTCGAGCATCAT
GCTTAGCTGTGATGATGAATAAGAAAACAAGAAAAACATGCAAGTTTCCGGAGGAAGTC
AAACAGGAGCTAAAGCCTTATCTTACGACAGACGCTGAGCCTCTCTTCGAAGCTGACAA
AATTTTATGTCCCCAAGTTGGGAAAATGGATAACATCCGAACCGGATTGACTTCCTGTTG
GCATGACCTGGATTTCAATTACCATGTAAACAATGCAAAGTATCTTGACTGGATTTTGGA
GGGTACTCCTACCTCTCTTATACATAGCCATGAGCTTTCTAGAGTAAGTCTCCAGTACCG
AAAGGAGTGCTTGAAAGATGATGTGATTCAATCTTTATCCAGAGTTGTTACCAAGGAAAC
TGGTCTCTCAACGAACAATCAAGAAATTGAATTAGAACACGTCCTCCGTCTTGAGAGTGG
ACCGGAACTTGCAGGGGCAAGGACCGCTTGGAGGCCAAAATCTATCTGCCGACAAACTA
TAAATTAA,
Theobroma cacao palmitoyl-acyl carrier protein thioesterase,
chloroplastic, (TcFatB6), NCBI Reference Sequence:
XM_007013216.2, 1263 nt
SEQ ID NO: 120
ATGGTTGCTACTGCTGCATCATCTGCATTCTTTCCGGTCACTTCATCCCCGGACTCCTCTG
ACTCAAAAAACAAGAAGCTTGGAAGTGGATCTACTAACCTTGGAGGTATCAAGTCGAAA
CCATCTACTCCTTCTGGAATTTTGCAAGTCAAGGCAAATGCTCAAGCTCCTCCAAAAATA
AATGGTACCACGGTCGTGACAACTCCAGTGGAAAGTTTCAAGAATGAGGACACTGCTAG
TTCCCCTCCTCCCAGGACATTTATAAACCAGTTACCTGATTGGAGCATGCTTCTTGCTGCT
ATCACGACAATTTTCTTGGCTGCTGAGAAGCAGTGGATGATGCTTGATTGGAAACCCAGG
CGGCCTGACATGCTCATTGATCCATTTGGTATAGGGAGGATTGTTCAGGATGGTCTTGTT
TTCCGCCAGAATTTCTCTATAAGGTCTTACGAGATAGGTGCTGATCGGACAGCATCCATA
GAGACGCTAATGAATCATTTACAGGAAACGGCTATTAATCATTGTAGAAGCGCTGGACT
GCTTGGAGAAGGTTTTGGTTCAACCCCTGAGATGTGCAAGAAAAACCTAATATGGGTAG
TCACTCGCATGCAAGTIGTGGTTGATCGCTATCCTACATGGGGTGATGTTGTTCAAGTAG
ACACTTGGGCCAGTGCATCGGGAAAGAATGGTATGCGACGGGATTGGCTTGTCAGTGAT
AGTAAAACTGGTGAAATTTTAACAAGAGCCTCAAGTGTATGGGTGATGATGAATAAGCT
TACTAGAAGGTTATCTAAAATTCCAGAAGAGGTCCGAGGAGAAATAGAACCTTATTTTAT
GAATTCTGATCCTGTTGTGGCTGAGGATAGTAGGAAATTAGTGAAACTTGATGACAGCAC
AGCAGATTATGTCCGTAAAGGTTTAACTCCCAGATGGGGTGATTTGGATGTCAACCAGCA
TGTCAATAATGTGAAGTACATTGGCTGGATCCTTGAGAGTGCTCCACTGCCAATCTTGGA
GACTCACGAGCTTTCTTCAATGACACTGGAATATAGGAGGGAGTGTGGGAGAGACGGTG
TGCTGCAGTCCCTAACTGCTGTCTCTGACTCTGGTGTGGGCAACTTGGTGAACTTTGGTG
AAATCGAGTGCCAGCACTTGCTCCGACTCGAGGATGGGTCTGAGATTGTGAGAGGGAGG
ACTGAGTGGAGGCCGAAGTATGCGAAAAGTTTTGGTAATGTGGGCCAACTTCCTGCTGA
AAGTGCATAG,
Theobroma cacao palmitoyl-acyl carrier protein thioesterase,
chloroplastic, (TcFatB6) truncated (corresponds to nt 400-1263
of SEQ ID NO: 120), 867 nt
SEQ ID NO: 121
ATGATTGTTCAGGATGGTCTTGTTTTCCGCCAGAATTTCTCTATAAGGTCTTACGAGATAG
GTGCTGATCGGACAGCATCCATAGAGACGCTAATGAATCATTTACAGGAAACGGCTATT
AATCATTGTAGAAGCGCTGGACTGCTTGGAGAAGGTTTTGGTTCAACCCCTGAGATGTGC
AAGAAAAACCTAATATGGGTAGTCACTCGCATGCAAGTTGTGGTTGATCGCTATCCTACA
TGGGGTGATGTTGTTCAAGTAGACACTTGGGCCAGTGCATCGGGAAAGAATGGTATGCG
ACGGGATTGGCTTGTCAGTGATAGTAAAACTGGTGAAATTTTAACAAGAGCCTCAAGTGT
ATGGGTGATGATGAATAAGCTTACTAGAAGGTTATCTAAAATTCCAGAAGAGGTCCGAG
GAGAAATAGAACCTTATTTTATGAATTCTGATCCTGTTGTGGCTGAGGATAGTAGGAAAT
TAGTGAAACTTGATGACAGCACAGCAGATTATGTCCGTAAAGGTTTAACTCCCAGATGG
GGTGATTTGGATGTCAACCAGCATGTCAATAATGTGAAGTACATTGGCTGGATCCTTGAG
AGTGCTCCACTGCCAATCTTGGAGACTCACGAGCTTTCTTCAATGACACTGGAATATAGG
AGGGAGTGTGGGAGAGACGGTGTGCTGCAGTCCCTAACTGCTGTCTCTGACTCTGGTGTG
GGCAACTTGGTGAACTTTGGTGAAATCGAGTGCCAGCACTTGCTCCGACTCGAGGATGG
GTCTGAGATTGTGAGAGGGAGGACTGAGTGGAGGCCGAAGTATGCGAAAAGTTTTGGTA
ATGTGGGCCAACTTCCTGCTGAAAGTGCATAG,

In some embodiments of any of the aspects, the amino acid sequence encoded by the functional thioesterase gene comprises one of SEQ ID NOs: 16-21, 68, 70, 123-139 or an amino acid sequence that is at least 80%, at least 85%, at least 90%, at least 91%, at least 92%, at least 93%, at least 94%, at least 95%, at least 96%, at least 97%, at least 98%, or at least 99% identical to the sequence of at least one of SEQ ID NOs: 16-21, 68, 70, 123-139, that maintains the same functions as at least one of SEQ ID NOs: 16-21, 68, 70, 123-139 (e.g., thioesterase).

Engineered chimera of C. palustris FatB1(aa 1-218) and FatB2 (aa 219-
316) thioesterase-Chimera 4 (326 aa)
SEQ ID NO: 16
MLLTAITTVFVAPEKRWTMFDRKSKRPNMLMDSFGLERVVQDGLVFRQSFSIRSYEICADRT
ASIETVMNHVQETSLNQCKSIGLLDDGFGRSPEMCKRDLIWVVTRMKIMVNRYPTWGDTIEV
STWLSQSGKIGMGRDWLISDCNTGEILVRATSVYAMMNQKTRRFSKLPHEVRQEFAPHFLDS
PPAIEDNDGKLQKFDVKTGDSIRKGLTPGWYDLDVNQHVNNVKYIGWILQSVPTEVFETQEL
CGLTLEYRRECGRDSVLESVTAMDPSKEGDRSLYQHLLRLEDGADIVKGRTEWRPKNAGAK
GAILTGKTSGGHHHHHH,
Cuphea palustris FatB1, GenBank: AAC49179.1, 411 aa; bolded text
corresponds to SEQ ID NO: 18 (e.g., residues 96-411 of SEQ ID NO: 17)
SEQ ID NO: 17
MVAAAASSACFPVPSPGASPKPGKLGNWSSSLSPSLKPKSIPNGGFQVKANASAHPKANG
SAVTLKSGSLNTQEDTLSSSPPPRAFFNQLPDWSMLLTAITTVFVAPEKRWTMFDRKSKR
PNMLMDSFGLERVVQDGLVFRQSFSIRSYEICADRTASIETVMNHVQETSLNQCKSIGLL
DDGFGRSPEMCKRDLIWVVTRMKIMVNRYPTWGDTIEVSTWLSQSGKIGMGRDWLIS
DCNTGEILVRATSVYAMMNQKTRRFSKLPHEVRQEFAPHFLDSPPAIEDNDGKLQKFDV
KTGD
SIRKGLTPGWYDLDVNQHVSNVKYIGWILESMPTEVLETQELCSLTLEYRRECGRDSVL
E
SVTSMDPSKVGDRFQYRHLLRLEDGADIMKGRTEWRPKNAGTNGAISTGKT,
Cuphea palustris FatB1, fragment, 316 aa, corresponds to bolded text
of SEQ ID NO: 17 (e.g., residues 96-411 of SEQ ID NO: 17); italicized
text corresponds to portion in SEQ ID NO: 21 (e.g., residues
1-218 of SEQ ID NO: 18)
SEQ ID NO: 18
LLTAITTVFVAPEKRWTMFDRKSKRPNMLMDSFGLERVVQDGLVFRQSFSIRSYEICADRTASIETV
MNHVQETSLNQCKSIGLLDDGFGRSPEMCKRDLIWVVTRMKIMVNRYPTWGDTIEVSTWLSQSGK
IGMGRDWLISDCNTGEILVRATSVYAMMNQKTRRFSKLPHEVRQEFAPHFLDSPPAIEDNDGKLQ
KFDVKTGDSIRKGLTPGWYDLDVNQHVSNVKYIGWILESMPTEVLETQELCSLTLEYRRECGR
DSVLESVTSMDPSKVGDRFQYRHLLRLEDGADIMKGRTEWRPKNAGTNGAISTGKT,
Cuphea palustris FatB2, GenBank: AAC49180.1, 411 aa; bolded text
corresponds to SEQ ID NO: 20 (e.g., residues 90-404 of SEQ ID NO: 19)
SEQ ID NO: 19
MVAAAASAAFFSVATPRTNISPSSLSVPFKPKSNHNGGFQVKANASAHPKANGSAVSLKS
GSLETQEDKTSSSSPPPRTFINQLPVWSMLLSAVTTVFGVAEKQWPMLDRKSKRPDMLVE
PLGVDRIVYDGVSFRQSFSIRSYEIGADRTASIETLMNMFQETSLNHCKIIGLLNDGFGR
TPEMCKRDLIWVVTKMQIEVNRYPTWGDTIEVNTWVSASGKHGMGRDWLISDCHTGE
ILI
RATSVWAMMNQKTRRLSKIPYEVRQEIEPQFVDSAPVIVDDRKFHKLDLKTGDSICNGL
T
PRWTDLDVNQHVNNVKYIGWILQSVPTEVFETQELCGLTLEYRRECGRDSVLESVTAM
DP
SKEGDRSLYQHLLRLEDGADIVKGRTEWRPKNAGAKGAILTGKT SNGNSIS,
Cuphea palustris FatB2, fragment, 315 aa, corresponds to bolded text
of SEQ ID NO: 19 (e.g., residues 90-404 of SEQ ID NO: 19); italicized
text corresponds to portion in SEQ ID NO: 21 (e.g., residues
218-315 of SEQ ID NO: 20)
SEQ ID NO: 20
LLSAVTTVFGVAEKQWPMLDRKSKRPDMLVEPLGVDRIVYDGVSFRQSFSIRSYEIGADRTA
SIETLMNMFQETSLNHCKIIGLLNDGFGRTPEMCKRDLIWVVTKMQIEVNRYPTWGDTIEVN
TWVSASGKHGMGRDWLISDCHTGEILIRATSVWAMMNQKTRRLSKIPYEVRQEIEPQFVDSA
PVIVDDRKFHKLDLKTGDSICNGLTPRWTDLDVNQHVNNVKYIGWILQSVPTEVFETQELCGLT
LEYRRECGRDSVLESVTAMDPSKEGDRSLYQHLLRLEDGADIVKGRTEWRPKNAGAKGAILTGKT,
Cuphea palustris FatB2-FatB1 hybrid, 316 aa; bolded text corresponds
to italicized text of SEQ ID NO: 18 (e.g., residues 1-218 of SEQ ID
NO: 18); and plain text corresponds to italicized text of SEQ
ID NO: 20 (e.g., residues 218-315 of SEQ ID NO: 20)
SEQ ID NO: 21
LLTAITTVFVAPEKRWTMFDRKSKRPNMLMDSFGLERVVQDGLVFRQSFSIRSYEICAD
RTASIETVMNHVQETSLNQCKSIGLLDDGFGRSPEMCKRDLIWVVTRMKIMVNRYPTW
GDTIEVSTWLSQSGKIGMGRDWLISDCNTGEILVRATSVYAMMNQKTRRFSKLPHEVR
QEFAPHFLDSPPAIEDNDGKLQKFDVKTGDSIRKGLTPGWYDLDVNQHVNNVKYIGWIL
QSVPTEVFETQELCGLTLEYRRECGRDSVLESVTAMDPSKEGDRSLYQHLLRLEDGADIVKG
RTEWRPKNAGAKGAILTGKT,
Arachis hypogaea Acyl-[acyl-carrier-protein] hydrolase OS = Arachis
hypogaea OX = 3818 GN = FatB2-1 PE = 2 SV = 1 (AhFatB2-1) Ref. No.
tr|A0A444X7E1|A0A444X7E1_ARAHY (corresponds to SEQ ID NO: 99 or 100),
414 aa
SEQ ID NO: 123
MVATAATSSFFPVTSRTGGEGGGGIPASLGGGLKQNHRSSSVKANAHAPSKINGTATKVPKS
MESMKLESSSTTGANAPRTFINQIPDWSMLLAAITTAFLAAEKQWMMIDWKPKRSDVLSDPF
GIGRIVQDGLAFRQNFSIRSYEIGADKTASIETLMNHLQETALNHVKTAGLLGDGFGSTPEMC
KKNLIWVVTRMQVEVDRYPTWGDVVQVDTWVSASGKNGMRRDWIIRDANTGEILTRASSI
WVMMNKVTRRLSKIPEEVRQEIASYFVDSPPVVEEDNRKLSKLDDTADHIRRGLSPRWSDLD
VNQHVNNVKYIGWLLESAPQAILESHELRAMTLEYRRECGKDSVLDSLTDVSGADIGNLAG
GGSLECKHLLRLEDGGEIVRGRTEWRPKPVNNFGAMNQVFPAEN,
Arachis hypogaea Acyl-[acyl-carrier-protein] hydrolase (AhFatB2-1)
truncated (corresponds to SEQ ID NO: 101 or 102; corresponds to aa
63-414 of SEQ ID NO: 123), 352 aa
SEQ ID NO: 124
MESMKLESSSTTGANAPRTFINQIPDWSMLLAAITTAFLAAEKQWMMIDWKPKRSDVLSDPF
GIGRIVQDGLAFRQNFSIRSYEIGADKTASIETLMNHLQETALNHVKTAGLLGDGFGSTPEMC
KKNLIWVVTRMQVEVDRYPTWGDVVQVDTWVSASGKNGMRRDWIIRDANTGEILTRASSI
WVMMNKVTRRLSKIPEEVRQEIASYFVDSPPVVEEDNRKLSKLDDTADHIRRGLSPRWSDLD
VNQHVNNVKYIGWLLESAPQAILESHELRAMTLEYRRECGKDSVLDSLTDVSGADIGNLAG
GGSLECKHLLRLEDGGEIVRGRTEWRPKPVNNFGAMNQVFPAEN,
Mangifera indica palmitoyl-acyl carrier protein thioesterase,
chloroplastic-like (MiFatA) Ref. No. XP_044494686.1 (corresponds
to SEQ ID NO: 103), 382 aa
SEQ ID NO: 125
MTSVACKIILSRELFKEEKKIKPMATAKVGLCSSGNLIRRKHGRHLLIASASNPNGLDMMKG
KKVNGIHHNEETHHQLLLKQRVSKAPLHACLLGRFVGDRFMYRQTFIIRSYEIGPDKTATME
TLLNLLQETALNHVTGSGLAGNGFGATREMSLRKLIWVVTRINIQVQRYSCWGDVVEIDTW
VDSSGKNAMRRDWIIRDYHTQEIITRATSTWVTMNRETRRLSKIPEQVKQEVFPFYLDRVAIA
KEQNDVGKIDKLTDETAERIRSGLAPRWNDMDANQHVNNVKYIGWILESVPIHVLKDYNMT
SMTLEYRRECRQSNLLESLTSSTASVTGDPNNNSNNRIADLKYTHLLRMQADKAEIVRARSE
WQSKQITQVIT,
Morella rubra Palmitoyl-acyl carrier protein thioesterase,
chloroplastic (MrFatA) Ref. No. KAB1217487.1 (corresponds to SEQ
ID NO: 104), 433 aa
SEQ ID NO: 126
MLETFIFCLLIRQREFEATSATLYFQTYILLGSTISRGIVYLSVQSAMTVMAALSNARLYFGGD
FCRGDKKNMAMARLGYYPSLNCAIKPKQPSLLVIASASTPPSIYTINGKKVNGINFGEAPFRS
NKYSDSAKESCVDAPLHAVLLGRFVEDQFVYRQTFIIRSYEIGPDKTATMETLMNLLQETAL
NHVTSSGLAGNGFGATREMSLRKLIWVVTRVHIQVQRYSCWGDVVEIDTWVDAEGKNGMR
RDWIIRDYHTQEIITRATSTWVIMNQETRRLSKIPEQVREEVVPFYLDRLAVSAEMNDSEKID
KLTDETAERIRSGLAPRWSDMDANQHVNNVKYIGWILESVPINVLGNYDLTSMTLEYRRECR
QSNLLESLTSSTANATEASYNSLHRSKPDLEFTHLLRMLADKAEIVRARTQWHSKQKRN,
Pistacia vera palmitoyl-acyl carrier protein thioesterase,
chloroplastic-like (PvFatA) Ref. No. XP_031247728.1 (corresponds
to SEQ ID NO: 105) 386 aa
SEQ ID NO: 127
MISVARTSYVRLSFPENLFKEEKEIVPMAMAKVGFCCSLNLIRPKHGRLLVIASASNAKSLDI
MNGKKVNGIHVNEETRHQRLLNQRVADAPLHACLLGKFVEDRFLYRQTFIVRSYEIGPDKTA
TMETLLNLLQETALNHVTSSGLAGNGFGATREMSVRKLIWVVTRINIQVQRYSCWGDVVEID
TWVDAAGKNAMRRDWIIRDYRTQEIITRATSTWVIMNRETRRLSKIPEQVRQEVLPFYLGRV
AIAKEQNDVGKIDKLTDETAERIRSGLAPRWNDMDANQHVNNVKYIGWILESVPIHVLKDY
NLTSMTLEYRRECRQSNLLESLTSSTASVTGDPNNNSNNRIADLEYTHLLRMQADKAEIVRA
RSEWQSKQITQAIT,
Theobroma cacao oleoyl-acyl carrier protein thioesterase 1,
chloroplastic ID = Tc01v2_p018360.1|Name = Tc01v2_p018360.1|
organism = Theobroma cacao|type = polypeptide|length = 375 bp
(TcFATA) Ref. No. Tc01v2_p018360.1, NCBI Reference Sequence:
XP_007049712.2 (corresponds to SEQ ID NO: 106 or 107), 375 aa
SEQ ID NO: 128
MLKLSSCNVTDQRQALAQCRFLAPPAPFSFRWRTPVVVSCSPSSRPNLSPLQVVLSGQQQAG
MELVESGSGSLADRLRLGSLTEDGLSYKEKFIVRCYEVGINKTATVETIANLLQEVGCNHAQS
VGYSTDGFATTRTMRKLHLIWVTARMHIEIYKYPAWSDVIEIETWCQSEGRIGTRRDWILKD
FGTGEVIGRATSKWVMMNQDTRRLQKVSDDVREEYLVFCPRELRLAFPEENNNSLKKIAKL
DDSFQYSRLGLMPRRADLDMNQHVNNVTYIGWVLESMPQEIIDTHELQTITLDYRRECQQD
DVVDSLTSPEQVEGTEKVSAIHGTNGSAAAREDKQDCRQFLHLLRLSSDGQEINRGRTEWRK
KPAR,
Theobroma cacao oleoyl-acyl carrier protein thioesterase 1,
chloroplastic (TcFATA) truncated (corresponds to SEQ ID NO: 108
or 109; corresponds to aa 82-375 of SEQ ID NO: 128), 295 aa
SEQ ID NO: 129
MLTEDGLSYKEKFIVRCYEVGINKTATVETIANLLQEVGCNHAQSVGYSTDGFATTRTMRKL
HLIWVTARMHIEIYKYPAWSDVIEIETWCQSEGRIGTRRDWILKDFGTGEVIGRATSKWVMM
NQDTRRLQKVSDDVREEYLVFCPRELRLAFPEENNNSLKKIAKLDDSFQYSRLGLMPRRADL
DMNQHVNNVTYIGWVLESMPQEIIDTHELQTITLDYRRECQQDDVVDSLTSPEQVEGTEKVS
AIHGTNGSAAAREDKQDCRQFLHLLRLSSDGQEINRGRTEWRKKPAR,
Theobroma cacao palmitoyl-acyl carrier protein thioesterase,
chloroplastic ID = Tc02v2_p018460.1|Name = Tc02v2_p018460.1|
organism = Theobroma cacao|type = polypeptide|length = 395 bp
(TcFatB1) Ref. No. Tc02v2_p018460.1, NCBI Reference
Sequence: XP_007044118.2 (corresponds to SEQ ID NO: 110), 395 aa
SEQ ID NO: 130
MATFSCPISFPFRCSFNGNHNHNHRVDIKINGTHTGPIKLDTLNEIAAVVKADSTPLANVHEN
GYICQEKIRQRIPTQKQLVDPYRQGLIIERGVGYRQTVVIRSYEVGPDKTATLESLLNLFQETA
LNHVWMSGLLSNGFGATHGMMRNNLIWVVSRMHVQVHHYPIWGEVVEIDTWVGASGKNG
MRRDWLIRSQATGITYARATSTWVMMNEQTRRLSKMPEEVRGEISPWFIEKQAIKEDAPEKI
VKLDDKAKYVNSDLKPKRSDLDMNHHVNNVKYVRWMLETIPDKFLESHQLSSIVLEYRREC
GSSDKVQSLCQPDEDRILANGLEQSLLENIVLASGIMLGNGHLGSLGMKTYGFTHLLQIKGDS
QNDEIVRGRTRWKKKQSTTPYST,
Theobroma cacao palmitoyl-acyl carrier protein thioesterase,
chloroplastic (TcFatB1) truncated (corresponds to SEQ ID NO: 111;
corresponds to aa 94-395 of SEQ ID NO: 130), 303 aa
SEQ ID NO: 131
MGVGYRQTVVIRSYEVGPDKTATLESLLNLFQETALNHVWMSGLLSNGFGATHGMMRNNL
IWVVSRMHVQVHHYPIWGEVVEIDTWVGASGKNGMRRDWLIRSQATGITYARATSTWVM
MNEQTRRLSKMPEEVRGEISPWFIEKQAIKEDAPEKIVKLDDKAKYVNSDLKPKRSDLDMNH
HVNNVKYVRWMLETIPDKFLESHQLSSIVLEYRRECGSSDKVQSLCQPDEDRILANGLEQSLL
ENIVLASGIMLGNGHLGSLGMKTYGFTHLLQIKGDSQNDEIVRGRTRWKKKQSTTPYST,
Theobroma cacao palmitoyl-acyl carrier protein thioesterase,
chloroplastic isoform X1 ID = Tc03v2_p010930.1|Name = Tc03v2_p010930.1|
organism = Theobroma cacao|type = polypeptide|length = 465bp (TcFatB2)
Ref. No. Tc03v2_p010930.1, NCBI Reference
Sequence: XP_017972388.1 (corresponds to SEQ ID NO: 112), 465 aa
SEQ ID NO: 132
MHQCHSNAPLFACKLTTEVSRFLPHPLEVVELQIASAPLDAGARELGTAICFFFRTRKFASLAL
FFCSRVSKCEALTMASMAKASNVTSLFLGGVCKEEKTKNVAMAKLGFYSSWNLIKPKRKGL
LLIASAKNPHNLDMINGKKVNGIFVGEAPYTGKKSTVLIKEHVPYKQAHAASLVGRFVEDRH
VYRQTFIIRSYETGPDKTATMETVMNLLQETALNHVRSSGLAGNGFGATREMSLRKLIWVVT
RIHVQVERYSCWGDVVEIDTWVDAAGKNAMRRDWIIRDYNTQEIITRATSTWVIMNHETRR
LTKIPEQVRQEVIPFYLNRIAIAEEKNDIGKIDKLTDENAERIRSGLAPRWSDMDANQHVNNV
KYIGWILESVPMDVLEEYRLTSMTLEYRRECRKSNLLESLTSSTANVTEDSNNNSNNRKADL
EYTHLLRMQDDVAEIVRARSEWQSKDKHSW,
Theobroma cacao palmitoyl-acyl carrier protein thioesterase,
chloroplastic isoform X1 (TcFatB2) truncated (corresponds to SEQ
ID NO: 113; corresponds to aa 183-465 of SEQ ID NO: 132), 284 aa
SEQ ID NO: 133
MVEDRHVYRQTFIIRSYETGPDKTATMETVMNLLQETALNHVRSSGLAGNGFGATREMSLR
KLIWVVTRIHVQVERYSCWGDVVEIDTWVDAAGKNAMRRDWIIRDYNTQEIITRATSTWVI
MNHETRRLTKIPEQVRQEVIPFYLNRIAIAEEKNDIGKIDKLTDENAERIRSGLAPRWSDMDA
NQHVNNVKYIGWILESVPMDVLEEYRLTSMTLEYRRECRKSNLLESLTSSTANVTEDSNNNS
NNRKADLEYTHLLRMQDDVAEIVRARSEWQSKDKHSW,
Theobroma cacao palmitoyl-acyl carrier protein thioesterase,
chloroplastic isoform X2, ID = Tc03v2_p010930.2|Name = Tc03v2_p010930.2|
organism = Theobroma cacao|type = polypeptide|length = 388 bp (TcFatB3)
Ref. No. Tc03v2_p010930.2 (corresponds to SEQ ID NO: 114; corresponds
to aa 78-465 of SEQ ID NO: 132), NCBI Reference Sequence:
XP_017972389.1, 388 aa
SEQ ID NO: 134
MASMAKASNVTSLFLGGVCKEEKTKNVAMAKLGFYSSWNLIKPKRKGLLLIASAKNPHNLD
MINGKKVNGIFVGEAPYTGKKSTVLIKEHVPYKQAHAASLVGRFVEDRHVYRQTFIIRSYET
GPDKTATMETVMNLLQETALNHVRSSGLAGNGFGATREMSLRKLIWVVTRIHVQVERYSC
WGDVVEIDTWVDAAGKNAMRRDWIIRDYNTQEIITRATSTWVIMNHETRRLTKIPEQVRQE
VIPFYLNRIAIAEEKNDIGKIDKLTDENAERIRSGLAPRWSDMDANQHVNNVKYIGWILESVP
MDVLEEYRLTSMTLEYRRECRKSNLLESLTSSTANVTEDSNNNSNNRKADLEYTHLLRMQD
DVAEIVRARSEWQSKDKHSW,
Theobroma cacao palmitoyl-acyl carrier protein thioesterase,
chloroplastic isoform X2, (TcFatB3) truncated (corresponds to
SEQ ID NO: 115; corresponds to aa 106-388 of SEQ ID NO: 134), 284 aa
SEQ ID NO: 135
MVEDRHVYRQTFIIRSYETGPDKTATMETVMNLLQETALNHVRSSGLAGNGFGATREMSLR
KLIWVVTRIHVQVERYSCWGDVVEIDTWVDAAGKNAMRRDWIIRDYNTQEIITRATSTWVI
MNHETRRLTKIPEQVRQEVIPFYLNRIAIAEEKNDIGKIDKLTDENAERIRSGLAPRWSDMDA
NQHVNNVKYIGWILESVPMDVLEEYRLTSMTLEYRRECRKSNLLESLTSSTANVTEDSNNNS
NNRKADLEYTHLLRMQDDVAEIVRARSEWQSKDKHSW,
Theobroma cacao palmitoyl-acyl carrier protein thioesterase,
chloroplastic isoform X3 ID=Tc03v2_p010930.3|Name = Tc03v2_p010930.3|
organism = Theobroma cacao|type = polypeptide|length = 385 bp
(TcFatB4) Ref. No. Tc03v2_p010930.3, NCBI Reference
Sequence: XP_017972390.1 (corresponds to SEQ ID NO: 116) 385 aa
SEQ ID NO: 136
MHQCHSNAPLFACKLTTEVSRFLPHPLEVVELQIASAPLDAGARELGTAICFFFRTRKFASLAL
FFCSRVSKCEALTMASMAKASNVTSLFLGGVCKEEKTKNVAMAKLGFYSSWNLIKPKRKGL
LLIASAKNPHNLDMINGKKVNGIFVGEAPYTGKKSTVLIKEHVPYKQAHAASLVGRFVEDRH
VYRQTFIIRSYETGPDKTATMETVMNLLQETALNHVRSSGLAGNGFGATREMSLRKLIWVVT
RIHVQVERYSCWGDVVEIDTWVDAAGKNAMRRDWIIRDYNTQEIITRATSTWVIMNHETRR
LTKIPEQVRQEVIPFYLNRIAIAEEKNDIGKIDKLTDENAERIRSGLAPRWSDMDANQHVNNV
KYIGWILEAFTN,
Theobroma cacao palmitoyl-acyl carrier protein thioesterase,
chloroplastic isoform X3 (TcFatB4) truncated (corresponds to SEQ
ID NO: 117; corresponds to aa 183-385 of SEQ ID NO: 136), 204 aa
SEQ ID NO: 137
MVEDRHVYRQTFIIRSYETGPDKTATMETVMNLLQETALNHVRSSGLAGNGFGATREMSLR
KLIWVVTRIHVQVERYSCWGDVVEIDTWVDAAGKNAMRRDWIIRDYNTQEIITRATSTWVI
MNHETRRLTKIPEQVRQEVIPFYLNRIAIAEEKNDIGKIDKLTDENAERIRSGLAPRWSDMDA
NQHVNNVKYIGWILEAFTN,
Theobroma cacao palmitoyl-acyl carrier protein thioesterase,
chloroplastic, ID = Tc06v2_p006710.1|Name = Tc06v2_p006710.1|
organism = Theobroma cacao|type = polypeptide|length = 376 bp
(TcFatB5) Ref. No. Tc06v2_p006710.1, NCBI Reference Sequence:
XP_007024037.2 (corresponds to SEQ ID NO: 118), 376 aa
SEQ ID NO: 138
MAASSNIMTSKFFMATSPSSWNSTNKSKICLQQIDRSSNTNGKMVKFTRDSSLKVKLQAQAL
LTNDSRATSMIESLKDEEMMTSPPATMEHLTTEGRLINDGLVFQQNFSIRSFEIDSEYKVSARA
IMNYLQESSLNHRKKMGMSSDSLVGVTPEMIKRDLLWIFRGMCIEVDRYPSWADVVQIYHRI
YTSGRTGLRLEWIVNDSKTGETLVRASCLAVMMNKKTRKTCKFPEEVKQELKPYLTTDAEP
LFEADKILCPQVGKMDNIRTGLTSCWHDLDFNYHVNNAKYLDWILEGTPTSLIHSHELSRVS
LQYRKECLKDDVIQSLSRVVTKETGLSTNNQEIELEHVLRLESGPELAGARTAWRPKSICRQT
IN
Theobroma cacao palmitoyl-acyl carrier protein thioesterase,
chloroplastic, (TcFatB5) truncated (corresponds to SEQ ID NO: 119;
corresponds to aa 98-376 of SEQ ID NO: 138), 280 aa
SEQ ID NO: 139
MLINDGLVFQQNFSIRSFEIDSEYKVSARAIMNYLQESSLNHRKKMGMSSDSLVGVTPEMIKR
DLLWIFRGMCIEVDRYPSWADVVQIYHRIYTSGRTGLRLEWIVNDSKTGETLVRASCLAVM
MNKKTRKTCKFPEEVKQELKPYLTTDAEPLFEADKILCPQVGKMDNIRTGLTSCWHDLDFN
YHVNNAKYLDWILEGTPTSLIHSHELSRVSLQYRKECLKDDVIQSLSRVVTKETGLSTNNQEI
ELEHVLRLESGPELAGARTAWRPKSICRQTIN,
Theobroma cacao palmitoyl-acyl carrier protein thioesterase,
chloroplastic, ID = Tc09v2_p009980.1|Name = Tc09v2_p009980.1|organism =
Theobroma cacao|type = polypeptide|length = 420 bp (TcFatB6)
Ref. No. Tc09v2_p009980.1, NCBI Reference Sequence: XP_007013278.2
(corresponds to SEQ ID NO: 120), 420 aa
SEQ ID NO: 68
MVATAASSAFFPVTSSPDSSDSKNKKLGSGSTNLGGIKSKPSTPSGILQVKANAQAPPKINGTT
VVTTPVESFKNEDTASSPPPRTFINQLPDWSMLLAAITTIFLAAEKQWMMLDWKPRRPDMLI
DPFGIGRIVQDGLVFRQNFSIRSYEIGADRTASIETLMNHLQETAINHCRSAGLLGEGFGSTPE
MCKKNLIWVVTRMQVVVDRYPTWGDVVQVDTWASASGKNGMRRDWLVSDSKTGEILTR
ASSVWVMMNKLTRRLSKIPEEVRGEIEPYFMNSDPVVAEDSRKLVKLDDSTADYVRKGLTP
RWGDLDVNQHVNNVKYIGWILESAPLPILETHELSSMTLEYRRECGRDGVLQSLTAVSDSGV
GNLVNFGEIECQHLLRLEDGSEIVRGRTEWRPKYAKSFGNVGQLPAESA,
Theobroma cacao palmitoyl-acyl carrier protein thioesterase,
chloroplastic, (TcFatB6) truncated (corresponds to SEQ ID NO: 121;
corresponds to aa 134-420 of SEQ ID NO: 68), 288 aa
SEQ ID NO: 70
MIVQDGLVFRQNFSIRSYEIGADRTASIETLMNHLQETAINHCRSAGLLGEGFGSTPEMCKKN
LIWVVTRMQVVVDRYPTWGDVVQVDTWASASGKNGMRRDWLVSDSKTGEILTRASSVWV
MMNKLTRRLSKIPEEVRGEIEPYFMNSDPVVAEDSRKLVKLDDSTADYVRKGLTPRWGDLD
VNQHVNNVKYIGWILESAPLPILETHELSSMTLEYRRECGRDGVLQSLTAVSDSGVGNLVNF
GEIECQHLLRLEDGSEIVRGRTEWRPKYAKSFGNVGQLPAESA,

In some embodiments of any of the aspects, the functional heterologous thioesterase is from a bacterial species (e.g., Marvinbryantia formatexigens or Limosilactobacillus reuteri). In some embodiments of any of the aspects, the functional heterologous thioesterase gene comprises a Marvinbryantia thioesterase gene. In some embodiments of any of the aspects, the functional heterologous thioesterase gene comprises a Limosilactobacillus thioesterase gene. In some embodiments of any of the aspects, the functional heterologous thioesterase gene comprises a Marvinbryantia formatexigens thioesterase gene. In some embodiments of any of the aspects, the functional heterologous thioesterase gene comprises a Limosilactobacillus reuteri thioesterase gene. In some embodiments of any of the aspects, the engineered bacterium comprises at least one exogenous copy of at least one functional thioesterase gene comprising one of SEQ ID NOs: 22-23, SEQ ID NO: 98, or a nucleic acid sequence that is at least 80%, at least 85%, at least 90%, at least 91%, at least 92%, at least 93%, at least 94%, at least 95%, at least 96%, at least 97%, at least 98%, or at least 99% identical to the sequence of one of SEQ ID NOs: 22-23 or SEQ ID NO: 98, that maintains the same functions as one of SEQ ID NO: 22-23 or SEQ ID NO: 98 (e.g., thioesterase).

In some embodiments of any of the aspects, the amino acid sequence encoded by the functional thioesterase gene comprises one of SEQ ID NO: 24, SEQ ID NO: 122, or an amino acid sequence that is at least 80%, at least 85%, at least 90%, at least 91%, at least 92%, at least 93%, at least 94%, at least 95%, at least 96%, at least 97%, at least 98%, or at least 99% identical to the sequence of SEQ ID NO: 24 or SEQ ID NO: 122, that maintains the same functions as SEQ ID NO: 24 or SEQ ID NO: 122 (e.g., thioesterase).

Marvinbryantia formatexigens thioesterase (MfTE), Marvinbryantia
formatexigens DSM 14469 B_formatexigens-1.0.1_Cont6.1, whole genome
shotgun sequence, GenBank: ACCL02000007.1, REGION: 41936-42652, 717 bp
SEQ ID NO: 22
ATGATTTATATGGCATATCAATACCGCAGCCGCATCCGCTACAGCGAAATTGGCGAGGA
CAAAAAGCTTACGCTGCCCGGTCTGGTGAATTATTTCCAGGACTGCAGCACCTTCCAGTC
GGAGGCACTCGGCATAGGGCTGGACACGCTGGGAGCGCGCCAGCGGGCATGGCTTCTGG
CGTCCTGGAAAATTGTAATAGACAGGCTGCCGCGGCTTGGGGAGGAGGTTGTGACGGAG
ACCTGGCCATATGGCTTTAAGGGCTTCCAGGGAAACCGCAACTTCCGTATGCTGGACCAG
GAGGGACATACACTGGCTGCAGCGGCATCCGTCTGGATTTATTTAAATGTGGAAAGCGG
GCATCCGTGCCGGATTGACGGGGATGTTCTGGAGGCATATGAGCTGGAAGAGGAGCTGC
CGCTCGGTCCGTTTTCGCGCAAGATTCCGGTTCCGGAGGAAAGCACGGAGCGGGACAGC
TTTCTGGTGATGCGCAGTCACCTGGACACCAATCACCATGTCAACAACGGGCAGTATATA
CTGATGGCGGAGGAATATCTGCCGGAGGGCTTTAAAGTAAAGCAGATACGCGTGGAGTA
CCGCAAAGCCGCCGTTCTGCACGATACGATTGTGCCGTTTGTGTGCACAGAGCCGCAGCG
CTGCACGGTCAGCCTTTGCGGAAGTGATGAAAAGCCGTTTGCCGTCGTAGAATTTTCGGA
ATAA,
CnDNA_MfTE, codon-optimized, 714 bp
SEQ ID NO: 23
ATGATCTACATGGCCTACCAGTACCGCTCGCGCATCCGCTACTCGGAGATCGGCGAGGAC
AAGAAGCTGACCCTGCCGGGCCTGGTGAACTACTTCCAGGACTGCTCGACCTTCCAGTCG
GAGGCCCTGGGCATCGGCCTGGACACCCTGGGCGCCCGCCAGCGCGCCTGGCTGCTGGC
CTCGTGGAAGATCGTGATCGACCGCCTGCCGCGCCTGGGCGAGGAGGTGGTGACCGAGA
CCTGGCCGTACGGCTTCAAGGGCTTCCAGGGCAACCGCAACTTCCGCATGCTGGACCAG
GAGGGCCACACCCTGGCCGCCGCCGCCTCGGTGTGGATCTACCTGAACGTGGAGTCGGG
CCACCCGTGCCGCATCGACGGCGACGTGCTGGAGGCCTACGAGCTGGAGGAGGAGCTGC
CGCTGGGCCCGTTCTCGCGCAAGATCCCGGTGCCGGAGGAGTCGACCGAGCGCGACTCG
TTCCTGGTGATGCGCTCGCACCTGGACACCAACCACCACGTGAACAACGGCCAGTACATC
CTGATGGCCGAGGAGTACCTGCCGGAGGGCTTCAAGGTGAAGCAGATCCGCGTGGAGTA
CCGCAAGGCCGCCGTGCTGCACGACACCATCGTGCCGTTCGTGTGCACCGAGCCGCAGC
GCTGCACCGTGTCGCTGTGCGGCTCGGACGAGAAGCCGTTCGCCGTGGTGGAGTTCTCGG
AG,
Acyl-ACP thioesterase [Marvinbryantia formatexigens DSM 14469],
GenBank: EET61113.1, 238 aa (corresponds to SEQ ID NOs: 22-23)
SEQ ID NO: 24
MIYMAYQYRSRIRYSEIGEDKKLTLPGLVNYFQDCSTFQSEALGIGLDTLGARQRAWLLASW
KIVIDRLPRLGEEVVTETWPYGFKGFQGNRNFRMLDQEGHTLAAAASVWIYLNVESGHPCRI
DGDVLEAYELEEELPLGPFSRKIPVPEESTERDSFLVMRSHLDTNHHVNNGQYILMAEEYLPE
GFKVKQIRVEYRKAAVLHDTIVPFVCTEPQRCTVSLCGSDEKPFAVVEFSE,
Limosilactobacillus reuteri (also referred to as Lactobacillus reuteri)
Acyl-ACP thioesterase (LreuTE), NC_009513, region: 379328-380089, 762 nt
SEQ ID NO: 98
ATGGAGTTAAAGCAAGAAGCACAAACATTCGAAATGCCGCACTTACTAACATATTATGA
GTGTGATGAAACAAGTCATCCAAGCCTAAGCATGATATTAAGTATGATTTCCATGGTATC
CGATGAGCATAGTATGTCTTTAGGAATGGGCACCAAAGAAATACAATCTACTGGCGGTA
CATGGGTAGTAAGCGGCTATGAAGGACATCTTTCCGCAAAGCAACCTTCTTTTGGCGAAA
CAGTTATTTTAGGAACAAAAGCTGTTTCCTATAACCGCTTTTTTGCTGTTCGTGATTTTTG
GATAACGGGTAAAGAACACCAGATTGAATATGCACGAATTAGGTCGATTTTTGTGTTTAT
GAATCTAAAGACGCGGCGAATGCAATCAATTCCACCAGCTTTAATTGAACCGTTTAATGC
TCCCGTTGCGAAAAGAATTCCTCGTCTAAAGCGACCTCAAAAATTGGATGAAAATGCTTC
GGTAATAAAGAAGAATTATCAAGTTCGTTATTTTGACCTTGATGCTAATCACCATGTTAA
TAATGCTCGTTACTTTGATTGGCTTCTTGATCCTCTTGGCCGTGATTTTTTACGCGGAAAT
CAGATAAAGAGATTTAATTTGCAGTATCTTCAAGAGGTACGCAATGGAGAAATGGTAGA
AAGTAAAGTTAATAAGCTTCAAACAAATGATGAAAAGGTAACTTATCATCAGATTGGTG
TTGGTGAGCAAATTGATGCAATTGCTGAGATAGAATGGTATTAA,
Limosilactobacillus reuteri (also referred to as Lactobacillus reuteri)
Acyl-ACP thioesterase, OS = Lactobacillus reuteri (strain DSM 20016)
OX = 557436 GN = Lreu_0335 PE = 3 SV = 1 (LreuTE) Ref. No.
tr|A5VID1|A5VID1_LACRD, NCBI Reference Sequence: WP_003667392.1
(corresponds to SEQ ID NO: 98), 253 aa
SEQ ID NO: 122
MELKQEAQTFEMPHLLTYYECDETSHPSLSMILSMISMVSDEHSMSLGMGTKEIQSTGGTWV
VSGYEGHLSAKQPSFGETVILGTKAVSYNRFFAVRDFWITGKEHQIEYARIRSIFVFMNLKTR
RMQSIPPALIEPFNAPVAKRIPRLKRPQKLDENASVIKKNYQVRYFDLDANHHVNNARYFDW
LLDPLGRDFLRGNQIKRFNLQYLQEVRNGEMVESKVNKLQTNDEKVTYHQIGVGEQIDAIAE
IEWY,

In some embodiments of any of the aspects, the engineered bacterium comprises a Cuphea palustris FatB1 gene or polypeptide (e.g., SEQ ID NOs: 9, 17, 18), a Cuphea palustris FatB2 gene or polypeptide (e.g., SEQ ID NOs: 10, 19, 20), a Cuphea palustris FatB2-FatB1 hybrid gene or polypeptide (e.g., SEQ ID NOs: 11, 16, 21), a Marvinbryantia formatexigens thioesterase gene or polypeptide (e.g., SEQ ID NOs: 22-24), a Limosilactobacillus reuteri thioesterase gene or polypeptide (e.g., SEQ ID NOs: 98, 122), a Arachis hypogaea thioesterase gene or polypeptide (e.g., SEQ ID NOs: 99-102, 123-124), a Mangifera indica thioesterase gene or polypeptide (e.g., SEQ ID NOs: 103, 125), a Morella rubra thioesterase gene or polypeptide (e.g., SEQ ID NOs: 104, 126), a Pistacia vera thioesterase gene or polypeptide (e.g., SEQ ID NOs: 105, 127), or a Theobroma cacao thioesterase gene or polypeptide (e.g., SEQ ID NOs: 68, 70 106-121, 128-139).

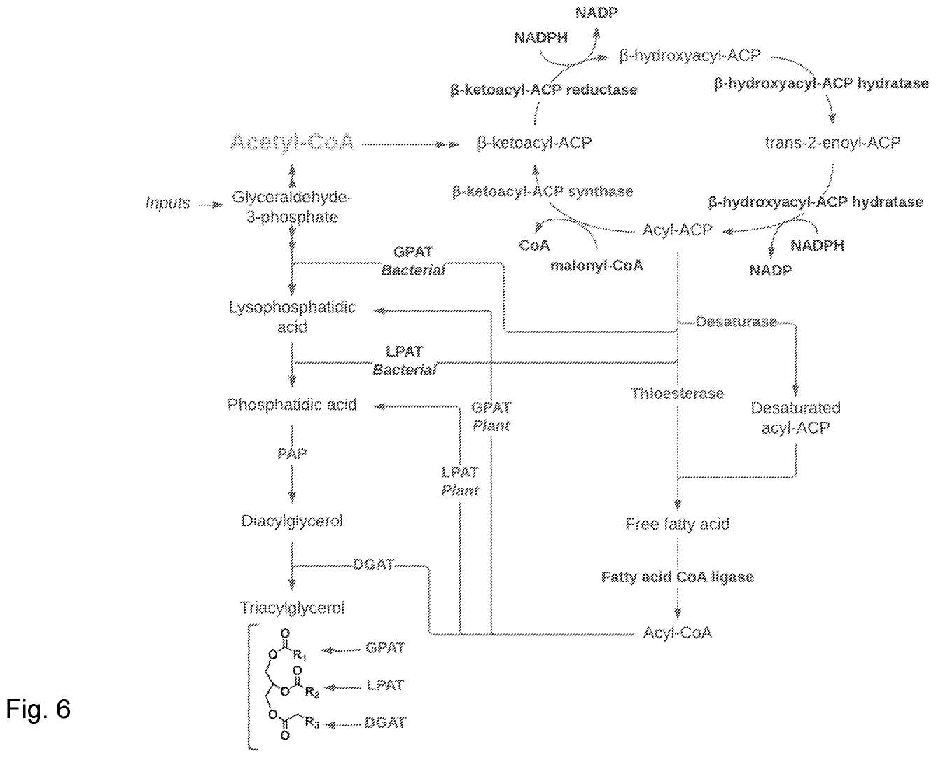

In some embodiments of any of the aspects, the engineered bacterium comprises at least one exogenous copy of at least one functional acyltransferase gene. An acyltransferase is a type of transferase enzyme that acts upon acyl groups. In general, acyltransferases share the ability to transfer thioester-activated acyl substrates to a hydroxyl or amine acceptor to form an ester or amide bond. Non-limiting examples of acyltransferases that can be used for TAG synthesis in the engineered bacteria described herein include diglyceride acyltransferase (DGAT), wax synthase (WS), a hybrid of a DGAT and a WS, lysophosphatidic acid acyltransferase (LPAT), and glycerol-3-phosphate acyltransferase (GPAT) (see e.g., FIG. 6). In some embodiments of any of the aspects, the acyltransferase catalyzes transesterification of the sn3 OH group, the sn2 OH group, or the sn1 OH group of a TAG precursor (e.g., diacylglycerol, lysophosphatidic acid, or glyceraldehyde-3-phosphate) with a fatty acid. In some embodiments of any of the aspects, the fatty acid is esterified with acyl carrier protein (ACP) or with acetyl-CoA. In some embodiments of any of the aspects, the acetyltransferase is a bacterial acetyltransferase. In some embodiments of any of the aspects, the acetyltransferase is a plant acetyltransferase. In some embodiments of any of the aspects, an acyltransferase polypeptide as described herein (e.g., DGAT, WS, DGAT-WS hybrid, LPAT, or GPAT) is truncated to remove an organelle targeting sequence(s); in some embodiments, such a targeting sequence can contribute to poor expression of the acyltransferase polypeptide, e.g., in the engineered bacteria described herein. See e.g., Table 7 for exemplary combinations of exogenous acyltransferase(s) in the engineered bacteria.

TABLE 7
Exemplary combinations of exogenous acyltransferase

| | | | | DGAT-WS |
|---|---|---|---|---|
| DGAT | LPAT | GPAT | WS | hybrid |
| X | | | | |
| | X | | | |
| X | X | | | |
| | | X | | |
| X | | X | | |
| | X | X | | |
| X | X | X | | |
| | | | X | |
| X | | | X | |
| | X | | X | |
| X | X | | X | |
| | | X | X | |
| X | | X | X | |
| | X | X | X | |
| X | X | X | X | |
| | | | | X |
| X | | | | X |
| | X | | | X |
| X | X | | | X |
| | | X | | X |
| X | | X | | X |
| | X | X | | X |
| X | X | X | | X |
| | | | X | X |
| X | | | X | X |
| | X | | X | X |
| X | X | | X | X |
| | | X | X | X |
| X | | X | X | X |
| | X | X | X | X |
| X | X | X | X | X |

In some embodiments of any of the aspects, the acyltransferase catalyzes transesterification of the sn3 OH group of diacylglycerol with a fatty acid. As a non-limiting example, such an acyltransferase is diglyceride acyltransferase (DGAT; E.C. 2.3.1.20; also referred to as O-acyltransferase or acyl-CoA:diacylglycerol acyltransferase). DGAT catalyzes the formation of triglycerides from diacylglycerol and Acyl-CoA. The reaction catalyzed by DGAT is considered the terminal and only committed step in triglyceride synthesis. In competition assays, DGATs can show preferences for fatty acyl-CoA substrates of specific chain length and desaturation. In some embodiments of any of the aspects, the functional DGAT gene preferentially produces or leads to the production of TAGs comprising a specific type of fatty acid R group (e.g., C16). As such, the functional DGAT gene can be selected from any DGAT gene from any species that preferentially produces or leads to the production of TAGs comprising a specific type of fatty acid R group (e.g., C16). In some embodiments of any of the aspects, the DGAT is a bacterial DGA T. In some embodiments of any of the aspects, the DGAT is a plant DGAT.

In some embodiments of any of the aspects, the acyltransferase is a wax synthase. In some embodiments of any of the aspects, the acyltransferase comprises a wax synthase. In some embodiments of any of the aspects, the DGAT comprises a wax synthase. In some embodiments of any of the aspects, the DGAT is a bifunctional Wax Ester Synthase/Diacylglycerol Acyltransferase (WS/DGAT), which can also be referred to as a DGAT-WS hybrid. A wax synthase can also be referred to herein as acyl-CoA:long-chain-alcohol O-acyltransferase, wax-ester synthase, or a long-chain-alcohol O-fatty-acyltransferase (EC 2.3.1.75). A wax synthase is an enzyme that catalyzes the chemical reaction acyl-CoA+a long-chain alcohol→CoA+a long-chain ester. Thus, the two substrates of this enzyme are acyl-CoA and long-chain alcohol, whereas its two products are CoA and long-chain ester. This enzyme belongs to the family of transferases, specifically those acyltransferases transferring groups other than aminoacyl groups. In general, wax synthases naturally accept acyl groups with carbon chain lengths of C16 or C18 and linear alcohols with carbon chain lengths ranging from C12 to C20.

In some embodiments of any of the aspects, the engineered bacterium comprises at least one exogenous copy of at least one functional DGAT gene. In some embodiments of any of the aspects, the engineered bacterium does not comprise a functional endogenous DGAT gene. In some embodiments of any of the aspects, the functional DGAT gene is heterologous. In some embodiments of any of the aspects, the functional heterologous DGAT gene comprises a Acinetobacter DGAT gene. In some embodiments of any of the aspects, the functional heterologous DGAT gene comprises a Thermomonospora DGAT gene. In some embodiments of any of the aspects, the functional heterologous DGAT gene comprises a Theobroma DGAT gene. In some embodiments of any of the aspects, the functional heterologous DGAT gene comprises a Rhodococcus DGAT gene.

In some embodiments of any of the aspects, the functional heterologous DGAT gene comprises a Acinetobacter baylyi DGAT gene, a Thermomonospora curvata DGAT gene, a Theobroma cacao DGAT gene, or a Rhodococcus opacus DGAT gene. In some embodiments of any of the aspects, the engineered bacterium comprises at least one exogenous copy of at least one functional DGAT gene comprising one of SEQ ID NOs: 25-28 or SEQ ID NOs: 37-45, or a nucleic acid sequence that is at least 80%, at least 85%, at least 90%, at least 91%, at least 92%, at least 93%, at least 94%, at least 95%, at least 96%, at least 97%, at least 98%, or at least 99% identical to the sequence of at least one of SEQ ID NOs: 25-28 or SEQ ID NOs: 37-45, that maintains the same functions as at least one of SEQ ID NOs: 25-28 or SEQ ID NOs: 37-45 (e.g., diglyceride acyltransferase).

In some embodiments of any of the aspects, the amino acid sequence encoded by the functional DGAT gene comprises one of SEQ ID NOs: 29-30 or SEQ ID NOs: 46-51, or an amino acid sequence that is at least 80%, at least 85%, at least 90%, at least 91%, at least 92%, at least 93%, at least 94%, at least 95%, at least 96%, at least 97%, at least 98%, or at least 99% identical to the sequence of at least one of SEQ ID NOs: 29-30 or SEQ ID NOs: 46-51, that maintains the same functions as at least one of SEQ ID NOs: 29-30 or SEQ ID NOs: 46-51 (e.g., diglyceride acyltransferase).

Acinetobacter baylyi dgaT (AbDGAT), bifunctional wax ester
synthase/diacylglycerol acyltransferase (Acinetobacter baylyi ADP1),
Gene ID: 45233297, NCBI Reference Sequence: NC_005966.1, REGION:
complement (819360-820736), 1377 bp
SEQ ID NO: 25
ATGCGCCCATTACATCCGATTGATTTTATATTCCTGTCACTAGAAAAAAGACAACAGCCT
ATGCATGTAGGTGGTTTATTTTTGTTTCAGATTCCTGATAACGCCCCAGACACCTTTATTC
AAGATCTGGTGAATGATATCCGGATATCAAAATCAATCCCTGTTCCACCATTCAACAATA
AACTGAATGGGCTTTTTTGGGATGAAGATGAAGAGTTTGATTTAGATCATCATTTTCGTC
ATATTGCACTGCCTCATCCTGGTCGTATTCGTGAATTGCTTATTTATATTTCACAAGAGCA
CAGTACGCTGCTAGATCGGGCAAAGCCCTTGTGGACCTGCAATATTATTGAAGGAATTGA
AGGCAATCGTTTTGCCATGTACTTCAAAATTCACCATGCGATGGTCGATGGCGTTGCTGG
TATGCGGTTAATTGAAAAATCACTCTCCCATGATGTAACAGAAAAAAGTATCGTGCCACC
TTGGTGTGTTGAGGGAAAACGTGCAAAGCGCTTAAGAGAACCTAAAACAGGTAAAATTA
AGAAAATCATGTCTGGTATTAAGAGTCAGCTTCAGGCGACACCCACAGTCATTCAAGAG
CTTTCTCAGACAGTATTTAAAGATATTGGACGTAATCCTGATCATGTTTCAAGCTTTCAGG
CGCCTTGTTCTATTTTGAATCAGCGTGTGAGCTCATCGCGACGTTTTGCAGCACAGTCTTT
TGACCTAGATCGTTTTCGTAATATTGCCAAATCGTTGAATGTGACCATTAATGATGTTGTA
CTAGCGGTATGTTCTGGTGCATTACGTGCGTATTTGATGAGTCATAATAGTTTGCCTTCAA
AACCATTAATTGCCATGGTTCCAGCCTCTATTCGCAATGACGATTCAGATGTCAGCAACC
GTATTACGATGATTCTGGCAAATTTGGCAACCCACAAAGATGATCCTTTACAACGTCTTG
AAATTATCCGCCGTAGTGTTCAAAACTCAAAGCAACGCTTCAAACGTATGACCAGCGATC
AGATTCTAAATTATAGTGCTGTCGTATATGGCCCTGCAGGACTCAACATAATTTCTGGCA
TGATGCCAAAACGCCAAGCCTTCAATCTGGTTATTTCCAATGTGCCTGGCCCAAGAGAGC
CACTTTACTGGAATGGTGCCAAACTTGATGCACTCTACCCAGCTTCAATTGTATTAGACG
GTCAAGCATTGAATATTACAATGACCAGTTATTTAGATAAACTTGAAGTTGGTTTGATTG
CATGCCGTAATGCATTGCCAAGAATGCAGAATTTACTGACACATTTAGAAGAAGAAATT
CAACTATTTGAAGGCGTAATTGCAAAGCAGGAAGATATTAAAACAGCCAATTAA,
CnDNA_AbDGAT, codon-optimized, 1374 bp
SEQ ID NO: 26
ATGCGCCCGCTGCACCCGATCGACTTCATCTTCCTGTCGCTGGAGAAGCGCCAGCAGCCG
ATGCACGTGGGCGGCCTGTTCCTGTTCCAGATCCCGGACAACGCCCCGGACACCTTCATC
CAGGACCTGGTGAACGACATCCGCATCTCGAAGTCGATCCCGGTGCCGCCGTTCAACAA
CAAGCTGAACGGCCTGTTCTGGGACGAGGACGAGGAGTTCGACCTGGACCACCACTTCC
GCCACATCGCCCTGCCGCACCCGGGCCGCATCCGCGAGCTGCTGATCTACATCTCGCAGG
AGCACTCGACCCTGCTGGACCGCGCCAAGCCGCTGTGGACCTGCAACATCATCGAGGGC
ATCGAGGGCAACCGCTTCGCCATGTACTTCAAGATCCACCACGCCATGGTGGACGGCGT
GGCCGGCATGCGCCTGATCGAGAAGTCGCTGTCGCACGACGTGACCGAGAAGTCGATCG
TGCCGCCGTGGTGCGTGGAGGGCAAGCGCGCCAAGCGCCTGCGCGAGCCGAAGACCGGC
AAGATCAAGAAGATCATGTCGGGCATCAAGTCGCAGCTGCAGGCCACCCCGACCGTGAT
CCAGGAGCTGTCGCAGACCGTGTTCAAGGACATCGGCCGCAACCCGGACCACGTGTCGT
CGTTCCAGGCCCCGTGCTCGATCCTGAACCAGCGCGTGTCGTCGTCGCGCCGCTTCGCCG
CCCAGTCGTTCGACCTGGACCGCTTCCGCAACATCGCCAAGTCGCTGAACGTGACCATCA
ACGACGTGGTGCTGGCCGTGTGCTCGGGCGCCCTGCGCGCCTACCTGATGTCGCACAACT
CGCTGCCGTCGAAGCCGCTGATCGCCATGGTGCCGGCCTCGATCCGCAACGACGACTCG
GACGTGTCGAACCGCATCACCATGATCCTGGCCAACCTGGCCACCCACAAGGACGACCC
GCTGCAGCGCCTGGAGATCATCCGCCGCTCGGTGCAGAACTCGAAGCAGCGCTTCAAGC
GCATGACCTCGGACCAGATCCTGAACTACTCGGCCGTGGTGTACGGCCCGGCCGGCCTG
AACATCATCTCGGGCATGATGCCGAAGCGCCAGGCCTTCAACCTGGTGATCTCGAACGTG
CCGGGCCCGCGCGAGCCGCTGTACTGGAACGGCGCCAAGCTGGACGCCCTGTACCCGGC
CTCGATCGTGCTGGACGGCCAGGCCCTGAACATCACCATGACCTCGTACCTGGACAAGCT
GGAGGTGGGCCTGATCGCCTGCCGCAACGCCCTGCCGCGCATGCAGAACCTGCTGACCC
ACCTGGAGGAGGAGATCCAGCTGTTCGAGGGCGTGATCGCCAAGCAGGAGGACATCAAG
ACCGCCAAC,
Thermomonospora curvata DGAT (TcDGAT), Thermomonospora
curvata DSM 43183, complete sequence, NC_013510 REGION complement
(4367068-4368516), 1449 bp
SEQ ID NO: 27
ATGCGCCAGCTGACGGCGGTGGACGCCAACTTCCTGAACGTCGAGACCGGCACCACGCA
CGCCCATATCGCCGGCCTGGGGATCCTCGACCCGGTCGCCTGCCCCGGCGGCCGGCTCAC
CGCCGAGGACCTCATCGAGGTGATCCGCGAACGCGCCCACCTGGCCCCCCGGCCGCTGC
GGATGCGGCTGGCCGCGGTGCCGCTGGGCATCGACCGGCCCTACTGGGAGGACGACCCG
GACTTCGACCCGGCCCGCCACGTCTTCGAGGTGGGGCTGCCGGCCCCGGGCAACGCCGC
CCAGCTCGCCGACGTCGTGGCGATGCTGCACGAACGGCCGCTGGACCGCGCCCGGCCGC
TGTGGGAGGCGGTGGTCATCCAGGGCCTGGAGGGCGGCCGCACCGCCGTCTACATCAAG
GTCCACCACGCCGCGGTGGACGGGGTCCTGGCCACCGAGACCCTGGCCGCCCTGCTGGA
CCTGTCCCCGCAGCCGCGCGAGCTGCCCCCCGACGACACCGTGCCGCAGCAGGCGCCGG
CCCTGGCCGAACGGGTGCGCACCGGGCTGCTGCGCGCGCTCGCCCACCCGGTGCGCGGC
GCCCGCATGCTGGCCCGCACCGCCCCCTACCTGGATGAGATCCCCGGCCTTGCGCAACTG
CCGGGAGTGCAGCCGCTGGCCCGGGCGATCCAGGGGGCGCTCGGCCGCGACGGCGTCGT
GCCGCTGCCCCGCACCGTCGCCCCGCCCACCCCGTTCAACGGGACGATCAGCGCCCGCCG
GGCGGTGGCCTTCGGCGAGCTGCCGCTGGCGGAGATCCGGCGCATCCGCCGGGAGCTGG
GCGGCAGCGTCAACGACGTGGTGATGGCGCTGGTGGCCACGGCGCTGCACCGCTGGCTG
GACAAGCGCGGCGAGCTGCCCGACCGGCCGCTGGTGGCGGCGGTGCCGGTGTCGCTGCG
CCGCGGCCGGGACGGCGATGCGGCGGGCGGCAACCGGATGTCGGCGATGGTGACGCCGC
TGGCCACCCATCTGGCCGACCCGGCCGAGCGCTTCGCCGCGATCCGCGGCGACCTGGCG
GCGGCCAAACGGCGCTTCGCCCGCTCCTCGGGCGCCTGGCTGGAGGGGCTGAGCGAACT
GGTGCCCGCCCCGCTGGCCGGCCCGCTGCTGCGGCTGGCCCTGCAGGCCCGGCCGGGCG
AGTACCTGCGCCCGGTCAATCTGCTGGTCTCCAACGTGCCCGGCCCGGACTTCCCGCTGT
ACCTGCGCGGCGCCCGGGTGCTCGGCTACTTCCCGATCTCGGTGGTCAGCGACCTGACCG
GCGGGCTGAACATCACCGTGCTGTCCTATGACGGCAAGCTCGACGTCGGCATCGTGACCT
GCCGCCAGATGATCCCCGACCCCTGGGAGATCATGGACCACCTCGACGACGCCCTGGGG
GAGCTGAGGGGCCTCATCGACGGCTGA,
CnDNA_TcDGAT, codon-optimized, 1446 bp
SEQ ID NO: 28
ATGCGCCAGCTGACCGCCGTGGACGCCAACTTCCTGAACGTGGAGACCGGCACCACCCA
CGCCCACATCGCCGGCCTGGGCATCCTGGACCCGGTGGCCTGCCCGGGCGGCCGCCTGA
CCGCCGAGGACCTGATCGAGGTGATCCGCGAGCGCGCCCACCTGGCCCCGCGCCCGCTG
CGCATGCGCCTGGCCGCCGTGCCGCTGGGCATCGACCGCCCGTACTGGGAGGACGACCC
GGACTTCGACCCGGCCCGCCACGTGTTCGAGGTGGGCCTGCCGGCCCCGGGCAACGCCG
CCCAGCTGGCCGACGTGGTGGCCATGCTGCACGAGCGCCCGCTGGACCGCGCCCGCCCG
CTGTGGGAGGCCGTGGTGATCCAGGGCCTGGAGGGCGGCCGCACCGCCGTGTACATCAA
GGTGCACCACGCCGCCGTGGACGGCGTGCTGGCCACCGAGACCCTGGCCGCCCTGCTGG
ACCTGTCGCCGCAGCCGCGCGAGCTGCCGCCGGACGACACCGTGCCGCAGCAGGCCCCG
GCCCTGGCCGAGCGCGTGCGCACCGGCCTGCTGCGCGCCCTGGCCCACCCGGTGCGCGG
CGCCCGCATGCTGGCCCGCACCGCCCCGTACCTGGACGAGATCCCGGGCCTGGCCCAGCT
GCCGGGCGTGCAGCCGCTGGCCCGCGCCATCCAGGGCGCCCTGGGCCGCGACGGCGTGG
TGCCGCTGCCGCGCACCGTGGCCCCGCCGACCCCGTTCAACGGCACCATCTCGGCCCGCC
GCGCCGTGGCCTTCGGCGAGCTGCCGCTGGCCGAGATCCGCCGCATCCGCCGCGAGCTG
GGCGGCTCGGTGAACGACGTGGTGATGGCCCTGGTGGCCACCGCCCTGCACCGCTGGCT
GGACAAGCGCGGCGAGCTGCCGGACCGCCCGCTGGTGGCCGCCGTGCCGGTGTCGCTGC
GCCGCGGCCGCGACGGCGACGCCGCCGGCGGCAACCGCATGTCGGCCATGGTGACCCCG
CTGGCCACCCACCTGGCCGACCCGGCCGAGCGCTTCGCCGCCATCCGCGGCGACCTGGCC
GCCGCCAAGCGCCGCTTCGCCCGCTCGTCGGGCGCCTGGCTGGAGGGCCTGTCGGAGCT
GGTGCCGGCCCCGCTGGCCGGCCCGCTGCTGCGCCTGGCCCTGCAGGCCCGCCCGGGCG
AGTACCTGCGCCCGGTGAACCTGCTGGTGTCGAACGTGCCGGGCCCGGACTTCCCGCTGT
ACCTGCGCGGCGCCCGCGTGCTGGGCTACTTCCCGATCTCGGTGGTGTCGGACCTGACCG
GCGGCCTGAACATCACCGTGCTGTCGTACGACGGCAAGCTGGACGTGGGCATCGTGACC
TGCCGCCAGATGATCCCGGACCCGTGGGAGATCATGGACCACCTGGACGACGCCCTGGG
CGAGCTGCGCGGCCTGATCGACGGC,
Theobroma cacao TcDGAT1, GenBank: KX982582.1, 1506 nt
SEQ ID NO: 37
ATGGCCATTTCTGATTCCCCAGAAATTTTGGGTTCTACTGCTACTGTTACCTCCTCTTCTC
ATTCCGATTCTGATTTGAACTTGTTGTCCATCAGAAGAAGAACTTCTACTACTGCTGCTGG
TAGAGCACCAGATAGAGATGATTCTGGTAATGGTGAAGCTGTTGATGATAGAGATCAAG
TTGAATCCGCTAACTTGATGTCTAACGTTGCTGAAAATGCTAACGAAATGCCAAACTCTT
CTGATACCAGATTCACTTACAGACCAAGAGTTCCAGCTCACAGAAGAATCAAAGAATCT
CCATTATCTTCCGGTGCCATCTTCAAACAATCTCATGCTGGTTTGTTCAACTTGTGCATCG
TTGTTTTGGTTGCCGTTAACTCCAGATTGATCATCGAAAACTTGATGAAGTACGGTTGGTT
GATCAGATCTGGTTTTTGGTTCTCTTCCAGATCTTTGTCTGATTGGCCATTATTCATGTGTT
GTTTGACCTTGCCAATTTTCCCATTGGCTGCTTTTGTTGTTGAAAAGTTGGTCCAAAGAAA
CTACATCTCCGAACCAGTTGTTGTTTTCTTGCATGCCATTATTTCTACAACCGCTGTCTTG
TATCCAGTCATCGTTAATTTGAGATGCGATTCCGCTTTTTTGTCTGGTGTTGCTTTGATGTT
GTTCGCTTGTATCGTTTGGTTGAAGTTGGTTTCTTACGCTCATACCAACAACGATATGAGA
GCTTTGGCTAAATCTGCTGAAAAGGGTGATGTTGATCCATCCTACGATGTTTCTTTTAAGT
CCTTGGCTTACTTCATGGTTGCTCCAACTTTGTGTTACCAACAATCTTATCCAAGAACCCC
AGCTGTTAGAAAATCTTGGGTTGTTAGACAATTCATTAAGTTGATCGTTTTCACCGGTTTG
ATGGGTTTCATCATCGAACAATATATCAACCCAATCGTCCAAAACTCCCAACATCCATTG
AAAGGTAATTTGTTGTACGCCATCGAAAGAGTCTTGAAGTTGTCTGTTCCAAACTTGTAT
GTCTGGTTGTGCATGTTCTACTGTTTCTTCCATTTGTGGTTGAACATCTTGGCCGAATTAT
TGAGATTCGGTGACAGAGAATTTTACAAGGATTGGTGGAATGCTAAGACCGTCGAAGAA
TATTGGAGAATGTGGAATATGCCAGTTCACAAGTGGATGGTTAGACATATCTACTTCCCA
TGTTTGAGAAACGGTATTCCAAAAGGTGTTGCTATCGTTATTGCCTTCTTGGTTTCTGCTG
TTTTCCACGAATTGTGTATTGCTGTTCCATGCCATATTTTCAAGTTGTGGGCTTTCATTGG
TATCATGTTCCAAGTTCCATTGGTCTTGATTACCAACTACTTGCAAGATAAGTTCAGATCC
TCTATGGTCGGTAACATGATTTTCTGGTTCATCTTCTCCATTTTGGGTCAACCTATGTGTG
TCTTGTTGTACTACCATGATTTGATGAACAGAAAGGGTAAGGCCGATTGA,
Theobroma cacao TcDGAT1, truncated (e.g., to remove organelle
targeting sequences), 1332 nt
SEQ ID NO: 38
ATGCAAGTTGAATCCGCTAACTTGATGTCTAACGTTGCTGAAAATGCTAACGAAATGCCA
AACTCTTCTGATACCAGATTCACTTACAGACCAAGAGTTCCAGCTCACAGAAGAATCAAA
GAATCTCCATTATCTTCCGGTGCCATCTTCAAACAATCTCATGCTGGTTTGTTCAACTTGT
GCATCGTTGTTTTGGTTGCCGTTAACTCCAGATTGATCATCGAAAACTTGATGAAGTACG
GTTGGTTGATCAGATCTGGTTTTTGGTTCTCTTCCAGATCTTTGTCTGATTGGCCATTATTC
ATGTGTTGTTTGACCTTGCCAATTTTCCCATTGGCTGCTTTTGTTGTTGAAAAGTTGGTCC
AAAGAAACTACATCTCCGAACCAGTTGTTGTTTTCTTGCATGCCATTATTTCTACAACCGC
TGTCTTGTATCCAGTCATCGTTAATTTGAGATGCGATTCCGCTTTTTTGTCTGGTGTTGCTT
TGATGTTGTTCGCTTGTATCGTTTGGTTGAAGTTGGTTTCTTACGCTCATACCAACAACGA
TATGAGAGCTTTGGCTAAATCTGCTGAAAAGGGTGATGTTGATCCATCCTACGATGTTTC
TTTTAAGTCCTTGGCTTACTTCATGGTTGCTCCAACTTTGTGTTACCAACAATCTTATCCA
AGAACCCCAGCTGTTAGAAAATCTTGGGTTGTTAGACAATTCATTAAGTTGATCGTTTTC
ACCGGTTTGATGGGTTTCATCATCGAACAATATATCAACCCAATCGTCCAAAACTCCCAA
CATCCATTGAAAGGTAATTTGTTGTACGCCATCGAAAGAGTCTTGAAGTTGTCTGTTCCA
AACTTGTATGTCTGGTTGTGCATGTTCTACTGTTTCTTCCATTTGTGGTTGAACATCTTGG
CCGAATTATTGAGATTCGGTGACAGAGAATTTTACAAGGATTGGTGGAATGCTAAGACC
GTCGAAGAATATTGGAGAATGTGGAATATGCCAGTTCACAAGTGGATGGTTAGACATAT
CTACTTCCCATGTTTGAGAAACGGTATTCCAAAAGGTGTTGCTATCGTTATTGCCTTCTTG
GTTTCTGCTGTTTTCCACGAATTGTGTATTGCTGTTCCATGCCATATTTTCAAGTIGTGGG
CTTTCATTGGTATCATGTTCCAAGTTCCATTGGTCTTGATTACCAACTACTTGCAAGATAA
GTTCAGATCCTCTATGGTCGGTAACATGATTTTCTGGTTCATCTTCTCCATTTTGGGTCAA
CCTATGTGTGTCTTGTTGTACTACCATGATTTGATGAACAGAAAGGGTAAGGCCGATTGA,
Theobroma cacao TcDGAT2, GenBank: KX982583.1, 984 nt
SEQ ID NO: 39
ATGATGGGTGAAGAAATGGAAGAAAGAAAAGCTACCGGTTACAGAGAATTTTCCGGTAG
ACATGAATTCCCATCTAACACTATGCATGCTTTGTTGGCTATGGGTATTTGGTTGGGTGCT
ATTCATTTTAACGCCTTGTTGTTGTTATTCTCCTTCTTGTTCTTGCCATTCTCCAAGTTCTT
GGTTGTTTTCGGTTTGTTGTTGTTGTTCATGATCTTGCCAATCGACCCATACTCTAAGTTT
GGTAGAAGATTGTCTAGATATATCTGCAAGCACGCTTGTTCCTACTTTCCAATTACATTGC
ACGTTGAAGATATCCATGCTTTCCATCCAGATAGAGCTTACGTTTTTGGTTTCGAACCAC
ATTCCGTTTTGCCAATTGGTGTTGTTGCTTTGGCTGATTTGACTGGTTTTATGCCATTGCC
AAAGATTAAGGTTTTGGCTTCTTCTGCTGTTTTCTACACTCCATTCTTGAGACATATTTGG
ACATGGTTGGGTTTGACTCCAGCTACTAAGAAGAATTTCTCCTCTTTGTTGGATGCTGGTT
ACTCCTGTATTTTGGTTCCAGGTGGTGTTCAAGAAACTTTTCATATGGAACCAGGTTCCG
AAATTGCTTTCTTGAGAGCTAGAAGAGGTTTCGTTAGAATTGCTATGGAAATGGGTTCTC
CTTTGGTTCCTGTTTTTTGTTTCGGTCAATCCCATGTTTACAAATGGTGGAAACCAGGTGG
TAAGTTCTACTTGCAATTTTCCAGAGCTATTAAGTTCACCCCAATCTTTTTCTGGGGTATT
TTTGGTTCTCCATTGCCATATCAACATCCAATGCATGTTGTTGTCGGTAAGCCAATTGATG
TCAAGAAAAATCCACAACCTATCGTCGAAGAAGTTATCGAAGTTCACGATAGATTTGTCG
AAGCCTTGCAAGATTTGTTCGAAAGACATAAGGCTCAAGTTGGTTTTGCCGATTTGCCAT
TGAAGATCTTGTGA,
Rhodococcus opacus PD630 diacylglycerol O-acyltransferase
(RoDGAT_atf1) codon-optimized, 1419 nt
SEQ ID NO: 40
ATGACCGACGTGTCGACCACCAACCAGCGCTACATGACCCAGACCGACTTCATGTCGTG
GCGCATGGAGGAGGACCCGATCCTGCGCTCGACCATCGTGGCCGTGGCCCTGCTGGACC
GCTCGCCGGACCAGTCGCGCTTCGTGGACATGATGCGCCGCGCCGTGGACCTGGTGCCGC
TGTTCCGCCGCACCGCCATCGAGGCCCCGATGGGCTTCGCCCCGCCGCGCTGGGCCGACG
ACCACGACTTCGACCTGTCGTGGCACCTGCGCCGCTACACCCTGCCGGAGCCGCGCACCT
GGGACGGCGTGCTGGACTTCGCCCGCACCGCCGAGATGACCGCCTTCGACAAGCGCCGC
CCGCTGTGGGAGTTCACCGTGCTGGACGGCCTGCACGACGGCCGCTCGGCCCTGGTGATG
AAGGTGCACCACTCGCTGACCGACGGCGTGTCGGGCATGCAGATCGCCCGCGAGATCGT
GGACTTCACCCGCGACGGCGGCCCGCGCCCGGACCGCACCGACCACCGCACCGCCGCCC
CGAACGGCGAGTCGCCGACCCCGCCGGGCCGCCTGTCGTGGTACCGCAACACCGCCACC
GACGTGGCCCGCCGCGCCTCGAACACCCTGGGCCGCAACTCGGTGCGCCTGGTGCGCAC
CCCGCGCGCCACCTGGCGCGACGCCGCCGCCCTGGCCGGCTCGACCCTGCGCCTGACCCG
CCCGGTGGTGTCGACCCTGTCGCCGGTGATGAAGAAGCGCTCGACCCGCCGCCACTGCG
CCGTGCTGGACGTGCCGGTGGAGGCCCTGGCCCAGGCCGCCGCCGCCGGCGCCGGCTCG
ATCAACGACGCCTTCCTGGCCGCCGTGCTGCTGGGCATGGCCAAGTACCACCGCCTGCAC
GGCGCCGAGATCTCGGAGCTGCGCATGACCCTGCCGATCTCGCTGCGCGCCGAGACCGA
CCCGGTGGGCGGCAACCGCATCACCCTGGCCCGCTTCGCCCTGCCGGCCGACATCGACG
ACCCGGCCGAGCTGATGCACCGCGTGCACGCCACCGTGGACGCCTGGCGCCACGAGCCG
GCCATCCCGCTGTCGCCGACCATCGCCGGCGCCCTGAACCTGCTGCCGGCCTCGACCCTG
GGCAACATGCTGAAGCACGTGGACTTCGTGGCCTCGAACGTGGTGGGCTCGCCGGTGCC
GCTGTTCATCGCCGGCTCGGAGGTGCTGCACTACTACGCCTTCTCGCCGACCCTGGGCTC
GGCCTTCAACGTGACCCTGATGTCGTACACCACCCGCTGCTGCGTGGGCATCAACGCCGA
CACCGACGCCATCCCGGACCTGGCCACCCTGACCGACTCGATCGCCGACGGCTTCCGCGC
CGTGCTGGGCCTGTGCACCAAGACCACCGACACCCGCGTGGTGGTGGCCTCG,
Rhodococcus opacus PD630 GenBank: CP080954.1 reverse
complement 4246604-4248022 (RODGAT_atfl), 1419 nt
SEQ ID NO: 41
TTGACCGACGTGAGCACGACGAATCAGCGCTACATGACCCAGACGGACTTCATGTCGTG
GCGGATGGAGGAGGACCCGATCCTCCGGTCGACCATCGTGGCGGTCGCACTGCTCGACC
GCAGTCCGGATCAGAGCCGATTCGTCGACATGATGCGCAGGGCGGTCGACCTGGTTCCC
CTCTTCCGGCGGACGGCGATCGAGGCTCCGATGGGCTTTGCGCCGCCGAGGTGGGCGGA
CGATCACGATTTCGATCTCAGCTGGCACCTGCGCCGCTACACCCTCCCCGAACCACGAAC
ATGGGACGGGGTTCTCGACTTCGCCCGGACCGCCGAGATGACCGCCTTCGACAAGCGCC
GTCCGCTCTGGGAGTTCACCGTTCTCGACGGACTCCACGACGGCAGGTCCGCCCTCGTGA
TGAAGGTGCATCACTCTCTCACCGACGGTGTCAGTGGAATGCAGATTGCCCGGGAGATC
GTGGATTTCACCCGCGATGGCGGGCCACGGCCGGACCGGACCGACCACCGCACGGCCGC
GCCGAACGGTGAATCGCCCACTCCGCCGGGCCGCCTCTCCTGGTACCGGAACACGGCCA
CCGACGTGGCCCGCCGGGCATCGAACACGCTGGGCCGCAACAGTGTTCGACTGGTTCGG
ACTCCGAGGGCCACCTGGCGCGACGCAGCCGCACTAGCCGGCTCCACACTGCGCCTCAC
GCGTCCCGTGGTCTCCACACTGTCCCCGGTCATGAAAAAACGCAGCACGCGGCGTCACTG
TGCGGTGCTCGACGTGCCGGTGGAGGCGCTCGCTCAGGCGGCCGCGGCGGGTGCCGGTT
CGATCAACGACGCGTTTCTCGCTGCTGTCCTGCTGGGAATGGCGAAGTACCACCGACTGC
ACGGCGCCGAGATCAGCGAACTGCGGATGACGCTGCCGATCAGCCTGCGTGCCGAGACG
GACCCGGTGGGAGGCAACCGCATCACCCTGGCACGTTTTGCACTACCCGCCGACATCGA
CGACCCCGCCGAGTTGATGCACCGGGTGCACGCCACGGTGGATGCCTGGCGCCACGAGC
CCGCGATTCCGCTGTCACCGACGATCGCCGGCGCCTTGAACCTGCTTCCGGCCTCGACAC
TCGGAAACATGCTCAAACACGTCGACTTCGTCGCCTCGAACGTCGTCGGATCTCCGGTAC
CACTGTTCATCGCCGGGTCGGAGGTTCTGCACTACTACGCGTTCAGCCCCACACTCGGAT
CGGCGTTCAACGTCACCTTGATGTCCTACACCACGCGATGCTGTGTCGGGATCAACGCCG
ACACGGACGCAATCCCGGACCTCGCAACCCTGACCGATTCGATAGCGGACGGGTTTCGC
GCCGTCCTCGGACTGTGCACGAAGACAACCGATACGAGGGTGGTCGTGGCCTCG,
Rhodococcus opacus PD630 wax ester synthase/diacylglycerol
acyltransferase (RoDGAT_atf2) codon-optimized, 1359 nt
SEQ ID NO: 42
ATGCCGGTGACCGACTCGATCTTCCTGCTGGGCGAGTCGCGCGAGCACCCGATGCACGTG
GGCTCGCTGGAGCTGTTCACCCCGCCGGAGGACGCCGGCCCGGACTACGTGAAGTCGAT
GCACGAGACCCTGCTGAAGCACACCGACGTGGACCCGACCTTCCGCAAGAAGCCGGCCG
GCCCGGTGGGCTCGCTGGGCAACCTGTGGTGGGCCGACGAGTCGGACGTGGACCTGGAG
TACCACGTGCGCCACTCGGCCCTGCCGGCCCCGTACCGCGTGCGCGAGCTGCTGACCCTG
ACCTCGCGCCTGCACGGCACCCTGCTGGACCGCCACCGCCCGCTGTGGGAGATGTACCTG
ATCGAGGGCCTGTCGGACGGCCGCTTCGCCATCTACACCAAGCTGCACCACTCGCTGATG
GACGGCGTGTCGGGCCTGCGCCTGCTGATGCGCACCCTGTCGACCGACCCGGACGTGCG
CGACGCCCCGCCGCCGTGGAACCTGCCGCGCCGCGCCTCGGCCAACGGCGCCGCCCCGG
CCCCGGACCTGTGGTCGGTGGTGAACGGCGTGCGCCGCACCGTGGGCGAGGTGGCCGGC
CTGGCCCCGGCCTCGCTGCGCATCGCCCGCACCGCCATGGGCCAGCACGACATGCGCTTC
CCGTACGAGGCCCCGCGCACCATGCTGAACGTGCCGATCGGCGGCGCCCGCCGCTTCGC
CGCCCAGTCGTGGCCGCTGGAGCGCGTGCACGCCGTGCGCAAGGCCGCCGGCGTGTCGG
TGAACGACGTGGTGATGGCCATGTGCGCCGGCGCCCTGCGCGGCTACCTGGAGGAGCAG
AAGGCCCTGCCGGACGAGCCGCTGATCGCCATGGTGCCGGTGTCGCTGCGCGACGAGCA
GAAGGCCGACGCCGGCGGCAACGCCGTGGGCGTGACCCTGTGCAACCTGGCCACCGACG
TGGACGACCCGGCCGAGCGCCTGACCGCCATCTCGGCCTCGATGTCGCAGGGCAAGGAG
CTGTTCGGCTCGCTGACCTCGATGCAGGCCCTGGCCTGGTCGGCCTTCAACATGTCGCCG
ATCGCCCTGACCCCGGTGCCGGGCTTCGTGCGCTTCACCCCGCCGCCGTTCAACGTGATC
ATCTCGAACGTGCCGGGCCCGCGCAAGACCATGTACTGGAACGGCTCGCGCCTGGACGG
CATCTACCCGACCTCGGTGGTGCTGGACGGCCAGGCCCTGAACATCACCCTGACCACCAA
CGGCGGCAACCTGGACTTCGGCGTGATCGGCTGCCGCCGCTCGGTGCCGTCGCTGCAGCG
CATCCTGTTCTACCTGGAGACCGCCCTGGGCGAGCTGGAGGCCGCCCTGCTG,
Rhodococcus opacus PD630 wax ester synthase/diacylglycerol
acyltransferase (RoDGAT_atf2), GenBank: JH377359.1 2198124-2199485,
1362 nt
SEQ ID NO: 43
ATGCCGGTTACCGATTCGATATTCCTTCTCGGCGAATCGCGAGAGCATCCGATGCACGTG
GGATCGCTCGAATTGTTCACACCGCCGGAGGATGCCGGCCCCGACTACGTGAAGTCGAT
GCACGAAACTCTGCTGAAGCACACGGACGTCGACCCCACTTTCCGCAAGAAGCCAGCGG
GCCCCGTGGGCAGTCTCGGGAATCTGTGGTGGGCCGACGAGTCGGACGTCGATCTCGAA
TACCACGTGCGTCATTCGGCCCTGCCGGCCCCGTACCGGGTCCGGGAACTGCTGACGCTG
ACGTCCCGGTTGCACGGCACGCTCCTGGACCGTCATCGCCCGCTGTGGGAGATGTATCTG
ATCGAGGGGCTCAGCGACGGCCGGTTCGCGATCTACACCAAGCTGCACCATTCGCTGAT
GGACGGGGTCTCCGGTTTGCGGCTGCTGATGCGGACGCTGTCGACCGACCCGGACGTGC
GCGACGCACCGCCGCCGTGGAACCTGCCGCGCAGGGCGTCGGCCAATGGTGCCGCCCCC
GCTCCCGACCTCTGGTCGGTGGTGAACGGGGTCCGTCGCACGGTCGGTGAGGTGGCCGG
TCTCGCGCCGGCGTCGCTGCGCATCGCCCGCACCGCGATGGGGCAGCACGACATGAGGT
TTCCGTACGAGGCGCCTCGCACCATGCTGAACGTCCCGATCGGGGGCGCGCGCCGGTTCG
CCGCGCAGTCCTGGCCGCTCGAACGCGTCCACGCCGTGCGGAAGGCAGCCGGGGTCAGT
GTCAACGACGTCGTGATGGCCATGTGCGCCGGGGCGTTGCGGGGTTACCTCGAGGAACA
GAAGGCGCTACCGGACGAGCCGCTGATCGCGATGGTTCCGGTGTCCCTGCGCGACGAGC
AGAAGGCCGACGCCGGCGGCAACGCGGTCGGGGTCACGTTGTGCAACCTGGCGACCGAC
GTCGACGACCCGGCCGAACGTCTGACGGCGATCTCCGCCTCCATGTCCCAGGGGAAGGA
ACTGTTCGGCAGCCTCACCTCGATGCAGGCGCTGGCGTGGTCCGCGTTCAACATGTCGCC
GATCGCCCTGACGCCCGTGCCCGGGTTCGTCCGCTTCACACCGCCGCCGTTCAACGTGAT
CATCTCCAACGTCCCGGGACCGCGGAAGACCATGTACTGGAACGGGTCCCGGCTGGACG
GCATCTACCCGACATCGGTGGTGCTGGACGGGCAGGCACTCAACATCACACTCACCACC
AACGGCGGCAACCTCGATTTCGGTGTCATCGGGTGCCGCCGCTCGGTGCCGAGCCTGCAA
CGCATCCTCTTCTATCTCGAGACGGCTCTGGGCGAACTCGAGGCGGCATTGCTCTGA,
Rhodococcus opacus PD630 acyltransferase 8
(RODGAT_atf8) codon-optimized, 1389 nt
SEQ ID NO: 44
ATGCCGCTGCCGATGTCGCCGCTGGACTCGATGTTCCTGCTGGGCGAGTCGCGCGAGCAC
CCGATGCACGTGGGCTGCGTGGAGATCTTCCAGCTGCCGGAGGGCGCCGACACCTACGA
CATGCGCGCCATGCTGGACCGCGCCCTGGCCGACGGCGACGGCATCGTGACCCCGCGCC
TGGCCAAGCGCGCCCACCGCTCGTTCTCGACCCTGGGCCAGTGGTCGTGGGAGACCGTG
GACGACATCGACCTGGGCCACCACATCCGCCACGACGCCCTGCCGGCCCCGGGCGGCGA
GGCCGAGCTGATGGCCCTGTGCTCGCGCCTGCACGGCTCGCTGCTGGACCGCTCGCGCCC
GCTGTGGGAGATGCACCTGATCGAGGGCCTGTCGGACGGCCGCTTCGCCGTGTACACCA
AGATCCACCACGCCGTGGCCGACGGCGTGACCGCCATGAAGATGCTGCGCAACGCCTTC
TCGGAGAACTCGGAGGACCGCGACGTGCCGGCCCCGTGGCAGCCGCGCGGCCCGCGCCG
CCAGCGCACCCCGTCGAAGGCCTTCTCGCTGTCGGGCCTGGCCGGCTCGACCTTCCGCGC
CGCCCGCGACACCGTGGGCGAGGTGGCCGGCCTGGTGCCGGCCCTGGCCGGCACCGTGT
CGCGCGCCTTCCGCGACCAGGGCGGCCCGCTGGCCCTGTCGGCCCCGAAGACCCCGTTCA
ACGTGCCGATCACCGGCGCCTGCCAGTTCGCCGCCCAGTCGTGGCCGCTGGAGCGCCTGC
GCCTGGTGGCCAAGCTGTCGGACACCGCCATCAACGACGTGGTGCTGGCCATGTCGTCG
GGCGCCCTGCGCTCGTACCTGGAGGACCAGAACGCCCTGCCGGCCGAGCCGCTGATCGC
CATGGTGCCGGTGTCGCTGAAGTCGCAGCGCGAGGCCTCGAACGGCAACAACATCGGCG
TGCTGATGTGCAACCTGGGCACCCACCTGCCGGACCTGGCCGACCGCCTGGACACCATCC
GCACCTCGATGCGCGAGGGCAAGGAGGCCTACGAGACCCTGTCGGCCACCCAGATCCTG
GCCATGTCGGCCCTGGGCGCCGCCCCGATCGGCGCCTCGATGCTGTTCGGCCACAACTCG
CGCGTGCGCCCGCCGTTCAACCTGATCATCTCGAACGTGCCGGGCCCGTCGTCGCCGCTG
TACTGGAACGGCGCCCGCCTGGACGCCATCTACCCGCTGTCGGTGCCGGTGGACGGCCA
GGGCCTGAACATCACCTGCACCTCGAACGACGACATCATCTCGTTCGGCGTGACCGGCTG
CCGCTCGGCCGTGCCGGACCTGAAGTCGATCCCGGCCCGCCTGGGCCACGAGCTGCGCG,
Rhodococcus opacus PD630 acyltransferase 8 (RODGAT_atf8),
GenBank: GU067777.1, 1392 nt
SEQ ID NO: 45
ATGCCGCTCCCCATGTCCCCTCTCGACTCGATGTTCCTTCTCGGAGAGTCCCGTGAGCACC
CGATGCATGTTGGCTGCGTAGAGATCTTCCAGCTCCCGGAAGGCGCCGACACCTACGAC
ATGCGCGCGATGCTCGACCGCGCGCTTGCCGACGGCGACGGGATCGTCACGCCCCGGCT
CGCCAAGCGAGCCCACCGGTCCTTCTCGACGCTCGGTCAGTGGAGCTGGGAGACCGTCG
ACGACATCGACCTCGGTCACCACATCCGGCACGATGCGCTGCCCGCCCCCGGGGGCGAG
GCCGAGCTGATGGCACTGTGCTCGCGGCTGCACGGATCGCTGCTCGACCGCAGCCGCCC
GCTGTGGGAGATGCACCTGATCGAGGGACTGAGCGACGGACGGTTCGCCGTCTACACCA
AGATCCACCACGCGGTCGCCGACGGCGTCACCGCGATGAAGATGCTGCGCAACGCGTTC
AGCGAGAACTCCGAGGACCGGGACGTGCCGGCCCCGTGGCAGCCGCGGGGACCGCGGC
GACAGCGGACGCCGTCGAAGGCGTTCAGCCTGTCGGGACTGGCCGGTTCCACGTTCCGC
GCCGCCCGCGACACCGTCGGTGAGGTCGCCGGGCTCGTGCCCGCGCTCGCTGGCACCGT
GTCCCGCGCCTTCCGCGACCAGGGCGGCCCGCTCGCCTTGTCCGCACCCAAGACCCCGTT
CAACGTGCCGATCACCGGTGCCTGCCAGTTCGCGGCGCAGTCGTGGCCGCTCGAACGTCT
CCGGCTCGTCGCCAAGCTGTCCGACACCGCCATCAACGACGTCGTGCTCGCGATGTCCTC
GGGAGCACTCCGCAGCTATCTCGAGGATCAGAACGCCCTGCCCGCCGAGCCGCTGATCG
CGATGGTGCCGGTGTCGCTGAAGAGTCAGCGCGAGGCGTCGAACGGCAACAACATCGGG
GTGCTCATGTGCAACCTCGGCACCCACCTCCCCGACCTGGCGGATCGCCTCGACACCATC
CGGACGTCGATGCGCGAGGGCAAGGAGGCGTACGAGACGCTGAGTGCGACGCAGATCCT
CGCGATGAGCGCTCTCGGCGCGGCACCGATCGGCGCGAGCATGCTGTTCGGGCACAACT
CGCGGGTGCGCCCGCCGTTCAACCTCATCATCTCCAATGTTCCGGGTCCCAGCTCGCCGC
TGTATTGGAACGGGGCACGGCTCGATGCCATCTACCCGCTGTCGGTGCCCGTCGACGGCC
AGGGCCTGAACATCACCTGCACCAGCAACGACGACATCATCTCGTTCGGGGTCACCGGC
TGCCGCAGCGCGGTGCCCGACCTGAAGTCGATCCCCGCCCGGCTGGGGCACGAACTGCG
CGCCCTCGAGCGCGCGGTCGGAATCTGA,
Acinetobacter baylyi DGAT (AbDGAT), e.g., strain ADP1,
bifunctional wax ester synthase/diacylglycerol acyltransferase,
AAO17391.1, NCBI Reference Sequence: WP_004922247.1, 458 aa
(corresponds to SEQ ID NOs: 25-26)
SEQ ID NO: 29
MRPLHPIDFIFLSLEKRQQPMHVGGLFLFQIPDNAPDTFIQDLVNDIRISKSIPVPPFNNKLNGLF
WDEDEEFDLDHHFRHIALPHPGRIRELLIYISQEHSTLLDRAKPLWTCNIIEGIEGNRFAMYFKI
HHAMVDGVAGMRLIEKSLSHDVTEKSIVPPWCVEGKRAKRLREPKTGKIKKIMSGIKSQLQA
TPTVIQELSQTVFKDIGRNPDHVSSFQAPCSILNQRVSSSRRFAAQSFDLDRFRNIAKSLNVTIN
DVVLAVCSGALRAYLMSHNSLPSKPLIAMVPASIRNDDSDVSNRITMILANLATHKDDPLQR
LEIIRRSVQNSKQRFKRMTSDQILNYSAVVYGPAGLNIISGMMPKRQAFNLVISNVPGPREPLY
WNGAKLDALYPASIVLDGQALNITMTSYLDKLEVGLIACRNALPRMQNLLTHLEEEIQLFEG
VIAKQEDIKTAN,
Thermomonospora curvata DGAT, wax ester/triacylglycerol synthase
family O-acyltransferase, NCBI Reference Sequence: WP_012854133.1,
482 aa (corresponds to SEQ ID NOs: 27-28)
SEQ ID NO: 30
MRQLTAVDANFLNVETGTTHAHIAGLGILDPVACPGGRLTAEDLIEVIRERAHLAPRPLRMRL
AAVPLGIDRPYWEDDPDFDPARHVFEVGLPAPGNAAQLADVVAMLHERPLDRARPLWEAV
VIQGLEGGRTAVYIKVHHAAVDGVLATETLAALLDLSPQPRELPPDDTVPQQAPALAERVRT
GLLRALAHPVRGARMLARTAPYLDEIPGLAQLPGVQPLARAIQGALGRDGVVPLPRTVAPPT
PFNGTISARRAVAFGELPLAEIRRIRRELGGSVNDVVMALVATALHRWLDKRGELPDRPLVA
AVPVSLRRGRDGDAAGGNRMSAMVTPLATHLADPAERFAAIRGDLAAAKRRFARSSGAWL
EGLSELVPAPLAGPLLRLALQARPGEYLRPVNLLVSNVPGPDFPLYLRGARVLGYFPISVVSD
LTGGLNITVLSYDGKLDVGIVTCRQMIPDPWEIMDHLDDALGELRGLIDG,
Theobroma cacao TcDGAT1, Ref No. XP_007012778.1, 501 aa
(corresponds to SEQ ID NO: 37)
SEQ ID NO: 46
MAISDSPEILGSTATVTSSSHSDSDLNLLSIRRRTSTTAAGRAPDRDDSGNGEAVDDRDQVES
ANLMSNVAENANEMPNSSDTRFTYRPRVPAHRRIKESPLSSGAIFKQSHAGLFNLCIVVLVAV
NSRLIIENLMKYGWLIRSGFWFSSRSLSDWPLFMCCLTLPIFPLAAFVVEKLVQRNYISEPVVV
FLHAIISTTAVLYPVIVNLRCDSAFLSGVALMLFACIVWLKLVSYAHTNNDMRALAKSAEKG
DVDPSYDVSFKSLAYFMVAPTLCYQQSYPRTPAVRKSWVVRQFIKLIVFTGLMGFIIEQYINPI
VQNSQHPLKGNLLYAIERVLKLSVPNLYVWLCMFYCFFHLWLNILAELLRFGDREFYKDWW
NAKTVEEYWRMWNMPVHKWMVRHIYFPCLRNGIPKGVAIVIAFLVSAVFHELCIAVPCHIFK
LWAFIGIMFQVPLVLITNYLQDKFRSSMVGNMIFWFIFSILGQPMCVLLYYHDLMNRKGKAD,
Theobroma cacao TcDGATI truncated, 443 aa (corresponds to SEQ ID
NO: 38; corresponds to aa 60-501 of SEQ ID NO: 46)
SEQ ID NO: 47
MQVESANLMSNVAENANEMPNSSDTRFTYRPRVPAHRRIKESPLSSGAIFKQSHAGLFNLCIV
VLVAVNSRLIIENLMKYGWLIRSGFWFSSRSLSDWPLFMCCLTLPIFPLAAFVVEKLVQRNYIS
EPVVVFLHAIISTTAVLYPVIVNLRCDSAFLSGVALMLFACIVWLKLVSYAHTNNDMRALAK
SAEKGDVDPSYDVSFKSLAYFMVAPTLCYQQSYPRTPAVRKSWVVRQFIKLIVFTGLMGFIIE
QYINPIVQNSQHPLKGNLLYAIERVLKLSVPNLYVWLCMFYCFFHLWLNILAELLRFGDREFY
KDWWNAKTVEEYWRMWNMPVHKWMVRHIYFPCLRNGIPKGVAIVIAFLVSAVFHELCIAV
PCHIFKLWAFIGIMFQVPLVLITNYLQDKFRSSMVGNMIFWFIFSILGQPMCVLLYYHDLMNR
KGKAD,
Theobroma cacao TcDGAT2, Ref No. XP_007046425.1, 327 aa
(corresponds to SEQ ID NO: 39)
SEQ ID NO: 48
MMGEEMEERKATGYREFSGRHEFPSNTMHALLAMGIWLGAIHFNALLLLFSFLFLPFSKFLV
VFGLLLLFMILPIDPYSKFGRRLSRYICKHACSYFPITLHVEDIHAFHPDRAYVFGFEPHSVLPI
GVVALADLTGFMPLPKIKVLASSAVFYTPFLRHIWTWLGLTPATKKNFSSLLDAGYSCILVPG
GVQETFHMEPGSEIAFLRARRGFVRIAMEMGSPLVPVFCFGQSHVYKWWKPGGKFYLQFSR
AIKFTPIFFWGIFGSPLPYQHPMHVVVGKPIDVKKNPQPIVEEVIEVHDRFVEALQDLFERHKA
QVGFADLPLKIL,
Rhodococcus opacus diacylglycerol O-acyltransferase RODGAT_atfl,
Ref No. EHI42943.1, 473 aa (corresponds to SEQ ID NO: 40 or
SEQ ID NO: 41)
SEQ ID NO: 49
MTDVSTTNQRYMTQTDFMSWRMEEDPILRSTIVAVALLDRSPDQSRFVDMMRRAVDLVPLF
RRTAIEAPMGFAPPRWADDHDFDLSWHLRRYTLPEPRTWDGVLDFARTAEMTAFDKRRPL
WEFTVLDGLHDGRSALVMKVHHSLTDGVSGMQIAREIVDFTRDGGPRPDRTDHRTAAPNGE
SPTPPGRLSWYRNTATDVARRASNTLGRNSVRLVRTPRATWRDAAALAGSTLRLTRPVVSTL
SPVMKKRSTRRHCAVLDVPVEALAQAAAAGAGSINDAFLAAVLLGMAKYHRLHGAEISELR
MTLPISLRAETDPVGGNRITLARFALPADIDDPAELMHRVHATVDAWRHEPAIPLSPTIAGAL
NLLPASTLGNMLKHVDFVASNVVGSPVPLFIAGSEVLHYYAFSPTLGSAFNVTLMSYTTRCC
VGINADTDAIPDLATLTDSIADGFRAVLGLCTKTTDTRVVVAS,
Rhodococcus opacus wax ester synthase/diacylglycerol acyltransferase
RODGAT atf2, Ref No. EHI41112.1, 453 aa (corresponds to SEQ ID NO: 42
or SEQ ID NO: 43)
SEQ ID NO: 50
MPVTDSIFLLGESREHPMHVGSLELFTPPEDAGPDYVKSMHETLLKHTDVDPTFRKKPAGPV
GSLGNLWWADESDVDLEYHVRHSALPAPYRVRELLTLTSRLHGTLLDRHRPLWEMYLIEGL
SDGRFAIYTKLHHSLMDGVSGLRLLMRTLSTDPDVRDAPPPWNLPRRASANGAAPAPDLWS
VVNGVRRTVGEVAGLAPASLRIARTAMGQHDMRFPYEAPRTMLNVPIGGARRFAAQSWPLE
RVHAVRKAAGVSVNDVVMAMCAGALRGYLEEQKALPDEPLIAMVPVSLRDEQKADAGGN
AVGVTLCNLATDVDDPAERLTAISASMSQGKELFGSLTSMQALAWSAFNMSPIALTPVPGFV
RFTPPPFNVIISNVPGPRKTMYWNGSRLDGIYPTSVVLDGQALNITLTINGGNLDFGVIGCRRS
VPSLQRILFYLETALGELEAALL,
Rhodococcus opacus acyltransferase 8, RODGAT_atf8, Ref No.
ACY38595.1, 463 aa (corresponds to SEQ ID NO: 44 or SEQ ID NO: 45)
SEQ ID NO: 51
MPLPMSPLDSMFLLGESREHPMHVGCVEIFQLPEGADTYDMRAMLDRALADGDGIVTPRLA
KRAHRSFSTLGQWSWETVDDIDLGHHIRHDALPAPGGEAELMALCSRLHGSLLDRSRPLWE
MHLIEGLSDGRFAVYTKIHHAVADGVTAMKMLRNAFSENSEDRDVPAPWQPRGPRRQRTPS
KAFSLSGLAGSTFRAARDTVGEVAGLVPALAGTVSRAFRDQGGPLALSAPKTPFNVPITGAC
QFAAQSWPLERLRLVAKLSDTAINDVVLAMSSGALRSYLEDQNALPAEPLIAMVPVSLKSQR
EASNGNNIGVLMCNLGTHLPDLADRLDTIRTSMREGKEAYETLSATQILAMSALGAAPIGAS
MLFGHNSRVRPPFNLIISNVPGPSSPLYWNGARLDAIYPLSVPVDGQGLNITCTSNDDIISFGVT
GCRSAVPDLKSIPARLGHELRALERAVGI,

In some embodiments of any of the aspects, the engineered bacterium comprises a Acinetobacter baylyi DGAT gene or polypeptide (e.g., SEQ ID NOs: 25, 26, or 29) or a Thermomonospora curvata DGAT gene or polypeptide (e.g., SEQ ID NOs: 27, 28, or 30).

In some embodiments of any of the aspects, the acyltransferase catalyzes transesterification of the sn2 OH group of a lysophosphatidic acid with a fatty acid. As a non-limiting example, such an acyltransferase is lysophosphatidic acid acyltransferase (LPAT or LPAAT; E.C. 2.3.1.51; also referred to as acyl-CoA: 1-acylglycerol-sn-3-phosphate acyltransferase (AGPAT) or 1-acyl-sn-glycerol-3-phosphate acyltransferase). LPAT catalyzes acylation of the sn-2 position on lysophosphatidic acid by an acyl CoA substrate to produce phosphatidic acid, which is a precursor of triacylglycerols (TAGs), as well as polar glycerolipids and. LPAT catalyzes an important step of the de novo phospholipid biosynthesis pathway and thus has a strong flux control in the biosynthesis of TAG or phospholipids. In competition assays, LPATs can show preferences for fatty acyl-CoA substrates of specific chain length and desaturation. In some embodiments of any of the aspects, the functional LPAT gene preferentially produces or leads to the production of TAGs comprising a specific type of fatty acid R group (e.g., C16). As such, the functional LPAT gene can be selected from any LPAT gene from any species that preferentially produces or leads to the production of TAGs comprising a specific type of fatty acid R group (e.g., C16). In some embodiments of any of the aspects, the LPAT is a bacterial LPAT. In some embodiments of any of the aspects, the LPAT is a plant LPAT.

In some embodiments of any of the aspects, the engineered bacterium comprises at least one exogenous copy of at least one functional LPAT gene. In some embodiments of any of the aspects, the engineered bacterium does not comprise a functional endogenous LPAT gene. In some embodiments of any of the aspects, the functional LPAT gene is heterologous. In some embodiments of any of the aspects, the functional heterologous LPAT gene comprises a Theobroma LPAT gene.

In some embodiments of any of the aspects, the functional heterologous LPAT gene comprises a Theobroma cacao LPAT gene. In some embodiments of any of the aspects, the engineered bacterium comprises at least one exogenous copy of at least one functional LPAT gene comprising one of SEQ ID NOs: 52-58, or a nucleic acid sequence that is at least 80%, at least 85%, at least 90%, at least 91%, at least 92%, at least 93%, at least 94%, at least 95%, at least 96%, at least 97%, at least 98%, or at least 99% identical to the sequence of at least one of SEQ ID NOs: 52-58, that maintains the same functions as at least one of SEQ ID NOs: 52-58 (e.g., lysophosphatidic acid acyltransferase).

In some embodiments of any of the aspects, the amino acid sequence encoded by the functional LPAT gene comprises one of SEQ ID NOs: 59-63, or an amino acid sequence that is at least 80%, at least 85%, at least 90%, at least 91%, at least 92%, at least 93%, at least 94%, at least 95%, at least 96%, at least 97%, at least 98%, or at least 99% identical to the sequence of at least one of SEQ ID NOs: 59-63, that maintains the same functions as at least one of SEQ ID NOs: 59-63 (e.g., lysophosphatidic acid acyltransferase).

Theobroma cacao 1-acyl-sn-glycerol-3-phosphate acyltransferase 1,
chloroplastic (TcLPAT1), XM_007011850.2 301-1380, 1080 nt
SEQ ID NO: 52
ATGGAGCTCTCTTCCCTCCCTTCCGTTTCTTCTTTCTCGCTATGTCACCCAAAGCCCAGGA
GTTCTGCTACGTTGCCTTTCTTGCCTTTTTCGAATCGTAAAGGAGCTTACTTTGGCTATTCT
CTGTGTAAACGGGCAACTTTGAGAAATTCATGTAACTCTGCTCAAAATAACTTTTTGCGT
ATTTCAAGGACGCATGATGGTGTATCTCGGTGTTATTTTAATCAAAAGGAAGGTTTAAAT
AGATCGTATTACAATAGTAAATTACATAACGAGAACAAATTGTCCAGATACATAGTTGC
GAGATCTGAATTTGCTGGGACTGGGACCCCTGATGCTGCCTATTCTTTATCAGAAATTAA
ACCGGGTTCAAAAGTTAGAGGAGTATGCTTTTATGCTGTTACAGCAATAGCAGCCATTTT
ACTTATTTGGTTTATGCTGGTTTTGCATCCCTTTGTGCTCTTGTTTGATCGCTACAGAAGA
AAAGCTCAGCATTTTATTGCCAAACTTTGGGCTATGGCAACAGTTGCTCCTTTTTTTAAAA
TTGAGTTTGAAGGATTGGAGAATCTGCCTCCACAGGATGTTCCTGCCGTATATGTTTCCA
ACCACCAGAGTTTTTTAGACATCTATACACTTCTAACTCTTGGAAGAAGCTTCAAGTTCA
TCAGCAAGACGGGGATATTTCTTTATCCCATTATTGGGTGGGCCATGTCTATGATGGGTC
TAATTCCATTAAAGCGCATGGACAGCAGAAGCCAGTTGGACTGTCTTAAGAGATGTATG
GATCTCATCAGGAACGGTGCCTCTGTCTTTTTCTTCCCAGAAGGCACACGGAGTAAGGAT
GGGAAGCTAGGTGCTTTCAAGAAAGGTGCATTTAGTGTTGCAGCAAAAACTGGAGTGCC
TGTCGTTCCTATGACCCTAATTGGCACAGGCAAAATCATGCCTTTAGGACTGGAGGGTGT
CATAAATTCAGGATCCGTAAAAGTTGTTATTCACAAGCCGATCAAAGGAAGTGATCCAG
AAATATTATGCAATGAAGCTAGAAACACAATAGCAGATACGCTTAAGCATCAATGCTGA,
Theobroma cacao 1-acyl-sn-glycerol-3-phosphateacyltransferase
(TcLPAT2), codon-optimized, 933 nt
SEQ ID NO: 53
ATGGAGTCGTCGGGCTCGGGCTCGTTCCTGCGCAACCGCCGCCTGGGCTCGTTCCTGGAC
ACCAACTCGGACCCGAACGTGCGCGAGACCCAGAAGGTGCTGTCGAAGGGCGGCGCCCG
CCAGCGCCCGAAGACCGACGACGCCTTCGTGGACGACGACGGCTGGATCTGCTCGCTGA
TCTCGTGCGTGCGCATCGTGGCCTGCTTCCTGACCATGATGGTGACCACCTTCATCTGGG
CCCTGATCATGCTGCTGCTGCTGCCGTGGCCGTCGCAGCGCATCCGCCAGGGCAACATCT
ACGGCCACGTGACCGGCCGCCTGCTGATGTGGATCCTGGGCAACCCGATCAAGATCGAG
GGCACCGAGTTCTCGAACGAGCGCGCCATCTACATCTGCAACCACGCCTCGCCGATCGAC
ATCTTCCTGATCATGTGGCTGACCCCGACCGGCACCGTGGGCATCGCCAAGAAGGAGAT
CATCTGGTACCCGCTGTTCGGCCAGCTGTACGTGCTGGCCAACCACCTGCGCATCGACCG
CTCGAACCCGTCGACCGCCATCCAGTCGATGAAGGAGGCCGTGCAGGCCGTGATCAAGC
ACAACCTGTCGCTGATCATCTTCCCGGAGGGCACCCGCTCGAAGAACGGCCGCCTGCTGC
CGTTCAAGAAGGGCTTCGTGCACCTGGCCCTGCAGTCGCACATCCCGATCGTGCCGATCG
TGCTGACCGGCACCCACCTGGCCTGGCGCAAGGGCTCGCTGCACGTGCGCCCGGCCCCG
ATCTCGGTGAAGTACCTGCCGCCGATCTCGACCGACTCGTGGAAGGACGACAAGATCGA
CGACTACATCAAGATGGTGCACGACATCTACGTGGAGAACCTGCCGGAGCCGCAGAAGC
CGATCGTGTCGGAGGACACCACCAACTCGTCGCGCTCGTAA,
Theobroma cacao 1-acyl-sn-glycerol-3-phosphate acyltransferase
(TcLPAT2), NCBI Reference Sequence: XM_007020795.2 141-1073, 933 nt
SEQ ID NO: 54
ATGGAGAGTTCTGGAAGTGGTTCTTTCTTGAGGAACAGAAGATTAGGGAGCTTCCTCGAT
ACAAATTCTGATCCAAATGTGAGAGAAACTCAGAAAGTTTTGAGCAAAGGAGGAGCAAG
ACAGAGGCCTAAGACTGATGATGCTTTTGTTGATGATGACGGATGGATTTGTTCATTGAT
ATCTTGTGTAAGGATTGTTGCATGTTTCCTGACAATGATGGTCACAACATTCATTTGGGC
ATTGATCATGCTCTTGCTCCTTCCTTGGCCTTCCCAGCGAATCAGGCAAGGAAATATTTAT
GGCCATGTGACTGGTAGATTGCTGATGTGGATCTTAGGAAATCCTATAAAGATTGAAGG
AACAGAGTTCTCCAATGAGAGAGCCATTTATATCTGCAATCATGCATCTCCCATAGACAT
TTTCCTCATTATGTGGTTGACTCCAACAGGGACTGTTGGCATTGCAAAGAAAGAGATCAT
TTGGTATCCCCTATTTGGACAACTATATGTTCTAGCGAATCATCTCCGCATTGATCGATCT
AACCCTAGTACAGCCATTCAGTCCATGAAAGAGGCAGTTCAGGCTGTGATAAAACACAA
CCTATCTTTGATTATTTTTCCTGAGGGCACAAGGTCAAAAAATGGACGATTGCTCCCCTTT
AAAAAGGGCTTTGTTCATTTGGCCTTGCAGTCACACATTCCAATAGTTCCAATAGTCTTG
ACAGGTACTCATCTAGCATGGAGGAAAGGTAGCTTGCATGTTCGACCGGCTCCTATATCT
GTAAAATATCTCCCTCCGATAAGTACCGATAGTTGGAAAGATGACAAGATTGATGACTA
CATAAAAATGGTGCATGACATATATGTCGAAAACCTCCCTGAGCCTCAAAAGCCTATTGT
ATCAGAAGACACCACTAACAGTTCAAGATCATAA,
Theobroma cacao (TcLPAT2) truncated, codon-optimized (corresponds
to nt 136-933 of SEQ ID NO: 53), 798 nt
SEQ ID NO: 55
GACGACGCCTTCGTGGACGACGACGGCTGGATCTGCTCGCTGATCTCGTGCGTGCGCATC
GTGGCCTGCTTCCTGACCATGATGGTGACCACCTTCATCTGGGCCCTGATCATGCTGCTG
CTGCTGCCGTGGCCGTCGCAGCGCATCCGCCAGGGCAACATCTACGGCCACGTGACCGG
CCGCCTGCTGATGTGGATCCTGGGCAACCCGATCAAGATCGAGGGCACCGAGTTCTCGA
ACGAGCGCGCCATCTACATCTGCAACCACGCCTCGCCGATCGACATCTTCCTGATCATGT
GGCTGACCCCGACCGGCACCGTGGGCATCGCCAAGAAGGAGATCATCTGGTACCCGCTG
TTCGGCCAGCTGTACGTGCTGGCCAACCACCTGCGCATCGACCGCTCGAACCCGTCGACC
GCCATCCAGTCGATGAAGGAGGCCGTGCAGGCCGTGATCAAGCACAACCTGTCGCTGAT
CATCTTCCCGGAGGGCACCCGCTCGAAGAACGGCCGCCTGCTGCCGTTCAAGAAGGGCT
TCGTGCACCTGGCCCTGCAGTCGCACATCCCGATCGTGCCGATCGTGCTGACCGGCACCC
ACCTGGCCTGGCGCAAGGGCTCGCTGCACGTGCGCCCGGCCCCGATCTCGGTGAAGTAC
CTGCCGCCGATCTCGACCGACTCGTGGAAGGACGACAAGATCGACGACTACATCAAGAT
GGTGCACGACATCTACGTGGAGAACCTGCCGGAGCCGCAGAAGCCGATCGTGTCGGAGG
ACACCACCAACTCGTCGCGCTCGTAA,
Theobroma cacao (TcLPAT2) truncated (e.g., to remove organelle
targeting sequences) (corresponds to nt 136-933 of SEQ ID NO: 54),
798 nt
SEQ ID NO: 56
GATGATGCTTTTGTTGATGATGACGGATGGATTTGTTCATTGATATCTTGTGTAAGGATTG
TTGCATGTTTCCTGACAATGATGGTCACAACATTCATTTGGGCATTGATCATGCTCTTGCT
CCTTCCTTGGCCTTCCCAGCGAATCAGGCAAGGAAATATTTATGGCCATGTGACTGGTAG
ATTGCTGATGTGGATCTTAGGAAATCCTATAAAGATTGAAGGAACAGAGTTCTCCAATGA
GAGAGCCATTTATATCTGCAATCATGCATCTCCCATAGACATTTTCCTCATTATGTGGTTG
ACTCCAACAGGGACTGTTGGCATTGCAAAGAAAGAGATCATTTGGTATCCCCTATTTGGA
CAACTATATGTTCTAGCGAATCATCTCCGCATTGATCGATCTAACCCTAGTACAGCCATT
CAGTCCATGAAAGAGGCAGTTCAGGCTGTGATAAAACACAACCTATCTTTGATTATTTTT
CCTGAGGGCACAAGGTCAAAAAATGGACGATTGCTCCCCTTTAAAAAGGGCTTTGTTCAT
TTGGCCTTGCAGTCACACATTCCAATAGTTCCAATAGTCTTGACAGGTACTCATCTAGCA
TGGAGGAAAGGTAGCTTGCATGTTCGACCGGCTCCTATATCTGTAAAATATCTCCCTCCG
ATAAGTACCGATAGTTGGAAAGATGACAAGATTGATGACTACATAAAAATGGTGCATGA
CATATATGTCGAAAACCTCCCTGAGCCTCAAAAGCCTATTGTATCAGAAGACACCACTAA
CAGTTCAAGATCATAA,
Theobroma cacao 1-acyl-sn-glycerol-3-phosphate acyltransferase
4 (TcLPAT3), XM_007017391.2 348-1493, 1146 nt
SEQ ID NO: 57
ATGGAAGTTTGCAGGCCCCTCAAACCTGATGATAAATTAAAGCACCGCCCTTTGACTCCT
TTTAGGTTTTTAAGGGGTCTGATATGTTTAGTGGTGTTTCTCTTGACTGCTTTTATGTTTCT
AGCGTATTTAGGACCTGGGGCTGTCCTATTGCGATTTTTCAGCCTACACTACTGTAGGAA
GGCAACATCCTTCTTCTTTGGCCTATGGCTAGCTTTGTGGCCCTTTCTTTTTGAAAAAATA
AACAGGACTAAAGTGGTTTTCTCTGGGGATAATGCTCCACAGAAGGAACGTGTTTTACTT
ATTGTCAATCACAGGACTGAAGTTGATTGGATGTACCTCTGGGATCTTGCAATGCGAAAG
GGCTGCCTGGGCTACATCAAATATATTCTTAAGAGCAGCCTGATGAAACTACCTGTCCTT
GGTTGGGGATTTCACATCTTGGAGTTCATTTCAGTAGATAGGAAGTGGGAAACTGATGAA
AATGTCCTGCGCCAAATGCTTTCAACCTTTAAGAATCCTCGAGATCCTTTATGGCTTGCTC
TTTTCCCTGAAGGAACCGATTTTACCGAAGAAAAATGCAGGAACAGTCAGAAGTTTGCA
GCTGAAGTTGGATTGCCTGTGTTGACAAATGTGCTGCTACCGAGAACAAGGGGGTTTTGC
CTTTGCTTAGAAACACTTAGGGACTCTTTGGATGCAGTTTACGATTTGAGTATTGCATATA
AGCACCAATGCCCCTTCTTTCTGGACAATGTTTTTGGTGTGGATCCATCAGAGGTTCACAT
TCATGTTCGACGTATCCCAGTTAAGGAGATCCCAACATCTAATGCAGAGGCTGCTGCTTG
GTTAATTGATACATTCAAGCTCAAGGACCAGTTGCTCTCAGATTTCAAATCTCAGGGACA
TTTTCCTAACCAAGGAACTCAACAAGAACTTTCTTCTTTGAAGTCCTTATTAAATCTAACA
GTGATAATATCCTTGACAGCCATATTCACTTATCTTACCTTTTCTTCCAATTTGTACATGA
TATATGTAAGCTTAGCTTGTCTATACCTTGCTTACATTACTCATTATAAAATTCGCCCAAT
GCCAGTTCTAAGCTCTGTAAAACCGCTGTCTTACCCAAAGGGCAAGAGAGATGAATAA,
Theobroma cacao Lysophosphatidyl acyltransferase 5 (TcLPAT4),
GenBank: CM001880.1 REGION: 6183094-6185435 with CDS: 1-563,
806-936, 1918-2342; 1119 nt
SEQ ID NO: 58
ATGGAAGTTCCTAGTGCAAATCATGAAATGAGGCATCGTTCATTGACCCCGCTAAGGGTG
TTTAGGGGTCTAATATGTTTGCTAGTGCTGTTTTCAACAGCTTTTATGATGATAGTGTATT
GTGGCTTTCTTACCACTGTTATATTCAGGCTTTTCAGCATACATTACAGTCGGAAAGCAA
CTTCTTTCTTCTTTAGTGCTTGGCTGTCTTTATGGCCCTTTTTATTTGAGAAAATAAACAA
AACAAAAGTCATTTTTTCTGGAGATGATGTTCCTCCAAGGGAACGCGTTTTGCTTATTTGC
AACCACAGAACCGAGGTTGACTGGATGTACTTGTGGGACTTTGCATTGCGGAAAGGTTG
CCTGGGATACATAAAGTATATCCTTAAGAGCAGCTTGATGAAATTACCTGTATTTGGTTG
GGCTTTCCATATTTTAGAGTTCATCCCTGTGGAAAGGAAGTGGGAGGTTGATGAATCTAA
CATGCGCAACATGCTTTCAACATTCAAAGATCCTCAAGATCCTCTCTGGCTTGTTCTCTTT
CCCGAAGGAACAGATTTCACTGAGCAAAAATGCTTAAGAAGTCAAAAATATGCAGCTGA
AAATGGCTTACCTATCCTAAAGAATTTGCTGCTTCCAAAATCAAAGGGTTTTTTCGCCTG
CTTGGAAGATTTGAGGAGCTCTTTGGATGCAGTTTATGATGTGACCATTGGATATAAGCA
TTGCTGCCCATCCTTCTTGGACAATGTCTTCGGGGTAGACCCTTCTGAAGTTCATATTCAC
ATCAGACGCATTACCCTGGATGACATTCCAATATCTGAAAGGGAGTTAACCGCTTGGTTA
ATGGATACATTTCAACATAAAGATCAATTGCTTTCTAATTTCAAGTCTGAAGGTTATTTCC
CTCGGCAAGGACCGGAAGTAAACCTCTCTGCAGTGAAGTGCATTGTAGACGTTGTGCTG
GTGCTTTTCTTGACTAGTGCATTCATATTTTTCACCTTTTTCTCATCCATTTGGTTTAAGAT
ATTTGTATCTTTATCTTGTGCCTATATGACTTCTGCAACTTATTTAAACACCCGTCCAGTA
CCAGTCTTCAGCCTTGTGAAAACTTGTGTCTAA,
Theobroma cacao 1-acyl-sn-glycerol-3-phosphate acyltransferase 1,
chloroplastic (TcLPAT1), Ref. No. XP_007011912.2, 359 aa (corresponds
to SEQ ID NO: 52)
SEQ ID NO: 59
MELSSLPSVSSFSLCHPKPRSSATLPFLPFSNRKGAYFGYSLCKRATLRNSCNSAQNNFLRISR
THDGVSRCYFNQKEGLNRSYYNSKLHNENKLSRYIVARSEFAGTGTPDAAYSLSEIKPGSKV
RGVCFYAVTAIAAILLIWFMLVLHPFVLLFDRYRRKAQHFIAKLWAMATVAPFFKIEFEGLEN
LPPQDVPAVYVSNHQSFLDIYTLLTLGRSFKFISKTGIFLYPIIGWAMSMMGLIPLKRMDSRSQ
LDCLKRCMDLIRNGASVFFFPEGTRSKDGKLGAFKKGAFSVAAKTGVPVVPMTLIGTGKIMP
LGLEGVINSGSVKVVIHKPIKGSDPEILCNEARNTIADTLKHQC,
Theobroma cacao 1-acyl-sn-glycerol-3-phosphate acyltransferase
(TcLPAT2), Ref. No. XP_007020857.2 (corresponds to SEQ ID NO: 53
or SEQ ID NO: 54), 310 aa
SEQ ID NO: 60
MESSGSGSFLRNRRLGSFLDTNSDPNVRETQKVLSKGGARQRPKTDDAFVDDDGWICSLISC
VRIVACFLTMMVTTFIWALIMLLLLPWPSQRIRQGNIYGHVTGRLLMWILGNPIKIEGTEFSNE
RAIYICNHASPIDIFLIMWLTPTGTVGIAKKEIIWYPLFGQLYVLANHLRIDRSNPSTAIQSMKE
AVQAVIKHNLSLIIFPEGTRSKNGRLLPFKKGFVHLALQSHIPIVPIVLTGTHLAWRKGSLHVR
PAPISVKYLPPISTDSWKDDKIDDYIKMVHDIYVENLPEPQKPIVSEDTTNSSRS,
Theobroma cacao TcLPAT2 truncated (corresponds to SEQ ID NO: 55
or SEQ ID NO: 56; corresponds to aa 46-310 of SEQ ID NO: 60), 266 aa
SEQ ID NO: 61
MDDAFVDDDGWICSLISCVRIVACFLTMMVTTFIWALIMLLLLPWPSQRIRQGNIYGHVTGR
LLMWILGNPIKIEGTEFSNERAIYICNHASPIDIFLIMWLTPTGTVGIAKKEIIWYPLFGQLYVLA
NHLRIDRSNPSTAIQSMKEAVQAVIKHNLSLIIFPEGTRSKNGRLLPFKKGFVHLALQSHIPIVPI
VLTGTHLAWRKGSLHVRPAPISVKYLPPISTDSWKDDKIDDYIKMVHDIYVENLPEPQKPIVS
EDTTNSSRS,
Theobroma cacao 1-acyl-sn-glycerol-3-phosphate acyltransferase 4
(TcLPAT3) Ref. No. XP_007017453.1 (corresponds to SEQ ID NO: 57),
381 aa
SEQ ID NO: 62
MEVCRPLKPDDKLKHRPLTPFRFLRGLICLVVFLLTAFMFLAYLGPGAVLLRFFSLHYCRKAT
SFFFGLWLALWPFLFEKINRTKVVFSGDNAPQKERVLLIVNHRTEVDWMYLWDLAMRKGCL
GYIKYILKSSLMKLPVLGWGFHILEFISVDRKWETDENVLRQMLSTFKNPRDPLWLALFPEGT
DFTEEKCRNSQKFAAEVGLPVLTNVLLPRTRGFCLCLETLRDSLDAVYDLSIAYKHQCPFFLD
NVFGVDPSEVHIHVRRIPVKEIPTSNAEAAAWLIDTFKLKDQLLSDFKSQGHFPNQGTQQELS
SLKSLLNLTVIISLTAIFTYLTFSSNLYMIYVSLACLYLAYITHYKIRPMPVLSSVKPLSYPKGK
RDE,
Theobroma cacao Lysophosphatidyl acyltransferase 5 (TcLPAT4) Ref.
No. EOX98557.1 (corresponds to SEQ ID NO: 58), 372 aa
SEQ ID NO: 63
MEVPSANHEMRHRSLTPLRVFRGLICLLVLFSTAFMMIVYCGFLTTVIFRLFSIHYSRKATSFF
FSAWLSLWPFLFEKINKTKVIFSGDDVPPRERVLLICNHRTEVDWMYLWDFALRKGCLGYIK
YILKSSLMKLPVFGWAFHILEFIPVERKWEVDESNMRNMLSTFKDPQDPLWLVLFPEGTDFTE
QKCLRSQKYAAENGLPILKNLLLPKSKGFFACLEDLRSSLDAVYDVTIGYKHCCPSFLDNVFG
VDPSEVHIHIRRITLDDIPISERELTAWLMDTFQHKDQLLSNFKSEGYFPRQGPEVNLSAVKCI
VDVVLVLFLTSAFIFFTFFSSIWFKIFVSLSCAYMTSATYLNTRPVPVFSLVKTCV,

In some embodiments of any of the aspects, the acyltransferase catalyzes transesterification of the sn1 OH group of a glyceraldehyde-3-phosphate with a fatty acid. As a non-limiting example, such an acyltransferase is glycerol-3-phosphate acyltransferase (GPAT; E.C. 2.3.1.15). GPAT transfers an acyl-group from acyl-ACP to the sn-1 position of glycerol-3-phosphate producing a lysophosphatidic acid (LPA), an essential step for the triacylglycerol (TAG) and glycerophospholipids. In competition assays, GPATs can show preferences for fatty acyl-CoA substrates of specific chain length and desaturation. In some embodiments of any of the aspects, the functional GPAT gene preferentially produces or leads to the production of TAGs comprising a specific type of fatty acid R group (e.g., C16). As such, the functional GPAT gene can be selected from any GPAT gene from any species that preferentially produces or leads to the production of TAGs comprising a specific type of fatty acid R group (e.g., C16). In some embodiments of any of the aspects, the GPAT is a bacterial GPAT. In some embodiments of any of the aspects, the GPAT is a plant GPAT.

In some embodiments of any of the aspects, the engineered bacterium comprises at least one exogenous copy of at least one functional GPAT gene. In some embodiments of any of the aspects, the engineered bacterium does not comprise a functional endogenous GPAT gene. In some embodiments of any of the aspects, the functional GPAT gene is heterologous. In some embodiments of any of the aspects, the functional heterologous GPAT gene comprises a Durio GPAT gene. In some embodiments of any of the aspects, the functional heterologous GPAT gene comprises a Gossypium GPAT gene. In some embodiments of any of the aspects, the functional heterologous GPAT gene comprises a Hibiscus GPAT gene. In some embodiments of any of the aspects, the functional heterologous GPAT gene comprises a Theobroma GPAT gene.

In some embodiments of any of the aspects, the functional heterologous GPAT gene comprises a Durio zibethinus GPAT gene, Gossypium arboreum GPAT gene, Hibiscus syriacus GPAT gene, or a Theobroma cacao GPAT gene. In some embodiments of any of the aspects, the engineered bacterium comprises at least one exogenous copy of at least one functional GPAT gene comprising one of SEQ ID NOs: 64-67, 69, 71-79, or a nucleic acid sequence that is at least 80%, at least 85%, at least 90%, at least 91%, at least 92%, at least 93%, at least 94%, at least 95%, at least 96%, at least 97%, at least 98%, or at least 99% identical to the sequence of at least one of SEQ ID NOs: 64-67, 69, 71-79, that maintains the same functions as at least one of SEQ ID NOs: 64-67, 69, 71-79 (e.g., glycerol-3-phosphate acyltransferase).

In some embodiments of any of the aspects, the amino acid sequence encoded by the functional GPAT gene comprises one of SEQ ID NOs: 80-89, or an amino acid sequence that is at least 80%, at least 85%, at least 90%, at least 91%, at least 92%, at least 93%, at least 94%, at least 95%, at least 96%, at least 97%, at least 98%, or at least 99% identical to the sequence of at least one of SEQ ID NOs: 80-89, that maintains the same functions as at least one of SEQ ID NOs: 80-89 (e.g., glycerol-3-phosphate acyltransferase).

Durio zibethinus glycerol-3-phosphate acyltransferase 8 isoform X1
(DzGPAT) XM_022914718.1 216-1718, 1503 nt
SEQ ID NO: 64
ATGGCTCCACTGAAAGCGGCGCAAAGCTTTCCATCGATAACGGAATGCGACGGTTCTAC
GTACGAATCGATCGCCGCTGATCTCGATGGCACGCTTTTAATCTCTCGAAGCTCTTTTCCT
TACTTCATGCTCGTCGCCGTGGAAGCGGGAAGCCTCTTTCGAGGTCTTATCCTTCTTCTCT
CTCTGCCTCTGATAATTATTTCTTATCTCTTCGTTTCCGAAGCTATTGGTATCCAAATCCTC
ATTTTTATCTCCTTCGCTGGACTCAAGATCCGCGACATCGAGCTCGTCTCCCGCGCTGTTC
TTCCCAGATTTTATGCTGCGAATGTGAGGAAGGAAAGTTTCGAAGTGTTTGACAGATGCA
AGAGGAAGGTGGTGGTGACGGCGAATCCGACATTCATGGTTGAGCCGTTTGTGAAGGAT
TTTCTAGGTGGAGATAAGGTTTTGGGCACGGAGATTGAAGTGAACCCTAAAACAAAGAA
GGCCACGGGGTTTGTCAAGAAGCCAGGGGTTTTAGTAGGAAAGTTGAAGAGATTGGCCA
TTTTCAAGGAGTTCGGTGATGAATCACCTGATCTTGGAATCGGAGACCGTGAATCTGATC
ACGATTTCATGTCAATTTGCAAGGAGGGTTACATGGTGCACCCTAGTAAGTCAGCAACAC
CGGTACAACTGGATCGTTTAAAAAGCCGCATCATCTTTCATGATGGTCGCTTTGTTCAGC
GCCCGGACCCACTCAATGCCTTAATCACTTATATTTGGCTGCCATTTGGCTTCATCCTATC
CATCATTCGAGTTTACTTCAATCTACCTTTACCAGAGCGTATTGTACGCTACACATACGA
AATGCTGGGTATCCACCTCGTGATCCGTGGGAAGAGACCTCCCCCACCATCTCCTGGGAC
TCCAGGTAACCTCTACGTCTGCAATCACCGTTCAGCTCTTGATCCAATTGTGATCGCCATC
GCACTCGGACGCAAAGTTTCGTGTGTTACATACAGCGTAAGCCGTCTCTCGAGGTTCCTT
TCCCCAATCCCAGCGATTGCTTTAACTCGTGATCGTGCGGCTGATGCTGCCCGAATTTCA
GAACTATTACAAAAAGGTGATTTAGTGGTGTGTCCAGAGGGGACCACGTGCCGCGAGCA
GTTCTTATTACGATTCAGTGCTTTGTTTGCAGAAATGAGCGATAGGATAGTGCCCGTGGC
GGTCAATTGCAGGCAAAACATGTTTTATGGGACGACCGTGAGAGGGGTCAAATTTTGGG
ACCCTTATTTTTTCTTCATGAATCCTAGGCCAACATACGAGGTCACTTTCCTTGATCGATT
GCCGGAGGAGATGACGGTGAAGGCCGGAGGGAAATCGGCTATTGAGGTGGCTAATCAC
GTGCAGAAGGTGCTGGGTGATGTCCTGGGGTTTGAGTGCACTGGATTGACAAGGAAGGA
TAAATATATGTTGCTTGGGGGAAATGATGGTAAGGTTGAATCGATTTACAATGCAAAGA
AATAA
Gossypium arboreum glycerol-3-phosphate acyltransferase 8-like
protein (GaGPAT), GenBank: KN449683 REGION: 14316-18632, CDS join (1-311, 432-744, 3439-
4317), 1503 nt
SEQ ID NO: 65
ATGGCTCCACCGAAAGCAGGGAAAACCTTTCCATCGATAACGGAATGCGATGGATTGAA
GTATGAATCGATCGCCGCCGATCTGGATGGCACGCTTTTAATCTCACGAAGCTCTTTCCC
CTACTTCATGCTTATTGCCGTCGAAGCGGGAAGCCTCCTTCGAGGTCTTATCCTTCTTCTT
TCTCTGCCTCTGGTCATCATATCTTATCTCTTCATTTCCGAAGCTATTGGTATTCAAATCCT
CATTTTCATCTCCTTCGCTGGACTCAAGATCCGCGACATCGAGCTGGTATCTCGCGCTGTT
CTTCCCAGATTCTATGCTGCAAATGTAAGGAAGGAAAGTTTCGAGGTATTTGACAGATGC
AAGAGGAAAGTAGTGGTAACGGCGAATCCGACGTTCATGGTGGAGCCGTTTGTGAAGGA
TTTTCTCGGCGGAGATAAAGTTTTAGGCACAGAGATTGAAGTGAACCCTAAAACAAAGA
AGGCGACGGGATTTGTGAAGAATCCCGGGGTTTTAGTAGGGAAGTTTAAGAGATTAGCC
ATTTTGAAGGAGTTTGGTGATGAATCGCCTGATCTTGGAATCGGAGACCGTGAATCTGAT
CATGATTTCATGTCAATTTGCAAGGAGGGGTACATGGTGCACCCTAGCAAATCAGCATCA
CCAGTACCGCTTGATCGCCTAAAGAGCCGCATTATCTTCCACGATGGTCGCTTTGTCCAA
CGTCCGGATCCACTCAATGCCTGGCTAACCTACCTTTGGCTGCCATTTGGCTTCATCCTCT
CCATTATTCGTGTCTACTTCAATCTACCTTTACCCGAGCGTATCGTACGCTACACTTACGA
GATGCTCGGCATTCACCTCGTGATCCGCGGAAAGCGACCACCCCCACCATCTGCGGGGA
CCCCAGGCAACCTCTACGTCTGCAATCACCGCACAGCTCTTGACCCGATTGTGATCGCCA
TCGCACTTGGACGCAAAGTCTCGTGTGTCACATACAGCGTAAGCCGTCTCTCGAGGTTCC
TATCTCCAATCCCAGCCATTGCTTTAACTCGTGATCGTGCGGCCGATGCTGCCCGAATTTC
AGAACTGTTGCAAAAAGGTGATCTAGTAGTTTGTCCGGAGGGGACCACGTGTCGTGAGC
AATTCTTGTTGAGGTTCAGTGCTTTGTTCGCAGAAATGAGCGATAGGATCGTCCCCGTTG
CCGTCAATTGCAAGCAGAGCATGTTCTACGGGACGACCGTGAGAGGGGTCAAATTCTGG
GACCCTTATTTCTTCTTCATGAATCCAAGGCCAACATACGAGGTCACGTTCCTTGATCGAT
TGCCGGAAGAGATGACAGTGAAGGCCGGAGGGAAATCGGCGATCGAGGTAGCGAATCA
CGTGCAGAAGGTGTTGGGTGATGTCCTGGGGTTTGAATGCACTGGATTGACTAGGAAAG
ATAAATACATGTTGCTTGGAGGAAACGATGGTAAGGTAGAATCGATGTACAATGGCAAG
AAATAA
Hibiscus syriacus glycerol-3-phosphate acyltransferase 8 (HsGPAT),
NCBI Reference Sequence: XM_039207737.1 52-1554, 1503 nt
SEQ ID NO: 66
ATGACTCCACTAAGAGCTGGGAGAAGGTTTCCTTCAATAACGGAATGCAACGGATCGAC
ATACGAATCGATCGCCGCCGATCTCGATGGCACGCTTTTAATCTCACGTAGCTCTTTTCCG
TATTTCATGCTCATTGCCGTCGAAGCGGGAAGCCTCCTTCGAGGTCTTATCCTTCTTCTTT
CACTGCCTCTTGTCATTGTATCTTATCTCTTCATTTCCGAAGCTATTGGTATACAAATCCT
CATTTTCATCTCCTTCGCTGGACTCAAGATCCGCGACATCGAGTTGGTCTCCCGCGCTATT
CTTCCCAGATTTTATGCTGCGAATGTGAGGAAGGAAAGTTTCGAAGTATTTGACAGATGC
AAGAGGAAAGTGGTAGTGACGGCGAATCCGACGTTCATGGTGGAGCCGTTTGTGAAGGA
TTTTCTTGGTGGAGATAAGGTTTTAGGCACAGAGATCGAAGTGAACCCTAAAACAAAGA
AGGCCACGGGATTTGTTAAGAAGCCAGGCGTTTTAGTAAGCGAATTGAAGAGATTGGCC
ATTCTGAAGGAGTTTGGTGACGATTCGCCCGATCTTGGAATCGGAGACCGTGAATCTGAT
CACGATTTCATGTCAATTTGCAAGGAGGGCTACATGGTGCACCCTAGCAAATCAGCATCA
CCGGTACCACTTGATCGCCTGAGAAGCCGCATCATCTTCCACGATGGTCGGTTTGTTCAA
CGCCCGGACCCACTCAATGCCTTGATCACCTACATTTGGCTGCCATTCGGCTTCATCTTGT
CCATCATTCGCGTCTACTTCAATCTACCTTTACCCGAGCGCATCGTACGCTACACATACG
AAATGTTGGGCATTCACCTCGTGATCCGCGGGAAGCGACCGTCGCCGCCGTCTCCGGGTA
CCCCAGGCAACCTCTACGTCTGTAATCACCGTTCAGCACTTGATCCAATCGTCATCGCCA
TCGCTCTCGGACGTAAAGTTTCGTGTGTCACATACAGTGTAAGCCGTCTCTCGAGGTTCC
TATCACCAATCCCAGCCATCGCTTTAACTCGTGATCGTGCGGCTGATGCCGCCCGAATTT
CAGAGCTATTACAAAAAGGTGATCTAGTAGTTTGTCCGGAGGGGACCACGTGTCGCGAG
CCATTCCTGTTGCGATTCAGTGCTTTGTTCGCAGAAATGAGCGATAGAATCGTCCCCGTC
GCCGTCAATTGCAAGCAGAGCATGTTCTACGGGACGACCGTGAGAGGGGTAAAATTCTG
GGACCCTTACTTTTTCTTCATGAACCCAAGGCCAACATACGAGGTCACGTTCCTCGACCG
GTTGCCGGAAGAGATGACGGTGAAGGCCGGAGGAAAATCGGCTATCGAGGTGGCGAAT
CACGTGCAGAAGGTGTTGGGCGATGTACTGGGCTTTGAATGCACGGGATTGACTAGGAA
AGACAAGTATATGTTGCTCGGTGGAAATGATGGTAAGGTCGAATCCATGTACAATGGCA
AGAAATAA
Theobroma cacao Glycerol-3-phosphate acyltransferase 8 (TcGPAT1),
GenBank: CM001879.1 region: complement (36085880-36088399), CDS join (1-311, 448-760, 1642-
2520), 1503 nt
SEQ ID NO: 67
ATGGCTCCAGCGAAATCGGGGCGAAGCTTTCCTTCGATAACGAAATGCGAAGGTTCGAC
GTACGAATCGATAGCCGCCGACCTCGACGGCACGCTTTTAATCTCTCGAAGCTCTTTTCC
TTACTTCATGCTTGTCGCCGTCGAAGCTGGAAGCCTCCTTCGAGGTCTTATCCTTCTTCTC
TCTCTGCCTCTGGTCATTGTTTCTTATCTCTTCATTTCCGAAGCTATTGGCATCCAAATCCT
CATATTTATCTCCTTCGCTGGACTCAAGATCCGCGACATCGAGCTCGTCTCCCGTGCTGTT
CTTCCCAGGTTTTATGCTGCGAATGTGAGGAAGGAAAGTTTCGAGGTGTTTGACAGATGT
AAGAGGAAAGTGGTGGTGACGGCGAATCCGACGTTCATGGTGGAGCCGTTTGTGAAGGA
TTTTCTAGGTGGAGACAAGGTTTTAGGCACAGAAATTGAAGTGAACCCCAAAACAAAGA
AGGCCACGGGGTTTGTCAAGAAGCCTGGGGTTTTAGTCTCGGAGTTGAAGAGACTGGCC
ATTTTGAAGGAGTTTGGAGAGGAATCACCTGATCTTGGAATCGGAGACCGTGAATCTGAT
CATGATTTCATGTCAATTTGCAAGGAGGGCTACATGGTGCACCCTAGCAAATCAGCAACA
CCAGTACCTCTTGATCGCCTAAAGAGCCGCATCATCTTCCATGATGGTCGCTTTGTCCAG
CGCCCGGACCCGCTCAATGCCTTGATCACCTATTTGTGGCTGCCATTTGGCTTCATCCTAT
CCATCGTTCGCGTTTACTTCAATCTGCCTTTACCAGAGCGTATCGTACGCTACACATACGA
AATGCTGGGCATCCACCTCGTGATCCGCGGGAAGCGTCCTCCCCCTCCATCCCCTGGGAC
CCCAGGCAACCTCTACGTCTGCAATCACCGTTCAGCTCTTGATCCAATCGTGATTGCCAT
CGCACTCGGTCGCAAAGTTTCGTGTGTCACGTACAGCGTAAGCCGCCTCTCGAGGTTCCT
GTCTCCAATCCCAGCCGTCGCTTTAACTCGTGATCGTGCGGCTGATGCTGCTAGAATTTC
AGAACTATTGCAAAAAGGTGATCTAGTTGTGTGCCCAGAGGGGACCACGTGCCGCGAGC
AGTTCCTGTTGCGATTCAGTGCTTTGTTCGCAGAGTTGAGCGATAGGATCGTGCCCGTGG
CGGTCAATTGCAGGCAGAACATGTTTTATGGTACGACCGTGAGAGGGGTCAAATTTTGG
GACCCTTATTTTTTCTTCATGAATCCTAGGCCAACGTACGAGGTCACTTTCCTTGATCGTT
TGCCGGAGGAGATGACGGTTAAGGCCGGAGGGAAATCGTCGATTGAGGTGGCCAATCAC
GTGCAGAAGGTGCTGGGCGATGTCCTGGGGTTTGAGTGCACTGGATTGACTAGGAAGGA
TAAATATCTGTTGCTTGGAGGAAACGACGGTAAGGTTGAATCAATGTACAATGCCAAGA
AATAA
Theobroma cacao Glycerol-3-phosphate acyltransferase 9 isoform 1
(TcGPAT2), GenBank: CM001880.1 region: complement (2403687-2408244) CDS join (1-242, 364-
421, 746-864, 1029-1128, 1370-1465, 1653-1749, 2474-2561, 2642-2738, 2834-2919, 3493-3612,
3752-3850, 4510-4558), 1251 nt
SEQ ID NO: 69
ATGAAGAAGGAAAAGCTGTTTGGTTTTATCAATATAAAGAAAAAAGAAAAAGAAGAAA
AAAAACCAAAACCAAACAAAAATCCCAACAGCCAAACCAAATCAGAAAGCGAAAGGGA
AGGCATGAGTAGCAGAGGAGGGAAGCTGAGCTCATCCAGCTCCGAATTGGACTTGGATG
GACCTAACATCGAAGATTATCTCCCGTCCGGATCCTCCATTAACGAACCTCGCGGGAAGC
TTCGCCTACGCGATTTGCTTGATATTTCTCCCACTTTAACTGAAGCTGCTGGTGCCATTGT
TGATGATTCATTCACACGATGTTTTAAGTCGAATCCCCCTGAACCATGGAACTGGAATGT
ATATTTGTTCCCACTGTGGTGTTTTGGTGTGGCAGTTCGGTACTTGATTTTATTCCCTGCG
AGGGTTGTGGTGTTGACAATAGGATGGATAATATTTCTTTCATCCTTCATTCCCGTACACT
TTCTACTCAAAGGGCATGATAAGTTGCGGAAAAAGATGGAGAGGGTTTTGGTGGAGCTA
ATGTGCAGCTTCTTTGTTGCATCGTGGACTGGAGTTGTGAAGTACCATGGACCACGGCCT
AGCATTCGGCCCAAGCAGGTGTTTGTGGCCAATCATACTTCTATGATCGATTTCATCATA
TTAGAACAGATGACTGCATTTGCTGTCATTATGCAGAAGCACCCTGGATGGGTTGGACTG
TTGCAGAGCACTATTTTAGAGAGTGTGGGGTGTATTTGGTTCAACCGTTCAGAGGCAAAA
GATCGGGAAATTGTAGCAAAGAAGTTAAGGGATCATGTTCAGGGGGTTGACAATAACCC
GCTTCTCATTTTTCCTGAAGGGACCTGCATAAACAATCAGTACAGTGTCATGTTTAAGAA
GGGTGCATTTGAACTCGGTTGCACAGTTTGTCCGATTGCAATCAAGTACAATAAAATTTT
TGTTGATGCGTTTTGGAATAGCCGGAAGCAGTCCTTCACGATGCATCTGTTGCAGCTTAT
GACATCCTGGGCTGTTGTTTGTGATGTGTGGTACCTAGAACCCCAAAATCTAAGGCCTGG
AGAAACACCAATTGAATTTGCAGAGAGGGTCAGAGACATAATATCTGTTCGAGCAGGTC
TTAAAAAGGTTCCATGGGATGGATATTTGAAGTACTCTCGCCCTAGCCCTAAGCATAGAG
AGCGAAAGCAACAAAGCTTTGCTGAGTCCGTGCTTCTGCGACTGGAGGAAAAGTGA
Theobroma cacao Glycerol-3-phosphate acyltransferase 9 isoform 1
(TcGPAT2) truncated (corresponds to nt 211-1251 of SEQ ID NO: 69), 1044 nt
SEQ ID NO: 71
ATGTCCATTAACGAACCTCGCGGGAAGCTTCGCCTACGCGATTTGCTTGATATTTCTCCC
ACTTTAACTGAAGCTGCTGGTGCCATTGTTGATGATTCATTCACACGATGTTTTAAGTCGA
ATCCCCCTGAACCATGGAACTGGAATGTATATTTGTTCCCACTGTGGTGTTTTGGTGTGGC
AGTTCGGTACTTGATTTTATTCCCTGCGAGGGTTGTGGTGTTGACAATAGGATGGATAAT
ATTTCTTTCATCCTTCATTCCCGTACACTTTCTACTCAAAGGGCATGATAAGTTGCGGAAA
AAGATGGAGAGGGTTTTGGTGGAGCTAATGTGCAGCTTCTTTGTTGCATCGTGGACTGGA
GTTGTGAAGTACCATGGACCACGGCCTAGCATTCGGCCCAAGCAGGTGTTTGTGGCCAAT
CATACTTCTATGATCGATTTCATCATATTAGAACAGATGACTGCATTTGCTGTCATTATGC
AGAAGCACCCTGGATGGGTTGGACTGTTGCAGAGCACTATTTTAGAGAGTGTGGGGTGT
ATTTGGTTCAACCGTTCAGAGGCAAAAGATCGGGAAATTGTAGCAAAGAAGTTAAGGGA
TCATGTTCAGGGGGTTGACAATAACCCGCTTCTCATTTTTCCTGAAGGGACCTGCATAAA
CAATCAGTACAGTGTCATGTTTAAGAAGGGTGCATTTGAACTCGGTTGCACAGTTTGTCC
GATTGCAATCAAGTACAATAAAATTTTTGTTGATGCGTTTTGGAATAGCCGGAAGCAGTC
CTTCACGATGCATCTGTTGCAGCTTATGACATCCTGGGCTGTTGTTTGTGATGTGTGGTAC
CTAGAACCCCAAAATCTAAGGCCTGGAGAAACACCAATTGAATTTGCAGAGAGGGTCAG
AGACATAATATCTGTTCGAGCAGGTCTTAAAAAGGTTCCATGGGATGGATATTTGAAGTA
CTCTCGCCCTAGCCCTAAGCATAGAGAGCGAAAGCAACAAAGCTTTGCTGAGTCCGTGCT
TCTGCGACTGGAGGAAAAGTGA
Theobroma cacao Glycerol-3-phosphate acyltransferase 1 (TcGPAT3)
codon-optimized, 1623 nt
SEQ ID NO: 72
ATGGTGTTCCCGGTGGTGTTCCTGAAGCTGGCCGACTGGGTGCTGTACCAGCTGCTGGCC
AACTCGTGCTACCGCGCCGCCCGCAAGATGCGCAACTACGGCTTCTTCCTGCGCAACCAG
ACCCTGCGCTCGCCGCCGCAGCAGCAGGCCGCCTCGCTGTTCCCGTCGGTGACCAAGTGC
GACGTGGGCAACTCGCGCCGCTTCGACACCCTGGTGTGCGACATCCACGGCGTGCTGCTG
GGCTCGGACACCTTCTTCCCGTACTTCATGCTGGTGGCCTTCGAGGGCGGCTCGATCGTG
CGCGCCTTCCTGCTGCTGCTGTCGTGCTCGTTCCTGTGGGTGCTGGACTCGGAGCTGAAG
CTGCGCATCATGATCTTCATCTCGTTCTGCGGCCTGCGCAAGAAGGACATCGAGTCGGTG
GGCCGCGCCGTGCTGCCGAAGTTCTACCTGGAGAACCTGAACCTGCAGGTGTACGAGGT
GTGGTCGAAGACCTCGTCGCGCGTGGTGTTCACCTCGATCCCGCGCGTGATGGTGGAGGG
CTTCCTGCACGAGTACATGTCGGCCTCGGGCGTGGTGGGCACCGAGCTGCACACCGTGG
GCAACCGCTTCACCGGCCTGCTGTCGTCGTCGGGCCTGCTGGTGAAGCACAACGCCCTGA
AGGAGCACTTCGGCGACAAGAAGCCGGACGTGGGCCTGGGCTCGTCGTCGCTGCACGAC
CAGTACTTCATCTCGCTGTGCAAGGAGGCCTACGTGGTGAACATGGAGGACGGCAAGTC
GAACCTGTCGTCGTTCATGCCGCGCGACAAGTACCCGAAGCCGCTGATCTTCCACGACGG
CCGCCTGGCCTTCCTGCCGACCCCGTTCGCCACCCTGTCGATGTTCCTGTGGCTGCCGTTC
GGCATCGTGCTGTCGATCCTGCGCATCTTCGTGGGCATCTGCCTGCCGTACAAGCTGGCC
GTGATCTGCGCCACCCTGTCGGGCGTGCAGCTGAAGTTCCAGGGCTGCTTCCCGTCGTCG
AACTCGCAGCACAAGAAGGGCGTGCTGTACGTGTGCACCCACCGCACCCTGCTGGACCC
GGTGTTCCTGTCGACCGCCCTGTGCAAGCCGCTGACCGCCGTGACCTACTCGCTGTCGAA
GATGTCGGAGCTGATCGCCCCGATCAAGACCGTGCGCCTGACCCGCGACCGCAAGCAGG
ACGGCGAGACCATGCAGAAGCTGCTGTCGGAGGGCGACCTGGTGGTGTGCCCGGAGGGC
ACCACCTGCCGCGAGCCGTACCTGCTGCGCTTCTCGTCGCTGTTCGCCGAGCTGGCCGAC
GAGATCGTGCCGGTGGCCATCAACGCCCACGTGTCGATGTTCTACGGCACCACCGCCTCG
GGCCTGAAGTGCCTGGACCCGATCTTCTTCCTGATGAACCCGCGCCCGTCGTACCACGTG
CAGATCCTGGGCAAGGTGCCGCAGGAGTTCACCTGCGCCGGCGGCCGCTCGTCGCTGGA
GGTGGCCAACTACATCCAGCGCAAGCTGGCCGACGCCCTGGGCTTCGAGTGCACCACCC
TGACCCGCCGCGACAAGTACCTGATGCTGGCCGGCAACGAGGGCGTGGTGCACGAGAAC
AAGCGCAACTAA
Theobroma cacao Glycerol-3-phosphate acyltransferase 1 (TcGPAT3),
GenBank: CM001879.1 region: 9957750-9959837, CDS join (1-741, 1207-2088), 1623 nt
SEQ ID NO: 73
ATGGTTTTCCCTGTGGTATTTCTGAAGCTAGCAGACTGGGTCTTGTACCAGCTGCTGGCC
AACTCATGTTATAGAGCTGCAAGGAAGATGAGAAACTACGGGTTCTTTCTAAGGAACCA
AACTCTTAGGTCACCACCACAGCAACAAGCTGCTTCTTTGTTCCCTAGTGTTACCAAGTG
TGATGTAGGCAATAGTAGAAGGTTTGATACATTGGTCTGTGATATCCATGGAGTCTTGTT
AGGATCAGACACATTTTTTCCTTACTTCATGCTAGTTGCTTTTGAAGGTGGTAGCATTGTG
AGAGCCTTTCTGTTGCTTTTATCATGCTCCTTTTTGTGGGTATTGGACTCAGAGCTCAAGT
TGAGGATTATGATTTTTATTTCCTTTTGTGGGCTTAGGAAGAAGGACATCGAGAGTGTTG
GCAGGGCTGTTTTGCCAAAGTTTTATCTCGAGAATCTAAATCTCCAAGTCTATGAAGTTT
GGTCTAAAACAAGTTCAAGGGTTGTCTTTACAAGTATACCTAGAGTAATGGTGGAAGGA
TTTCTCCACGAATACATGAGTGCTAGTGGTGTTGTAGGCACCGAATTGCACACTGTTGGG
AACCGATTCACAGGTTTGTTGTCCAGCTCCGGGTTGCTTGTAAAGCATAATGCTTTAAAG
GAACACTTCGGAGATAAAAAGCCTGATGTTGGCCTCGGAAGTTCAAGCCTCCATGACCA
ATACTTTATCTCCCTTTGCAAGGAAGCCTATGTGGTGAACATGGAAGATGGCAAAAGCA
ATCTAAGCAGTTTCATGCCAAGGGACAAGTACCCGAAGCCTCTTATATTTCATGATGGGA
GGCTAGCTTTCTTGCCAACTCCATTTGCAACTCTTTCAATGTTCCTGTGGCTTCCATTTGG
AATAGTTCTTTCCATTCTTAGGATTTTCGTTGGTATCTGCCTGCCTTACAAGCTAGCTGTT
ATCTGCGCTACTCTGAGTGGTGTACAGCTGAAATTCCAAGGGTGCTTCCCTTCATCAAAT
TCACAACATAAAAAAGGGGTTCTTTATGTTTGTACCCATAGAACTCTTCTGGACCCAGTT
TTCCTTAGCACAGCATTATGCAAGCCTTTGACTGCAGTTACTTATAGTCTAAGCAAAATG
TCTGAACTGATAGCTCCCATCAAAACAGTTAGGTTGACAAGGGACAGGAAACAAGATGG
AGAAACCATGCAAAAATTGCTCAGTGAAGGTGATTTGGTAGTGTGCCCGGAAGGAACCA
CATGCAGAGAGCCTTACTTGTTAAGGTTTAGCTCATTGTTTGCTGAGCTAGCCGATGAGA
TAGTCCCAGTGGCCATAAACGCACATGTAAGCATGTTTTACGGGACAACTGCAAGTGGG
TTAAAATGCTTGGATCCTATCTTCTTTCTGATGAACCCCAGACCTAGCTACCATGTTCAAA
TCCTTGGGAAGGTGCCTCAAGAGTTTACATGTGCAGGGGGCAGGTCTAGCCTTGAAGTG
GCAAATTATATTCAGAGAAAGCTGGCTGATGCTCTGGGATTTGAGTGCACTACCCTTACA
AGGAGAGACAAGTACTTGATGCTAGCAGGCAATGAAGGGGTTGTCCATGAAAATAAGAG
AAATTAA
Theobroma cacao Glycerol-3-phosphate acyltransferase 1 (TcGPAT3)
truncated codon-optimized (corresponds to nt 61-1623 of SEQ ID NO: 72), 1566 nt
SEQ ID NO: 74
ATGAACTCGTGCTACCGCGCCGCCCGCAAGATGCGCAACTACGGCTTCTTCCTGCGCAAC
CAGACCCTGCGCTCGCCGCCGCAGCAGCAGGCCGCCTCGCTGTTCCCGTCGGTGACCAAG
TGCGACGTGGGCAACTCGCGCCGCTTCGACACCCTGGTGTGCGACATCCACGGCGTGCTG
CTGGGCTCGGACACCTTCTTCCCGTACTTCATGCTGGTGGCCTTCGAGGGCGGCTCGATC
GTGCGCGCCTTCCTGCTGCTGCTGTCGTGCTCGTTCCTGTGGGTGCTGGACTCGGAGCTG
AAGCTGCGCATCATGATCTTCATCTCGTTCTGCGGCCTGCGCAAGAAGGACATCGAGTCG
GTGGGCCGCGCCGTGCTGCCGAAGTTCTACCTGGAGAACCTGAACCTGCAGGTGTACGA
GGTGTGGTCGAAGACCTCGTCGCGCGTGGTGTTCACCTCGATCCCGCGCGTGATGGTGGA
GGGCTTCCTGCACGAGTACATGTCGGCCTCGGGCGTGGTGGGCACCGAGCTGCACACCG
TGGGCAACCGCTTCACCGGCCTGCTGTCGTCGTCGGGCCTGCTGGTGAAGCACAACGCCC
TGAAGGAGCACTTCGGCGACAAGAAGCCGGACGTGGGCCTGGGCTCGTCGTCGCTGCAC
GACCAGTACTTCATCTCGCTGTGCAAGGAGGCCTACGTGGTGAACATGGAGGACGGCAA
GTCGAACCTGTCGTCGTTCATGCCGCGCGACAAGTACCCGAAGCCGCTGATCTTCCACGA
CGGCCGCCTGGCCTTCCTGCCGACCCCGTTCGCCACCCTGTCGATGTTCCTGTGGCTGCCG
TTCGGCATCGTGCTGTCGATCCTGCGCATCTTCGTGGGCATCTGCCTGCCGTACAAGCTG
GCCGTGATCTGCGCCACCCTGTCGGGCGTGCAGCTGAAGTTCCAGGGCTGCTTCCCGTCG
TCGAACTCGCAGCACAAGAAGGGCGTGCTGTACGTGTGCACCCACCGCACCCTGCTGGA
CCCGGTGTTCCTGTCGACCGCCCTGTGCAAGCCGCTGACCGCCGTGACCTACTCGCTGTC
GAAGATGTCGGAGCTGATCGCCCCGATCAAGACCGTGCGCCTGACCCGCGACCGCAAGC
AGGACGGCGAGACCATGCAGAAGCTGCTGTCGGAGGGCGACCTGGTGGTGTGCCCGGAG
GGCACCACCTGCCGCGAGCCGTACCTGCTGCGCTTCTCGTCGCTGTTCGCCGAGCTGGCC
GACGAGATCGTGCCGGTGGCCATCAACGCCCACGTGTCGATGTTCTACGGCACCACCGCC
TCGGGCCTGAAGTGCCTGGACCCGATCTTCTTCCTGATGAACCCGCGCCCGTCGTACCAC
GTGCAGATCCTGGGCAAGGTGCCGCAGGAGTTCACCTGCGCCGGCGGCCGCTCGTCGCT
GGAGGTGGCCAACTACATCCAGCGCAAGCTGGCCGACGCCCTGGGCTTCGAGTGCACCA
CCCTGACCCGCCGCGACAAGTACCTGATGCTGGCCGGCAACGAGGGCGTGGTGCACGAG
AACAAGCGCAACTAA
Theobroma cacao Glycerol-3-phosphate acyltransferase 1 (TcGPAT3)
truncated (corresponds to nt 61-1623 of SEQ ID NO: 73), 1566 nt
SEQ ID NO: 75
ATGAACTCATGTTATAGAGCTGCAAGGAAGATGAGAAACTACGGGTTCTTTCTAAGGAA
CCAAACTCTTAGGTCACCACCACAGCAACAAGCTGCTTCTTTGTTCCCTAGTGTTACCAA
GTGTGATGTAGGCAATAGTAGAAGGTTTGATACATTGGTCTGTGATATCCATGGAGTCTT
GTTAGGATCAGACACATTTTTTCCTTACTTCATGCTAGTTGCTTTTGAAGGTGGTAGCATT
GTGAGAGCCTTTCTGTTGCTTTTATCATGCTCCTTTTTGTGGGTATTGGACTCAGAGCTCA
AGTTGAGGATTATGATTTTTATTTCCTTTTGTGGGCTTAGGAAGAAGGACATCGAGAGTG
TTGGCAGGGCTGTTTTGCCAAAGTTTTATCTCGAGAATCTAAATCTCCAAGTCTATGAAG
TTTGGTCTAAAACAAGTTCAAGGGTTGTCTTTACAAGTATACCTAGAGTAATGGTGGAAG
GATTTCTCCACGAATACATGAGTGCTAGTGGTGTTGTAGGCACCGAATTGCACACTGTTG
GGAACCGATTCACAGGTTTGTTGTCCAGCTCCGGGTTGCTTGTAAAGCATAATGCTTTAA
AGGAACACTTCGGAGATAAAAAGCCTGATGTTGGCCTCGGAAGTTCAAGCCTCCATGAC
CAATACTTTATCTCCCTTTGCAAGGAAGCCTATGTGGTGAACATGGAAGATGGCAAAAGC
AATCTAAGCAGTTTCATGCCAAGGGACAAGTACCCGAAGCCTCTTATATTTCATGATGGG
AGGCTAGCTTTCTTGCCAACTCCATTTGCAACTCTTTCAATGTTCCTGTGGCTTCCATTTG
GAATAGTTCTTTCCATTCTTAGGATTTTCGTTGGTATCTGCCTGCCTTACAAGCTAGCTGT
TATCTGCGCTACTCTGAGTGGTGTACAGCTGAAATTCCAAGGGTGCTTCCCTTCATCAAA
TTCACAACATAAAAAAGGGGTTCTTTATGTTTGTACCCATAGAACTCTTCTGGACCCAGT
TTTCCTTAGCACAGCATTATGCAAGCCTTTGACTGCAGTTACTTATAGTCTAAGCAAAAT
GTCTGAACTGATAGCTCCCATCAAAACAGTTAGGTTGACAAGGGACAGGAAACAAGATG
GAGAAACCATGCAAAAATTGCTCAGTGAAGGTGATTTGGTAGTGTGCCCGGAAGGAACC
ACATGCAGAGAGCCTTACTTGTTAAGGTTTAGCTCATTGTTTGCTGAGCTAGCCGATGAG
ATAGTCCCAGTGGCCATAAACGCACATGTAAGCATGTTTTACGGGACAACTGCAAGTGG
GTTAAAATGCTTGGATCCTATCTTCTTTCTGATGAACCCCAGACCTAGCTACCATGTTCAA
ATCCTTGGGAAGGTGCCTCAAGAGTTTACATGTGCAGGGGGCAGGTCTAGCCTTGAAGT
GGCAAATTATATTCAGAGAAAGCTGGCTGATGCTCTGGGATTTGAGTGCACTACCCTTAC
AAGGAGAGACAAGTACTTGATGCTAGCAGGCAATGAAGGGGTTGTCCATGAAAATAAGA
GAAATTAA
Theobroma cacao Glycerol-3-phosphate acyltransferase 3 (TcGPAT4)
codon-optimized, 1614 nt
SEQ ID NO: 76
ATGGCCAAGCTGTCGATGGAGTTCTCGTTCTTCCAGACCCTGTTCTTCCTGTTCTGCCGCG
TGGTGTTCCGCCAGTCGAAGAACCACAAGTCGCTGCACCGCAACGTGTCGAACATCCAC
GCCAACGAGGGCAAGTACCACAAGTACCCGTCGTTCGTGCACCGCTCGAACCTGTCGAA
CCAGACCCTGGTGTTCTCGGTGGAGGAGGCCCTGCTGAAGTCGTCGTCGCTGTTCCCGTA
CTTCATGCTGGTGGCCTTCGAGGCCGGCGGCCTGCTGCGCGCCTTCATCCTGTTCGTGCTG
TACCCGATCCTGTGCCTGGTGTCGGAGGAGATGGGCCTGAAGATCATGGTGCTGGTGTGC
TTCTTCGGCATCAAGAAGAAGTCGTTCCGCGTGGGCTCGGCCGTGCTGCCGAAGTTCTTC
CTGGAGGACGTGGGCCTGGAGCCGTTCGAGATGCTGAAGAAGGGCGGCAAGAAGGTGG
CCGTGTCGAAGATCCCGCAGGTGATGATCGAGTCGTTCCTGAAGGACTACCTGGAGATC
GACTTCGTGGTGGGCCGCGAGCTGAAGGAGTTCTGCGGCTACTTCCTGGGCGTGATGGA
GGAGAAGAAGCGCTCGAAGGCCGCCCTGGACGAGATCATCGGCTCGGAGTCGATGGGCT
TCGACGTGATCGGCATCTCGGGCCTGAAGAAGTCGCTGGACTACCACTTCTTCTCGCACT
GCAAGGAGATCTACCAGGTGCGCAAGGCCGACAAGCGCAACTGGCGCCACGTGCCGCGC
CAGGAGTACTCGAAGCCGCTGATCTTCCACGACGGCCGCCTGGCCCTGCGCCCGACCCTG
GTGGCCTCGCTGACCATGTTCGTGTGGTTCCCGTTCGGCCTGGCCCTGTCGATCCTGCGCG
CCGTGGTGGGCCTGATGCTGCCGTACAAGATCTCGATCCCGCTGCTGGCCTACTCGGGCC
TGCACCTGTTCCTGTCGACCCCGGAGTCGTCGCTGCACCCGCTGTCGCTGCCGAACTCGA
AGAAGCAGAACCCGAAGGGCCGCCTGTACGTGTGCAACCACCGCACCCTGCTGGACCCG
GTGTACCTGTCGTTCGCCCTGCAGAAGGACCTGACCGCCGTGACCTACTCGCTGTCGCGC
ATCTCGGAGCTGCTGGCCCCGATCAAGACCGTGCGCCTGGCCCGCGACCGCGACCAGGA
CGGCAAGATGATGGAGAAGATGCTGAACCTGGGCGACCTGGTGGTGTGCCCGGAGGGCA
CCACCTGCCGCGAGCCGTACCTGCTGCGCTTCTCGCCGCTGTTCTCGGAGATGTCGGACG
ACATCGTGCCGGTGGCCATGGACTCGAACGTGTCGCTGTTCTACGGCACCACCGCCTCGG
GCCTGAAGTGCCTGGACCCGCTGTTCTTCCTGATGAACCCGCGCCCGATCTACACCGTGC
AGATCCTGGACGGCGTGTCGGGCCTGTACACCTGCCACGACGGCCAGCGCTCGCGCTTCA
AGGTGGCCAACCAGGTGCAGAACGAGATCGGCAAGGCCCTGGGCTTCGAGTGCACCAAG
CTGACCCGCCGCGACAAGTACCTGATCATGGCCGGCAACGAGGGCATCATCTCGCAGAC
CTAA
Theobroma cacao Glycerol-3-phosphate acyltransferase 3 (TcGPAT4),
GenBank: CM001879.1 region: 33774144-33776095, CDS join (1-720, 1059-1952), 1614 nt
SEQ ID NO: 77
ATGGCTAAACTTTCTATGGAGTTTTCCTTTTTCCAAACCCTTTTCTTCTTATTCTGTCGAGT
TGTCTTTAGACAGTCTAAGAATCACAAATCTCTCCACAGAAATGTCAGCAATATCCATGC
AAACGAGGGTAAATATCATAAGTATCCTTCTTTTGTTCATAGATCAAACCTGTCAAACCA
AACTCTGGTCTTCAGTGTAGAAGAGGCTTTGTTGAAATCCTCATCATTGTTTCCTTACTTC
ATGCTTGTAGCCTTTGAAGCTGGAGGGCTCTTGAGGGCCTTTATTCTTTTTGTTTTATACC
CAATTCTATGCTTAGTTAGCGAAGAGATGGGGTTGAAGATAATGGTCCTGGTTTGCTTCT
TTGGGATTAAGAAAAAGAGCTTCAGAGTTGGAAGTGCTGTTCTGCCGAAGTTCTTCTTGG
AGGATGTTGGCTTGGAACCATTTGAGATGTTGAAGAAAGGCGGGAAAAAGGTGGCTGTC
AGTAAAATTCCTCAAGTGATGATCGAGAGTTTCTTGAAGGATTACCTGGAAATTGATTTT
GTAGTTGGAAGAGAGCTGAAGGAGTTCTGTGGGTACTTTTTGGGAGTCATGGAAGAGAA
GAAGAGAAGTAAGGCTGCTTTGGACGAGATAATTGGAAGCGAAAGTATGGGCTTTGATG
TTATTGGCATCAGTGGCCTCAAGAAATCTCTTGACTATCATTTTTTTTCTCATTGCAAGGA
AATATACCAGGTGAGAAAAGCAGACAAAAGAAACTGGCGACACGTCCCAAGACAGGAG
TATTCCAAACCACTCATTTTCCATGACGGGAGACTAGCTCTCAGGCCAACTCTGGTGGCC
TCCCTGACCATGTTCGTGTGGTTCCCCTTCGGCTTGGCTCTTTCCATACTCAGAGCTGTTG
TCGGCTTAATGCTTCCGTACAAGATTTCCATCCCCTTATTAGCCTACAGCGGGTTGCACCT
GTTTCTTTCAACCCCGGAAAGCTCCCTGCATCCTCTTTCACTTCCGAACTCGAAGAAACA
AAACCCAAAAGGCCGCCTTTATGTTTGCAATCATAGAACACTGCTGGATCCCGTCTACCT
TTCTTTTGCATTACAGAAAGACCTCACTGCTGTTACTTACAGCTTAAGCAGGATATCAGA
GCTGTTAGCTCCAATCAAAACCGTTCGATTAGCAAGGGATCGTGATCAAGATGGTAAAA
TGATGGAAAAGATGCTAAATTTAGGGGACCTAGTCGTATGCCCGGAAGGGACTACGTGT
AGGGAGCCCTATCTCTTAAGGTTCAGCCCTTTATTTTCAGAGATGAGCGACGATATAGTC
CCTGTTGCGATGGACAGCAATGTGAGTTTGTTCTATGGGACAACAGCCAGTGGCCTGAAA
TGTCTGGACCCACTTTTCTTTCTCATGAACCCACGACCAATCTACACTGTCCAGATACTTG
ATGGTGTATCTGGCTTGTACACTTGTCATGATGGTCAAAGATCAAGGTTTAAAGTGGCTA
ATCAGGTTCAGAATGAGATTGGTAAGGCTCTGGGTTTTGAGTGCACCAAGCTTACGAGA
AGAGACAAGTACCTAATCATGGCTGGCAACGAAGGAATAATTAGCCAAACCTAA
Theobroma cacao Glycerol-3-phosphate acyltransferase 3 (TcGPAT4)
truncated codon-optimized (corresponds to nt 73-1614 of SEQ ID NO: 76), 1545 nt
SEQ ID NO: 78
ATGCAGTCGAAGAACCACAAGTCGCTGCACCGCAACGTGTCGAACATCCACGCCAACGA
GGGCAAGTACCACAAGTACCCGTCGTTCGTGCACCGCTCGAACCTGTCGAACCAGACCCT
GGTGTTCTCGGTGGAGGAGGCCCTGCTGAAGTCGTCGTCGCTGTTCCCGTACTTCATGCT
GGTGGCCTTCGAGGCCGGCGGCCTGCTGCGCGCCTTCATCCTGTTCGTGCTGTACCCGAT
CCTGTGCCTGGTGTCGGAGGAGATGGGCCTGAAGATCATGGTGCTGGTGTGCTTCTTCGG
CATCAAGAAGAAGTCGTTCCGCGTGGGCTCGGCCGTGCTGCCGAAGTTCTTCCTGGAGGA
CGTGGGCCTGGAGCCGTTCGAGATGCTGAAGAAGGGCGGCAAGAAGGTGGCCGTGTCGA
AGATCCCGCAGGTGATGATCGAGTCGTTCCTGAAGGACTACCTGGAGATCGACTTCGTGG
TGGGCCGCGAGCTGAAGGAGTTCTGCGGCTACTTCCTGGGCGTGATGGAGGAGAAGAAG
CGCTCGAAGGCCGCCCTGGACGAGATCATCGGCTCGGAGTCGATGGGCTTCGACGTGAT
CGGCATCTCGGGCCTGAAGAAGTCGCTGGACTACCACTTCTTCTCGCACTGCAAGGAGAT
CTACCAGGTGCGCAAGGCCGACAAGCGCAACTGGCGCCACGTGCCGCGCCAGGAGTACT
CGAAGCCGCTGATCTTCCACGACGGCCGCCTGGCCCTGCGCCCGACCCTGGTGGCCTCGC
TGACCATGTTCGTGTGGTTCCCGTTCGGCCTGGCCCTGTCGATCCTGCGCGCCGTGGTGG
GCCTGATGCTGCCGTACAAGATCTCGATCCCGCTGCTGGCCTACTCGGGCCTGCACCTGT
TCCTGTCGACCCCGGAGTCGTCGCTGCACCCGCTGTCGCTGCCGAACTCGAAGAAGCAGA
ACCCGAAGGGCCGCCTGTACGTGTGCAACCACCGCACCCTGCTGGACCCGGTGTACCTGT
CGTTCGCCCTGCAGAAGGACCTGACCGCCGTGACCTACTCGCTGTCGCGCATCTCGGAGC
TGCTGGCCCCGATCAAGACCGTGCGCCTGGCCCGCGACCGCGACCAGGACGGCAAGATG
ATGGAGAAGATGCTGAACCTGGGCGACCTGGTGGTGTGCCCGGAGGGCACCACCTGCCG
CGAGCCGTACCTGCTGCGCTTCTCGCCGCTGTTCTCGGAGATGTCGGACGACATCGTGCC
GGTGGCCATGGACTCGAACGTGTCGCTGTTCTACGGCACCACCGCCTCGGGCCTGAAGTG
CCTGGACCCGCTGTTCTTCCTGATGAACCCGCGCCCGATCTACACCGTGCAGATCCTGGA
CGGCGTGTCGGGCCTGTACACCTGCCACGACGGCCAGCGCTCGCGCTTCAAGGTGGCCA
ACCAGGTGCAGAACGAGATCGGCAAGGCCCTGGGCTTCGAGTGCACCAAGCTGACCCGC
CGCGACAAGTACCTGATCATGGCCGGCAACGAGGGCATCATCTCGCAGACCTAA
Theobroma cacao Glycerol-3-phosphate acyltransferase 3 (TcGPAT4)
truncated (corresponds to nt 73-1614 of SEQ ID NO: 77), 1545 nt
SEQ ID NO: 79
ATGCAGTCTAAGAATCACAAATCTCTCCACAGAAATGTCAGCAATATCCATGCAAACGA
GGGTAAATATCATAAGTATCCTTCTTTTGTTCATAGATCAAACCTGTCAAACCAAACTCT
GGTCTTCAGTGTAGAAGAGGCTTTGTTGAAATCCTCATCATTGTTTCCTTACTTCATGCTT
GTAGCCTTTGAAGCTGGAGGGCTCTTGAGGGCCTTTATTCTTTTTGTTTTATACCCAATTC
TATGCTTAGTTAGCGAAGAGATGGGGTTGAAGATAATGGTCCTGGTTTGCTTCTTTGGGA
TTAAGAAAAAGAGCTTCAGAGTTGGAAGTGCTGTTCTGCCGAAGTTCTTCTTGGAGGATG
TTGGCTTGGAACCATTTGAGATGTTGAAGAAAGGCGGGAAAAAGGTGGCTGTCAGTAAA
ATTCCTCAAGTGATGATCGAGAGTTTCTTGAAGGATTACCTGGAAATTGATTTTGTAGTT
GGAAGAGAGCTGAAGGAGTTCTGTGGGTACTTTTTGGGAGTCATGGAAGAGAAGAAGAG
AAGTAAGGCTGCTTTGGACGAGATAATTGGAAGCGAAAGTATGGGCTTTGATGTTATTG
GCATCAGTGGCCTCAAGAAATCTCTTGACTATCATTTTTTTTCTCATTGCAAGGAAATATA
CCAGGTGAGAAAAGCAGACAAAAGAAACTGGCGACACGTCCCAAGACAGGAGTATTCC
AAACCACTCATTTTCCATGACGGGAGACTAGCTCTCAGGCCAACTCTGGTGGCCTCCCTG
ACCATGTTCGTGTGGTTCCCCTTCGGCTTGGCTCTTTCCATACTCAGAGCTGTTGTCGGCT
TAATGCTTCCGTACAAGATTTCCATCCCCTTATTAGCCTACAGCGGGTTGCACCTGTTTCT
TTCAACCCCGGAAAGCTCCCTGCATCCTCTTTCACTTCCGAACTCGAAGAAACAAAACCC
AAAAGGCCGCCTTTATGTTTGCAATCATAGAACACTGCTGGATCCCGTCTACCTTTCTTTT
GCATTACAGAAAGACCTCACTGCTGTTACTTACAGCTTAAGCAGGATATCAGAGCTGTTA
GCTCCAATCAAAACCGTTCGATTAGCAAGGGATCGTGATCAAGATGGTAAAATGATGGA
AAAGATGCTAAATTTAGGGGACCTAGTCGTATGCCCGGAAGGGACTACGTGTAGGGAGC
CCTATCTCTTAAGGTTCAGCCCTTTATTTTCAGAGATGAGCGACGATATAGTCCCTGTTGC
GATGGACAGCAATGTGAGTTTGTTCTATGGGACAACAGCCAGTGGCCTGAAATGTCTGG
ACCCACTTTTCTTTCTCATGAACCCACGACCAATCTACACTGTCCAGATACTTGATGGTGT
ATCTGGCTTGTACACTTGTCATGATGGTCAAAGATCAAGGTTTAAAGTGGCTAATCAGGT
TCAGAATGAGATTGGTAAGGCTCTGGGTTTTGAGTGCACCAAGCTTACGAGAAGAGACA
AGTACCTAATCATGGCTGGCAACGAAGGAATAATTAGCCAAACCTAA
Durio zibethinus glycerol-3-phosphate acyltransferase 8 isoform X1
(DzGPAT), Ref. No. XP_022770453.1 (corresponds to SEQ ID NO: 64), 500 aa
SEQ ID NO: 80
MAPLKAAQSFPSITECDGSTYESIAADLDGTLLISRSSFPYFMLVAVEAGSLFRGLILLLSLPLIII
SYLFVSEAIGIQILIFISFAGLKIRDIELVSRAVLPRFYAANVRKESFEVFDRCKRKVVVTANPTF
MVEPFVKDFLGGDKVLGTEIEVNPKTKKATGFVKKPGVLVGKLKRLAIFKEFGDESPDLGIG
DRESDHDFMSICKEGYMVHPSKSATPVQLDRLKSRIIFHDGRFVQRPDPLNALITYIWLPFGFI
LSIIRVYFNLPLPERIVRYTYEMLGIHLVIRGKRPPPPSPGTPGNLYVCNHRSALDPIVIAIALGR
KVSCVTYSVSRLSRFLSPIPAIALTRDRAADAARISELLQKGDLVVCPEGTTCREQFLLRFSAL
FAEMSDRIVPVAVNCRQNMFYGTTVRGVKFWDPYFFFMNPRPTYEVTFLDRLPEEMTVKAG
GKSAIEVANHVQKVLGDVLGFECTGLTRKDKYMLLGGNDGKVESIYNAKK
Gossypium arboreum glycerol-3-phosphate acyltransferase 8-like
protein (GaGPAT) Ref. No. KHG29408.1 (corresponds to SEQ ID NO: 65), 500 aa
SEQ ID NO: 81
MAPPKAGKTFPSITECDGLKYESIAADLDGTLLISRSSFPYFMLIAVEAGSLLRGLILLLSLPLVI
ISYLFISEAIGIQILIFISFAGLKIRDIELVSRAVLPRFYAANVRKESFEVFDRCKRKVVVTANPTF
MVEPFVKDFLGGDKVLGTEIEVNPKTKKATGFVKNPGVLVGKFKRLAILKEFGDESPDLGIG
DRESDHDFMSICKEGYMVHPSKSASPVPLDRLKSRIIFHDGRFVQRPDPLNAWLTYLWLPFGF
ILSIIRVYFNLPLPERIVRYTYEMLGIHLVIRGKRPPPPSAGTPGNLYVCNHRTALDPIVIAIALG
RKVSCVTYSVSRLSRFLSPIPAIALTRDRAADAARISELLQKGDLVVCPEGTTCREQFLLRFSA
LFAEMSDRIVPVAVNCKQSMFYGTTVRGVKFWDPYFFFMNPRPTYEVTFLDRLPEEMTVKA
GGKSAIEVANHVQKVLGDVLGFECTGLTRKDKYMLLGGNDGKVESMYNGKK
Hibiscus syriacus glycerol-3-phosphate acyltransferase 8 (HsGPAT)
Ref. No. XP_039063668.1 (corresponds to SEQ ID NO: 66), 500 aa
SEQ ID NO: 82
MTPLRAGRRFPSITECNGSTYESIAADLDGTLLISRSSFPYFMLIAVEAGSLLRGLILLLSLPLVI
VSYLFISEAIGIQILIFISFAGLKIRDIELVSRAILPRFYAANVRKESFEVFDRCKRKVVVTANPTF
MVEPFVKDFLGGDKVLGTEIEVNPKTKKATGFVKKPGVLVSELKRLAILKEFGDDSPDLGIG
DRESDHDFMSICKEGYMVHPSKSASPVPLDRLRSRIIFHDGRFVQRPDPLNALITYIWLPFGFIL
SIIRVYFNLPLPERIVRYTYEMLGIHLVIRGKRPSPPSPGTPGNLYVCNHRSALDPIVIAIALGRK
VSCVTYSVSRLSRFLSPIPAIALTRDRAADAARISELLQKGDLVVCPEGTTCREPFLLRFSALFA
EMSDRIVPVAVNCKQSMFYGTTVRGVKFWDPYFFFMNPRPTYEVTFLDRLPEEMTVKAGGK
SAIEVANHVQKVLGDVLGFECTGLTRKDKYMLLGGNDGKVESMYNGKK
Theobroma cacao Glycerol-3-phosphate acyltransferase 8 (TcGPAT1)
Ref. No. XP_007051782.1 or EOX95939.1 (corresponds to SEQ ID NO: 67), 500 aa
SEQ ID NO: 83
MAPAKSGRSFPSITKCEGSTYESIAADLDGTLLISRSSFPYFMLVAVEAGSLLRGLILLLSLPLVI
VSYLFISEAIGIQILIFISFAGLKIRDIELVSRAVLPRFYAANVRKESFEVFDRCKRKVVVTANPT
FMVEPFVKDFLGGDKVLGTEIEVNPKTKKATGFVKKPGVLVSELKRLAILKEFGEESPDLGIG
DRESDHDFMSICKEGYMVHPSKSATPVPLDRLKSRIIFHDGRFVQRPDPLNALITYLWLPFGFI
LSIVRVYFNLPLPERIVRYTYEMLGIHLVIRGKRPPPPSPGTPGNLYVCNHRSALDPIVIAIALG
RKVSCVTYSVSRLSRFLSPIPAVALTRDRAADAARISELLQKGDLVVCPEGTTCREQFLLRFSA
LFAELSDRIVPVAVNCRQNMFYGTTVRGVKFWDPYFFFMNPRPTYEVTFLDRLPEEMTVKA
GGKSSIEVANHVQKVLGDVLGFECTGLTRKDKYLLLGGNDGKVESMYNAKK
Theobroma cacao Glycerol-3-phosphate acyltransferase 9 isoform 1
(TcGPAT2) Ref. No. XP_007041647.1 or EOX97478.1 (corresponds to SEQ ID NO: 69), 416 aa
SEQ ID NO: 84
MKKEKLFGFINIKKKEKEEKKPKPNKNPNSQTKSESEREGMSSRGGKLSSSSSELDLDGPNIE
DYLPSGSSINEPRGKLRLRDLLDISPTLTEAAGAIVDDSFTRCFKSNPPEPWNWNVYLFPLWCF
GVAVRYLILFPARVVVLTIGWIIFLSSFIPVHFLLKGHDKLRKKMERVLVELMCSFFVASWTG
VVKYHGPRPSIRPKQVFVANHTSMIDFIILEQMTAFAVIMQKHPGWVGLLQSTILESVGCIWF
NRSEAKDREIVAKKLRDHVQGVDNNPLLIFPEGTCINNQYSVMFKKGAFELGCTVCPIAIKYN
KIFVDAFWNSRKQSFTMHLLQLMTSWAVVCDVWYLEPQNLRPGETPIEFAERVRDIISVRAG
LKKVPWDGYLKYSRPSPKHRERKQQSFAESVLLRLEEK
Theobroma cacao Glycerol-3-phosphate acyltransferase 9 isoform 1 (TcGPAT2) truncated
(corresponds to SEQ ID NO: 71; corresponds to aa 71-416 of SEQ ID NO: 84), 347 aa
SEQ ID NO: 85
MSINEPRGKLRLRDLLDISPTLTEAAGAIVDDSFTRCFKSNPPEPWNWNVYLFPLWCFGVAVR
YLILFPARVVVLTIGWIIFLSSFIPVHFLLKGHDKLRKKMERVLVELMCSFFVASWTGVVKYH
GPRPSIRPKQVFVANHTSMIDFIILEQMTAFAVIMQKHPGWVGLLQSTILESVGCIWFNRSEAK
DREIVAKKLRDHVQGVDNNPLLIFPEGTCINNQYSVMFKKGAFELGCTVCPIAIKYNKIFVDA
FWNSRKQSFTMHLLQLMTSWAVVCDVWYLEPQNLRPGETPIEFAERVRDIISVRAGLKKVP
WDGYLKYSRPSPKHRERKQQSFAESVLLRLEEK
Theobroma cacao Glycerol-3-phosphate acyltransferase 1 (TcGPAT3)
Ref. No. EOX93017.1 (corresponds to SEQ ID NO: 72 or SEQ ID NO: 73), 540 aa
SEQ ID NO: 86
MVFPVVFLKLADWVLYQLLANSCYRAARKMRNYGFFLRNQTLRSPPQQQAASLFPSVTKCD
VGNSRRFDTLVCDIHGVLLGSDTFFPYFMLVAFEGGSIVRAFLLLLSCSFLWVLDSELKLRIMI
FISFCGLRKKDIESVGRAVLPKFYLENLNLQVYEVWSKTSSRVVFTSIPRVMVEGFLHEYMSA
SGVVGTELHTVGNRFTGLLSSSGLLVKHNALKEHFGDKKPDVGLGSSSLHDQYFISLCKEAY
VVNMEDGKSNLSSFMPRDKYPKPLIFHDGRLAFLPTPFATLSMFLWLPFGIVLSILRIFVGICLP
YKLAVICATLSGVQLKFQGCFPSSNSQHKKGVLYVCTHRTLLDPVFLSTALCKPLTAVTYSLS
KMSELIAPIKTVRLTRDRKQDGETMQKLLSEGDLVVCPEGTTCREPYLLRFSSLFAELADEIVP
VAINAHVSMFYGTTASGLKCLDPIFFLMNPRPSYHVQILGKVPQEFTCAGGRSSLEVANYIQR
KLADALGFECTTLTRRDKYLMLAGNEGVVHENKRN
Theobroma cacao Glycerol-3-phosphate acyltransferase 1 (TcGPAT3 )truncated (corresponds
to SEQ ID NO: 74 or SEQ ID NO: 75; corresponds to aa 21-540 of SEQ ID NO: 86), 521 aa
SEQ ID NO: 87
MNSCYRAARKMRNYGFFLRNQTLRSPPQQQAASLFPSVTKCDVGNSRRFDTLVCDIHGVLL
GSDTFFPYFMLVAFEGGSIVRAFLLLLSCSFLWVLDSELKLRIMIFISFCGLRKKDIESVGRAVL
PKFYLENLNLQVYEVWSKTSSRVVFTSIPRVMVEGFLHEYMSASGVVGTELHTVGNRFTGLL
SSSGLLVKHNALKEHFGDKKPDVGLGSSSLHDQYFISLCKEAYVVNMEDGKSNLSSFMPRDK
YPKPLIFHDGRLAFLPTPFATLSMFLWLPFGIVLSILRIFVGICLPYKLAVICATLSGVQLKFQG
CFPSSNSQHKKGVLYVCTHRTLLDPVFLSTALCKPLTAVTYSLSKMSELIAPIKTVRLTRDRK
QDGETMQKLLSEGDLVVCPEGTTCREPYLLRFSSLFAELADEIVPVAINAHVSMFYGTTASGL
KCLDPIFFLMNPRPSYHVQILGKVPQEFTCAGGRSSLEVANYIQRKLADALGFECTTLTRRDK
YLMLAGNEGVVHENKRN
Theobroma cacao Glycerol-3-phosphate acyltransferase 3 (TcGPAT4)
Ref. No. EOX95331.1 (corresponds to SEQ ID NO: 76 or SEQ ID NO: 77), 537 aa
SEQ ID NO: 88
MAKLSMEFSFFQTLFFLFCRVVFRQSKNHKSLHRNVSNIHANEGKYHKYPSFVHRSNLSNQT
LVFSVEEALLKSSSLFPYFMLVAFEAGGLLRAFILFVLYPILCLVSEEMGLKIMVLVCFFGIKK
KSFRVGSAVLPKFFLEDVGLEPFEMLKKGGKKVAVSKIPQVMIESFLKDYLEIDFVVGRELKE
FCGYFLGVMEEKKRSKAALDEIIGSESMGFDVIGISGLKKSLDYHFFSHCKEIYQVRKADKRN
WRHVPRQEYSKPLIFHDGRLALRPTLVASLTMFVWFPFGLALSILRAVVGLMLPYKISIPLLA
YSGLHLFLSTPESSLHPLSLPNSKKQNPKGRLYVCNHRTLLDPVYLSFALQKDLTAVTYSLSRI
SELLAPIKTVRLARDRDQDGKMMEKMLNLGDLVVCPEGTTCREPYLLRFSPLFSEMSDDIVP
VAMDSNVSLFYGTTASGLKCLDPLFFLMNPRPIYTVQILDGVSGLYTCHDGQRSRFKVANQV
QNEIGKALGFECTKLTRRDKYLIMAGNEGIISQT
Theobroma cacao Glycerol-3-phosphate acyltransferase 3 (TcGPAT4) truncated (corresponds
to SEQ ID NO: 78 or SEQ ID NO: 79; corresponds to aa 25-537 of SEQ ID NO: 88), 514 aa
SEQ ID NO: 89
MQSKNHKSLHRNVSNIHANEGKYHKYPSFVHRSNLSNQTLVFSVEEALLKSSSLFPYFMLVA
FEAGGLLRAFILFVLYPILCLVSEEMGLKIMVLVCFFGIKKKSFRVGSAVLPKFFLEDVGLEPF
EMLKKGGKKVAVSKIPQVMIESFLKDYLEIDFVVGRELKEFCGYFLGVMEEKKRSKAALDEII
GSESMGFDVIGISGLKKSLDYHFFSHCKEIYQVRKADKRNWRHVPRQEYSKPLIFHDGRLAL
RPTLVASLTMFVWFPFGLALSILRAVVGLMLPYKISIPLLAYSGLHLFLSTPESSLHPLSLPNSK
KQNPKGRLYVCNHRTLLDPVYLSFALQKDLTAVTYSLSRISELLAPIKTVRLARDRDQDGKM
MEKMLNLGDLVVCPEGTTCREPYLLRFSPLFSEMSDDIVPVAMDSNVSLFYGTTASGLKCLD
PLFFLMNPRPIYTVQILDGVSGLYTCHDGQRSRFKVANQVQNEIGKALGFECTKLTRRDKYLI
MAGNEGIISQT

In some embodiments of any of the aspects, the engineered bacterium comprises a Durio zibethinus GPAT gene or polypeptide (e.g., SEQ ID NOs: 64, 80). In some embodiments of any of the aspects, the engineered bacterium comprises a Gossypium arboreum GPAT gene or polypeptide (e.g., SEQ ID NOs: 65, 81). In some embodiments of any of the aspects, the engineered bacterium comprises a Hibiscus syriacus GPAT gene or polypeptide (e.g., SEQ ID NOs: 66, 82). In some embodiments of any of the aspects, the engineered bacterium comprises a Theobroma cacao GPAT gene or polypeptide (e.g., SEQ ID NOs: 67, 69, 71-79, 83-89).

In some embodiments of any of the aspects, the engineered bacterium comprises at least one exogenous copy of at least one functional phosphatidic acid (PA) phosphatase gene. Phosphatidic acid (PA) phosphatases catalyze dephosphorylation at the sn3 position of phosphatidic acid (PA). As a non-limiting example, such a phosphatase is phosphatidate phosphatase (PAP) (E.C. 3.1.3.4), which is a key regulatory enzyme in lipid metabolism, catalyzing the conversion of phosphatidate to diacylglycerol. PAP belongs to the family of enzymes known as hydrolases, and more specifically to the hydrolases that act on phosphoric monoester bonds. The two substrates of PAP are phosphatidate and H₂O, and its two products are diacylglycerol and phosphate. When PAP is active, diacylglycerols formed by PAP can go on to form any of several products, including phosphatidylethanolamine, phosphatidylcholine, phosphatidylserine, and triacylglycerol. The systematic name of the PAP enzyme class is diacylglycerol-3-phosphate phosphohydrolase. Other names in common use include: phosphatidic acid phosphatase (PAP), 3-sn-phosphatidate phosphohydrolase, acid phosphatidyl phosphatase, phosphatidic acid phosphohydrolase, phosphatidate phosphohydrolase, and lipid phosphate phosphohydrolase (LPP).

In some embodiments of any of the aspects, the functional PAP gene preferentially produces or leads to the production of TAGs comprising a specific type of fatty acid R group (e.g., C16). As such, the functional PAP gene can be selected from any PAP gene from any species that preferentially produces or leads to the production of TAGs comprising a specific type of fatty acid R group (e.g., C16). In some embodiments of any of the aspects, the engineered bacterium comprises at least one exogenous copy of at least one functional PAP gene. In some embodiments of any of the aspects, the engineered bacterium does not comprise a functional endogenous PAP gene. In some embodiments of any of the aspects, the functional PAP is heterologous. In some embodiments of any of the aspects, a PAP polypeptide as described herein is truncated to remove an organelle targeting sequence(s); in some embodiments, such a targeting sequence can contribute to poor expression of the PAP polypeptide, e.g., in the engineered bacteria described herein.

In some embodiments of any of the aspects, the functional heterologous PAP gene comprises a Rhodococcus PAP gene. In some embodiments of any of the aspects, the functional heterologous PAP gene comprises a Rhodococcus opacus PAP gene, or a Rhodococcus jostii PAP gene. In some embodiments of any of the aspects, the engineered bacterium comprises at least one exogenous copy of at least one functional PAP gene comprising one of SEQ ID NOs: 31-34, or a nucleic acid sequence that is at least 80%, at least 85%, at least 90%, at least 91%, at least 92%, at least 93%, at least 94%, at least 95%, at least 96%, at least 97%, at least 98%, or at least 99% identical to the sequence of at least one of SEQ ID NOs: 31-34, that maintains the same functions as at least one of SEQ ID NOs: 31-34 (e.g., phosphatidate phosphatase).

In some embodiments of any of the aspects, the amino acid sequence encoded by the functional PAP gene comprises one of SEQ ID NOs: 35-36, or an amino acid sequence that is at least 80%, at least 85%, at least 90%, at least 91%, at least 92%, at least 93%, at least 94%, at least 95%, at least 96%, at least 97%, at least 98%, or at least 99% identical to the sequence of at least one of SEQ ID NOs: 35-36, that maintains the same functions as at least one of SEQ ID NOs: 35-36 (e.g., phosphatidate phosphatase).

Rhodococcus opacus PAP (RoPAP), Rhodococcus opacus PD630,
complete genome, GenBank: CP003949.1, REGION: 4275960-4276643, 684 bp
SEQ ID NO: 31
ATGCCCCACACCTCTGCCGCTCACGCCGGGCTTCGTATGTCCGCCCTGACGCTGATATTG
GCCGTGCTCTGCGTGCAGGTCCACGACGGCGGCCCACTGACCGGTGCCGACGTGCCCGT
GACGTCGTGGGCCGTCGGAAACCGTTCCGCGGTCCTCGACCACGCGGCGCTCCTCGTCAC
CGACCTCGGCAGCCCCGTCGCCACCGTGGCCCTCGCCGTGATCTGCGGGCTCGCGCTCGC
GTGGCATCGGCGTTCCGCGATTCCCGCCGTCCTCGTCGTCGGAACGGTCGGGGCCGCCAC
CACGGCAAGCACGGCCCTGAAGCTGGTGGTCGGGCGCAGCCGGCCGGCCCTCGACCTGC
AGGAGGTCCTGGAGACGGACTACTCGTTCCCGTCCGGACATGTCACGGGTACGGTGGCTT
TGCTCGGCATCACGGCCGCGATCCTGCTCGGCCGCCGGCGCCGCCTCGTCCGGGTGTGTG
GCGGGGCCGTGGTCGGGTGCGGGGTGGTGATCGTTGCCGCGACCCGCGTGTACCTCGGT
GTCCACTGGCTCACCGATGTTGTCGCCGGCGCGATCCTGGGCGCGGTGTTCGTGACGGTG
GGGGCCGCCACGTACGAGCGTGTCCACCCGCCGGTCGGCACCCCCGCTCCGTCGAAACC
GCTCGCGGCCGTCGACCGAGTGGGAGGC
CnDNA_RoPAP, codon-optimized, 684 bp
SEQ ID NO: 32
ATGCCGCACACCTCGGCCGCCCACGCCGGCCTGCGCATGTCGGCCCTGACCCTGATCCTG
GCCGTGCTGTGCGTGCAGGTGCACGACGGCGGCCCGCTGACCGGCGCCGACGTGCCGGT
GACCTCGTGGGCCGTGGGCAACCGCTCGGCCGTGCTGGACCACGCCGCCCTGCTGGTGA
CCGACCTGGGCTCGCCGGTGGCCACCGTGGCCCTGGCCGTGATCTGCGGCCTGGCCCTGG
CCTGGCACCGCCGCTCGGCCATCCCGGCCGTGCTGGTGGTGGGCACCGTGGGCGCCGCC
ACCACCGCCTCGACCGCCCTGAAGCTGGTGGTGGGCCGCTCGCGCCCGGCCCTGGACCTG
CAGGAGGTGCTGGAGACCGACTACTCGTTCCCGTCGGGCCACGTGACCGGCACCGTGGC
CCTGCTGGGCATCACCGCCGCCATCCTGCTGGGCCGCCGCCGCCGCCTGGTGCGCGTGTG
CGGCGGCGCCGTGGTGGGCTGCGGCGTGGTGATCGTGGCCGCCACCCGCGTGTACCTGG
GCGTGCACTGGCTGACCGACGTGGTGGCCGGCGCCATCCTGGGCGCCGTGTTCGTGACCG
TGGGCGCCGCCACCTACGAGCGCGTGCACCCGCCGGTGGGCACCCCGGCCCCGTCGAAG
CCGCTGGCCGCCGTGGACCGCGTGGGCGGC
Rhodococcus jostii PAP (RjPAP), Rhodococcus jostii RHA1, complete
sequence, NCBI Reference Sequence: NC_008268.1, REGION: 83452-84138, 687 bp
SEQ ID NO: 33
ATGCCCCACACCTCCATCGCCACCGCCGGGCTTCGTGTGTCCGCGCTGACGCTGATCCTG
GCCGTGCTCTGTGTGCAGGTCCGCGACGGCGGCCCACTGACCGGTGCCGACGTGCCCGC
GACGTCGTGGGTCGTCGGTCACCGCTCCGCCACCCTTGACCATGTGGCTCTCCTCGTCAC
CGCCCTCGGCAGCCCCGTCGCCACCGTGGCCCTCGCCGTGATCTGCGGGCTCGCCCTCGC
CTGGCGCCGGCGTTCCGCGATCCCCGCCGCCGTCGTCGTCGGGACGGTCGGGGCCGCCAC
CGCGGCGAGCACGGCCCTGAAGCTGGTCGTCGAACGCAGCCGGCCGGCCGTCGACCTGC
AGGAGGTCCTGGAGACGGACTACTCGTTCCCGTCCGGGCACGTCACGGGCACCGCGGCC
CTGCTCGGGGTCACGGCGGCGATCCTGCTCGGCCGCCGGCATCTCCGCGTCCGGGTGTGC
GGCGCGGCGGTGGCCGGGTGCGGGGTGGTGATCGTTGCCGTGACCCGCGTCTACCTCGG
TGTCCACTGGATGTCCGACGTCGTCGCCGGCGCGATCCTCGGCGCGGTGTTCGTGACGGT
AGGCGCCGCCACGTACGAGCGTGTCCACCGTCCGCTCGTCGCCGTCGCTCCGTCGAAACC
GCTCGCGGTCCTCGACCGAGTGGGAGGCTGA
CnDNA_RjPAP, codon-optimized, 684 bp
SEQ ID NO: 34
ATGCCGCACACCTCGATCGCCACCGCCGGCCTGCGCGTGTCGGCCCTGACCCTGATCCTG
GCCGTGCTGTGCGTGCAGGTGCGCGACGGCGGCCCGCTGACCGGCGCCGACGTGCCGGC
CACCTCGTGGGTGGTGGGCCACCGCTCGGCCACCCTGGACCACGTGGCCCTGCTGGTGAC
CGCCCTGGGCTCGCCGGTGGCCACCGTGGCCCTGGCCGTGATCTGCGGCCTGGCCCTGGC
CTGGCGCCGCCGCTCGGCCATCCCGGCCGCCGTGGTGGTGGGCACCGTGGGCGCCGCCA
CCGCCGCCTCGACCGCCCTGAAGCTGGTGGTGGAGCGCTCGCGCCCGGCCGTGGACCTG
CAGGAGGTGCTGGAGACCGACTACTCGTTCCCGTCGGGCCACGTGACCGGCACCGCCGC
CCTGCTGGGCGTGACCGCCGCCATCCTGCTGGGCCGCCGCCACCTGCGCGTGCGCGTGTG
CGGCGCCGCCGTGGCCGGCTGCGGCGTGGTGATCGTGGCCGTGACCCGCGTGTACCTGG
GCGTGCACTGGATGTCGGACGTGGTGGCCGGCGCCATCCTGGGCGCCGTGTTCGTGACCG
TGGGCGCCGCCACCTACGAGCGCGTGCACCGCCCGCTGGTGGCCGTGGCCCCGTCGAAG
CCGCTGGCCGTGCTGGACCGCGTGGGCGGC
Rhodococcus opacus PAP, phosphatase PAP2 family protein, NCBI
Reference Sequence: WP_005246202.1, 228 aa (corresponds to SEQ ID NOs: 31-32)
SEQ ID NO: 35
MPHTSAAHAGLRMSALTLILAVLCVQVHDGGPLTGADVPVTSWAVGNRSAVLDHAALLVT
DLGSPVATVALAVICGLALAWHRRSAIPAVLVVGTVGAATTASTALKLVVGRSRPALDLQE
VLETDYSFPSGHVTGTVALLGITAAILLGRRRRLVRVCGGAVVGCGVVIVAATRVYLGVHW
LTDVVAGAILGAVFVTVGAATYERVHPPVGTPAPSKPLAAVDRVGG
Rhodococcus jostii PAP, RHA1_RS00400 phosphatase PAP2 family
protein (Rhodococcus jostii RHA1), NCBI Reference Sequence: WP_011593404.1,
tr|Q0SKM5|Q0SKM5_RHOJR, Phosphatidic acid phosphatase, type 2 OS = Rhodococcus jostii
(strainRHA1) OX = 101510 GN = RHA1_ro00075 PE = 4 SV = 1, 228 aa (corresponds to SEQ ID
NOs: 33-34)
SEQ ID NO: 36
MPHTSIATAGLRVSALTLILAVLCVQVRDGGPLTGADVPATSWVVGHRSATLDHVALLVTA
LGSPVATVALAVICGLALAWRRRSAIPAAVVVGTVGAATAASTALKLVVERSRPAVDLQEV
LETDYSFPSGHVTGTAALLGVTAAILLGRRHLRVRVCGAAVAGCGVVIVAVTRVYLGVHW
MSDVVAGAILGAVFVTVGAATYERVHRPLVAVAPSKPLAVLDRVGG

In some embodiments of any of the aspects, the engineered bacterium comprises a Rhodococcus opacus PAP gene or polypeptide (e.g., SEQ ID NOs: 31, 32, or 35) or a Rhodococcus jostii PAP gene or polypeptide (e.g., SEQ ID NOs: 33, 34, or 36).

In some embodiments of any of the aspects, the engineered bacterium comprises any combination of phaC inactivation, Marvinbryantia formatexigens thioesterase, Cuphea palustris thioesterase, Acinetobacter baylyi DGAT, Thermomonospora curvata DGAT, Rhodococcus opacus PAP, or Rhodococcus jostii PAP (see e.g., Table 4).

TABLE 4
Non-Limiting Exemplary Combinations, eg., in C. necator; “ΔphaC” indicates
inactivation (e.g., genetic or chemical) of phaC; “Mf TE” indicates Marvinbryantia
formatexigens thioesterase; “Cp TE” indicates Cuphea palustris thioesterase (e.g.,
CpFatB1, CpFatB2, and/or Cp FatB2-B1 hybrid); “Ab DG” indicates Acinetobacter
baylyi DGAT; “Tc DG” indicates Thermomonospora curvata DGAT; “Ro PAP”
indicates Rhodococcus opacus PAP; “Rj PAP” indicates Rhodococcus jostii PAP.

| | Mf | Cp | Ab | Tc | Ro | Rj | | Mf | Cp | Ab | Tc | Ro | Rj |
|---|---|---|---|---|---|---|---|---|---|---|---|---|---|
| ΔphaC | TE | TE | DG | DG | PAP | PAP | ΔphaC | TE | TE | DG | DG | PAP | PAP |
| | | | | | | | | | | | | | X |
| X | | | | | | | X | | | | | | X |
| | X | | | | | | | X | | | | | X |
| X | X | | | | | | X | X | | | | | X |
| | | X | | | | | | | X | | | | X |
| X | | X | | | | | X | | X | | | | X |
| | X | X | | | | | | X | X | | | | X |
| X | X | X | | | | | X | X | X | | | | X |
| | | | X | | | | | | | X | | | X |
| X | | | X | | | | X | | | X | | | X |
| | X | | X | | | | | X | | X | | | X |
| X | X | | X | | | | X | X | | X | | | X |
| | | X | X | | | | | | X | X | | | X |
| X | | X | X | | | | X | | X | X | | | X |
| | X | X | X | | | | | X | X | X | | | X |
| X | X | X | X | | | | X | X | X | X | | | X |
| | | | | X | | | | | | | X | | X |
| X | | | | X | | | X | | | | X | | X |
| | X | | | X | | | | X | | | X | | X |
| X | X | | | X | | | X | X | | | X | | X |
| | | X | | X | | | | | X | | X | | X |
| X | | X | | X | | | X | | X | | X | | X |
| | X | X | | X | | | | X | X | | X | | X |
| X | X | X | | X | | | X | X | X | | X | | X |
| | | | X | X | | | | | | X | X | | X |
| X | | | X | X | | | X | | | X | X | | X |
| | X | | X | X | | | | X | | X | X | | X |
| X | X | | X | X | | | X | X | | X | X | | X |
| | | X | X | X | | | | | X | X | X | | X |
| X | | X | X | X | | | X | | X | X | X | | X |
| | X | X | X | X | | | | X | X | X | X | | X |
| X | X | X | X | X | | | X | X | X | X | X | | X |
| | | | | | X | | | | | | | X | X |
| X | | | | | X | | X | | | | | X | X |
| | X | | | | X | | | X | | | | X | X |
| X | X | | | | X | | X | X | | | | X | X |
| | | X | | | X | | | | X | | | X | X |
| X | | X | | | X | | X | | X | | | X | X |
| | X | X | | | X | | | X | X | | | X | X |
| X | X | X | | | X | | X | X | X | | | X | X |
| | | | X | | X | | | | | X | | X | X |
| X | | | X | | X | | X | | | X | | X | X |
| | X | | X | | X | | | X | | X | | X | X |
| X | X | | X | | X | | X | X | | X | | X | X |
| | | X | X | | X | | | | X | X | | X | X |
| X | | X | X | | X | | X | | X | X | | X | X |
| | X | X | X | | X | | | X | X | X | | X | X |
| X | X | X | X | | X | | X | X | X | X | | X | X |
| | | | | X | X | | | | | | X | X | X |
| X | | | | X | X | | X | | | | X | X | X |
| | X | | | X | X | | | X | | | X | X | X |
| X | X | | | X | X | | X | X | | | X | X | X |
| | | X | | X | X | | | | X | | X | X | X |
| X | | X | | X | X | | X | | X | | X | X | X |
| | X | X | | X | X | | | X | X | | X | X | X |
| X | X | X | | X | X | | X | X | X | | X | X | X |
| | | | X | X | X | | | | | X | X | X | X |
| X | | | X | X | X | | X | | | X | X | X | X |
| | X | | X | X | X | | | X | | X | X | X | X |
| X | X | | X | X | X | | X | X | | X | X | X | X |
| | | X | X | X | X | | | | X | X | X | X | X |
| X | | X | X | X | X | | X | | X | X | X | X | X |
| | X | X | X | X | X | | | X | X | X | X | X | X |
| X | X | X | X | X | X | | X | X | X | X | X | X | X |

In some embodiments of any of the aspects, the engineered bacterium comprises (i) at least one endogenous diacylglycerol kinase gene (E.C. 2.7.1.174) comprising at least one engineered inactivating modification; and/or (ii) at least one exogenous inhibitor of an endogenous diacylglycerol kinase gene or gene product. In some embodiments of any of the aspects, the engineered bacterium comprises at least one endogenous diacylglycerol kinase gene comprising at least one engineered inactivating modification. In some embodiments of any of the aspects, the engineered bacterium comprises at least one exogenous inhibitor of at least one endogenous diacylglycerol kinase enzyme. In some embodiments of any of the aspects, the engineered bacterium comprises at least one endogenous diacylglycerol kinase gene comprising at least one engineered inactivating modification and an inhibitor of an endogenous diacylglycerol kinase enzyme. Diacylglycerol kinases perform the reverse reaction to phosphatidate phosphatase (PAP). By knocking out dgkA, the precursor pool for TAGs (e.g., DAGs) is increased and therefore TAG production is increased.

In some embodiments of any of the aspects, the engineered inactivating modification of the endogenous diacylglycerol kinase comprises one or more of i) deletion of the entire coding sequence, ii) deletion of the promoter of the gene, iii) a frameshift mutation, iv) a nonsense mutation (i.e., a premature termination codon), v) a point mutation, vi) a deletion, vii) or an insertion.

In some embodiments of any of the aspects, the endogenous diacylglycerol kinase comprises diacylglycerol kinase A (dgkA). Diacylglycerol kinase converts diacylglycerol/DAG into phosphatidic acid/phosphatidate/PA and regulates the respective levels of these two bioactive lipids.

In some embodiments of any of the aspects, the engineered bacterium comprises an engineered inactivating modification of the endogenous Cupriavidus necator dgkA gene. In some embodiments of any of the aspects, the nucleic acid sequence of the endogenous Cupriavidus necator dgkA gene comprises SEQ ID NO: 90 or a sequence that is at least 95% (e.g., at least 95%, at least 96%, at least 97%, at least 98%, or at least 99%) identical to the sequence of SEQ ID NO: 90 that maintains the same functions as SEQ ID NO: 90 (e.g., diacylglycerol kinase).

In some embodiments of any of the aspects, the amino acid sequence encoded by the endogenous Cupriavidus necator dgkA gene comprises SEQ ID NO: 91 or an amino acid sequence that is at least 95% (e.g., at least 95%, at least 96%, at least 97%, at least 98%, or at least 99%) identical to the sequence of SEQ ID NO: 91 that maintains the same functions as SEQ ID NO: 91 (e.g., diacylglycerol kinase).

Cupriavidus necator dgkA gene, GenBank: CP039287.1 region
1123858 to 1124343, 486 nt (see e.g., Kennedy pathway)
SEQ ID NO: 90
atgcccaaaccccatccggaactgccgtccgacccgcctctgcagaggccgccgcaggcagtccagagcgctgactattcgatcgagcagaac
ccccacaaggccaaccgcggcctgacgcgcgcctggcatgcggccatcaattcgctgtcggggctgcgctatgcggtgctcgaggaaagcgcg
ttccgccaggagctgacgctggtggcaatcctggcgccgtgggcattcctgctgccggtggacgtggtcgagcgcatcctgctgctgggcacgct
gctggtggtgctgatcgtcgagttgctcaattccagcgtcgaggcggcaatcgaccgcatttcgctggagcggcacagcctgtccaagcgtgcca
aggatttcggcagcgccgcggtaatgctggcgctggtgctgtgcggcggcacctgggtcgccatcgccgggccgcacgtggtgcgctgggtgc
ggacgctggcgggctga
Cupriavidus necator dgkA polypeptide, NCBI Reference Sequence:
WP_010809153.1, 161 aa
SEQ ID NO: 91
MPKPHPELPSDPPLQRPPQAVQSADYSIEQNPHKANRGLTRAWHAAINSLSGLRYAVLEESAF
RQELTLVAILAPWAFLLPVDVVERILLLGTLLVVLIVELLNSSVEAAIDRISLERHSLSKRAKDF
GSAAVMLALVLCGGTWVAIAGPHVVRWVRTLAG

In some embodiments of any of the aspects, the engineered inactivating modification of an endogenous diacylglycerol kinase gene comprises a deletion of the entire coding sequence (e.g., a knockout of an endogenous dgkA gene, denoted herein as ΔdgkA). In some embodiments of any of the aspects, the engineered inactivating modification of an endogenous diacylglycerol kinase gene comprises at least one exogenous inhibitor of an endogenous diacylglycerol kinase gene or gene product.

In some embodiments of any of the aspects, the engineered bacterium comprises (i) at least one endogenous beta-oxidation gene comprising at least one engineered inactivating modification; and/or (ii) at least one exogenous inhibitor of an endogenous beta-oxidation gene or gene product. In some embodiments of any of the aspects, the engineered bacterium comprises at least one endogenous beta-oxidation gene comprising at least one engineered inactivating modification. In some embodiments of any of the aspects, the engineered bacterium comprises at least one exogenous inhibitor of at least one endogenous beta-oxidation enzyme. In some embodiments of any of the aspects, the engineered bacterium comprises at least one endogenous beta-oxidation gene comprising at least one engineered inactivating modification and an inhibitor of an endogenous beta-oxidation enzyme.

Beta-oxidation is the catabolic process by which fatty acid molecules are broken down to generate acetyl-CoA. Beta-oxidation thus counteracts the formation of TAGs, and as such can be inhibited in order to increase TAG synthesis. Thus inhibition of beta oxidation increases the flux of fatty acids into TAG biosynthesis. Inhibition of beta-oxidation also prevents re-uptake of TAGs. Non-limiting examples of enzymes involved in beta oxidation include acyl-CoA ligase (or synthetase), acyl CoA dehydrogenase, enoyl CoA hydratase, 3-hydroxyacyl-CoA dehydrogenase, and β-ketothiolase. In some embodiments of any of the aspects, an engineered bacterium comprises an engineered inactivating modification and/or an inhibitor of an endogenous acyl-CoA ligase (or synthetase), acyl CoA dehydrogenase, an enoyl CoA hydratase, a 3-hydroxyacyl-CoA dehydrogenase, and/or a β-ketothiolase.

In some embodiments of any of the aspects, the endogenous beta-oxidation gene is an acyl-coenzyme A dehydrogenase (also referred to as acyl-CoA dehydrogenase; EC:1.3.8.8; e.g., fadE or a gene with a FadE-like function, e.g., a FadE homolog). Acyl-coenzyme A dehydrogenase catalyzes the dehydrogenation of acyl-coenzymes A (acyl-CoAs) to 2-enoyl-CoAs, the first step of the beta-oxidation cycle of fatty acid degradation.

In some embodiments of any of the aspects, the endogenous beta-oxidation gene is a 3-hydroxyacyl-CoA dehydrogenase (EC:1.1.1.35; e.g., fadB or a gene with a FadB-like function, e.g., a FadB homolog). 3-hydroxyacyl-CoA dehydrogenase is involved in the aerobic and anaerobic degradation of long-chain fatty acids via beta-oxidation cycle. 3-hydroxyacyl-CoA dehydrogenase catalyzes the formation of 3-oxoacyl-CoA from enoyl-CoA via L-3-hydroxyacyl-CoA. FadB can also use D-3-hydroxyacyl-CoA and cis-3-enoyl-CoA as substrate.

In some embodiments of any of the aspects, the engineered bacterium comprises an engineered inactivating modification of the endogenous Cupriavidus necator 3-hydroxyacyl-CoA dehydrogenase gene. In some embodiments of any of the aspects, the nucleic acid sequence of the endogenous Cupriavidus necator 3-hydroxyacyl-CoA dehydrogenase gene comprises one of SEQ ID NO: 92-94 or a sequence that is at least 95% (e.g., at least 95%, at least 96%, at least 97%, at least 98%, or at least 99%) identical to the sequence of SEQ ID NO: 92-94 that maintains the same functions as SEQ ID NO: 92-94 (e.g., beta-oxidation, acyl-CoA dehydrogenase, or 3-hydroxyacyl-CoA dehydrogenase).

In some embodiments of any of the aspects, the amino acid sequence encoded by the endogenous Cupriavidus necator 3-hydroxyacyl-CoA dehydrogenase gene comprises SEQ ID NO: 95-97 or an amino acid sequence that is at least 95% (e.g., at least 95%, at least 96%, at least 97%, at least 98%, or at least 99%) identical to the sequence of SEQ ID NO: 95-97 that maintains the same functions as SEQ ID NO: 95-97 (e.g., beta-oxidation, acyl-CoA dehydrogenase, or 3-hydroxyacyl-CoA dehydrogenase).

Cupriavidus necator N-1, acyl-CoA dehydrogenase fadE: A0460,
GenBank: CP039287.1 region 483888 to 485675, 1788 nt
SEQ ID NO: 92
atgggccagtacaccgcaccgttgcgcgacatgcagttcgtgctccatgaactgctcggcgccgaagccgaactcaaggcgatgccgccgcacg
cggacatcgacgcggacaccatcaaccaggttatcgaggaagccggcaagttctgctcggacgtggtgttcccgctcaaccaggtgggcgaccg
cgaaggctgcacctacgtcggcgacggcgtggtcaaggctcccaccggcttcaaggaagcctaccagcagtatgtcgaggccggctggccggc
gctggcgtgcgatcccgagttcggcggccagggcctgccgatcgtgatcaacaatgtggtctacgagatgctgaactcggccggccaggcctgg
accatgtacccgggcctgtcgcacggcgcgtacgaggcgctgcacgcgcacggcacgccagaactgcagcagacctacctgcccaagctggtc
tccggcgtgtggaccggcaccatgtgcctgaccgagccgcactgcggcaccgacctcggcatcctgcgttccaaggcagagccgcaggccgac
ggttcctacctgatctcgggcaccaagatcttcatctcggccggcgagcacgacatggccgagaacatcatccacctggtgctggcacgcctgc
cggacgcgccgggcggcaccaagggcatctcgctgttcgtggtgcccaagttcatccccgatgccaacggcaacccgggcgagcgcaacggcat
caagtgcggctcgatcgagcacaagatgggcatccacggcaacgccacctgcgtgatgaacctggacggcgcgcgcggctggatggtgggcg
agcccaacaagggcctgaacgccatgttcgtgatgatgaacgccgcgcgcctgggcgtgggcgcgcagggcctggggctgaccgaagtggcg
taccagaactcgctggcctacgccaaggaccgcctgcagatgcgcgcgctgaccggcccgaaggcgccggacaagcccgccgacccgatcat
cgtgcacccggacgtgcgccgcatgctgctgacgcagaaggcctacgccgaaggcggccgcgccttcagctactggaccgcgctgcagatcga
ccgcgagttgtcgcaccctgacgaagccgtgcgcaagcaggccggcgacctggtcgcgctgctcacgccggtgatcaaggccttcctgaccga
caacgccttcacgtccaccaatgagggcatgcaggtgttcggcggccacggctatatcgccgagtggggcatggagcagtatgtgcgcgatgcg
cgcatcaacatgatctacgaaggcaccaacaccatccaggcgctggacctgctgggccgcaagatcctgggcgacatgggcgccaggatgaag
gccttcggcaagatcgtgcaggaattcgttgaagccgaaggcaccaacgaagccatgcaggagttcatcaacccgctcgccgacattggcgaca
aggtgcagaagctcaccatggaaatcggcatgaaggcgatgggcaatgccgacgaggtgggcgctgccgcggtgccgtacctgcgcgtggtg
ggccacctggtgttctcatacttctgggcccgcatggccaagatcgcgctggagaaggaagcgagcggcgacaagttctacaccgccaagctgg
ccacggcacgcttctactttgccaggctgctgccggaaaccgccgccgagatccgcaaggcgcgcgccggttcggccacgctgatggcgctgg
acgcagacctgttctga
Cupriavidus necator N-1, acyl-CoA dehydrogenase fadE: A1530,
GenBank: CP039287.1 region 1662438 to 1664300, 1863 nt
SEQ ID NO: 93
atgtcgatgatcctatcccgccgcgacctgaatttcgtgctgtacgaatggctcaaggtcgacgagctcacgcgcatcccgcgctatgccgacc
actcgcgcgagactttcgacgccgcgctggacacctgcgagaaaatcgccaccgacctgttcgcgccgcacaacaagaagaacgaccagcaaga
gccgcatttcgacggcgagaccgtcagcatcatccccgaggtcagcaccgcgctgaaggccttctgcgaggccggcttgatggccgccggccag
gactatgaactgggcggcatgcaactgccggtggtggtcgagaaggctggctttgcctacttcaagggtgccaacgtcggcaccagctcgtaccc
gttcctgaccatcggcaacgccaacctgctgctgacgcacggcacgccggcgcaggtcgagaccttcgtcaagccggagatggacggtcgcttc
ttcggcaccatgtgcctgtccgagccgcaggcgggctcgtcgctgtcggacatcaccacgcgcgccgagtacgagggcgaatcgccgctgggc
gcgcagtaccggctgcgcggcaacaagatgtggatctctgccggcgagcacgagctgtcggaaaatatcgtccacctggtgctggccaagatcc
ccggcccggacggcaagctgatcccgggcgtgaagggcatctcgctcttcatcgtgcccaagtacctggtcaatgaagacggctcgctgggcga
gcacaacgacgtggtgctggccggcctgaaccacaagatgggctaccgcggcaccaccaactgcctgctcaacttcggcgaaggcatgaagta
ccggccgggcggcaaggccggcgcgatcggctacctggtgggcgagccgcacaagggcctggcctgcatgttccacatgatgaacgaggcg
cgcatcggcgtgggcctgggcgcggtgatgctgggctataccggctacctgcacgcgctggactacgcgcgcaaccgcccgcaaggccgcgc
ggtcggccccggcggcaaggatgcggccagcccgcaggtgaagctggtcgagcacgccgatatccgccgcatgctgctggcgcagaagagct
atgtcgaaggcggcctggcgctgaacctctattgcgcgcgcctggtcgacgaggaagaggccgctgcggccgccggcgaccaagccgcgcat
gcgcgcctggcgctattgctcgatatcctgaccccgatcgccaagagctggccgtcgcaatggtgcctggaggccaacaacctggcgatccaggt
gcatggcggctacggctatacgcgcgagtacaacgtcgagcagttctaccgcgacaaccgcctcaacccgatccacgaaggcacgcacggcat
ccagggcctggacctgctgggccgcaaggtggtgatgaaggacggcgccgccttcaagctgctgggcgagcgcgtgcaggacaccatcaccc
gcgcgctcgccgccggcaatgccgagttgtcgcagcaggcaggcgccctcggcaccgccaccaagcgcctggccgaggtcacgcaggcgct
gtggagcgcgggcgaccccaacgtgacgctggccaatgcctcggtctacctggaggccttcgggcacgtggtggtggcgtggatctggctgga
acaggcactgctggcgcaagccgcgctgccgcgcgcgaacggcaaggaagacgaggacttctaccgcggcaagctggccgcggcggcctac
ttcttccgctgggagctgcccaaggtcggcccgcagctggcgctgctcgagtcgctcgaccgcaccacgctcgacatgcaggacgcgtggttctg
a
Cupriavidus necator N-1, 3-hydroxyacyl-CoA dehydrogenase (fadB),
NCBI Reference Sequence: NC_015727.1, REGION: complement (968973-971117), 2145 bp
SEQ ID NO: 94

| | |
|---|---|
| 1 | atgcaagccc cgattcagta ccacaagacc gacgacggca tcgtcacgct gacgttcgat |
| 61 | gcgcctgagc aaagcgtcaa taccatgacc gatgagatgc ggcaatgtct ggcggacatg |
| 121 | gtgagccggc tggaagcgga gaaggaagcg gttagcggcg tcattcttac ctcggccaag |
| 181 | gagacgttct ttgcgggagg caatctcaat cgcctgtaca agctgcagcc ggcggatgcg |
| 241 | gctacgcagt tcgatgcctc ggagcgtgcc aagtctgcgc tgaggcggct cgaaacgctg |
| 301 | ggcaagccgg tggtggcggc gctcaatggc acggcgctgg gtggcggctt cgaaattgcg |
| 361 | ctggcctgcc accatcgcat tgcgctggac aagcccaaag tgcaattcgg cctgcccgaa |
| 421 | gcgacgctgg gcctgatgcc gggggcgggc ggcgtcgtgc gtctgaatcg gctgctgggg |
| 481 | cttgctgcga gccagcctta tttgcaggac agcaagctca tgtcgccggc agaggcgacc |
| 541 | aaggttgggc tggtgcatga gcttgcggac acacccgcgg cactgctgga gaaggcacga |
| 601 | gcatggatcg cggcccaccc ggaaagcaag cagccgtggg acaaggccgg ctacacgccg |
| 661 | ccgggaggct gggccgatgc gagtgaggcg cggcgctgga tctccacggc cgccgcgcag |
| 721 | gtgcgcgcca agaccaaagg ttgctaccct gcgccggaag ccatcttgtg cgcttcggtc |
| 781 | gaaggcatgc aggtggactt cgacaccgct agccgcattg agacgcgcta cttcgtgaag |
| 841 | cttgtgactg gccaggttgc gaagaacatc atcagcacct tctggttcca cgccaacggc |
| 901 | atcaagtcag gcgcgcagcg tcctgcaggg gtggccaagg gcaagatcaa gacggtgggc |
| 961 | gtgctgggcg cagggatgat gggcaagggg attgcgtatg tggcggcctc gcgtggtatc |
| 102 | gaggtgtggg tcaaggatgc cacccttgcg caggccgaag gggcacgtgc caatgcggac |
| 108 | caactgctgg ccaagcgtga ggagaagggg gaaattgatg ccgcgacccg ccgacagatt |
| 1141 | gtcgagcgca ttcacgcgac tgaccgctat gaggactttg cccatgtcga cctggtggtg |
| 1201 | gaagccatcc cagagaaccc tgcgcttaag gcggagatca cccggcaggc cgagcccgtg |
| 1261 | ctcggagatg gggcgatctg ggcctccaac acctcgacgc tgcccatcac cggcctggcc |
| 1321 | aaggcatcga gccggcccga gcgcttcgtc gggctgcact tcttctcgcc ggtgcaccgc |
| 1381 | atgcagttgg tggaagtgat taagggccag cagacctcgc cggagaccct ggcccatgcg |
| 1441 | ctggacttcg tgatgcagct tggcaagacg ccgatcgtcg tcaacgacaa ccgcggcttc |
| 1501 | tttaccagcc gggtattcag tactttcaca cgcgaagcag tggcgatgct gggtgagggg |
| 1561 | caggacccgg ccgccatcga ggcggcggcc atcctgtcag ggttccctgc cgggccgctg |
| 1621 | gcggtgctgg acgaggtcag cttgagcttg aactacaaca accggctcga gacgctcagg |
| 1681 | gcgcatgcgg aggagggtcg tccgctgccg ccacatccgg ccgacgcagt gatggagcgc |
| 1741 | atgctcaatg aattcggccg caaggggcgt gccgcgggtg gcggcttcta cgattatccg |
| 1801 | gccgacggca agaaggtgtt ctggagcggt ctggctaagc acttcctgcg cccggccgaa |
| 1861 | cagattccac agcgtgacaa acaggatcgg ttgttgttct gcatggccct ggagtcggtg |
| 1921 | cgtgtactgc aggatggcgt gctggacagc gcgggggacg gcaacattgg ctcggtactg |
| 1981 | gggattggct tcccgcgctg gagcggcggc gtgttccagt tcctgaacca gtatgggctg |
| 2041 | gaaaaggccg tggcacgtgc ggagtacctg gccgagcatt atggcgaacg gttcacgcca |
| 2101 | ccgcaattgc tacgggaaaa ggccaaacga gccgagccat tctga |

Cupriavidus necator N-1, acyl-CoA dehydrogenase fadE: A0460,
NCBI Reference Sequence: WP_011615135.1, NCBI Reference Sequence: WP_010813929.1, 595 aa
SEQ ID NO: 95
MGQYTAPLRDMQFVLHELLGAEAELKAMPPHADIDADTINQVIEEAGKFCSDVVFPLNQVG
DREGCTYVGDGVVKAPTGFKEAYQQYVEAGWPALACDPEFGGQGLPIVINNVVYEMLNSA
GQAWTMYPGLSHGAYEALHAHGTPELQQTYLPKLVSGVWTGTMCLTEPHCGTDLGILRSK
AEPQADGSYLISGTKIFISAGEHDMAENIIHLVLARLPDAPGGTKGISLFVVPKFIPDANGNPGE
RNGIKCGSIEHKMGIHGNATCVMNLDGARGWMVGEPNKGLNAMFVMMNAARLGVGAQG
LGLTEVAYQNSLAYAKDRLQMRALTGPKAPDKPADPIIVHPDVRRMLLTQKAYAEGGRAFS
YWTALQIDRELSHPDEAVRKQAGDLVALLTPVIKAFLTDNAFTSTNEGMQVFGGHGYIAEW
GMEQYVRDARINMIYEGTNTIQALDLLGRKILGDMGARMKAFGKIVQEFVEAEGTNEAMQE
FINPLADIGDKVQKLTMEIGMKAMGNADEVGAAAVPYLRVVGHLVFSYFWARMAKIALEK
EASGDKFYTAKLATARFYFARLLPETAAEIRKARAGSATLMALDADLF
Cupriavidus necator N-1, acyl-CoA dehydrogenase fadE: A1530, 620 aa
SEQ ID NO: 96
MSMILSRRDLNFVLYEWLKVDELTRIPRYADHSRETFDAALDTCEKIATDLFAPHNKKNDQQ
EPHFDGETVSIIPEVSTALKAFCEAGLMAAGQDYELGGMQLPVVVEKAGFAYFKGANVGTSS
YPFLTIGNANLLLTHGTPAQVETFVKPEMDGRFFGTMCLSEPQAGSSLSDITTRAEYEGESPL
GAQYRLRGNKMWISAGEHELSENIVHLVLAKIPGPDGKLIPGVKGISLFIVPKYLVNEDGSLG
EHNDVVLAGLNHKMGYRGTTNCLLNFGEGMKYRPGGKAGAIGYLVGEPHKGLACMFHMM
NEARIGVGLGAVMLGYTGYLHALDYARNRPQGRAVGPGGKDAASPQVKLVEHADIRRMLL
AQKSYVEGGLALNLYCARLVDEEEAAAAAGDQAAHARLALLLDILTPIAKSWPSQWCLEAN
NLAIQVHGGYGYTREYNVEQFYRDNRLNPIHEGTHGIQGLDLLGRKVVMKDGAAFKLLGER
VQDTITRALAAGNAELSQQAGALGTATKRLAEVTQALWSAGDPNVTLANASVYLEAFGHV
VVAWIWLEQALLAQAALPRANGKEDEDFYRGKLAAAAYFFRWELPKVGPQLALLESLDRT
TLDMQDAWF
3-hydroxyacyl-CoA dehydrogenase (fadB) [Cupriavidus necator],
NCBI Reference Sequence: WP_013959369.1, 714 aa
SEQ ID NO: 97

| | |
|---|---|
| 1 | mqapiqyhkt ddgivtltfd apeqsvntmt demrqcladm vsrleaekea vsgviltsak |
| 61 | etffaggnln rlyklqpada atqfdasera ksalrrletl gkpvvaalng talgggfeia |
| 121 | lachhriald kpkvqfglpe atlglmpgag gvvrlnrllg laasqpylqd sklmspaeat |
| 181 | kvglvhelad tpaallekar awiaahpesk qpwdkagytp pggwadasea rrwistaaaq |
| 241 | vraktkgcyp apeailcasv egmqvdfdta srietryfvk lvtgqvakni istfwfhang |
| 301 | iksgaqrpag vakgkiktvg vlgagmmgkg iayvaasrgi evwvkdatla qaegaranad |
| 361 | qllakreekg eidaatrrqi verihatdry edfahvdlvv eaipenpalk aeitrqaepv |
| 421 | lgdgaiwasn tstlpitgla kassrperfv glhffspvhr mqlvevikgq qtspetlaha |
| 481 | ldfvmqlgkt pivvndnrgf ftsrvfstft reavamlgeg qdpaaieaaa ilsgfpagpl |
| 541 | avldevslsl nynnrletlr ahaeegrplp phpadavmer mlnefgrkgr aagggfydyp |
| 601 | adgkkvfwsg lakhflrpae qipqrdkqdr llfcmalesv rvlqdgvlds agdgnigsvl |
| 661 | gigfprwsgg vfqflnqygl ekavaraeyl achygerftp pqllrekakr aepf |

In some embodiments of any of the aspects, the engineered inactivating modification of an endogenous beta-oxidation gene comprises a deletion of the entire coding sequence (e.g., a knockout of an endogenous fadB gene, denoted herein as ΔfadB).

In some embodiments of any of the aspects, the engineered bacterium comprises an inhibitor of an endogenous beta-oxidation enzyme. In some embodiments of any of the aspects, the inhibitor of an endogenous beta-oxidation enzyme is acrylic acid. In some embodiments of any of the aspects, the inhibitor of an endogenous beta-oxidation enzyme comprises enzymes that catalyze the production of acrylic acid (e.g., malonyl-CoA reductase (MCR), malonate semialdehyde reductase (MSR), 3-hydroxypropionyl-CoA synthetase (3HPCS), and 3-hydroxypropionyl-CoA dehydratase (3HPCD) from Metallosphaera sedula; overexpressed succinyl-CoA synthetase (SCS) from E. coli). In some embodiments of any of the aspects, the engineered bacterium comprises at least one functional exogenous gene that catalyzes the production of acrylic acid (e.g., M sedula MCR, M sedula MSR, M sedula 3HPCS, M sedula 3HPCD, and/or E. coli SCS). See e.g., Liu and Liu, Production of acrylic acid and propionic acid by constructing a portion of the 3-hydroxypropionate/4-hydroxybutyrate cycle from Metallosphaera sedula in Escherichia coli; J Ind Microbiol Biotechnol. 2016 December, 43(12):1659-1670. Epub 2016 Oct. 8; the content of which is incorporated herein by reference in its entirety.

In some embodiments of any of the aspects, the inhibitor of an endogenous beta-oxidation enzyme is 2-bromooctanoic acid or 4-pentenoic acid; see e.g., Lee et al., Appl Environ Microbiol. 2001 November; 67(11):4963-74. Additional non-limiting examples of beta oxidation inhibitors include an inhibitory RNA (e.g., siRNA, miRNA) against a beta oxidation gene (e.g., FadB, a 3-hydroxyacyl-CoA dehydrogenase gene), a small molecule inhibitor of a beta oxidation gene (e.g., FadB, a 3-hydroxyacyl-CoA dehydrogenase gene), and the like.

Described herein are methods of sustainably producing TAGs, the general structure of which is shown in Formula I above or FIG. 1A. In one aspect, the method comprises: (a) culturing an engineered bacterium as described herein in a culture medium comprising CO₂ and/or H₂; and (b) isolating, collecting, or concentrating TAGs from said engineered bacterium or from the culture medium of said engineered bacterium. In another aspect, described herein are methods of sustainably producing TAGs comprising: (a) culturing an engineered bacterium as described herein in a culture medium comprising a simple organic carbon source (e.g., glycerol) and/or H₂; and (b) isolating, collecting, or concentrating TAGs from said engineered bacterium or from the culture medium of said engineered bacterium. In some embodiments of any of the aspects, the culture medium comprises CO₂ and glycerol.

TAGs can comprise any combination of fatty acid R groups. Varying the expression of different thioesterases (TE) can lead to the production of TAGs with specific chain-length or composition fatty acid R groups. In some embodiments, all three R group fatty acids of the TAG are the same fatty acids. In some embodiments, the engineered bacteria uses short-chain fatty acids (SCFAs), which are fatty acids with aliphatic tails of five or fewer carbons (e.g. butyric acid), to produce short-chain triglycerides. In some embodiments, the engineered bacteria uses medium-chain fatty acids (MCFA), which are fatty acids with aliphatic tails of 6 to 12 carbons, to form medium-chain triglycerides. In some embodiments, the engineered bacteria uses long-chain fatty acids (LCFA), which are fatty acids with aliphatic tails of 13 to 21 carbons, to produce long-chain triglycerides. In some embodiments, the engineered bacteria uses very long chain fatty acids (VLCFA), which are fatty acids with aliphatic tails of 22 or more carbons, to produce very-long-chain triglycerides.

In some embodiments, the fatty acids used to produce the TAG comprise C4-C18 fatty acids (e.g., C4, C5, C6, C7, C8, C9, C10, C11, C12, C13, C14, C15, C16, etc.). Naturally occurring fatty acids generally have an even number of carbons arranged in a straight chain (e.g., C4, C6, C8, C10, C12, C14, C16, etc.), but fatty acids can also comprise an odd number of carbon atoms in a straight chain (e.g., C5, C7, C9, C11, C13, C15, C17, etc.) In some embodiments, the three fatty acids used to produce the TAG can all comprise C4-C18 fatty acids, either saturated or non-saturated fatty acids. In some embodiments of any of the aspects, the TAG produced by the engineered bacterium comprises R group fatty acids which are 4 to 18 carbons long (C4-C18); such produced TAGs can be referred to herein as “C4-C18 TAGs.” In some embodiments of any of the aspects, the major product of the engineered bacterium is C4-C18 TAG. In some embodiments of any of the aspects, the isolated TAG comprises a majority of C4-C18 TAG. In some embodiments of any of the aspects, the total TAG isolated comprises at least 50%, at least 55%, at least 60%, at least 65%, at least 70%, at least 75%, at least 80%, at least 85%, at least 90%, at least 95%, at least 96%, at least 97%, at least 98%, or least 99% C4-C18 TAG.

In some embodiments, the fatty acids used to produce the TAG comprise C4-C8 fatty acids e.g., C4, C5, C6, C7, C8, etc.). Such short-medium chain-length fatty acids (e.g., C4-C8) predominate in animal fats, compared to longer chain-length fatty acids in plants. In particular, dairy fats, such as those produced by the engineered bacteria, can comprise TAGs with odd-number-length fatty acids and/or significant amounts of short-chain fatty acids. In some embodiments, the three fatty acids used to produce the TAG can all comprise C4-C8 fatty acids, either saturated or non-saturated fatty acids. In some embodiments of any of the aspects, the TAG produced by the engineered bacterium comprises R group fatty acids which are 4 to 8 carbons long (C4-C8); such produced TAGs can be referred to herein as “C4-C8 TAGs.” In some embodiments of any of the aspects, the major product of the engineered bacterium is C4-C8 TAG. In some embodiments of any of the aspects, the isolated TAG comprises a majority of C4-C8 TAG. In some embodiments of any of the aspects, the total TAG isolated comprises at least 50%, at least 55%, at least 60%, at least 65%, at least 70%, at least 75%, at least 80%, at least 85%, at least 90%, at least 95%, at least 96%, at least 97%, at least 98%, or least 99% C4-C8 TAG.

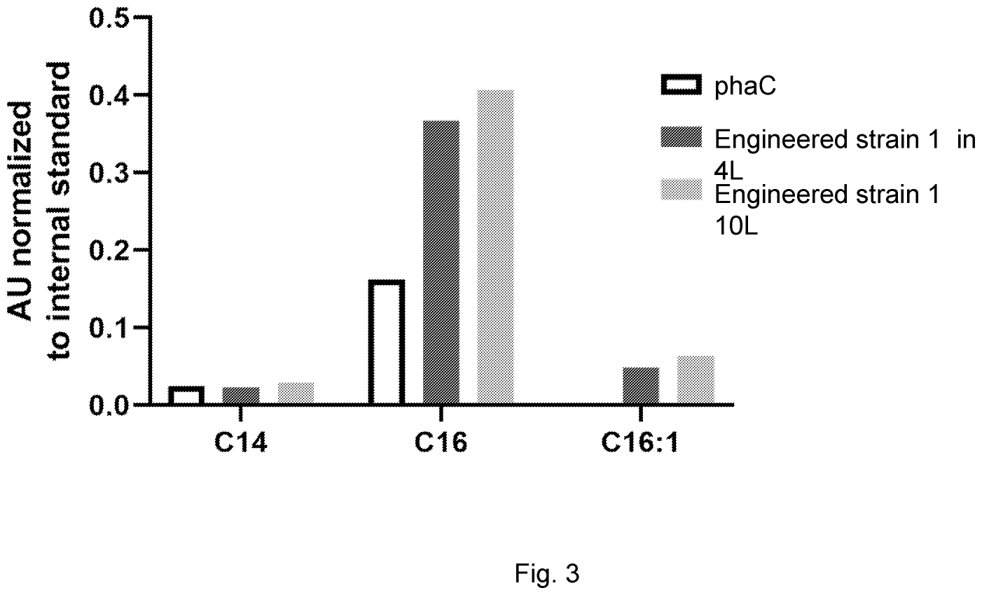

In some embodiments, the fatty acids used to produce the TAG comprise C16 fatty acids. In some embodiments, the three fatty acids used to produce the TAG can all comprise C16 fatty acids, such as saturated C16 fatty acids (e.g., palmitic acid) or unsaturated C16 fatty acids. In some embodiments of any of the aspects, the TAG produced by the engineered bacterium comprises R group fatty acids which are 16 carbons long (C16); such produced TAGs can be referred to herein as “C16 TAGs.” In some embodiments of any of the aspects, the major product of the engineered bacterium is TAG. In some embodiments of any of the aspects, the major product of the engineered bacterium is C16 TAG. In some embodiments of any of the aspects, the isolated TAG comprises a majority of C16 TAG (see e.g., FIG. 3). In some embodiments of any of the aspects, the total TAG isolated comprises at least 50% C16 TAG, at least 55% C16 TAG, at least 60% C16 TAG, at least 65% C16 TAG, at least 70% C16 TAG, at least 75% C16 TAG, at least 80% C16 TAG, at least 85% C16 TAG, at least 90% C16 TAG, at least 95% C16 TAG, at least 96% C16 TAG, at least 97% C16 TAG, at least 98% C16 TAG, or least 99% C16 TAG.

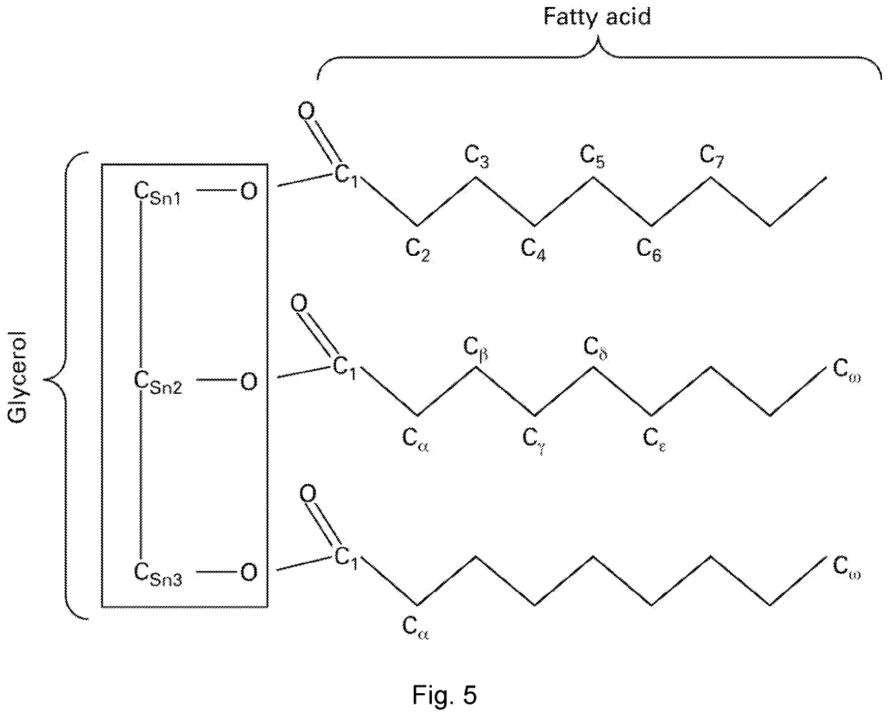

In some embodiments of any of the aspects, the TAG produced by the engineered bacterium comprises specific fatty acids attached at a specific position of the glycerol backbone (see e.g., FIG. 5), e.g., the sn1 carbon, the sn2 carbon, or the sn3 carbon. In some embodiments of any of the aspects, the TAG produced by the engineered bacterium comprises C12-C16 fatty acids on positions sn1 and sn2. In some embodiments of any of the aspects, the TAG produced by the engineered bacterium comprises C4-C10 fatty acids on position sn3. In some embodiments of any of the aspects, the TAG produced by the engineered bacterium comprises C12-C16 fatty acids on positions sn1 and sn2, and C4-C10 fatty acids on position sn3.

In some embodiments of any of the aspects, the cells can be maintained in culture. As used herein, “maintaining” refers to continuing the viability of a cell or population of cells. A maintained population of cells will have at least a subpopulation of metabolically active cells.

As used herein, the term “sustainable” refers to a method of harvesting or using a resource so that the resource is not depleted or permanently damaged. In some embodiments of any of the aspects, the resource is a product that is produced by an engineered bacterium as described herein. In some embodiments of any of the aspects, the engineered bacterium sustainably produces TAGs using a minimal culture medium that comprises CO₂ as the sole carbon source and H₂ as the sole energy source.

As used herein the term “culture medium” refers to a solid, liquid or semi-solid designed to support the growth of microorganisms or cells. In some embodiments of any of the aspects, the culture medium is a liquid. In some embodiments of any of the aspects, the culture medium comprises both the liquid medium and the bacterial cells within it.

In some embodiments of any of the aspects, the culture medium is a minimal medium. As used herein, the term “minimal medium” refers to a cell culture medium in which only few and necessary nutrients are supplied, such as a carbon source, a nitrogen source, salts and trace metals dissolved in water with a buffer. Non-limiting examples of components in a minimal medium include Na₂HPO₄ (e.g., 3.5 g/L), KH₂PO₄ (e.g., 1.5 g/L), (NH₄)₂SO₄ (e.g., 1.0 g/L), MgSO₄·7H₂O (e.g., 80 mg/L), CaSO₄·2H₂O (e.g., 1 mg/L), NiSO₄·7H₂O (e.g., 0.56 mg/L), ferric citrate (e.g., 0.4 mg/L), and NaHCO₃ (200 mg/L). In some embodiments of any of the aspects, a minimal medium can be used to promote lithotrophic growth, e.g., of a chemolithotroph. In some embodiments, (NH₄)Cl (e.g., 1.0 g/L) is used in addition to or instead of (NH₄)₂SO₂. In some embodiments, the minimal media comprises at least one trace metal from Table 5.

TABLE 5
Exemplary trace metals in culture media; see e.g.,
Mozumder et al., Modeling pure culture heterotrophic
production of polyhydroxybutyrate (PHB), Bioresour
Technol. 2014 March; 155: 272-80.

| | exemplary | |
|---|---|---|
| component | concentration | |
| FeSO₄*7H₂O | 10 | g/L |
| ZnSO₄*7H₂O | 2.25 | g/L |
| CuSO₄*5H₂O | 1 | g/L |
| MnSO₄*5H₂O | 0.5 | g/L |
| 35% HCl | 10 | mL/L |
| CaCl₂*2H₂O | 2 | g/L |
| Na₂B₄O₇*10H₂O | 0.23 | g/L |
| (NH₄)₆Mo₇O₂₄ | 0.1 | g/L |

In some embodiments of any of the aspects, the culture medium is a rich medium. As used herein, the term “rich medium” refers to a cell culture medium in which more than just a few and necessary nutrients are supplied, i.e., a non-minimal medium. In some embodiments of any of the aspects, rich culture medium can comprise nutrient broth (e.g., 17.5 g/L), yeast extract (7.5 g/L), and/or (NH₄)₂SO₄ (e.g., 5 g/L). In some embodiments of any of the aspects, a rich medium comprises glycerol. In some embodiments of any of the aspects, a rich medium comprises a minimal media, as described herein or known in the art, and additional nutrients (e.g., nutrient broth, yeast extract, etc.). In some embodiments of any of the aspects, a rich medium does necessarily promote lithotrophic growth. In some embodiments of any of the aspects, a rich medium does not necessarily promote lithotrophic growth. In some embodiments of any of the aspects, a rich medium promotes heterotrophic growth.

In some embodiments of any of the aspects, the culture medium, culture vessel, or environment surrounding the culture medium or culture vessel (e.g., an incubator) comprises approximately 30% H₂ and approximately 15% CO₂. In some embodiments of any of the aspects, the culture medium, culture vessel, or environment surrounding the culture medium or culture vessel (e.g., an incubator) comprises at most 10% H₂, at most 20% H₂, at most 30% H₂, at most 40% H₂, or at most 50% H₂. In some embodiments of any of the aspects, the culture medium, culture vessel, or environment surrounding the culture medium or culture vessel (e.g., an incubator) comprises at most 5% CO₂, at most 10% CO₂, at most 15% CO₂ at most 20% CO₂, or at most 25% CO₂.

In some embodiments of any of the aspects, the culture medium comprises CO₂ as the sole carbon source. In some embodiments of any of the aspects, CO₂ is at least 90%, at least 95%, at least 98%, at least 99% or more of the carbon sources present in the culture medium. In some embodiments of any of the aspects, the culture medium comprises CO₂ in the form of bicarbonate (e.g., HCO₃⁻, NaHCO₃) and/or dissolved CO₂ (e.g., atmospheric CO₂; e.g., CO₂ provided by a cell culture incubator). In some embodiments of any of the aspects, the culture medium does not comprise organic carbon as a carbon source. Non-limiting example of organic carbon sources include fatty acids, gluconate, acetate, fructose, decanoate; see e.g., Jiang et al. Int J Mol Sci. 2016 July; 17(7): 1157).

In some embodiments of any of the aspects, the culture medium comprises glycerol as the sole carbon source. In some embodiments of any of the aspects, glycerol is at least 90%, at least 95%, at least 98%, at least 99% or more of the carbon sources present in the culture medium. In some embodiments of any of the aspects, the culture medium comprises glycerol and CO₂ as the sole carbon sources. In some embodiments of any of the aspects, the glycerol and CO₂ is at least 90%, at least 95%, at least 98%, at least 99% or more of the carbon sources present in the culture medium.

In some embodiments of any of the aspects, the culture medium comprises H₂ as the sole energy source. In some embodiments of any of the aspects, H₂ is at least 90%, at least 95%, at least 98%, at least 99% or more of the energy sources present in the culture medium. In some embodiments of any of the aspects, H₂ is supplied by water-splitting electrodes in the culture medium. Accordingly, in one aspect described herein is a system comprising a reactor chamber with a solution (e.g., culture medium) contained therein. The solution may include hydrogen (H₂), carbon dioxide (CO₂), bioavailable nitrogen (e.g., ammonia, (NH₄)₂SO₄, amino acids), and an engineered bacterium as described herein. Gasses such as one or more of hydrogen (H₂), carbon dioxide (CO₂), nitrogen (N₂), and oxygen (O₂) may also be located within a headspace of the reactor chamber, though embodiments in which a reactor does not include a headspace such as in a flow through reactor are also contemplated. The system may also include a pair of electrodes immersed in the solution (e.g., culture medium). The electrodes are configured to apply a voltage potential to, and pass a current through, the solution to split water contained within the culture medium to form at least hydrogen (H₂) and oxygen (O₂) gasses in the solution. These gases may then become dissolved in the solution. During use, a concentration of the bioavailable nitrogen in the solution may be maintained below a threshold nitrogen concentration that causes the bacteria to produce a desired product (e.g., TAGs). This product may either by excreted from the bacteria and/or stored within the bacteria as the disclosure is not so limited (see e.g., US Patent Publication 2018/0265898, the contents of which are incorporated herein by reference in their entirety).

In some embodiments of any of the aspects, the culture medium does not comprise oxygen (O₂) gasses in the solution, i.e., the culture is grown under anaerobic conditions. In some embodiments of any of the aspects, the culture medium comprises low levels of oxygen (O₂) gasses in the solution, i.e., the culture is grown under hypoxic conditions. As a non-limiting example, the culture medium can comprise at most 30%, at most 20%, at most 15%, at most 10%, at most 5%, at most 4%, at most 3%, at most 2%, or at most 1% O₂ gasses in the solution.

In some embodiments of any of the aspects, the culture medium further comprises arabinose. In some embodiments of any of the aspects, arabinose acts as an inducer for genes in a pBAD vector. In some embodiments of any of the aspects, the culture medium further comprises at least 0.1% arabinose. As a non-limiting example, the culture medium further comprises at least 0.1% arabinose, at least 0.2% arabinose, at least 0.3% arabinose, at least 0.4% arabinose, at least 0.5% arabinose, 0.6% arabinose, at least 0.7% arabinose, at least 0.8% arabinose, at least 0.9% arabinose, or at least 1.0% arabinose.

In some embodiments of any of the aspects, methods described herein comprise isolating, collecting, or concentrating a product from an engineered bacterium or from the culture medium of an engineered bacterium. As used herein the terms “isolate,” “collect,” “concentrate”, “purify” and “extract” are used interchangeably and refer to a process whereby a target component (e.g., TAGs) is removed from a source, such as a fluid (e.g., culture medium). In some embodiments of any of the aspects, methods of isolation, collection, concentration, purification, and/or extraction comprise a reduction in the amount of at least one heterogeneous element (e.g., proteins, nucleic acids; i.e., a contaminant). In some embodiments of any of the aspects, methods of isolation, collection, concentration, purification, and/or extraction reduce by 1%, 2%, 3%, 4%, 5%, 10%, 15%, 20%, 25%, 30%, 35%, 40%, 45%, 50%, 55%, 60%, 65%, 70%, 75%, 80%, 85%, 90% or 95%, or more, the amount of heterogeneous elements, for example biological macromolecules such as proteins or DNA, that may be present in a sample comprising a molecule of interest. The presence of heterogeneous proteins can be assayed by any appropriate method including High-performance Liquid Chromatography (HPLC), gel electrophoresis and staining and/or ELISA assay. The presence of DNA and other nucleic acids can be assayed by any appropriate method including gel electrophoresis and staining and/or assays employing polymerase chain reaction.

Described herein are systems comprising at least one of the engineered bacteria as described herein. In one aspect, the system comprises at least one of the engineered bacteria and a support. In some embodiments of any of the aspects, the bacteria is linked to the support using intrinsic mechanisms (e.g., pili, biofilm, etc.) and/or extrinsic mechanisms (e.g., chemical crosslinking, antibiotics, opsonin, etc.). In some embodiments of any of the aspects, the system further comprises a container and a solution, in which the bacteria linked to the support are submerged. In some embodiments of any of the aspects, the system further comprises a pair of electrodes that split water contained within the solution to form hydrogen. In some embodiments of any of the aspects, the solution (e.g., a culture medium) comprises hydrogen (H₂) and carbon dioxide (CO₂). In some embodiments of any of the aspects, the solution (e.g., a culture medium) comprises hydrogen (H₂) and glycerol. In some embodiments of any of the aspects, the solution (e.g., a culture medium) comprises hydrogen (H₂), glycerol, and carbon dioxide (CO₂).

In some embodiments of any of the aspects, the support comprises a solid substrate. Examples of solid substrate can include, but are not limited to, film, beads or particles (including nanoparticles, microparticles, polymer microbeads, magnetic microbeads, and the like), filters, fibers, screens, mesh, tubes, hollow fibers, scaffolds, plates, channels, gold particles, magnetic materials, medical apparatuses (e.g., needles or catheters) or implants, dipsticks or test strips, filtration devices or membranes, hollow fiber cartridges, microfluidic devices, mixing elements (e.g., spiral mixers), extracorporeal devices, and other substrates commonly utilized in assay formats, and any combinations thereof. In some embodiments of any of the aspects, the solid substrate can be a magnetic particle or bead.

In several aspects, the system comprises a reactor chamber and at least one of the engineered bacteria as described herein. Accordingly, in one aspect, described herein is a system comprising: (a) a reactor chamber with a solution contained therein, wherein the solution comprises hydrogen (H₂) and carbon dioxide (CO₂); and (b) at least one engineered bacterium as described herein in the solution. Also described herein is a system comprising: (a) a reactor chamber with a solution contained therein, wherein the solution comprises hydrogen (H₂) and glycerol; and (b) at least one engineered bacterium as described herein in the solution. In some embodiments of any of the aspects, the system further comprises a pair of electrodes in contact with the solution that split water to form the hydrogen. In one aspect, described herein is a system comprising: (a) a reactor chamber; and (b) at least one engineered bacterium. In some embodiments of any of the aspects, the system further comprises a pair of electrodes in contact with reactor chamber.

In one aspect, described herein is a system comprising: (a) a reactor chamber with a solution contained therein, wherein the solution comprises hydrogen (H₂) and a carbon source; and (b) an engineered bacterium as described herein. In some embodiments of any of the aspects, the carbon source is carbon dioxide (CO₂) and/or glycerol. In some embodiments of any of the aspects, the system further comprises a pair of electrodes in contact with the solution that split water to form the hydrogen. In some embodiments of any of the aspects, the system (e.g., a system comprising a reactor chamber, a system comprising a support) can comprise any combination of engineered bacteria as described herein.

In one aspect, described herein is a system comprising: (a) a reactor chamber with a solution contained therein, wherein the solution comprises hydrogen (H₂) and carbon dioxide (CO₂); (b) an engineered TAG bacterium as described herein in the solution; and (c) a pair of electrodes in contact with the solution that split water to form the hydrogen.

In one aspect, described herein is a system comprising: (a) a reactor chamber with a solution contained therein, wherein the solution comprises hydrogen (H₂) and glycerol; (b) an engineered TAG bacterium as described herein in the solution; and (c) a pair of electrodes in contact with the solution that split water to form the hydrogen.

In one aspect, described herein is a system comprising: (a) a reactor chamber with a solution contained therein, wherein the solution comprises hydrogen (H₂), glycerol, and carbon dioxide (CO₂); (b) an engineered TAG bacterium as described herein in the solution; and (c) a pair of electrodes in contact with the solution that split water to form the hydrogen.

In some embodiments of any of the aspects, the pair of electrodes comprise a cathode including a cobalt-phosphorus alloy and an anode including cobalt phosphate. In some embodiments of any of the aspects, a concentration of the bioavailable nitrogen in the solution is below a threshold nitrogen concentration to cause the engineered bacteria to produce a product. In some embodiments of any of the aspects, the solution is also referred to as a culture medium and can comprise a minimal medium as described further herein.

In one embodiment, a system includes a reactor chamber containing a solution. The solution may include hydrogen (H₂), carbon dioxide (CO₂), bioavailable nitrogen, and an engineered bacteria. Gasses such as one or more of hydrogen (H₂), carbon dioxide (CO₂), nitrogen (N₂), and oxygen (O₂) may also be located within a headspace of the reactor chamber, though embodiments in which a reactor does not include a headspace such as in a flow through reactor are also contemplated. The system may also include a pair of electrodes immersed in the solution. The electrodes are configured to apply a voltage potential to, and pass a current through, the solution to split water contained within the solution to form at least hydrogen (H₂) and oxygen (O₂) gasses in the solution. These gases may then become dissolved in the solution. During use, a concentration of the bioavailable nitrogen in the solution may be maintained below a threshold nitrogen concentration that causes the bacteria to produce a desired product. This product may either by excreted from the bacteria and/or stored within the bacteria as the disclosure is not so limited.

Concentrations of the above noted gases both dissolved within a solution, and/or within a headspace above the solution, may be controlled in any number of ways including bubbling gases through the solution, generating the dissolved gases within the solution as noted above (e.g. electrolysis/water splitting), periodically refreshing a composition of gases located within a headspace above the solution, or any other appropriate method of controlling the concentration of dissolved gas within the solution. Additionally, the various methods of controlling concentration may either be operated in a steady-state mode with constant operating parameters, and/or a concentration of one or more of the dissolved gases may be monitored to enable a feedback process to actively change the concentrations, generation rates, or other appropriate parameter to change the concentration of dissolved gases to be within the desired ranges noted herein. Monitoring of the gas concentrations may be done in any appropriate manner including pH monitoring, dissolved oxygen meters, gas chromatography, or any other appropriate method.

As noted above, in one embodiment, the composition of a volume of gas located in a headspace of a reactor may include one or more of carbon dioxide, oxygen, hydrogen, and nitrogen. A concentration of the carbon dioxide may be between 10 volume percent (vol %) and 100 vol %. However, carbon dioxide may also be greater than equal to 0.04 vol % and/or any other appropriate concentration. For example, carbon dioxide may be between or equal to 0.04 vol % and 100 vol %. A concentration of the oxygen may be between 1 vol % and 99 vol % and/or any other appropriate concentration. A concentration of the hydrogen may be greater than or equal to 0.05 vol % and 99%. A concentration of the nitrogen may be between 0 vol % and 99 vol %.

As also noted, in one embodiment, a solution within a reactor chamber may include water as well as one or more of carbon dioxide, oxygen, and hydrogen dissolved within the water. A concentration of the carbon dioxide in the solution may be between 0.04 vol % to saturation within the solution. A concentration of the oxygen in the solution may be between 1 vol % to saturation within the solution. A concentration of the hydrogen in the solution may be between 0.05 vol % to saturation within the solution provided that appropriate concentrations of carbon dioxide and/or oxygen are also present.

As noted previously, and as described further below, production of a desired end product by bacteria located within the solution may be controlled by limiting a concentration of bioavailable nitrogen, such as in the form of ammonia, amino acids, or any other appropriate source of nitrogen useable by the bacteria within the solution to below a threshold nitrogen concentration. However, and without wishing to be bound by theory, the concentration threshold may be different for different bacteria and/or for different concentrations of bacteria. For example, a solution containing enough ammonia to support a Ralstonia eutropha (i.e., Cupriavidus necator) population up to an optical density (OD) of 2.3 produces product at molar concentrations less than or equal to 0.03 M while a population with an OD of 0.7 produces product at molar concentrations less than or equal to 0.9 mM. Accordingly, higher optical densities may be correlated with producing product at higher nitrogen concentrations while lower optical densities may be correlated with producing product at lower nitrogen concentrations. Further, bacteria may be used to produce product by simply placing them in solutions containing no nitrogen. In view of the above, an optical density of bacteria within a solution may be between or equal to 0.1 and 12, 0.7 and 12, or any other appropriate concentration including concentrations both larger and smaller than those noted above. Additionally, a concentration of nitrogen within the solution may be between or equal to 0 and 0.2 molar, 0.0001 and 0.1 molar, 0.0001 and 0.05 molar, 0.0001 and 0.03 molar, or any other appropriate composition including compositions greater and less than the ranges noted above.

While particular gasses and compositions have been detailed above, it should be understood that the gasses located with a headspace of a reactor as well as a solution within the reactor may include compositions and/or concentrations as the disclosure is not limited in this fashion.

Bacteria used in the systems and methods disclosed herein may be selected so that the bacteria both oxidize hydrogen as well as consume carbon dioxide. Accordingly, in some embodiments, the bacteria may include an enzyme capable of metabolizing hydrogen as an energy source such as with hydrogenase enzymes. Additionally, the bacteria may include one or more enzymes capable of performing carbon fixation such as Ribulose-1,5-bisphosphate carboxylase/oxygenase (RuBisCO). One possible class of bacteria that may be used in the systems and methods described herein to produce a product include, but are not limited to, chemolithoautotrophs. Additionally, appropriate chemolithoautotrophs may include any one or more of Ralstonia eutropha (R. eutropha) as well as Alcaligenes paradoxs I 360 bacteria, Alcaligenes paradoxs 12/X bacteria, Nocardia opaca bacteria, Nocardia autotrophica bacteria, Paracoccus denitrificans bacteria, Pseudomonas facilis bacteria, Arthrobacter species 1IX bacteria, Xanthobacter autotrophicus bacteria, Azospirillum lipferum bacteria, Derxia Gummosa bacteria, Rhizobium japonicum bacteria, Microcyclus aquaticus bacteria, Microcyclus ebruneus bacteria, Renobacter vacuolatum bacteria, and any other appropriate bacteria.

A bacterium in the system or bioreactor can either naturally include a TAG production pathway, or may be appropriately engineered, to include a TAG production pathway when placed under the appropriate growth conditions.

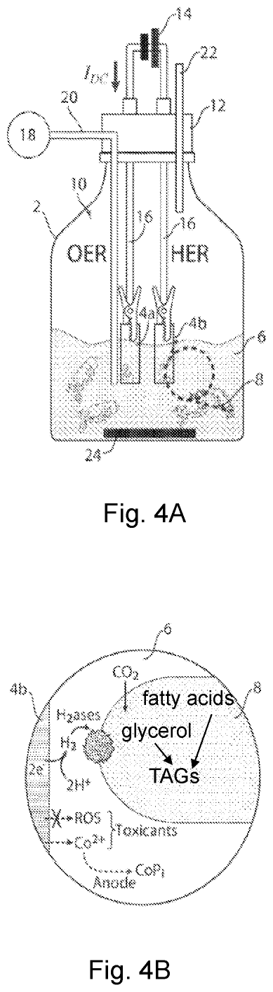

FIG. 4A shows a schematic of one embodiment of a system including one or more reactor chambers. In the depicted embodiment, a single-chamber reactor 2 houses one or more pairs of electrodes including an anode 4a and a cathode 4b immersed in a water based solution 6. Bacteria 8 are also included in the solution. A headspace 10 corresponding to a volume of gas that is isolated from an exterior environment is located above the solution within the reactor chamber. The gas volume may correspond to any appropriate composition including, but not limited to, carbon dioxide, nitrogen, hydrogen, oxygen, and any other appropriate gases as the disclosure is not so limited. Additionally, as detailed further below, the various gases may be present in any appropriate concentration as detailed previously. However, it should be understood that embodiments in which a reactor chamber is exposed to an external atmosphere that may either be a controlled composition and/or a normal atmosphere are also contemplated. The system may also include one or more temperature regulation devices such as a water bath, temperature controlled ovens, or other appropriate configurations and/or devices to maintain a reactor chamber at any desirable temperature range for bacterial growth.

In embodiments where a reactor chamber interior is isolated from an exterior environment, the system may include one or more seals 12. In the depicted embodiment, the seal corresponds to a cork, stopper, a threaded cap, a latched lid, or any other appropriate structure that seals an outlet from an interior of the reactor chamber. In this particular embodiment, a power source 14 is electrically connected to the anode and cathode via two or more electrical leads 16 that pass through one or more pass throughs in the seal to apply a potential to and pass a current IDC to split water within the solution into hydrogen and oxygen through an oxygen evolution reaction (OER) at the anode and a hydrogen evolution reaction (HER) at the cathode. While the leads have been depicted as passing through the seal, it should be understood that embodiments in which the leads pass through a different portion of the system, such as a wall of the reactor chamber, are also contemplated as the disclosure is so limited.

Depending on the particular embodiment, the above-described power source may correspond to any appropriate source of electrical current that is applied to the electrodes. However, in at least one embodiment, the power source may correspond to a renewable source of energy such as a solar cell, wind turbine, or any other appropriate source of current though embodiments in which a non-renewable energy source, such as a generator, battery, grid power, or other power source is used are also contemplated. In either case, a current from the power source is passed through the electrodes and solution to evolve hydrogen and oxygen. The current may be controlled to produce hydrogen and/or oxygen at a desired rate of production as noted above. In some embodiments of any of the aspects, a system comprising a renewable source of energy (e.g., a solar cell) can also be referred to as a “bionic leaf”.

Accordingly, in one aspect, described herein is a system comprising: (a) a reactor chamber with a solution contained therein, wherein the solution comprises hydrogen (H₂) and carbon dioxide (CO₂); (b) an engineered bacteria as described herein; (c) a pair of electrodes in contact with the solution that split water to form the hydrogen; and (d) comprising a power source comprising a renewable source of energy.

In another aspect, described herein is a system comprising: (a) a reactor chamber with a solution contained therein, wherein the solution comprises hydrogen (H₂) and glycerol; (b) an engineered bacteria as described herein; (c) a pair of electrodes in contact with the solution that split water to form the hydrogen; and (d) comprising a power source comprising a renewable source of energy.

In one aspect, described herein is a system comprising: (a) a reactor chamber with a solution contained therein, wherein the solution comprises hydrogen (H₂), glycerol and carbon dioxide (CO₂); (b) an engineered bacteria as described herein; (c) a pair of electrodes in contact with the solution that split water to form the hydrogen; and (d) comprising a power source comprising a renewable source of energy.

In some embodiments, the electrodes may be coated with, or formed from, a water splitting catalyst to further facilitate water splitting and/or reduce the voltage applied to the solution. In some embodiments, the catalysts may be coated onto an electrode substrate including, for example, carbon fabrics, porous carbon foams, porous metal foams, metal fabrics, solid electrodes, and/or any other appropriate geometry or material as the disclosure is not so limited. In another embodiment, the electrodes may simply be made from a desired catalyst material. Several appropriate materials for use as catalysts include, but are not limited to, one or more of a cobalt-phosphorus (Co—P) alloy, cobalt phosphate (CoPi), cobalt oxide, cobalt hydroxide, cobalt oxyhydroxide, a NiMoZn alloy, or any other appropriate material. As noted further below, certain catalysts offer additional benefits as well. For example, in one specific embodiment, the electrodes may correspond to a cathode including a cobalt-phosphorus alloy and an anode including cobalt phosphate, which may help to reduce the presence of reactive oxygen species and/or metal ions within a solution. A composition of the CoPi coating and/or electrode may include phosphorous compositions between or equal to 0 weight percent (wt %) and 50 wt %. Additionally, the Co—P alloy may include between 80 wt % and 99 wt % Co as well as 1 wt % and 20 wt % P. However, embodiments in which different element concentrations are used and/or other types of catalysts and/or electrodes are used are also contemplated as the disclosure is not so limited. For example, stainless steel, platinum, and/or other types of electrodes may be used.

As also shown in FIG. 4A, in some embodiments, it may be desirable to either continuously, or periodically, bubble, i.e. sparge or flush, one or more gases through a solution 6 and/or to refresh a composition of gases located within a head space 10 of the reactor chamber 2 above a surface of the solution. In such an embodiment, a gas source 18 may be in fluid communication with one or more gas inlets 20 that pass through either a seal 12 and/or another portion of the reactor chamber 2 such as a side wall to place the gas source in fluid communication with an interior of the reactor chamber. Additionally, in some embodiments, one or more inlets discharge a flow of gas into the solution so that the gas will bubble through the solution. However, embodiments in which the one or more gas inlets discharge a flow of gas into the headspace of the reactor chamber instead are also contemplated as the disclosure is not so limited. Additionally, one or more corresponding gas outlets 22 may be formed in a seal and/or another portion of the reactor chamber to permit a flow of gas to flow from an interior to an exterior of the reactor chamber. It should be noted that gas inlets and outlets may correspond to any appropriate structure including, but not limited to, tubes, pipes, flow passages, ports in direct fluid communication with the reactor chamber interior, or any other appropriate structure.

Gas sources may correspond to any appropriate gas source capable of providing a pressurized flow of gas to the chamber through the inlet including, for example, one or more pressurized gas cylinders. While a gas source may include any appropriate composition of one or more gasses, in one embodiment, a gas source may provide one or more of hydrogen, nitrogen, carbon dioxide, and oxygen. The flow of gas provided by the gas source may have a composition equivalent to the range of gas compositions described above for the gas composition with a headspace of the reactor chamber. Further, in some embodiments, the gas source may simply be a source of carbon dioxide. Of course embodiments in which a different mix of gases, other including different gases and/or different concentrations than those noted above, is bubbled through a solution or otherwise input into a reactor chamber are also contemplated as the disclosure is not so limited. Additionally, the gas source may be used to help maintain operation of a reactor at, below, and/or above atmospheric pressure as the disclosure is not limited to any particular pressure range.

The above noted one or more gas inlets and outlets may also include one or more valves located along a flow path between the gas source and an exterior end of the one or more outlets. These valves may include for example, manually operated valves, pneumatically or hydraulically actuated valves, unidirectional valves (i.e. check valves) may also be incorporated in the one or more inlets and/or outlets to selectively prevent the flow of gases into or out of the reactor either entirely or in the upstream direction into the chamber and/or towards the gas source.

While the use of inlet and/or outlet gas passages have been described above, embodiments in which there are no inlet and/or outlets for gasses are present are also contemplated. For example, in one embodiment, a system including a sealable reactor may simply be flushed with appropriate gasses prior to being sealed. The system may then be flushed with an appropriate composition of gasses at periodic intervals to refresh the desired gas composition in the solution and/or headspace prior to resealing the reactor chamber. Alternatively, the head space may be sized to contain a gas volume sufficient for use during an entire production run.

In instances where electrodes are run at high enough rates and/or for sufficient durations, concentration may be formed within a solution in a reactor chamber. Accordingly, it may be desirable to either prevent and/or mitigate the presence of concentration gradients in the solution. Therefore, in some embodiments, a system may include a mixer such as a stir bar 24 illustrated in FIG. 4A. Alternatively, a shaker table, and/or any other way of inducing motion in the solution to reduce the presence of concentration gradients may also be used as the disclosure is not so limited.

While the above embodiment has been directed to an isolated reactor chamber, embodiments in which a flow-through reaction chamber with two or more corresponding electrodes immersed in a solution that is flowed through the reaction chamber and past the electrodes are also contemplated. For example, one possible embodiment, one or more corresponding electrodes may be suspended within a solution flowing through a chamber, tube, passage, or other structure. Similar to the above embodiment, the electrodes are electrically coupled with a corresponding power source to perform water splitting as the solution flows past the electrodes. Such a system may either be a single pass flow through system and/or the solution may be continuously flowed passed the electrodes in a continuous loop though other configurations are also contemplated as well.

Without wishing to be bound by theory, FIG. 4B illustrates one possible pathway for a system to produce one or more desired products. In the depicted embodiment, the hydrogen evolution reaction occurs at the cathode 4b. During the reaction at the cathode, two hydrogen ions (H⁺) are combined with two electrons to form hydrogen gas H₂ that dissolves within the solution 6 along with carbon dioxide (CO₂), which dissolved in the solution as well. At the same time various toxicants such as reactive oxygen species (ROS) including, for example, hydrogen peroxide (H₂O₂), superoxides (O₂⁻), and/or hydroxyl radical (HO·) species as well as metallic ions may be generated at the cathode. For example, CO²⁺ ions may be dissolved into solution when a cobalt based cathode is used. As described further below, in some embodiments, the use of certain catalysts may help to reduce the production of ROS and the metallic ions leached into the solution may be deposited onto the anode using one or more elements located within the solution to form compounds such as a cobalt phosphate.

As also illustrated in FIG. 4B, once hydrogen and carbon dioxide are provided within a solution, bacteria 8 present within the solution may be used to transform these compounds into useful products (e.g., TAGs). For example, in one embodiment, the bacteria uses hydrogenase to metabolize the dissolved hydrogen gas and one or more appropriate enzymes, such as RuBisCO or other appropriate enzyme, to provide a carbon fixation pathway. This may include absorbing the carbon dioxide and forming Acetyl-CoA through the Calvin cycle. Further, depending on the concentration of nitrogen within the solution, the bacteria may either form biomass or one or more desired products. For instance, if a concentration of nitrogen within the solution is below a predetermined nitrogen concentration threshold, the bacteria may form one or more products such as TAGs, as depicted in the figure.

Depending on the embodiment, a solution placed in the chamber of a reactor may include water with one or more additional solvents, compounds, and/or additives. For example, the solution may include: inorganic salts such as phosphates including sodium phosphates and potassium phosphates; trace metal supplements such as iron, nickel, manganese, zinc, copper, and molybdenum; or any other appropriate component in addition to the dissolved gasses noted above. In one such embodiment, a phosphate may have a concentration between 9 and 90 mM, 9 and 72 mM, 9 and 50 mM, or any other appropriate concentration. In a particular embodiment, a water based solution may include one or more of the following in the listed concentrations: 12 mM to 123 mM of Na₂HPO₄, 11 mM to 33 mM of KH₂PO₄, 1.25 mM to 15 mM of (NH₄)₂SO₄, 0.16 mM to 0.64 mM of MgSO₄, 2.4 M to 5.8 μM of CaSO₄, 1 μM to 4 μM of NiSO₄, 0.81 μM to 3.25 μM molar concentration of Ferric Citrate, 60 mM to 240 mM molar concentration of NaHCO₃.

As noted above in regards to the discussion of FIG. 4B, reactive oxygen species (ROS) as well as metallic ions may be formed and/or dissolved into a solution during the hydrogen evolution reaction at the cathode. However, ROS and larger concentrations of the metallic ions within the solution may be detrimental to cell growth above certain concentrations. It is noted that the use of continuous hydrogen production within a reactor to form hydrogen for conversion into one or more desired products has been hampered by the production of these ROS and metallic ion concentrations because the bacteria used to form the desired products tend to be sensitive to these compounds and ions limiting the growth of, and above certain concentrations, killing the bacteria. Therefore, in some embodiments, it may be desirable to apply voltages, use electrodes that produce less ROS, remove and/or prevent the dissolution of metallic ions from the electrodes, and/or use bacteria that are resistant to the presence of these toxicants as detailed further below.

As noted above, it may be desirable to select one or more catalysts for use as the electrodes that produce fewer reactive oxygen species (ROS) during use. Specifically, a biocompatible catalyst system that is not toxic to the bacterium and lowers the overpotential for water splitting may be used in some embodiments. One such example of a catalyst includes a ROS-resistant cobalt-phosphorus (Co—P) alloy cathode. This cathode may be combined with a cobalt phosphate (CoPi) anode. This catalyst pair has the added benefit of the anode being self-healing. In other words, the catalyst pair helps to remove metallic Co²⁺ ions present with a solution in a reactor. Without wishing to be bound by theory, the electrode pair works in concert to remove extracted metal ions from the cathode by depositing them onto the anode which may help to maintain extraneous cobalt ions at relatively low concentrations within solution and to deliver a low applied electrical potential to split water to generate H₂. Without wishing to be bound by theory, it is believed that during electrolysis of the water, phosphorus and/or cobalt is extracted from the electrodes. The reduction potential of leached cobalt is such that formation of cobalt phosphate using phosphate available in the solution is energetically favored. Cobalt phosphate formed in solution then deposits onto the anode at a rate linearly proportional to free Co²⁺, providing a self-healing process for the electrodes. In view of the above, the cobalt-phosphorus (Co—P) alloy and cobalt phosphate (CoPi) catalysts may be used to help mitigate the presence of both ROS and metal ions within the solution to help promote growth of bacteria within the reactor chamber.

It should be understood that any appropriate voltage may be applied to a pair of electrodes immersed in a solution to split water into hydrogen and oxygen. However, in some embodiments, the applied voltage may be limited to fall between upper and lower voltage thresholds. For example, the self-healing properties of a cobalt phosphate and cobalt phosphorous based alloy electrode pair may function at voltage potentials greater than about 1.42 V. Additionally, the thermodynamic minimum potential for splitting water is about 1.23 V. Therefore, depending on the particular embodiment, the voltage applied to the electrodes may be greater than or equal to about 1.23 V, 1.42 V, 1.5 V, 2 V, 2.2 V, 2.4 V, or any other appropriate voltage. Additionally, the applied voltage may be less than or equal to about 10 V, 5 V, 4 V, 3 V, 2.9 V, 2.8 V, 2.7 V, 2.6 V, 2.5 V, or any other appropriate voltage. Combinations of the above noted voltage ranges are contemplated including, for example, a voltage applied to a pair of electrodes may be between 1.23 V and 10 V, 1.42 V and 5 V, 2 V and 3 V, 2.3 V and 2.7 V as well as other appropriate ranges. Additionally, it should be understood that voltages both greater than and less than those noted above, as well as different combinations of the above ranges, are also contemplated as the disclosure is not so limited. In addition to the applied voltages, any appropriate current may be passed through the electrodes to perform water splitting which will depend on the desired rate of hydrogen generation for a given volume of a reactor being used. For example, in some embodiments, a current used to split water may be controlled to generate hydrogen at a rate substantially equal to a rate of hydrogen consumption by bacteria in the solution. However, embodiments in which hydrogen is produced at rates both greater than or less than consumption by the bacteria are also contemplated.

In addition to using catalysts, controlling the solution pH, and applying appropriate driving potentials, and/or controlling any other appropriate parameter to reduce the presence of reactive oxygen species (ROS) within the solution in a reaction chamber, it may also be desirable to use bacteria that are resistant to the presence of ROS and/or metallic ions present within the solution as noted previously. Specifically, a chemolithoautotrophic bacterium that is resistant to reactive oxygen species may be used. Further, in some embodiments a R. eutropha bacteria that is resistant to ROS as compared to a wild-type H16 R. eutropha may be used. US 2018/0265898 and Table 2 below detail several genetic polymorphisms found between the wild-type H16 R. eutropha and a ROS-tolerant BC4 strain that was purposefully evolved. Mutations of the BC4 strain relative to the wild type bacteria are detailed further below.

TABLE 2
Mutations in ROS-tolerant BC4 strain

| Mutation | Position | Annotation | Gene | Description |
|---|---|---|---|---|
| G → T | 611,894 | R133R | acrC1 | cation/multidrug |
| | | | | efflux system outer |
| | | | | membrane protein |
| Δ45 bp | 611,905 | 344-388 of | acrC1 | cation/multidrug |
| | | 1494 nt | | efflux system outer |
| | | | | membrane protein |
| G → A | 2,563,281 | intergenic, | Hfq and | uncharacterized host |
| | | (−1/+210) | H16_A2360 | factor I protein/ |
| | | | | GTP-binding protein |
| Δ15 bp | 241,880 | 363-377 of | H16_B0214 | transcriptional |
| | | 957 nt | | regulator, |
| | | | | LysR-Family |

Two single nucleotide polymorphisms and two deletion events have been observed. Without wishing to be bound by theory, the large deletion from acrC1 may indicate a decrease in overall membrane permeability, possibly affecting superoxide entry to the cell resulting in the observed ROS resistance. The genome sequences are accessible at the NCBI SRA database under the accession number SRP073266 and specific mutations of the BC4 strain are listed below. The standard genome sequence for the wild-type H16 R. eutropha is also accessible at the RCSB Protein Data Bank under accession number AM260479 which the following mutations may also be referenced to.

In reference to the above table, an R. eutropha bacteria may include at least one to four mutations selected from the mutations noted above in Table 2 and may be selected in any combination. These specific mutations are listed below in more detail with mutations noted relative to the wild type R. eutropha bolded and underlined within the sequences given below.

The first noted mutation may correspond to the sequence listed below ranging from position 611790-611998 for Ralstonia eutropha H16 chromosome 1. The bolded, double underlined text indicates a mutation (e.g., nt 105 of SEQ ID NO: 12).

(209 nt)
SEQ ID NO: 12
GCCTCGCTGCTTTCCACCTGGCGCCGCACGCGGCCCCAGACGTCGA
TTTCCCAGGTTGCGCCCAGGGTCGCGCTCTGCCCGTTGAGCGTGCTGCCG CTGGCGCC G
CGCGCGCGCGAGGCGCCGGCCTGTGCGTCGACGGTCGGGAA
GAAGCCGGCGCGCGCGGCCTGCAGCGACGCCACCGCCTGGCGGTACTGCG
CCTCGGCGGCCTT
The second noted mutation may correspond to the sequence listed below ranging from
position 611905-613399 for Ralstonia eutropha H16 chromosome 1. The bolded, double
underlined text indicates a mutation (e.g., nt 345-390 of SEQ ID NO: 13).
(1495 nt)
SEQ ID NO: 13
AGGCGCCGGCCTGTGCGTCGACGGTCGGGAAGAAGCCGGCGCGCG
CGGCCTGCAGCGACGCCACCGCCTGGCGGTACTGCGCCTCGGCGGCCTTG
ATGTTCTGGTTCGAGATCTGCACCTCGGACATCAGCGCGTCGAGCTGCGC
ATCGCCGAACACGGTCCACCAGTCGGCGCGTGCCAGCGCATCCTGCGGCT
CGGCGGGCTTCCAGTCGCCGGTCCAGGCGGGGGTGGCGGCATCGGCTTCC
TTGAAGGATGCGGAAACCGGCGCGTCGGGGCGCTGGTAGTCGGGGCCGAC
GGCGCAGCCGGCCAGCAGCAGCGCGCAGGCCAGCGACACCGGCAGGGCA T
GGGTCAGGAGGCGGGAAAGAACTGTCATGTCGAGTCTTCGCAAAT CTAGA
CGGCGGCCGGCTGGTCAGGCGTGCCGGCACCACGGCGGCGCTGGCGCCAG
GCCTTGACCTTCAGGCGCCAGCGGTCCAGCGTCAGGTAGACCACCGGCGT
GGTGTACAGCGTCAGCAGCTGGCTTACCACCAGTCCGCCGACAATGGAGA
TGCCCAGCGGCGCGCGCAGTTCGGCGCCGTCGCCGCGGCCGATTGCCAGC
GGCACCGCGCCCAGCAGCGCGGCCATGGTGGTCATCAGGATCGGGCGGAA
GCGCAGCAGGCAGGCGCGGTAGATCGCGTCGCGCGGCGACAGGCCATCGC
GCCGTTCGGCATCGATGGCGAAGTCGATCATCATGATCGCGTTCTTTTTC
ACGATGCCGATCAGCAGGATCACGCCGATCAGCGCGATGATGCTGAAGTC
GGTCTTCGATGCCAGCAGCGCCAGCAGCGCGCCCACGCCGGCGGAGGGCA
GCGTCGACAGGATCGTCAGCGGATGCACATAGCTTTCATACAGCACGCCC
AGCACGATGTAGATCGTGATCAGCGCCGCCAGGATCAGGATCGGCTGACT
CTTGAGCGAATCCTGGAACGCCTTGGCGCCGCCCTGGAAGTTGGCGCGCA
GCGTCTCCGGCACGCCGATGCGCGCCATCTCGCGCGTGATCGCGTCGGTC
GCCTGCGACAGCGAAGTGCCCTCGGCCAGGTTGAACGAGATCGTCGAGGC
CGCGAACTGGCCCTGGTGGTTCACGCCCAGCGGCGTGCTGGACGGGGTCA
CGCGCGCGAACGCCGCCAGCGGCACGCGGTTGCCGTTGCCGGTGACCACG
TAGATGTCCTTGAGCGCATCGGGCCCTTGCAGGTATTCCTGGCTCAGCTC
CATCACCACGCGGTACTGGTTCAGCGGATGGTAGATGGTGGACACCAGCC
GCTGGCCGAAGGCATCGTTGAGCACCGCATCCACCTGCTGCGCGGTCACG
CCCAGGCGCGAGGCCGCGTCGCGGTCGATGATCACCGAGGTCTGCAGGCC
CTTGTCGTTGGTATCGGTGTCGATATCCTCCAGCCCCTTCAGGTTCGACA
ACGCGGCGCGCACCTTGGGCTCCCACGCGCGCAGCACTTCCAGGTCGTCC
The third noted mutation may correspond to the sequence listed below ranging from
position 2563181-2563281 for Ralstonia eutropha H16 chromosome 1. The bolded, double
underlined text indicates a mutation (e.g., nt 101 of SEQ ID NO: 14).
(201 nt)
SEQ ID NO: 14
GCAGCTTGATGCCATTGACGAGGTAGATGGAAACCGGCACGTGCTC
TTTGCGCAGCGCGTTCAGGAACGGGCCTTGTAGCAGTTGCCCTTTGTTGC TCAT G
GCACACTCCAAATTTATAGGTTTAGTGGTGAATGATGGGGATGGA
AATCCCCGGTTCAAGTCAGGCGGCGCAAAAACGCGCCAGAAAAAAGATCA AAAAC
The fourth noted mutation may correspond to the sequence listed below ranging from
position 241880-242243 for Ralstonia eutropha H16 chromosome 1. The bolded, double
underlined text indicates a mutation (e.g., nt 364-379 of SEQ ID NO: 15).
(479 nt)
SEQ ID NO: 15
GAGGATGCCATGTCCGAAGCGCCTGTCCTTGCCCCCTCGACCTCAA
CCCAGCCGCCCGCCGCCGGCCAGCTCAACCTGATCCGCCCGCAGCCATAT
GCCGACTGGGCGCCGCAGGTCACGGCCGAAGAACGCGCCACGCTGCGCCG
CGAGCTGGAGCAGGGCGCCGTGCTGTACTTCCCGAACCTGAATTTCCGCT
TCCAGCCGGGCGAAGAGCGCTTCCTTGACAGCCGCTATTCCGACGGCAAG
TCCAAGAACATCAACCTGCGCGCCGACGACACCGCGGTGCGCGGCGCCCA
GGGCAGTCCGCAGGACCTGGCGGACCTGTACACGCTGATCCGCCGCTACG
CCGACAACAGCGAATTG CTGGTGCGCACGCTGT TCCCTGAATACATCCCG
CACATGACGCGCGCCGGCACCTCGCTGCGGCCCAGCGAGATCGCCGGGCG
CCCGGTCAGCTGGCGCAAGGACGACACCCGCCT

In the above sequences, it should be understood that a bacteria may include changes in one or more base pairs relative to the mutation sequences noted above that still produce the same functionality and/or amino acid within the bacteria. For example, a bacteria may include 95%, 96%, 97%, 98%, 99%, or any other appropriate percentage of the same mutation sequences listed above while still providing the noted enhanced ROS resistance.

As elaborated on in the examples, the systems described herein are capable of undergoing intermittent production. For example, when a driving potential is applied to the electrodes to generate hydrogen, the bacteria produce the desired product. Correspondingly, when the potential is removed and hydrogen is no longer generated, production of the product is ceased once the available hydrogen is consumed and a reduction in overall biomass is observed until the potential is once again applied to the electrodes to generate hydrogen. The system will then resume biomass and/or product formation. Thus, while a system may be run continuously to produce a desired product, in some modes of operation a driving potential may be intermittently applied to the electrodes to intermittently split water to form hydrogen and correspondingly intermittently produce a desired product. A frequency of the intermittently applied potential may be any frequency and may either be uniform or non-uniform as the disclosure is not so limited. This ability to intermittently produce a product may be desirable in applications such as when intermittent renewable energy sources are used to provide the power applied to the electrodes including, but not limited to, intermittent power sources such as solar and wind energy.

In some embodiments of any of the aspects, the systems or compositions described herein can be scaled up to meet bioproduction needs. As used herein, the term “scale up” refers to an increase in production capacity (e.g., of a system as described herein). In some embodiments of the aspects, a system (e.g., a bioreactor system) as described herein can be scaled up by at least 1-fold, at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, at least 70-fold, at least 80-fold, at least 90-fold, or at least 100-fold. In some embodiments of the aspects, a bioreactor system as described herein can be scaled up to at least a 100 ml reactor, at least a 500 ml reactor, at least a 1000 mL reactor, at least a 2 L reactor, at least a 5 L reactor, at least a 10 L reactor, at least a 25 L reactor, at least a 50 L reactor, at least a 100 L reactor, at least a 500 L reactor, or at least a 1,000 L reactor.

In some embodiments, one or more of the genes described herein is expressed in a recombinant expression vector or plasmid. As used herein, the term “vector” refers to a polynucleotide sequence suitable for transferring transgenes into a host cell. The term “vector” includes plasmids, mini-chromosomes, phage, naked DNA and the like. See, for example, U.S. Pat. Nos. 4,980,285; 5,631,150; 5,707,828; 5,759,828; 5,888,783 and, 5,919,670, and, Sambrook et al, Molecular Cloning: A Laboratory Manual, 2nd Ed., Cold Spring Harbor Press (1989). One type of vector is a “plasmid,” which refers to a circular double stranded DNA loop into which additional DNA segments are ligated. Another type of vector is a viral vector, wherein additional DNA segments are ligated into the viral genome. Certain vectors are capable of autonomous replication in a host cell into which they are introduced (e.g., bacterial vectors having a bacterial origin of replication and episomal mammalian vectors). Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” is used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

A cloning vector is one which is able to replicate autonomously or integrated in the genome in a host cell, and which is further characterized by one or more endonuclease restriction sites at which the vector may be cut in a determinable fashion and into which a desired DNA sequence can be ligated such that the new recombinant vector retains its ability to replicate in the host cell. In the case of plasmids, replication of the desired sequence can occur many times as the plasmid increases in copy number within the host cell such as a host bacterium or just a single time per host before the host reproduces by mitosis. In the case of phage, replication can occur actively during a lytic phase or passively during a lysogenic phase.

An expression vector is one into which a desired DNA sequence can be inserted by restriction and ligation such that it is operably joined to regulatory sequences and can be expressed as an RNA transcript. Vectors can further contain one or more marker sequences suitable for use in the identification of cells which have or have not been transformed or transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). In certain embodiments, the vectors used herein are capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide.

When the nucleic acid molecule that encodes any of the polypeptides described herein is expressed in a cell, a variety of transcription control sequences (e.g., promoter/enhancer sequences) can be used to direct its expression. The promoter can be a native promoter, i.e., the promoter of the gene in its endogenous context, which provides normal regulation of expression of the gene. In some embodiments the promoter can be constitutive, i.e., the promoter is unregulated allowing for continual transcription of its associated gene. A variety of conditional promoters also can be used, such as promoters controlled by the presence or absence of a molecule.

The precise nature of the regulatory sequences needed for gene expression can vary between species or cell types, but in general can include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences can also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA). That heterologous DNA (RNA) is placed under operable control of transcriptional elements to permit the expression of the heterologous DNA in the host cell.

In some embodiments, the vector is pBadT. In some embodiments of any of the aspects, pBadT is an expression vector for at least one functional, heterologous gene. In some embodiments, the vector is arabinose-responsive promoter (e.g., P_{BAD} promoter).

Without limitations, the genes described herein can be included in one vector or separate vectors. For example, the functional heterologous thioesterase gene (e.g., a Cuphea palustris FatB1 gene, a Cuphea palustris FatB2 gene, a Cuphea palustris FatB2-FatB1 hybrid gene, or a Marvinbryantia formatexigens TE gene); the functional heterologous DGAT gene (e.g., Acinetobacter baylyi DGAT gene, or a Thermomonospora curvata DGAT gene); and the functional heterologous PAP gene (e.g., Rhodococcus opacus PAP gene, or a Rhodococcus jostii PAP gene) can be included in the same vector.

In some embodiments, the functional heterologous thioesterase gene (e.g., a Cuphea palustris FatB1 gene, a Cuphea palustris FatB2 gene, a Cuphea palustris FatB2-FatB1 hybrid gene, or a Marvinbryantia formatexigens TE gene) can be included in a first vector; the functional heterologous DGAT gene (e.g., Acinetobacter baylyi DGAT gene, or a Thermomonospora curvata DGAT gene) can be included in a second vector; and the functional heterologous PAP gene (e.g., Rhodococcus opacus PAP gene, or a Rhodococcus jostii PAP gene) can be included in a third vector.

In some other embodiments, the vector is pT18mobsacB. In some embodiments of any of the aspects, pT18mobsacB is an integration vector that can be used to engineer at least one inactivating modification of at least one endogenous gene in a bacterium, such as an endogenous polyhydroxyalkanoate (PHA) synthase gene (e.g., phaC).

In some embodiments, one or more of the recombinantly expressed gene can be integrated into the genome of the cell.

A nucleic acid molecule that encodes the enzyme of the claimed invention can be introduced into a cell or cells using methods and techniques that are standard in the art. For example, nucleic acid molecules can be introduced by standard protocols such as conjugation or transformation including chemical transformation and electroporation, transduction, particle bombardment, etc. Expressing the nucleic acid molecule encoding the enzymes of the claimed invention also may be accomplished by integrating the nucleic acid molecule into the genome.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

In microbiology, “16S sequencing” or “16S rRNA” or “16S-rRNA” or “16S” refers to sequence derived by characterizing the nucleotides that comprise the 16S ribosomal RNA gene(s). The bacterial 16S rDNA is approximately 1500 nucleotides in length and is used in reconstructing the evolutionary relationships and sequence similarity of one bacterial isolate to a second isolate using phylogenetic approaches. 16S sequences are used for phylogenetic reconstruction as they are in general highly conserved, but contain specific hypervariable regions that harbor sufficient nucleotide diversity to differentiate genera and species of most bacteria, as well as fungi.

The “V1-V9 regions” of the 16S rRNA refers to the first through ninth hypervariable regions of the 16S rRNA gene that are used for genetic typing of bacterial samples. These regions in bacteria are defined by nucleotides 69-99, 137-242, 433-497, 576-682, 822-879, 986-1043, 1117-1173, 1243-1294 and 1435-1465 respectively using numbering based on the E. coli system of nomenclature. Brosius et al., Complete nucleotide sequence of a 16S ribosomal RNA gene from Escherichia co/i, PNAS 75(10):4801-4805 (1978). In some embodiments, at least one of the V1, V2, V3, V4, V5, V6, V7, V8, and V9 regions are used to characterize an OTU. In one embodiment, the V1, V2, and V3 regions are used to characterize an OTU. In another embodiment, the V3, V4, and V5 regions are used to characterize an OTU. In another embodiment, the V4 region is used to characterize an OTU. A person of ordinary skill in the art can identify the specific hypervariable regions of a candidate 16S rRNA by comparing the candidate sequence in question to the reference sequence and identifying the hypervariable regions based on similarity to the reference hypervariable regions.

“Operational taxonomic unit (OTU, plural OTUs)” refers to a terminal leaf in a phylogenetic tree and is defined by a specific genetic sequence and all sequences that share a specified degree of sequence identity to this sequence at the level of species. A “type” or a plurality of “types” of bacteria includes an OTU or a plurality of different OTUs, and also encompasses a strain, species, genus, family or order of bacteria. The specific genetic sequence may be the 16S rRNA sequence or a portion of the 16S rRNA sequence, or it may be a functionally conserved housekeeping gene found broadly across the eubacterial kingdom. OTUs generally share at least 95%, 96%, 97%, 98%, or 99% sequence identity. OTUs are frequently defined by comparing sequences between organisms. Sequences with less than the specified sequence identity (e.g., less than 97%) are not considered to form part of the same OTU.

“Clade” refers to the set of OTUs or members of a phylogenetic tree downstream of a statistically valid node in a phylogenetic tree. The clade comprises a set of terminal leaves in the phylogenetic tree that is a distinct monophyletic evolutionary unit.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein. Preferably, the subject is a mammal. The mammal can be a human, non-human primate, mouse, rat, dog, cat, horse, or cow, but is not limited to these examples. A subject can be male or female. In some embodiments, the subject is a plant. In some embodiments, the subject is a bacterium.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wild-type reference polypeptide's activity according to the assays described below herein. A functional fragment can comprise conservative substitutions of the sequences disclosed herein. In some embodiments of any of the aspects, a polypeptide as described herein is truncated to remove an organelle targeting sequence(s); in some embodiments, such a targeting sequence can contribute to poor expression of the polypeptide, e.g., in the engineered bacteria described herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

A variant amino acid or DNA sequence can beat least 80%, at least 85%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

In some embodiments, the expression of a biomarker(s), target(s), or gene/polypeptide described herein is/are tissue-specific. In some embodiments, the expression of a biomarker(s), target(s), or gene/polypeptide described herein is/are global. In some embodiments, the expression of a biomarker(s), target(s), or gene/polypeptide described herein is systemic.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

In some embodiments, the methods described herein relate to measuring, detecting, or determining the level of at least one marker. As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments, a nucleic acid encoding a polypeptide as described herein (e.g. a TE polypeptide, a DGAT polypeptide, a LPAT polypeptide, a GPAT polypeptide, a PAP polypeptide) is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments of any of the aspects, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments of any of the aspects, the vector or nucleic acid described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments of any of the aspects, the expression system is an organism other than the source of the native/wild-type sequence (or a cell obtained from such organism). In some embodiments of any of the aspects, the vector and/or nucleic acid sequence described herein is codon-optimized for expression in a mammal or mammalian cell, e.g., a mouse, a murine cell, or a human cell. In some embodiments of any of the aspects, the vector and/or nucleic acid sequence described herein is codon-optimized for expression in a human cell. In some embodiments of any of the aspects, the vector and/or nucleic acid sequence described herein is codon-optimized for expression in a yeast or yeast cell. In some embodiments of any of the aspects, the vector and/or nucleic acid sequence described herein is codon-optimized for expression in a bacterial cell. In some embodiments of any of the aspects, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Some embodiments of the technology described herein can be defined according to any of the following numbered paragraphs:

1. An engineered Cupriavidus necator bacterium, comprising:
a) at least one exogenous copy of at least one functional acyltransferase gene; and/or
b) at least one exogenous copy of at least one functional phosphatidic acid (PA) phosphatase gene.

2. An engineered Cupriavidus necator bacterium, comprising:
a) at least one exogenous copy of at least one functional acyltransferase gene encoding an acyltransferase enzyme that catalyzes transesterification of the sn3 OH group of a diacylglycerol with a fatty acid; and/or
b) at least one exogenous copy of at least one functional phosphatidic acid (PA) phosphatase gene.

3. The engineered bacterium of paragraph 1, wherein the acyltransferase gene encodes for an acyltransferase enzyme that catalyzes transesterification of the sn3 OH group, the sn2 OH group, or the sn1 OH group of a triacylglycerol (TAG) precursor with a fatty acid

4. The engineered bacterium of paragraph 1, wherein the acyltransferase gene encodes an acyltransferase enzyme that catalyzes transesterification of the sn3 OH group of a diacylglycerol with a fatty acid.

5. The engineered bacterium of any one of paragraphs 1-4, wherein the acyltransferase gene is a functional diglyceride acyltransferase (DGAT) gene, a functional wax synthase (WS) gene, or a hybrid thereof.

6. The engineered bacterium of paragraph 5, wherein the functional DGAT gene is heterologous.

7. The engineered bacterium of paragraph 6, wherein the functional heterologous DGAT gene comprises a Acinetobacter baylyi DGAT gene, a Thermomonospora curvata DGAT gene, a Theobroma cacao DGAT gene, or a Rhodococcus opacus DGAT gene.

8. The engineered bacterium of paragraph 1, wherein the acyltransferase gene encodes an acyltransferase enzyme that catalyzes transesterification of the sn2 OH group of a lysophosphatidic acid with a fatty acid.

9. The engineered bacterium of paragraph 8, wherein the acyltransferase gene is a functional lysophosphatidic acid acyltransferase (LPAT) gene.

10. The engineered bacterium of paragraph 9, wherein the functional LPAT gene is heterologous.

11. The engineered bacterium of paragraph 10, wherein the functional heterologous LPAT gene comprises a Theobroma cacao LPAT gene.

12. The engineered bacterium of paragraph 1, wherein the acyltransferase gene encodes an acyltransferase enzyme that catalyzes transesterification of the sn1 OH group of a glyceraldehyde-3-phosphate with a fatty acid.

13. The engineered bacterium of paragraph 12, wherein the acyltransferase gene is a functional glycerol-3-phosphate acyltransferase (GPAT) gene.

14. The engineered bacterium of paragraph 13, wherein the functional GPAT gene is heterologous.

15. The engineered bacterium of paragraph 14, wherein the functional heterologous GPAT gene comprises a Durio zibethinus GPAT gene, Gossypium arboreum GPAT gene, Hibiscus syriacus GPAT gene, or a Theobroma cacao GPAT gene.

16. The engineered bacterium of any one of paragraphs 2-4, 8, or 12, wherein the fatty acid is esterified with acyl carrier protein (ACP) or with acetyl-CoA.

17. The engineered bacterium of paragraph 1, wherein the functional phosphatidic acid (PA) phosphatase gene encodes a phosphatidic acid (PA) phosphatase enzyme that catalyzes dephosphorylation at the sn3 position of phosphatidic acid (PA).

18. The engineered bacterium of paragraph 17, wherein the phosphatidic acid (PA) phosphatase gene is a functional phosphatidate phosphatase (PAP) gene.

19. The engineered bacterium of paragraph 18, wherein the functional PAP gene is heterologous.

20. The engineered bacterium of paragraph 19, wherein the functional heterologous PAP gene comprises a Rhodococcus opacus PAP gene, or a Rhodococcus jostii PAP gene.

21. The engineered bacterium of paragraph 1 or 2, further comprising: at least one exogenous copy of at least one functional thioesterase (TE) gene.

22. The engineered bacterium of paragraph 21, wherein the functional thioesterase gene is heterologous.

23. The engineered bacterium of paragraph 22, wherein the functional heterologous thioesterase gene is selected from the group consisting of: a Marvinbryantia formatexigens TE gene, a Cuphea palustris FatB1 gene, a Cuphea palustris FatB2 gene, a Cuphea palustris FatB2-FatB1 hybrid gene, a Arachis hypogaea FatB2-1 gene, a Mangifera indica FatA gene, a Morella rubra FatA gene, a Pistacia vera FatA gene, a Theobroma cacao FatA gene, a Theobroma cacao FatB gene (e.g., FatB1, FatB2, FatB3, BatB4, FatB5, or FatB6), or a Limosilactobacillus reuteri TE gene.

24. The engineered bacterium of paragraph 1 or 2, further comprising: (i) at least one endogenous polyhydroxyalkanoate (PHA) synthase gene comprising at least one engineered inactivating modification; or (ii) at least one exogenous inhibitor of an endogenous polyhydroxyalkanoate (PHA) synthase gene or gene product.

25. The engineered bacterium of paragraph 24, wherein the engineered inactivating modification of the endogenous PHA synthase comprises one or more of i) deletion of the entire coding sequence, ii) deletion of the promoter of the gene, iii) a frameshift mutation, iv) a nonsense mutation (i.e., a premature termination codon), v) a point mutation, vi) a deletion, vii) or an insertion.

26. The engineered bacterium of paragraph 24 or 25, wherein the endogenous PHA synthase comprises phaC.

27. The engineered bacterium of paragraph 1 or 2, further comprising: (i) at least one endogenous diacylglycerol kinase gene comprising at least one engineered inactivating modification; or (ii) at least one exogenous inhibitor of an endogenous diacylglycerol kinase gene or gene product.

28. The engineered bacterium of paragraph 27, wherein the engineered inactivating modification of the endogenous diacylglycerol kinase comprises one or more of i) deletion of the entire coding sequence, ii) deletion of the promoter of the gene, iii) a frameshift mutation, iv) a nonsense mutation (i.e., a premature termination codon), v) a point mutation, vi) a deletion, vii) or an insertion.

29. The engineered bacterium of paragraph 27 or 28, wherein the endogenous diacylglycerol kinase comprises dgkA.

30. The engineered bacterium of paragraph 1 or 2, further comprising: (i) at least one endogenous beta-oxidation gene comprising at least one engineered inactivating modification; or (ii) at least one exogenous inhibitor of an endogenous beta-oxidation gene or gene product.

31. The engineered bacterium of paragraph 30, wherein the engineered inactivating modification of the endogenous beta-oxidation gene comprises one or more of i) deletion of the entire coding sequence, ii) deletion of the promoter of the gene, iii) a frameshift mutation, iv) a nonsense mutation (i.e., a premature termination codon), v) a point mutation, vi) a deletion, vii) or an insertion.

32. The engineered bacterium of paragraph 30 or 31, wherein the endogenous beta-oxidation gene comprises FadE or FadB.

33. The engineered bacterium of any one of paragraphs 1-32, wherein said engineered bacteria is a chemoautotroph.

34. The engineered bacterium of any one of paragraphs 1-33, wherein said engineered bacteria uses CO₂ as its sole carbon source, and/or said engineered bacteria uses H₂ as its sole energy source.

35. The engineered bacterium of any one of paragraphs 1-34, wherein said engineered bacteria uses fructose as its sole carbon source.

36. The engineered bacterium of any one of paragraphs 1-35, wherein said engineered bacteria uses glycerol as its sole carbon source.

37. The engineered bacterium of any one of paragraphs 1-36, wherein said engineered bacteria produces triacylglycerides.

38. The engineered bacterium of any one of paragraphs 1-37, wherein said engineered bacteria produces animal triacylglycerides.

39. The engineered bacterium of any one of paragraphs 1-38, wherein said engineered bacteria produces milk fats.

40. A method of producing triacylglycerides (TAGs), comprising:
a) culturing the engineered bacterium of any of paragraphs 1-39 in a culture medium comprising CO₂ and/or H₂; and
b) isolating, collecting, or concentrating TAGs from said engineered bacterium or from the culture medium of said engineered bacterium.

41. The method of paragraph 40, wherein the culture medium comprises CO₂ as the sole carbon source, and/or the culture medium comprises H₂ as the sole energy source.

42. The method of any one of paragraphs 40-41, wherein the total TAG isolated comprises at least 50% TAGs comprising C4-C18 R-group fatty acids.

43. The method of any one of paragraphs 40-42, wherein the total TAG isolated comprises at least 50% TAGs comprising C4-C8 R-group fatty acids.

44. The method of any one of paragraphs 40-43, wherein the total TAG isolated comprises at least 50% TAGs comprising C16 R-group fatty acids.

45. A method of producing triacylglycerides (TAGs), comprising:
a) culturing the engineered bacterium of any of paragraphs 1-39 in a culture medium comprising fructose and/or H₂; and
b) isolating, collecting, or concentrating TAGs from said engineered bacterium or from the culture medium of said engineered bacterium.

46. The method of paragraph 45, wherein the culture medium comprises fructose as the sole carbon source, and/or the culture medium comprises H₂ as the sole energy source.

47. The method of any one of paragraphs 45-46, wherein the total TAG isolated comprises at least 50% TAGs comprising C4-C18 R-group fatty acids.

48. The method of any one of paragraphs 45-47, wherein the total TAG isolated comprises at least 50% TAGs comprising C4-C8 R-group fatty acids.

49. The method of any one of paragraphs 45-48, wherein the total TAG isolated comprises at least 50% TAGs comprising C16 R-group fatty acids.

50. A method of producing triacylglycerides (TAGs), comprising:
a) culturing the engineered bacterium of any of paragraphs 1-39 in a culture medium comprising glycerol and/or H₂; and
b) isolating, collecting, or concentrating TAGs from said engineered bacterium or from the culture medium of said engineered bacterium.

51. The method of paragraph 50, wherein the culture medium comprises glycerol as the sole carbon source, and/or the culture medium comprises H₂ as the sole energy source.

52. The method of any one of paragraphs 50-51, wherein the total TAG isolated comprises at least 50% TAGs comprising C4-C18 R-group fatty acids.

53. The method of any one of paragraphs 50-52, wherein the total TAG isolated comprises at least 50% TAGs comprising C4-C8 R-group fatty acids.

54. The method of any one of paragraphs 50-53, wherein the total TAG isolated comprises at least 50% TAGs comprising C16 R-group fatty acids.

55. A system comprising:
a) a reactor chamber with a solution contained therein, wherein the solution comprises hydrogen (H₂) and a carbon source; and
b) the engineered bacterium of any of paragraphs 1-39 in the solution.

56. The system of paragraph 55, further comprising a pair of electrodes in contact with the solution that split water to form the hydrogen.

57. The system of any one of paragraphs 55-56, wherein the carbon source is carbon dioxide (CO₂), fructose, and/or glycerol.

58. The system of any one of paragraphs 55-57, further comprising an isolated gas volume above a surface of the solution within a head space of a reactor chamber.

59. The system of any one of paragraphs 55-58, wherein the isolated gas volume comprises primarily carbon dioxide.

60. The system of any one of paragraphs 55-59, further comprising a power source comprising a renewable source of energy.

61. The system of any one of paragraphs 55-60, wherein the renewable source of energy comprises a solar cell, wind turbine, generator, battery, or grid power.

Some embodiments of the technology described herein can be defined according to any of the following numbered paragraphs:

101. An engineered Cupriavidus necator bacterium, comprising:
a) at least one exogenous copy of at least one functional thioesterase (TE) gene;
b) at least one exogenous copy of at least one functional diglyceride acyltransferase (DGAT) gene; and/or
c) at least one exogenous copy of at least one phosphatidate phosphatases (PAP) gene.

102. The engineered bacterium of paragraph 101, further comprising: (i) at least one endogenous polyhydroxyalkanoate (PHA) synthase gene comprising at least one engineered inactivating modification; or (ii) at least one exogenous inhibitor of an endogenous polyhydroxyalkanoate (PHA) synthase gene or gene product.

103. The engineered bacterium of any one of paragraphs 101-102, wherein said engineered bacteria is a chemoautotroph.

104. The engineered bacterium of any one of paragraphs 101-103, wherein said engineered bacteria uses CO₂ as its sole carbon source, and/or said engineered bacteria uses H₂ as its sole energy source.

105. The engineered bacterium of any one of paragraphs 101-104, wherein said engineered bacteria uses fructose as its sole carbon source.

106. The engineered bacterium of any one of paragraphs 101-105, wherein said engineered bacteria uses glycerol as its sole carbon source.

107. The engineered bacterium of any one of paragraphs 101-106, wherein the functional thioesterase gene is heterologous.

108. The engineered bacterium of paragraph 107, wherein the functional heterologous thioesterase gene comprises a Marvinbryantia formatexigens TE gene, a Cuphea palustris FatB1 gene, a Cuphea palustris FatB2 gene, or a Cuphea palustris FatB2-FatB1 hybrid gene.

109. The engineered bacterium of any one of paragraphs 101-108, wherein the functional DGAT gene is heterologous.

110. The engineered bacterium of paragraph 109, wherein the functional heterologous DGAT gene comprises a Acinetobacter baylyi DGAT gene, or a Thermomonospora curvata DGAT gene.

111. The engineered bacterium of any one of paragraphs 101-110, wherein the functional PAP gene is heterologous.

112. The engineered bacterium of paragraph 111, wherein the functional heterologous PAP gene comprises a Rhodococcus opacus PAP gene, or a Rhodococcus jostii PAP gene.

113. The engineered bacterium of any one of paragraphs 102-112, wherein the engineered inactivating modification of the endogenous PHA synthase comprises one or more of i) deletion of the entire coding sequence, ii) deletion of the promoter of the gene, iii) a frameshift mutation, iv) a nonsense mutation (i.e., a premature termination codon), v) a point mutation, vi) a deletion, vii) or an insertion.

114. The engineered bacterium of any one of paragraphs 102-113, wherein the endogenous PHA synthase comprises phaC.

115. The engineered bacterium of any one of paragraphs 101-114, wherein said engineered bacteria produces triacylglycerides.

116. The engineered bacterium of any one of paragraphs 101-115, wherein said engineered bacteria produces animal triacylglycerides.

117. The engineered bacterium of any one of paragraphs 101-116, wherein said engineered bacteria produces milk fats.

118. A method of producing triacylglycerides (TAGs), comprising:
a) culturing the engineered bacterium of any of paragraphs 101-117 in a culture medium comprising CO₂ and/or H₂; and
b) isolating, collecting, or concentrating TAGs from said engineered bacterium or from the culture medium of said engineered bacterium.

119. The method of paragraph 18, wherein the culture medium comprises CO₂ as the sole carbon source, and/or the culture medium comprises H₂ as the sole energy source.

120. The method of any one of paragraphs 118-119, wherein the total TAG isolated comprises at least 50% TAGs comprising C4-C18 R-group fatty acids.

121. The method of any one of paragraphs 118-120, wherein the total TAG isolated comprises at least 50% TAGs comprising C4-C8 R-group fatty acids.

122. The method of any one of paragraphs 118-121, wherein the total TAG isolated comprises at least 50% TAGs comprising C16 R-group fatty acids.

123. A method of producing triacylglycerides (TAGs), comprising:
a) culturing the engineered bacterium of any of paragraphs 101-117 in a culture medium comprising fructose and/or H₂; and
b) isolating, collecting, or concentrating TAGs from said engineered bacterium or from the culture medium of said engineered bacterium.

124. The method of paragraph 123, wherein the culture medium comprises fructose as the sole carbon source, and/or the culture medium comprises H₂ as the sole energy source.

125. The method of any one of paragraphs 123-124, wherein the total TAG isolated comprises at least 50% TAGs comprising C4-C18 R-group fatty acids.

126. The method of any one of paragraphs 123-125, wherein the total TAG isolated comprises at least 50% TAGs comprising C4-C8 R-group fatty acids.

127. The method of any one of paragraphs 123-126, wherein the total TAG isolated comprises at least 50% TAGs comprising C16 R-group fatty acids.

128. A method of producing triacylglycerides (TAGs), comprising:
a) culturing the engineered bacterium of any of paragraphs 101-117 in a culture medium comprising glycerol and/or H₂; and
b) isolating, collecting, or concentrating TAGs from said engineered bacterium or from the culture medium of said engineered bacterium.

129. The method of paragraph 128, wherein the culture medium comprises glycerol as the sole carbon source, and/or the culture medium comprises H₂ as the sole energy source.

130. The method of any one of paragraphs 128-129, wherein the total TAG isolated comprises at least 50% TAGs comprising C4-C18 R-group fatty acids.

131. The method of any one of paragraphs 128-130, wherein the total TAG isolated comprises at least 50% TAGs comprising C4-C8 R-group fatty acids.

132. The method of any one of paragraphs 128-131, wherein the total TAG isolated comprises at least 50% TAGs comprising C16 R-group fatty acids.

133. A system comprising:
a) a reactor chamber with a solution contained therein, wherein the solution comprises hydrogen (H₂) and a carbon source; and
b) the engineered bacterium of any of paragraphs 101-117 in the solution.

134. The system of paragraph 133, further comprising a pair of electrodes in contact with the solution that split water to form the hydrogen.

135. The system of any one of paragraphs 133-134, wherein the carbon source is carbon dioxide (CO₂), fructose, and/or glycerol.

136. The system of any one of paragraphs 133-135, further comprising an isolated gas volume above a surface of the solution within a head space of a reactor chamber.

137. The system of any one of paragraphs 133-136, wherein the isolated gas volume comprises primarily carbon dioxide.

138. The system of any one of paragraphs 133-137, further comprising a power source comprising a renewable source of energy.

139. The system of any one of paragraphs 133-138, wherein the renewable source of energy comprises a solar cell, wind turbine, generator, battery, or grid power.

Some embodiments of the technology described herein can be defined according to any of the following numbered paragraphs:

201. An engineered Cupriavidus necator bacterium, comprising:
a) at least one exogenous copy of at least one functional thioesterase (TE) gene;
b) at least one exogenous copy of at least one functional diglyceride acyltransferase (DGAT) gene; and/or
c) at least one exogenous copy of at least one phosphatidate phosphatases (PAP) gene.

202. The engineered bacterium of paragraph 201, further comprising: (i) at least one endogenous polyhydroxyalkanoate (PHA) synthase gene comprising at least one engineered inactivating modification; or (ii) at least one exogenous inhibitor of an endogenous polyhydroxyalkanoate (PHA) synthase gene or gene product.

203. The engineered bacterium of any one of paragraphs 201-202, wherein said engineered bacteria is a chemoautotroph.

204. The engineered bacterium of any one of paragraphs 201-203, wherein said engineered bacteria uses CO₂ as its sole carbon source, and/or said engineered bacteria uses H₂ as its sole energy source.

205. The engineered bacterium of any one of paragraphs 201-204, wherein said engineered bacteria uses glycerol as its sole carbon source.

206. The engineered bacterium of any one of paragraphs 201-205, wherein the functional thioesterase gene is heterologous.

207. The engineered bacterium of paragraph 206, wherein the functional heterologous thioesterase gene comprises a Marvinbryantia formatexigens TE gene, a Cuphea palustris FatB1 gene, a Cuphea palustris FatB2 gene, or a Cuphea palustris FatB2-FatB1 hybrid gene.

208. The engineered bacterium of any one of paragraphs 201-207, wherein the functional DGAT gene is heterologous.

209. The engineered bacterium of paragraph 208, wherein the functional heterologous DGAT gene comprises a Acinetobacter baylyi DGAT gene, or a Thermomonospora curvata DGAT gene.

210. The engineered bacterium of any one of paragraphs 201-209, wherein the functional PAP gene is heterologous.

211. The engineered bacterium of paragraph 2010, wherein the functional heterologous PAP gene comprises a Rhodococcus opacus PAP gene, or a Rhodococcus jostii PAP gene.

212. The engineered bacterium of any one of paragraphs 202-2011, wherein the engineered inactivating modification of the endogenous PHA synthase comprises one or more of i) deletion of the entire coding sequence, ii) deletion of the promoter of the gene, iii) a frameshift mutation, iv) a nonsense mutation (i.e., a premature termination codon), v) a point mutation, vi) a deletion, vii) or an insertion.

213. The engineered bacterium of any one of paragraphs 202-212, wherein the endogenous PHA synthase comprises phaC.

214. The engineered bacterium of any one of paragraphs 201-213, wherein said engineered bacteria produces triacylglycerides.

215. The engineered bacterium of any one of paragraphs 201-214, wherein said engineered bacteria produces animal triacylglycerides.

216. The engineered bacterium of any one of paragraphs 201-214, wherein said engineered bacteria produces milk fats.

217. A method of producing triacylglycerides (TAGs), comprising:
a) culturing the engineered bacterium of any of paragraphs 201-216 in a culture medium comprising CO₂ and/or H₂; and
b) isolating, collecting, or concentrating TAGs from said engineered bacterium or from the culture medium of said engineered bacterium.

218. The method of paragraph 217, wherein the culture medium comprises CO₂ as the sole carbon source, and/or the culture medium comprises H₂ as the sole energy source.

219. The method of any one of paragraphs 217-218, wherein the total TAG isolated comprises at least 50% TAGs comprising C16 R-group fatty acids.

220. A method of producing triacylglycerides (TAGs), comprising:
a) culturing the engineered bacterium of any of paragraphs 201-216 in a culture medium comprising glycerol and/or H₂; and
b) isolating, collecting, or concentrating TAGs from said engineered bacterium or from the culture medium of said engineered bacterium.

221. The method of paragraph 220, wherein the culture medium comprises glycerol as the sole carbon source, and/or the culture medium comprises H₂ as the sole energy source.

222. The method of any one of paragraphs 220-221, wherein the total TAG isolated comprises at least 50% TAGs comprising C16 R-group fatty acids.

223. A system comprising:
a) a reactor chamber with a solution contained therein, wherein the solution comprises hydrogen (H₂) and a carbon source; and
b) the engineered bacterium of any of paragraphs 201-216 in the solution.

224. The system of paragraph 223, further comprising a pair of electrodes in contact with the solution that split water to form the hydrogen.

225. The system of any one of paragraphs 223-224, wherein the carbon source is carbon dioxide (CO₂) and/or glycerol.

226. The system of any one of paragraphs 223-225, further comprising an isolated gas volume above a surface of the solution within a head space of a reactor chamber.

227. The system of any one of paragraphs 223-226, wherein the isolated gas volume comprises primarily carbon dioxide.

228. The system of any one of paragraphs 223-227, further comprising a power source comprising a renewable source of energy.

229. The system of any one of paragraphs 223-228, wherein the renewable source of energy comprises a solar cell, wind turbine, generator, battery, or grid power.

The technology described herein is further illustrated by the following examples which in no way should be construed as being further limiting.

### EXAMPLES

### Example 1: Production of Tailored Animal Triacylglycerides from C. necator

The area of animal-free replacements for lipids remains largely untapped. Milk fats are largely responsible for texture, flavor, energy content, and the solubility of some vitamins in dairy products. The possibility of biomanufacturing such animal-free milk fats to provide an alternative option for milk, butter, cheese, creams, ice cream, and meat represents a critical part of the solution for utilizing synthetic biology to lessen the environmental impacts of addressing humanity's increasing food production demands. Worldwide, cattle farming produces 11% of all greenhouse gas emissions and the dairy industry emits 3% annually. Current dairy alternatives are currently limited by the ability of plant fats to confer the same properties as dairy fats. Milk lipids are in a large part responsible for the taste and texture of dairy products, especially in the case of cheese and butter. Currently, there are no commercially available animal-free replacements for these fats, and plant-based options lack the physical properties for many applications as well as introduce unwanted flavors. By producing identical, or substantially similar, milk lipid replacements, the engineered bacteria and methods described herein permit a broader application of animal-free dairy. Without wishing to be bound by theory, the engineered bacteria and methods described herein are expected to have 120% lower GHG emissions, use 99% less land, and use half the amount of water needed in current dairy practices.

Described herein are engineered bacteria that produce triacylglycerides (TAGs), the major class of fats in dairy (see e.g., FIG. 1A). The composition of the TAGs can be tailored by changing specific enzymes in the biosynthetic pathway. Varying the expression of different thioesterases (TE) leads to the production of specific chain-length fatty acids. For those fatty acids to be added to the glycerol backbone, diglyceride acyltransferases (DGAT) can also be varied. Phosphatidate phosphatases (PAP) can be engineered to achieve specific TAGs (see e.g., FIG. 1B).

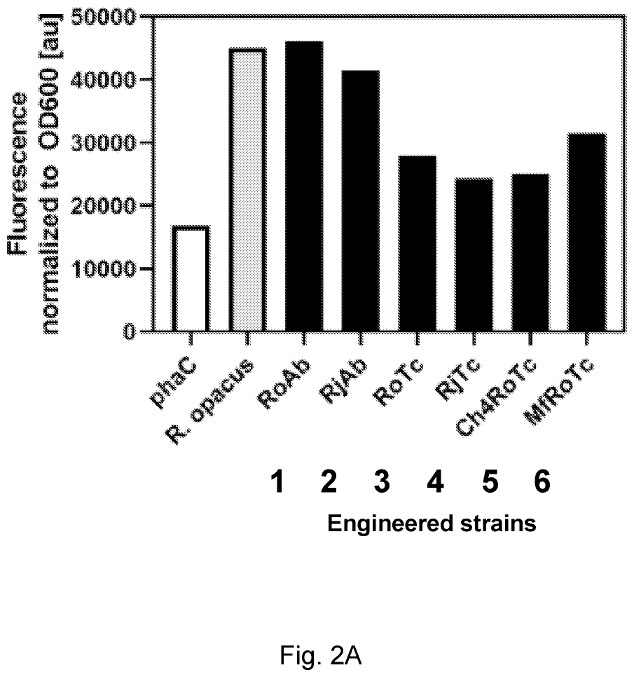

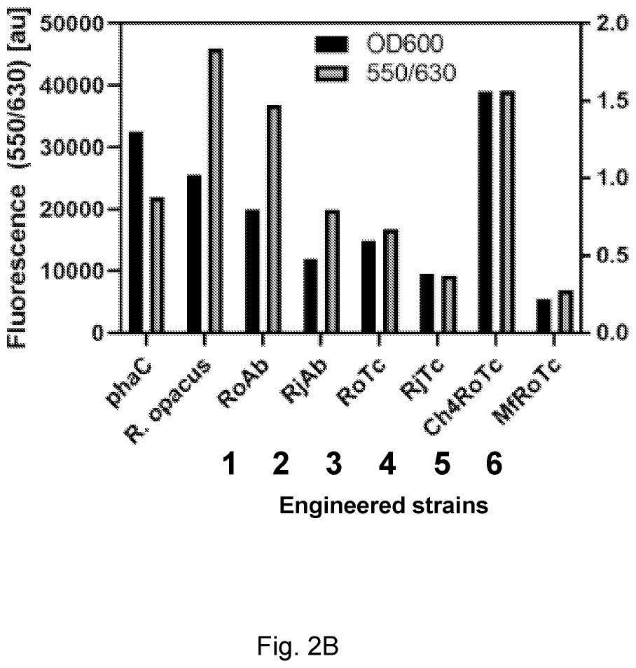

Using a chassis organism, C. necator, capable of both heterotrophic and autotrophic growth, provided herein is a proof-of-concept of this approach. Using a parent strain of a PHA synthesis deletion strain (ΔphaC), the following combinations were over-expressed: R. opacus PAP and A. baylyi DGAT (RoAb; “Strain 1”); R. jostii PAP and A. baylyi DGAT (RjAb; “Strain 2”); R. opacus PAP and T. curvata DGAT (RoTc; “Strain 3”); R. jostii PAP and T. curvata DGAT (RjTc; “Strain 4”); R. opacus PAP, T. curvata DGAT and Chimera 4 TE (Ch4RoTc; “Strain 5”); and R. opacus PAP, T. curvata (DGAT), and M. formatexigens (TE) (MfRoTc; “Strain 6”). Wild type R. opacus was used as a positive control since it is a model organism for natural TAG biosynthesis. In a Nile Red assay, observed fluorescence indicated lipid accumulation in all of the engineered strains (see e.g., FIG. 2A). The highest accumulation occurred in strains containing the A. baylyi DGAT. Optical density measurements indicated that strains containing the R. opacus PAP also grew to higher densities than those strains containing the R. jostii PAP (see e.g., FIG. 2B).

Growth of strain 1 (R. opacus PAP and A. baylyi DGAT (RoAb) in ΔphaC C. necator) resulted in a higher fatty acid content and an altered distribution compared to ΔphaC (see e.g., FIG. 3).

Without wishing to be bound by theory, it is estimated that the yield for TAG production using the engineered bacteria as described herein is about 10-20% (e.g., at least 10%, at least 11%, at least 12%, at least 13%, at least 14%, at least 15%, at least 16%, at least 17%, at least 18%, at least 19%, or at least 20% TAG yield). In some embodiments, percent yield can be calculated by dividing isolated lipids by total dry cell weight. In some embodiments, wild-type bacteria (e.g., C. necator) comprise at least 10% lipid yield. In some embodiments, engineered bacteria described herein comprise at least 20% lipid yield.

In some embodiments, percent yield can be calculated by dividing the actual yield (e.g., isolated amount of TAG) by the theoretical yield (which can be determined by the amount of each reactant and stoichiometric calculations to determine the expected amount of product).

### Methods

FIGS. 2A and 2B: Strains were cultured on rich broth agar plates from glycerol stock at 30° C. for 2 days. Single colonies were incubated in rich broth liquid media with antibiotics (e.g., kanamycin) overnight. 1 mL of overnight culture was inoculated in 50 mL of minimal media comprising fructose (e.g., 20 g/L; 2%), cultured at 30° C. while shaking until OD 0.4-0.6. Then cells were induced with 0.1% arabinose and cultured for another ˜20 hours. OD600 was measured and 200 uL of culture was subjected to Nile Red assay. For the Nile Red assay, 5 uL of 0.025 mg/mL Nile Red in DMSO was added to the culture, incubated for 10 min at room temperature in the dark and fluorescence was measured (excitation: 550 nm, emission: 630 nm).

FIG. 3: 1 mL aliquot was inoculated in 300-600 mL rich broth with antibiotics (e.g., kanamycin) and incubated at 30° C. while shaking for 24 hr. The next day, cells were diluted 1:20 in 4 L or 10 L fermenter in minimal media comprising 20 g/L fructose and 1.5 g/L ammonium chloride. Cells were induced after ˜18 hr with 0.1% arabinose and cultured for another 24 hr. Cells were harvested and pellets lyophilized. Lyophilized cells were then subjected to direct methanolysis for whole cell fatty acid analysis. For that analysis, lyophilized cells were suspended in equal volumes of chloroform and acidified methanol, and heated for 2 hr at 100° C. The organic mixture was added to water, vortexed and separated via centrifugation. The chloroform phase was separated and analyzed for fatty acid methyl esters (FAMEs) via gas chromatography-mass spectrometry (GC-MS).

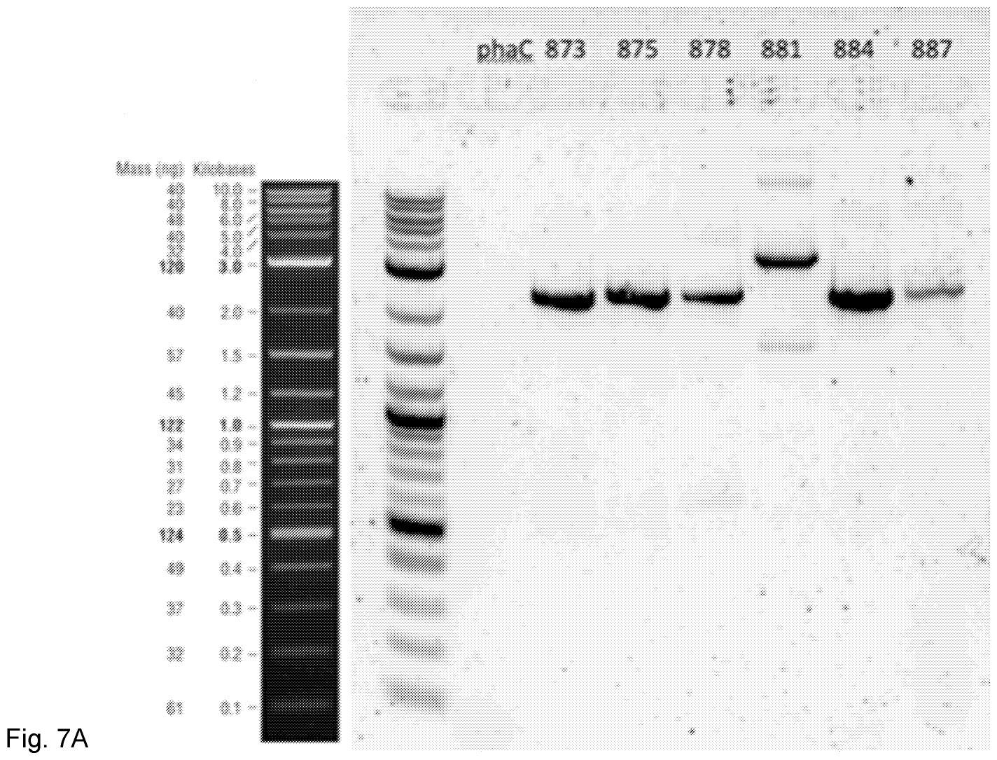

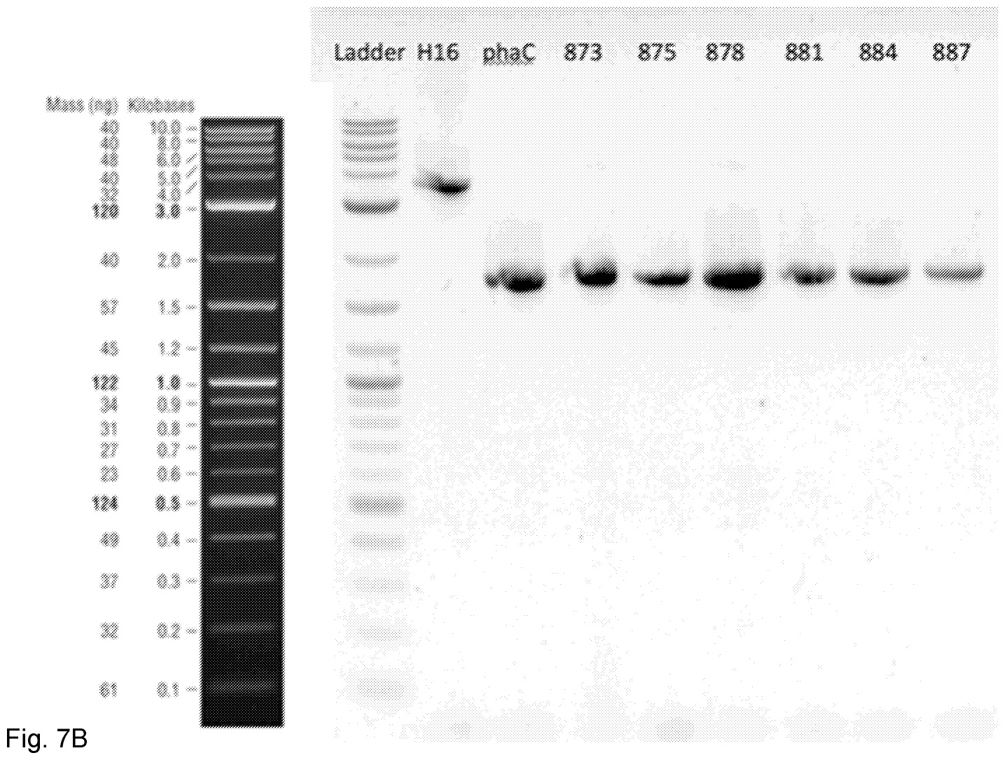

FIG. 7A-7B: For the PCR verification, standard PCR procedure was applied to cells diluted in ddH2O (see e.g., Table 6 below for strain designations used in FIG. 7A-7B).

TABLE 6
Exemplary Engineered TAG production strains.

| Designation | strain | Genotype | Added enzymes |
|---|---|---|---|
| H16 | Cupriavidus | wild type | |
| | necator H16 | | |
| phaC | Cupriavidus | ΔphaC1 | |
| | necator H16 | | |
| 873 | Cupriavidus | ΔphaC1 | R. opacus PAP, |
| | necator H16 | | A. baylyi DGAT |
| 875 | Cupriavidus | ΔphaC1 | R. jostii PAP, |
| | necator H16 | | T. curvata DGAT |
| 878 | Cupriavidus | ΔphaC1 | R. opacus PAP, |
| | necator H16 | | T. curvata DGAT |
| 881 | Cupriavidus | ΔphaC1 | R. opacus PAP, |
| | necator H16 | | T. curvata DGAT, |
| | | | Chimera 4 TE |
| 884 | Cupriavidus | ΔphaC1 | R. jostii PAP, |
| | necator H16 | | A. baylyi DGAT |
| 887 | Cupriavidus | ΔphaC1 | R. opacus PAP, |
| | necator H16 | | T. curvata DGAT, |
| | | | M. formatexigens TE |

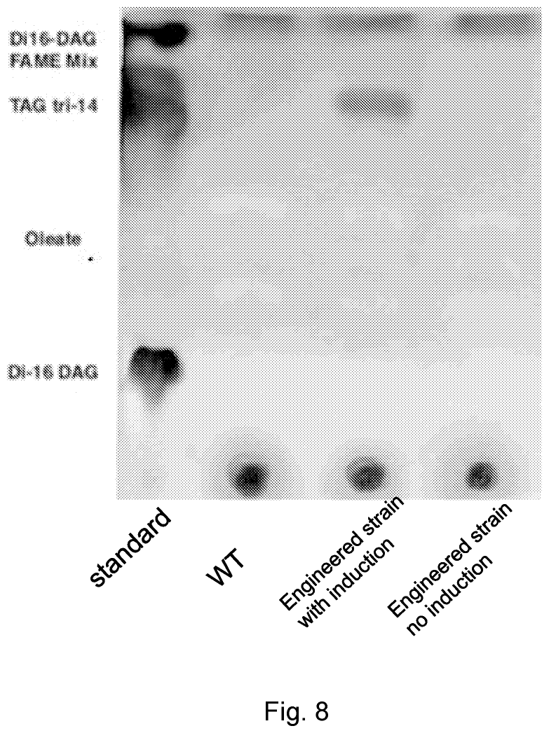

FIG. 8: For TLC, lipids were extracted from strain 873 (see e.g., Table 6) via the Bligh Dyer method. Briefly, equal amounts of chloroform and methanol were added to lyophilized biomass. Lipids were extracted via vortexing and separated by adding potassium chloride solution and centrifuging. The chloroform layer was then loaded onto a thin layer chromatographie plate, evolved using a hexane:diethyl ether:acetic acid mobile phase and visualized using primuline.

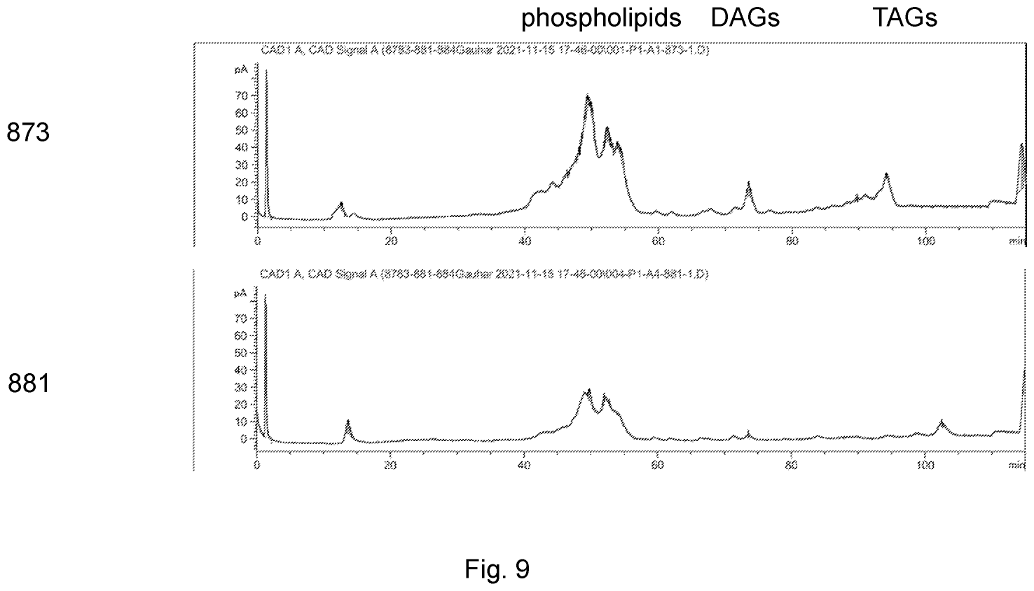

FIG. 9: For HPLC, extracted TAGs were loaded onto C18 column and separated using two mobile phases, where one mobile phase consisted of acetonitrile, ammonium formate and formic acid and another mobile phase of isopropanol, water and formic acid.

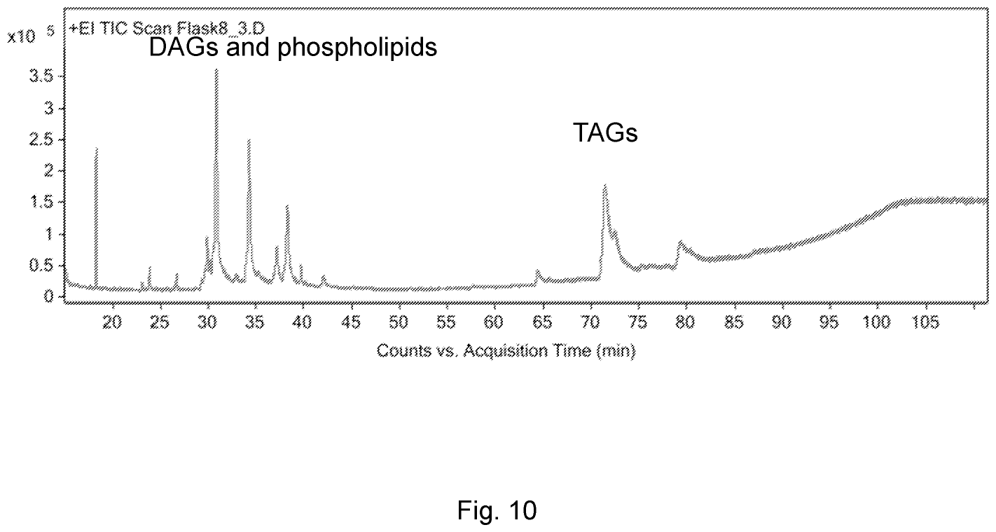

FIG. 10: For GC-MS, TAGs extracted from strain 873 (see e.g., Table 6) were loaded onto AGILENT CP-TAP column and analyzed via Mass Spectrometry.

1. An engineered Cupriavidus necator bacterium, comprising:
a) at least one exogenous copy of:
(i) a gene encoding an enzyme that catalyzes transesterification of the sn3 OH group of a diacylglycerol with a fatty acid;
(ii) a gene encoding an enzyme that catalyzes transesterification of the sn2 OH group of a lysophosphatidic acid with a fatty acid; and/or
(iii) a gene encoding an enzyme that catalyzes transesterification of the sn1 OH group of a glyceraldehyde-3-phosphate with a fatty acid; and/or
b) at least one exogenous copy of at least one functional phosphatidic acid (PA) phosphatase gene.

2. An engineered Cupriavidus necator bacterium, comprising:
a) at least one exogenous copy of at least one functional acyltransferase gene; and/or
b) at least one exogenous copy of at least one functional phosphatidic acid (PA) phosphatase gene.

3. The engineered bacterium of claim 2, wherein the acyltransferase gene encodes for an acyltransferase enzyme that catalyzes transesterification of the sn3 OH group, the sn2 OH group, or the sn1 OH group of a triacylglycerol (TAG) precursor with a fatty acid.

4. The engineered bacterium of claim 2, wherein the acyltransferase gene encodes an acyltransferase enzyme that catalyzes transesterification of:
a) the sn3 OH group of a diacylglycerol with a fatty acid;
b) the sn2 OH group of a lysophosphatidic acid with a fatty acid: or
c) the sn1 OH group of a glyceraldehyde-3-phosphate with a fatty acid.

5. The engineered bacterium of claim 2, wherein the acyltransferase gene is:
a) a functional heterologous diglyceride acyltransferase (DGAT) gene, a functional wax synthase (WS) gene, or a hybrid thereof;
b) a functional heterologous lysophosphatidic acid acyltransferase (LPAT) gene; or
c) a functional heterologous glycerol-3-phosphate acyltransferase (GPAT) gene.

6. (canceled)

7. The engineered bacterium of claim 5, wherein:
a) the functional heterologous DGAT gene comprises an Acinetobacter baylyi DGAT gene, a Thermomonospora curvata DGAT gene, a Theobroma cacao DGAT gene, or a Rhodococcus opacus DGAT gene,
b) the functional heterologous LPAT gene comprises a Theobroma cacao LPAT gene; or
c) the functional heterologous GPAT gene comprises a Durio zibethinus GPAT gene, Gossypium arboreum GPAT gene, Hibiscus syriacus GPAT gene, or a Theobroma cacao GPAT gene.

8-15. (canceled)

16. The engineered bacterium of claim 3, wherein the fatty acid is esterified with acyl carrier protein (ACP) or with acetyl-CoA.

17. The engineered bacterium of claim 1, wherein the functional phosphatidic acid (PA) phosphatase gene encodes a phosphatidic acid (PA) phosphatase enzyme that catalyzes dephosphorylation at the sn3 position of phosphatidic acid (PA).

18. The engineered bacterium of claim 17, wherein the phosphatidic acid (PA) phosphatase gene is a functional heterologous phosphatidate phosphatase (PAP) gene.

19. (canceled)

20. The engineered bacterium of claim 18, wherein the functional heterologous PAP gene comprises a Rhodococcus opacus PAP gene or a Rhodococcus jostii PAP gene.

21. The engineered bacterium of claim 1, further comprising:
a) at least one exogenous copy of at least one functional heterologous thioesterase (TE) gene;
b) at least one endogenous polyhydroxyalkanoate (PHA) synthase gene comprising at least one engineered inactivating modification: or at least one exogenous inhibitor of an endogenous polyhydroxyalkanoate (PHA) synthase gene or gene product;
c) at least one endogenous diacylglycerol kinase gene comprising at least one engineered inactivating modification; or at least one exogenous inhibitor of an endogenous diacylglycerol kinase gene or gene product; and/or
d) at least one endogenous beta-oxidation gene comprising at least one engineered inactivating modification; or at least one exogenous inhibitor of an endogenous beta-oxidation gene or gene product.

22. (canceled)

23. The engineered bacterium of claim 21, wherein:
a) the functional heterologous thioesterase gene is selected from the group consisting of: a Marvinbryantia formatexigens TE gene, a Cuphea palustris FatB1 gene, a Cuphea palustris FatB2 gene, a Cuphea palustris FatB2-FatB1 hybrid gene, a Arachis hypogaea FatB2-1 gene, a Mangifera indica FatA gene, a Morella rubra FatA gene, a Pistacia vera FatA gene, a Theobroma cacao FatA gene, a Theobroma cacao FatB gene, or a Limosilactobacillus reuteri TE gene,
b) the endogenous PHA synthase comprises phaC;
c) the endogenous diacylglycerol kinase comprises dgkA; and/or
d) the endogenous beta-oxidation gene comprises FadE or FadB.

24. (canceled)

25. The engineered bacterium of claim 21, wherein the engineered inactivating modification of the endogenous PHA synthase, the endogenous diacylglycerol kinase, or the endogenous beta-oxidation gene comprises one or more of i) deletion of the entire coding sequence, ii) deletion of the promoter of the gene, iii) a frameshift mutation, iv) a nonsense mutation, v) a point mutation, vi) a deletion, vii) or an insertion.

26-32. (canceled)

33. The engineered bacterium of claim 1, wherein said engineered bacteria is a chemoautotroph.

34. The engineered bacterium of claim 1, wherein said engineered bacteria uses CO₂ as its sole carbon source, and/or said engineered bacteria uses H₂ as its sole energy source.

35. The engineered bacterium of claim 1, wherein said engineered bacteria uses fructose, fatty acids, glucose, gluconate, acetate, decanoate or glycerol as its sole carbon source.

36. (canceled)

37. The engineered bacterium of claim 1, wherein said engineered bacteria produces triacylglycerides and/or animal fats.

38. The engineered bacterium of claim 37, wherein said engineered bacteria produces animal triacylglycerides.

39. (canceled)

40. A method of producing triacylglycerides (TAGs), comprising:
a) culturing the engineered bacterium of claim 1 in a culture medium comprising CO₂, fatty acids, gluconate, decanoate, acetate, fructose, glycerol and/or H₂; and
b) isolating, collecting, or concentrating TAGs from said engineered bacterium or from the culture medium of said engineered bacterium.

41-54. (canceled)

55. A system comprising:
a) a reactor chamber with a solution contained therein, wherein the solution comprises hydrogen (H₂) and a carbon source; and
b) the engineered bacterium of claim 1 in the solution.

56-61. (canceled)


SEQUENCE LISTING (139 sequences)

SEQ ID NO: 1 - DNA, 1356 bp, Cupriavidus necator
ttagattgaacgctggcggcatgccttacacatgcaagtcgaacggcagcacgggcttcggcctggtggcgagtggcgaacgggtgagtaatacatcggaacgtgccctgtagtgggggataactagtcgaaagattagctaataccgcatacgacctgagggtgaaagcgggggaccgcaaggcctcgcgctacaggagcggccgatgtctgattagctagttggtggggtaaaagcctaccaaggcgacgatcagtagctggtctgagaggacgatcagccacactgggactgagacacggcccagactcctacgggaggcagcagtggggaattttggacaatgggggcaaccctgatccagcaatgccgcgtgtgtgaagaaggccttcgggttgtaaagcacttttgtccggaaagaaatggctctggttaatacccggggtcgatgacggtaccggaagaataagcaccggctaactacgtgccagcagccgcggtaatacgtagggtgcgagcgttaatcggaattactgggcgtaaagcgtgcgcaggcggttttgtaagacaggcgtgaaatccccgagctcaacttgggaatggcgcttgtgactgcaaggctagagtatgtcagaggggggtagaattccacgtgtagcagtgaaatgcgtagagatgtggaggaataccgatggcgaaggcagccccctgggacgtcactgacgctcatgcacgaaagcgtggggagcaaacaggattagataccctggtagtccacgccctaaacgatgtcaactagttgttggggattcatttcttcagtaacgtagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatgccactaacgaagcagagatgcattaggtgcccgaaagggaaagtggacacaggtgctgcatggctgtcgtcagctcgtgtcgtgagatgttgggttaagtcccgcaacgagcgcaacccttgtctctagttgctacgaaagggcactctagagagactgccggtgacaaaccggaggaaggtggggatgacgtcaagtcctcatggcccttatgggtagggcttcacacgtcatacaatggtgcgtacagagggttgccaacccgcgagggggagctaatcccagaaaacgcatcgtagtccggatcgtagtctgcaactcgactacgtgaagctggaatcgctagtaatcgcggatcagcatgccgcggtgaatacgttcccggtct

SEQ ID NO: 2 - DNA, 1537 bp, Cupriavidus necator
agattgaactgaagagtttgatcctggctcagattgaacgctggcggcatgccttacacatgcaagtcgaacggcagcacgggcttcggcctggtggcgagtggcgaacgggtgagtaatacatcggaacgtgccctgtagtgggggataactagtcgaaagattagctaataccgcatacgacctgagggtgaaagcgggggaccgcaaggcctcgcgctacaggagcggccgatgtctgattagctagttggtggggtaaaagcctaccaaggcgacgatcagtagctggtctgagaggacgatcagccacactgggactgagacacggcccagactcctacgggaggcagcagtggggaattttggacaatgggggcaaccctgatccagcaatgccgcgtgtgtgaagaaggccttcgggttgtaaagcacttttgtccggaaagaaatggctctggttaatacccggggtcgatgacggtaccggaagaataagcaccggctaactacgtgccagcagccgcggtaatacgtagggtgcgagcgttaatcggaattactgggcgtaaagcgtgcgcaggcggttttgtaagacaggcgtgaaatccccgagctcaacttgggaatggcgcttgtgactgcaaggctagagtatgtcagagggggaagaattccacgtgtagcagtgaaatgcgtagagatgtggaggaataccgatggcgaaggcagccccctgggacgtcactgacgctcatgcacgaaagcgtggggagcaaacaggattagataccctggtagtccacgccctaaacgatgtcaactagttgttggggattcatttcttcagtaacgtagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatgccactaacgaagcagagatgcattaggtgcccgaaagggaaagtggacacaggtgctgcatggctgtcgtcagctcgtgtcgtgagatgttgggttaagtcccgcaacgagcgcaacccttgtctctagttgctacgaaagggcactctagagagactgccggtgacaaaccggaggaaggtggggatgacgtcaagtcctcatggcccttatgggtagggcttcacacgtcatacaatggtgcgtacagagggttgccaacccgcgagggggagctaatcccagaaaacgcatcgtagtccggatcgtagtctgcaactcgactacgtgaagctggaatcgctagtaatcgcggatcagcatgccgcggtgaatacgttcccgggtcttgtacacaccgcccgtcacaccatgggagtgggttttgccagaagtagttagcctaaccgcaaggagggcgattaccacggcagggttcatgactggggtgaagtcgtaacaaggtagccgtatcggaaggtgcggctggatcacctcctttc

SEQ ID NO: 3 - DNA, 1770 bp, Cupriavidus necator
atggcgaccggcaaaggcgcggcagcttccactcaggaaggcaagtcccaaccattcaagttcacgccggggccattcgatccagccacatggctggaatggtcccgccagtggcagggcactgaaggcaacggccacgcggccgcgtccggcattccgggcctggatgcgctggcaggcgtcaagatcgagccggcgcagctgggtgatatccagcagcgttacatgaaggacttctcagccctgtggcaggccatggccgagggcaaggccgaggccaccgggccgctgcacgaccggcgcttcgccggcgacgcgtggcgcaccaacctgccataccgcttcgctgccgcgttctacctgctcaatgcgcgcgccttgaccgagctggccgatgctgttgaggccgatgccaagacgcgccagcgcatccgctttgcgatctcgcaatgggtcgatgcgatgtcgcccgccaacttcctcgccacgaatcccgaggcgcagcgcctgctgatcgagtcgggcggcgaatcgctgcgtgccggcgtgcgcaacatgatggaagacctgacgcgcggcaagatctcgcagaccgacgagagcgcgtttgaggtcggccgcaatgtcgcggtgagcgaaggcgccgtagtcttcgagaacgaatacttccagctgttgcagtacaagccgctgaccgacaaggtgcatgcgcgcccgctgctgatggtgccgccgtgcatcaacaagtactacatcctggacctgcagccggagagctcgctggtgcgtcatgtggtggagcaggggcatacggtgttcctggtgtcgtggcgcaatccggacgccagcatggctggcagcacctgggacgactacatcgagcacgcggccatccgcgccatcgaagtcgcgcgcgacatcagcggccaggacaagatcaacgtgctcggcttctgcgtgggcggcaccattgtgtcgactgcgctggcggtgatggccgcgcgcggccagcacccggctgccagcgtcacgctgctgaccacgctgctggactttgccgacaccggcatcctcgacgtctttgtcgacgagggccatgtgcagctgcgcgaggccacgctgggcggcgccgccggcgcgccgtgcgcgctgctgcgcggccttgagctggccaataccttctcgttcctgcgcccgaacgacctggtgtggaactacgtggtcgacaactacctgaagggcaacacgccggtgccgttcgacctgctgttctggaacggcgacgccaccaacctgccggggccttggtactgctggtacctgcgccacacctacctgcaggacgagctcaaggtgccgggcaagctgactgtgtgcggcgtgcccgtggacctggccagcatcgacgtgccgacctacatctacggctcgcgcgaagaccatatcgtgccatggaccgcggcctatgcctcgaccgcgctgctggcgaacaagctgcgcttcgtgctgggtgcgtcgggccatatcgccggtgtgatcaacccgccggccaagaacaagcgcagccactggaccaacgatgcgctgccggagtcgccgcagcaatggctggctggcgccaccgagcatcacggcagctggtggccggactggaccgcatggctggcaggccaggccggcgcgaaacgtgccgcgcccgccaactacggcaatgcgcgctatcccgcgatcgaacccgcgcctgggcgatacgtcaaagccaaggcatga

SEQ ID NO: 4 - protein, 589 aa, Cupriavidus necator
MATGKGAAASTQEGKSQPFKFTPGPFDPATWLEWSRQWQGTEGNGHAAASGIPGLDALAGVKIEPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVSEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVMAARGQHPAASVTLLTTLLDFADTGILDVFVDEGHVQLREATLGGAAGAPCALLRGLELANTFSFLRPNDLVWNYVVDNYLKGNTPVPFDLLFWNGDATNLPGPWYCWYLRHTYLQDELKVPGKLTVCGVPVDLASIDVPTYIYGSREDHIVPWTAAYASTALLANKLRFVLGASGHIAGVINPPAKNKRSHWTNDALPESPQQWLAGATEHHGSWWPDWTAWLAGQAGAKRAAPANYGNARYPAIEPAPGRYVKAKA

SEQ ID NO: 5 - DNA, 1179 bp, Cupriavidus necator
atgactgacgttgtcatcgtatccgccgcccgcaccgcggtcggcaagtttggcggctcgctggccaagatcccggcaccggaactgggtgccgtggtcatcaaggccgcgctggagcgcgccggcgtcaagccggagcaggtgagcgaagtcatcatgggccaggtgctgaccgccggttcgggccagaaccccgcacgccaggccgcgatcaaggccggcctgccggcgatggtgccggccatgaccatcaacaaggtgtgcggctcgggcctgaaggccgtgatgctggccgccaacgcgatcatggcgggcgacgccgagatcgtggtggccggcggccaggaaaacatgagcgccgccccgcacgtgctgccgggctcgcgcgatggtttccgcatgggcgatgccaagctggtcgacaccatgatcgtcgacggcctgtgggacgtgtacaaccagtaccacatgggcatcaccgccgagaacgtggccaaggaatacggcatcacacgcgaggcgcaggatgagttcgccgtcggctcgcagaacaaggccgaagccgcgcagaaggccggcaagtttgacgaagagatcgtcccggtgctgatcccgcagcgcaagggcgacccggtggccttcaagaccgacgagttcgtgcgccagggcgccacgctggacagcatgtccggcctcaagcccgccttcgacaaggccggcacggtgaccgcggccaacgcctcgggcctgaacgacggcgccgccgcggtggtggtgatgtcggcggccaaggccaaggaactgggcctgaccccgctggccacgatcaagagctatgccaacgccggtgtcgatcccaaggtgatgggcatgggcccggtgccggcctccaagcgcgccctgtcgcgcgccgagtggaccccgcaagacctggacctgatggagatcaacgaggcctttgccgcgcaggcgctggcggtgcaccagcagatgggctgggacacctccaaggtcaatgtgaacggcggcgccatcgccatcggccacccgatcggcgcgtcgggctgccgtatcctggtgacgctgctgcacgagatgaagcgccgtgacgcgaagaagggcctggcctcgctgtgcatcggcggcggcatgggcgtggcgctggcagtcgagcgcaaa

SEQ ID NO: 6 - protein, 393 aa, Cupriavidus necator
MTDVVIVSAARTAVGKFGGSLAKIPAPELGAVVIKAALERAGVKPEQVSEVIMGQVLTAGSGQNPARQAAIKAGLPAMVPAMTINKVCGSGLKAVMLAANAIMAGDAEIVVAGGQENMSAAPHVLPGSRDGFRMGDAKLVDTMIVDGLWDVYNQYHMGITAENVAKEYGITREAQDEFAVGSQNKAEAAQKAGKFDEEIVPVLIPQRKGDPVAFKTDEFVRQGATLDSMSGLKPAFDKAGTVTAANASGLNDGAAAVVVMSAAKAKELGLTPLATIKSYANAGVDPKVMGMGPVPASKRALSRAEWTPQDLDLMEINEAFAAQALAVHQQMGWDTSKVNVNGGAIAIGHPIGASGCRILVTLLHEMKRRDAKKGLASLCIGGGMGVALAVERK

SEQ ID NO: 7 - DNA, 741 bp, Cupriavidus necator
atgactcagcgcattgcgtatgtgaccggcggcatgggtggtatcggaaccgccatttgccagcggctggccaaggatggctttcgtgtggtggccggttgcggccccaactcgccgcgccgcgaaaagtggctggagcagcagaaggccctgggcttcgatttcattgcctcggaaggcaatgtggctgactgggactcgaccaagaccgcattcgacaaggtcaagtccgaggtcggcgaggttgatgtgctgatcaacaacgccggtatcacccgcgacgtggtgttccgcaagatgacccgcgccgactgggatgcggtgatcgacaccaacctgacctcgctgttcaacgtcaccaagcaggtgatcgacggcatggccgaccgtggctggggccgcatcgtcaacatctcgtcggtgaacgggcagaagggccagttcggccagaccaactactccaccgccaaggccggcctgcatggcttcaccatggcactggcgcaggaagtggcgaccaagggcgtgaccgtcaacacggtctctccgggctatatcgccaccgacatggtcaaggcgatccgccaggacgtgctcgacaagatcgtcgcgacgatcccggtcaagcgcctgggcctgccggaagagatcgcctcgatctgcgcctggttgtcgtcggaggagtccggtttctcgaccggcgccgacttctcgctcaacggcggcctgcatatgggctga

SEQ ID NO: 8 - protein, 246 aa, Cupriavidus necator
MTQRIAYVTGGMGGIGTAICQRLAKDGFRVVAGCGPNSPRREKWLEQQKALGFDFIASEGNVADWDSTKTAFDKVKSEVGEVDVLINNAGITRDVVFRKMTRADWDAVIDTNLTSLFNVTKQVIDGMADRGWGRIVNISSVNGQKGQFGQTNYSTAKAGLHGFTMALAQEVATKGVTVNTVSPGYIATDMVKAIRQDVLDKIVATIPVKRLGLPEEIASICAWLSSEESGFSTGADFSLNGGLHMG

SEQ ID NO: 9 - DNA, 1236 bp, Cuphea palustris
atggtggctgctgcagcaagttctgcatgcttccctgttccatccccaggagcctcccctaaacctgggaagttaggcaactggtcatcgagtttgagcccttccttgaagcccaagtcaatccccaatggcggatttcaggttaaggcaaatgccagtgcgcatcctaaggctaacggttctgcagtaactctaaagtctggcagcctcaacactcaggaggacactttgtcgtcgtcccctcctccccgggctttttttaaccagttgcctgattggagtatgcttctgactgcaatcacaaccgtcttcgtggcaccagagaagcggtggactatgtttgataggaaatctaagaggcctaacatgctcatggactcgtttgggttggagagagttgttcaggatgggctcgtgttcagacagagtttttcgattaggtcttatgaaatatgcgctgatcgaacagcctctatagagacggtgatgaaccacgtccaggaaacatcactcaatcaatgtaagagtataggtcttctcgatgacggctttggtcgtagtcctgagatgtgtaaaagggacctcatttgggtggttacaagaatgaagataatggtgaatcgctatccaacttggggcgatactatcgaggtcagtacctggctctctcaatcggggaaaatcggtatgggtcgcgattggctaataagtgattgcaacacaggagaaattcttgtaagagcaacgagtgtgtatgccatgatgaatcaaaagacgagaagattctcaaaactcccacacgaggttcgccaggaatttgcgcctcattttctggactctcctcctgccattgaagacaacgacggtaaattgcagaagtttgatgtgaagactggtgattccattcgcaagggtctaactccggggtggtatgacttggatgtcaatcagcacgtaagcaacgtgaagtacattgggtggattctcgagagtatgccaacagaagttttggagactcaggagctatgttctctcacccttgaatataggcgggaatgcggaagggacagtgtgctggagtccgtgacctctatggatccctcaaaagttggagaccggtttcagtaccggcaccttctgcggcttgaggatggggctgatatcatgaagggaagaactgagtggcggccgaagaatgcaggaactaacggggcgatatcaacaggaaagacttga

SEQ ID NO: 10 - DNA, 1408 bp, Cuphea palustris
ccacgcgtccgctgagtttgctggttaccattttccctgcgaacaaacatggtggctgccgcagcaagtgctgcattcttctccgtcgcaaccccgcgaacaaacatttcgccatcgagcttgagcgtccccttcaagcccaaatcaaaccacaatggtggctttcaggttaaggcaaacgccagtgcccatcctaaggctaacggttctgcagtaagtctaaagtctggcagcctcgagactcaggaggacaaaacttcatcgtcgtcccctcctcctcggactttcattaaccagttgcccgtctggagtatgcttctgtctgcagtcacgactgtcttcggggtggctgagaagcagtggccaatgcttgaccggaaatctaagaggcccgacatgcttgtggaaccgcttggggttgacaggattgtttatgatggggttagtttcagacagagtttttcgattagatcttacgaaataggcgctgatcgaacagcctcgatagagaccctgatgaacatgttccaggaaacatctcttaatcattgtaagattatcggtcttctcaatgacggctttggtcgaactcctgagatgtgtaagagggacctcatttgggtggtcacgaaaatgcagatcgaggtgaatcgctatcctacttggggtgatactatagaggtcaatacttgggtctcagcgtcggggaaacacggtatgggtcgagattggctgataagtgattgccatacaggagaaattcttataagagcaacgagcgtgtgggctatgatgaatcaaaagacgagaagattgtcgaaaattccatatgaggttcgacaggagatagagcctcagtttgtggactctgctcctgtcattgtagacgatcgaaaatttcacaagcttgatttgaagaccggtgattccatttgcaatggtctaactccaaggtggactgacttggatgtcaatcagcacgttaacaatgtgaaatacatcgggtggattctccagagtgttcccacagaagttttcgagacgcaggagctatgtggcctcacccttgagtataggcgagaatgcggaagggacagtgtgctggagtccgtgaccgctatggatccatcaaaagagggagaccggtctctttaccagcaccttctccgactcgaggacggggctgatatcgtcaaggggagaaccgagtggcggccgaagaatgcaggagccaagggagcaatattaaccggaaagacctcaaatggaaactctatatcttagaaggaggaagggacctttccgagttgtgtgtttatttgctttgctttgattcactccattgtataataatactacggtcagccgtctttgtatttgctaagacaaatagcacagtcattaagtt

SEQ ID NO: 11 - DNA, 981 bp, Artificial Sequence
  synthetic polynucleotide
atgctgctgaccgccatcacgaccgtgttcgtggccccggagaagcgctggaccatgttcgaccgcaagtcgaagcgcccgaacatgctgatggactcgttcggcctggagcgcgtggtgcaggacggcctggtgttccgccagtcgttctcgatccgctcgtacgagatctgcgccgaccgcaccgcctcgatcgagaccgtgatgaaccacgtgcaggagacctcgctgaaccagtgcaagtcgatcggcctgctggacgacggcttcggccgttcgccggagatgtgcaagcgcgacctgatctgggtggtgacccgcatgaagatcatggtgaaccgctacccgacctggggcgacaccatcgaggtgagcacctggctgtcgcagtcgggcaagatcggcatgggccgcgattggctgatctcggactgcaacaccggcgagatcctggtgcgcgccacctcggtgtacgccatgatgaaccagaagacccgccgcttctcgaagctgccgcacgaggtgcgccaggagttcgccccgcacttcctggattcaccaccggccatcgaggacaatgacggcaagctgcagaagttcgacgtgaagaccggcgactcgatccgcaagggcctgaccccgggctggtacgacctggacgtgaaccagcacgtgaacaacgtgaagtacatcggctggatcctgcagtcggtgccgaccgaggtgttcgagacccaggagctgtgcggcctgaccctggagtatcgccgcgaatgcggccgcgattcggtgctggaatcggtgaccgccatggacccgtcgaaggagggcgatcgctcgctgtaccagcacctgctgcgcctggaagatggcgccgacatcgtgaagggccgcaccgaatggcgcccgaagaatgcaggcgcaaagggcgccattctgaccggcaagacctcaggcggccaccaccaccaccatcattga

SEQ ID NO: 12 - DNA, 209 bp, Ralstonia eutropha
gcctcgctgctttccacctggcgccgcacgcggccccagacgtcgatttcccaggttgcgcccagggtcgcgctctgcccgttgagcgtgctgccgctggcgccgcgcgcgcgcgaggcgccggcctgtgcgtcgacggtcgggaagaagccggcgcgcgcggcctgcagcgacgccaccgcctggcggtactgcgcctcggcggcctt

SEQ ID NO: 13 - DNA, 1495 bp, Ralstonia eutropha
aggcgccggcctgtgcgtcgacggtcgggaagaagccggcgcgcgcggcctgcagcgacgccaccgcctggcggtactgcgcctcggcggccttgatgttctggttcgagatctgcacctcggacatcagcgcgtcgagctgcgcatcgccgaacacggtccaccagtcggcgcgtgccagcgcatcctgcggctcggcgggcttccagtcgccggtccaggcgggggtggcggcatcggcttccttgaaggatgcggaaaccggcgcgtcggggcgctggtagtcggggccgacggcgcagccggccagcagcagcgcgcaggccagcgacaccggcagggcatgggtcaggaggcgggaaagaactgtcatgtcgagtcttcgcaaatctagacggcggccggctggtcaggcgtgccggcaccacggcggcgctggcgccaggccttgaccttcaggcgccagcggtccagcgtcaggtagaccaccggcgtggtgtacagcgtcagcagctggcttaccaccagtccgccgacaatggagatgcccagcggcgcgcgcagttcggcgccgtcgccgcggccgattgccagcggcaccgcgcccagcagcgcggccatggtggtcatcaggatcgggcggaagcgcagcaggcaggcgcggtagatcgcgtcgcgcggcgacaggccatcgcgccgttcggcatcgatggcgaagtcgatcatcatgatcgcgttctttttcacgatgccgatcagcaggatcacgccgatcagcgcgatgatgctgaagtcggtcttcgatgccagcagcgccagcagcgcgcccacgccggcggagggcagcgtcgacaggatcgtcagcggatgcacatagctttcatacagcacgcccagcacgatgtagatcgtgatcagcgccgccaggatcaggatcggctgactcttgagcgaatcctggaacgccttggcgccgccctggaagttggcgcgcagcgtctccggcacgccgatgcgcgccatctcgcgcgtgatcgcgtcggtcgcctgcgacagcgaagtgccctcggccaggttgaacgagatcgtcgaggccgcgaactggccctggtggttcacgcccagcggcgtgctggacggggtcacgcgcgcgaacgccgccagcggcacgcggttgccgttgccggtgaccacgtagatgtccttgagcgcatcgggcccttgcaggtattcctggctcagctccatcaccacgcggtactggttcagcggatggtagatggtggacaccagccgctggccgaaggcatcgttgagcaccgcatccacctgctgcgcggtcacgcccaggcgcgaggccgcgtcgcggtcgatgatcaccgaggtctgcaggcccttgtcgttggtatcggtgtcgatatcctccagccccttcaggttcgacaacgcggcgcgcaccttgggctcccacgcgcgcagcacttccaggtcgtcc

SEQ ID NO: 14 - DNA, 201 bp, Ralstonia eutropha
gcagcttgatgccattgacgaggtagatggaaaccggcacgtgctctttgcgcagcgcgttcaggaacgggccttgtagcagttgccctttgttgctcatggcacactccaaatttataggtttagtggtgaatgatggggatggaaatccccggttcaagtcaggcggcgcaaaaacgcgccagaaaaaagatcaaaaac

SEQ ID NO: 15 - DNA, 479 bp, Ralstonia eutropha
gaggatgccatgtccgaagcgcctgtccttgccccctcgacctcaacccagccgcccgccgccggccagctcaacctgatccgcccgcagccatatgccgactgggcgccgcaggtcacggccgaagaacgcgccacgctgcgccgcgagctggagcagggcgccgtgctgtacttcccgaacctgaatttccgcttccagccgggcgaagagcgcttccttgacagccgctattccgacggcaagtccaagaacatcaacctgcgcgccgacgacaccgcggtgcgcggcgcccagggcagtccgcaggacctggcggacctgtacacgctgatccgccgctacgccgacaacagcgaattgctggtgcgcacgctgttccctgaatacatcccgcacatgacgcgcgccggcacctcgctgcggcccagcgagatcgccgggcgcccggtcagctggcgcaaggacgacacccgcct

SEQ ID NO: 16 - protein, 326 aa, Artificial Sequence
  synthetic polypeptide
MLLTAITTVFVAPEKRWTMFDRKSKRPNMLMDSFGLERVVQDGLVFRQSFSIRSYEICADRTASIETVMNHVQETSLNQCKSIGLLDDGFGRSPEMCKRDLIWVVTRMKIMVNRYPTWGDTIEVSTWLSQSGKIGMGRDWLISDCNTGEILVRATSVYAMMNQKTRRFSKLPHEVRQEFAPHFLDSPPAIEDNDGKLQKFDVKTGDSIRKGLTPGWYDLDVNQHVNNVKYIGWILQSVPTEVFETQELCGLTLEYRRECGRDSVLESVTAMDPSKEGDRSLYQHLLRLEDGADIVKGRTEWRPKNAGAKGAILTGKTSGGHHHHHH

SEQ ID NO: 17 - protein, 411 aa, Cuphea palustris
MVAAAASSACFPVPSPGASPKPGKLGNWSSSLSPSLKPKSIPNGGFQVKANASAHPKANGSAVTLKSGSLNTQEDTLSSSPPPRAFFNQLPDWSMLLTAITTVFVAPEKRWTMFDRKSKRPNMLMDSFGLERVVQDGLVFRQSFSIRSYEICADRTASIETVMNHVQETSLNQCKSIGLLDDGFGRSPEMCKRDLIWVVTRMKIMVNRYPTWGDTIEVSTWLSQSGKIGMGRDWLISDCNTGEILVRATSVYAMMNQKTRRFSKLPHEVRQEFAPHFLDSPPAIEDNDGKLQKFDVKTGDSIRKGLTPGWYDLDVNQHVSNVKYIGWILESMPTEVLETQELCSLTLEYRRECGRDSVLESVTSMDPSKVGDRFQYRHLLRLEDGADIMKGRTEWRPKNAGTNGAISTGKT

SEQ ID NO: 18 - protein, 316 aa, Artificial Sequence
  synthetic polypeptide
LLTAITTVFVAPEKRWTMFDRKSKRPNMLMDSFGLERVVQDGLVFRQSFSIRSYEICADRTASIETVMNHVQETSLNQCKSIGLLDDGFGRSPEMCKRDLIWVVTRMKIMVNRYPTWGDTIEVSTWLSQSGKIGMGRDWLISDCNTGEILVRATSVYAMMNQKTRRFSKLPHEVRQEFAPHFLDSPPAIEDNDGKLQKFDVKTGDSIRKGLTPGWYDLDVNQHVSNVKYIGWILESMPTEVLETQELCSLTLEYRRECGRDSVLESVTSMDPSKVGDRFQYRHLLRLEDGADIMKGRTEWRPKNAGTNGAISTGKT

SEQ ID NO: 19 - protein, 411 aa, Cuphea palustris
MVAAAASAAFFSVATPRTNISPSSLSVPFKPKSNHNGGFQVKANASAHPKANGSAVSLKSGSLETQEDKTSSSSPPPRTFINQLPVWSMLLSAVTTVFGVAEKQWPMLDRKSKRPDMLVEPLGVDRIVYDGVSFRQSFSIRSYEIGADRTASIETLMNMFQETSLNHCKIIGLLNDGFGRTPEMCKRDLIWVVTKMQIEVNRYPTWGDTIEVNTWVSASGKHGMGRDWLISDCHTGEILIRATSVWAMMNQKTRRLSKIPYEVRQEIEPQFVDSAPVIVDDRKFHKLDLKTGDSICNGLTPRWTDLDVNQHVNNVKYIGWILQSVPTEVFETQELCGLTLEYRRECGRDSVLESVTAMDPSKEGDRSLYQHLLRLEDGADIVKGRTEWRPKNAGAKGAILTGKTSNGNSIS

SEQ ID NO: 20 - protein, 315 aa, Artificial Sequence
  synthetic polypeptide
LLSAVTTVFGVAEKQWPMLDRKSKRPDMLVEPLGVDRIVYDGVSFRQSFSIRSYEIGADRTASIETLMNMFQETSLNHCKIIGLLNDGFGRTPEMCKRDLIWVVTKMQIEVNRYPTWGDTIEVNTWVSASGKHGMGRDWLISDCHTGEILIRATSVWAMMNQKTRRLSKIPYEVRQEIEPQFVDSAPVIVDDRKFHKLDLKTGDSICNGLTPRWTDLDVNQHVNNVKYIGWILQSVPTEVFETQELCGLTLEYRRECGRDSVLESVTAMDPSKEGDRSLYQHLLRLEDGADIVKGRTEWRPKNAGAKGAILTGKT

SEQ ID NO: 21 - protein, 316 aa, Artificial Sequence
  synthetic polypeptide
LLTAITTVFVAPEKRWTMFDRKSKRPNMLMDSFGLERVVQDGLVFRQSFSIRSYEICADRTASIETVMNHVQETSLNQCKSIGLLDDGFGRSPEMCKRDLIWVVTRMKIMVNRYPTWGDTIEVSTWLSQSGKIGMGRDWLISDCNTGEILVRATSVYAMMNQKTRRFSKLPHEVRQEFAPHFLDSPPAIEDNDGKLQKFDVKTGDSIRKGLTPGWYDLDVNQHVNNVKYIGWILQSVPTEVFETQELCGLTLEYRRECGRDSVLESVTAMDPSKEGDRSLYQHLLRLEDGADIVKGRTEWRPKNAGAKGAILTGKT

SEQ ID NO: 22 - DNA, 717 bp, Marvinbryantia formatexigens
atgatttatatggcatatcaataccgcagccgcatccgctacagcgaaattggcgaggacaaaaagcttacgctgcccggtctggtgaattatttccaggactgcagcaccttccagtcggaggcactcggcatagggctggacacgctgggagcgcgccagcgggcatggcttctggcgtcctggaaaattgtaatagacaggctgccgcggcttggggaggaggttgtgacggagacctggccatatggctttaagggcttccagggaaaccgcaacttccgtatgctggaccaggagggacatacactggctgcagcggcatccgtctggatttatttaaatgtggaaagcgggcatccgtgccggattgacggggatgttctggaggcatatgagctggaagaggagctgccgctcggtccgttttcgcgcaagattccggttccggaggaaagcacggagcgggacagctttctggtgatgcgcagtcacctggacaccaatcaccatgtcaacaacgggcagtatatactgatggcggaggaatatctgccggagggctttaaagtaaagcagatacgcgtggagtaccgcaaagccgccgttctgcacgatacgattgtgccgtttgtgtgcacagagccgcagcgctgcacggtcagcctttgcggaagtgatgaaaagccgtttgccgtcgtagaattttcggaataa

SEQ ID NO: 23 - DNA, 714 bp, Artificial Sequence
  synthetic polynucleotide
atgatctacatggcctaccagtaccgctcgcgcatccgctactcggagatcggcgaggacaagaagctgaccctgccgggcctggtgaactacttccaggactgctcgaccttccagtcggaggccctgggcatcggcctggacaccctgggcgcccgccagcgcgcctggctgctggcctcgtggaagatcgtgatcgaccgcctgccgcgcctgggcgaggaggtggtgaccgagacctggccgtacggcttcaagggcttccagggcaaccgcaacttccgcatgctggaccaggagggccacaccctggccgccgccgcctcggtgtggatctacctgaacgtggagtcgggccacccgtgccgcatcgacggcgacgtgctggaggcctacgagctggaggaggagctgccgctgggcccgttctcgcgcaagatcccggtgccggaggagtcgaccgagcgcgactcgttcctggtgatgcgctcgcacctggacaccaaccaccacgtgaacaacggccagtacatcctgatggccgaggagtacctgccggagggcttcaaggtgaagcagatccgcgtggagtaccgcaaggccgccgtgctgcacgacaccatcgtgccgttcgtgtgcaccgagccgcagcgctgcaccgtgtcgctgtgcggctcggacgagaagccgttcgccgtggtggagttctcggag

SEQ ID NO: 24 - protein, 238 aa, Marvinbryantia formatexigens
MIYMAYQYRSRIRYSEIGEDKKLTLPGLVNYFQDCSTFQSEALGIGLDTLGARQRAWLLASWKIVIDRLPRLGEEVVTETWPYGFKGFQGNRNFRMLDQEGHTLAAAASVWIYLNVESGHPCRIDGDVLEAYELEEELPLGPFSRKIPVPEESTERDSFLVMRSHLDTNHHVNNGQYILMAEEYLPEGFKVKQIRVEYRKAAVLHDTIVPFVCTEPQRCTVSLCGSDEKPFAVVEFSE

SEQ ID NO: 25 - DNA, 1377 bp, Acinetobacter baylyi
atgcgcccattacatccgattgattttatattcctgtcactagaaaaaagacaacagcctatgcatgtaggtggtttatttttgtttcagattcctgataacgccccagacacctttattcaagatctggtgaatgatatccggatatcaaaatcaatccctgttccaccattcaacaataaactgaatgggcttttttgggatgaagatgaagagtttgatttagatcatcattttcgtcatattgcactgcctcatcctggtcgtattcgtgaattgcttatttatatttcacaagagcacagtacgctgctagatcgggcaaagcccttgtggacctgcaatattattgaaggaattgaaggcaatcgttttgccatgtacttcaaaattcaccatgcgatggtcgatggcgttgctggtatgcggttaattgaaaaatcactctcccatgatgtaacagaaaaaagtatcgtgccaccttggtgtgttgagggaaaacgtgcaaagcgcttaagagaacctaaaacaggtaaaattaagaaaatcatgtctggtattaagagtcagcttcaggcgacacccacagtcattcaagagctttctcagacagtatttaaagatattggacgtaatcctgatcatgtttcaagctttcaggcgccttgttctattttgaatcagcgtgtgagctcatcgcgacgttttgcagcacagtcttttgacctagatcgttttcgtaatattgccaaatcgttgaatgtgaccattaatgatgttgtactagcggtatgttctggtgcattacgtgcgtatttgatgagtcataatagtttgccttcaaaaccattaattgccatggttccagcctctattcgcaatgacgattcagatgtcagcaaccgtattacgatgattctggcaaatttggcaacccacaaagatgatcctttacaacgtcttgaaattatccgccgtagtgttcaaaactcaaagcaacgcttcaaacgtatgaccagcgatcagattctaaattatagtgctgtcgtatatggccctgcaggactcaacataatttctggcatgatgccaaaacgccaagccttcaatctggttatttccaatgtgcctggcccaagagagccactttactggaatggtgccaaacttgatgcactctacccagcttcaattgtattagacggtcaagcattgaatattacaatgaccagttatttagataaacttgaagttggtttgattgcatgccgtaatgcattgccaagaatgcagaatttactgacacatttagaagaagaaattcaactatttgaaggcgtaattgcaaagcaggaagatattaaaacagccaattaa

SEQ ID NO: 26 - DNA, 1374 bp, Artificial Sequence
  synthetic polynucleotide
atgcgcccgctgcacccgatcgacttcatcttcctgtcgctggagaagcgccagcagccgatgcacgtgggcggcctgttcctgttccagatcccggacaacgccccggacaccttcatccaggacctggtgaacgacatccgcatctcgaagtcgatcccggtgccgccgttcaacaacaagctgaacggcctgttctgggacgaggacgaggagttcgacctggaccaccacttccgccacatcgccctgccgcacccgggccgcatccgcgagctgctgatctacatctcgcaggagcactcgaccctgctggaccgcgccaagccgctgtggacctgcaacatcatcgagggcatcgagggcaaccgcttcgccatgtacttcaagatccaccacgccatggtggacggcgtggccggcatgcgcctgatcgagaagtcgctgtcgcacgacgtgaccgagaagtcgatcgtgccgccgtggtgcgtggagggcaagcgcgccaagcgcctgcgcgagccgaagaccggcaagatcaagaagatcatgtcgggcatcaagtcgcagctgcaggccaccccgaccgtgatccaggagctgtcgcagaccgtgttcaaggacatcggccgcaacccggaccacgtgtcgtcgttccaggccccgtgctcgatcctgaaccagcgcgtgtcgtcgtcgcgccgcttcgccgcccagtcgttcgacctggaccgcttccgcaacatcgccaagtcgctgaacgtgaccatcaacgacgtggtgctggccgtgtgctcgggcgccctgcgcgcctacctgatgtcgcacaactcgctgccgtcgaagccgctgatcgccatggtgccggcctcgatccgcaacgacgactcggacgtgtcgaaccgcatcaccatgatcctggccaacctggccacccacaaggacgacccgctgcagcgcctggagatcatccgccgctcggtgcagaactcgaagcagcgcttcaagcgcatgacctcggaccagatcctgaactactcggccgtggtgtacggcccggccggcctgaacatcatctcgggcatgatgccgaagcgccaggccttcaacctggtgatctcgaacgtgccgggcccgcgcgagccgctgtactggaacggcgccaagctggacgccctgtacccggcctcgatcgtgctggacggccaggccctgaacatcaccatgacctcgtacctggacaagctggaggtgggcctgatcgcctgccgcaacgccctgccgcgcatgcagaacctgctgacccacctggaggaggagatccagctgttcgagggcgtgatcgccaagcaggaggacatcaagaccgccaac

SEQ ID NO: 27 - DNA, 1449 bp, Thermomonospora curvata
atgcgccagctgacggcggtggacgccaacttcctgaacgtcgagaccggcaccacgcacgcccatatcgccggcctggggatcctcgacccggtcgcctgccccggcggccggctcaccgccgaggacctcatcgaggtgatccgcgaacgcgcccacctggccccccggccgctgcggatgcggctggccgcggtgccgctgggcatcgaccggccctactgggaggacgacccggacttcgacccggcccgccacgtcttcgaggtggggctgccggccccgggcaacgccgcccagctcgccgacgtcgtggcgatgctgcacgaacggccgctggaccgcgcccggccgctgtgggaggcggtggtcatccagggcctggagggcggccgcaccgccgtctacatcaaggtccaccacgccgcggtggacggggtcctggccaccgagaccctggccgccctgctggacctgtccccgcagccgcgcgagctgccccccgacgacaccgtgccgcagcaggcgccggccctggccgaacgggtgcgcaccgggctgctgcgcgcgctcgcccacccggtgcgcggcgcccgcatgctggcccgcaccgccccctacctggatgagatccccggccttgcgcaactgccgggagtgcagccgctggcccgggcgatccagggggcgctcggccgcgacggcgtcgtgccgctgccccgcaccgtcgccccgcccaccccgttcaacgggacgatcagcgcccgccgggcggtggccttcggcgagctgccgctggcggagatccggcgcatccgccgggagctgggcggcagcgtcaacgacgtggtgatggcgctggtggccacggcgctgcaccgctggctggacaagcgcggcgagctgcccgaccggccgctggtggcggcggtgccggtgtcgctgcgccgcggccgggacggcgatgcggcgggcggcaaccggatgtcggcgatggtgacgccgctggccacccatctggccgacccggccgagcgcttcgccgcgatccgcggcgacctggcggcggccaaacggcgcttcgcccgctcctcgggcgcctggctggaggggctgagcgaactggtgcccgccccgctggccggcccgctgctgcggctggccctgcaggcccggccgggcgagtacctgcgcccggtcaatctgctggtctccaacgtgcccggcccggacttcccgctgtacctgcgcggcgcccgggtgctcggctacttcccgatctcggtggtcagcgacctgaccggcgggctgaacatcaccgtgctgtcctatgacggcaagctcgacgtcggcatcgtgacctgccgccagatgatccccgacccctgggagatcatggaccacctcgacgacgccctgggggagctgaggggcctcatcgacggctga

SEQ ID NO: 28 - DNA, 1446 bp, Artificial Sequence
  synthetic polynucleotide
atgcgccagctgaccgccgtggacgccaacttcctgaacgtggagaccggcaccacccacgcccacatcgccggcctgggcatcctggacccggtggcctgcccgggcggccgcctgaccgccgaggacctgatcgaggtgatccgcgagcgcgcccacctggccccgcgcccgctgcgcatgcgcctggccgccgtgccgctgggcatcgaccgcccgtactgggaggacgacccggacttcgacccggcccgccacgtgttcgaggtgggcctgccggccccgggcaacgccgcccagctggccgacgtggtggccatgctgcacgagcgcccgctggaccgcgcccgcccgctgtgggaggccgtggtgatccagggcctggagggcggccgcaccgccgtgtacatcaaggtgcaccacgccgccgtggacggcgtgctggccaccgagaccctggccgccctgctggacctgtcgccgcagccgcgcgagctgccgccggacgacaccgtgccgcagcaggccccggccctggccgagcgcgtgcgcaccggcctgctgcgcgccctggcccacccggtgcgcggcgcccgcatgctggcccgcaccgccccgtacctggacgagatcccgggcctggcccagctgccgggcgtgcagccgctggcccgcgccatccagggcgccctgggccgcgacggcgtggtgccgctgccgcgcaccgtggccccgccgaccccgttcaacggcaccatctcggcccgccgcgccgtggccttcggcgagctgccgctggccgagatccgccgcatccgccgcgagctgggcggctcggtgaacgacgtggtgatggccctggtggccaccgccctgcaccgctggctggacaagcgcggcgagctgccggaccgcccgctggtggccgccgtgccggtgtcgctgcgccgcggccgcgacggcgacgccgccggcggcaaccgcatgtcggccatggtgaccccgctggccacccacctggccgacccggccgagcgcttcgccgccatccgcggcgacctggccgccgccaagcgccgcttcgcccgctcgtcgggcgcctggctggagggcctgtcggagctggtgccggccccgctggccggcccgctgctgcgcctggccctgcaggcccgcccgggcgagtacctgcgcccggtgaacctgctggtgtcgaacgtgccgggcccggacttcccgctgtacctgcgcggcgcccgcgtgctgggctacttcccgatctcggtggtgtcggacctgaccggcggcctgaacatcaccgtgctgtcgtacgacggcaagctggacgtgggcatcgtgacctgccgccagatgatcccggacccgtgggagatcatggaccacctggacgacgccctgggcgagctgcgcggcctgatcgacggc

SEQ ID NO: 29 - protein, 458 aa, Acinetobacter baylyi
MRPLHPIDFIFLSLEKRQQPMHVGGLFLFQIPDNAPDTFIQDLVNDIRISKSIPVPPFNNKLNGLFWDEDEEFDLDHHFRHIALPHPGRIRELLIYISQEHSTLLDRAKPLWTCNIIEGIEGNRFAMYFKIHHAMVDGVAGMRLIEKSLSHDVTEKSIVPPWCVEGKRAKRLREPKTGKIKKIMSGIKSQLQATPTVIQELSQTVFKDIGRNPDHVSSFQAPCSILNQRVSSSRRFAAQSFDLDRFRNIAKSLNVTINDVVLAVCSGALRAYLMSHNSLPSKPLIAMVPASIRNDDSDVSNRITMILANLATHKDDPLQRLEIIRRSVQNSKQRFKRMTSDQILNYSAVVYGPAGLNIISGMMPKRQAFNLVISNVPGPREPLYWNGAKLDALYPASIVLDGQALNITMTSYLDKLEVGLIACRNALPRMQNLLTHLEEEIQLFEGVIAKQEDIKTAN

SEQ ID NO: 30 - protein, 482 aa, Thermomonospora curvata
MRQLTAVDANFLNVETGTTHAHIAGLGILDPVACPGGRLTAEDLIEVIRERAHLAPRPLRMRLAAVPLGIDRPYWEDDPDFDPARHVFEVGLPAPGNAAQLADVVAMLHERPLDRARPLWEAVVIQGLEGGRTAVYIKVHHAAVDGVLATETLAALLDLSPQPRELPPDDTVPQQAPALAERVRTGLLRALAHPVRGARMLARTAPYLDEIPGLAQLPGVQPLARAIQGALGRDGVVPLPRTVAPPTPFNGTISARRAVAFGELPLAEIRRIRRELGGSVNDVVMALVATALHRWLDKRGELPDRPLVAAVPVSLRRGRDGDAAGGNRMSAMVTPLATHLADPAERFAAIRGDLAAAKRRFARSSGAWLEGLSELVPAPLAGPLLRLALQARPGEYLRPVNLLVSNVPGPDFPLYLRGARVLGYFPISVVSDLTGGLNITVLSYDGKLDVGIVTCRQMIPDPWEIMDHLDDALGELRGLIDG

SEQ ID NO: 31 - DNA, 684 bp, Rhodococcus opacus
atgccccacacctctgccgctcacgccgggcttcgtatgtccgccctgacgctgatattggccgtgctctgcgtgcaggtccacgacggcggcccactgaccggtgccgacgtgcccgtgacgtcgtgggccgtcggaaaccgttccgcggtcctcgaccacgcggcgctcctcgtcaccgacctcggcagccccgtcgccaccgtggccctcgccgtgatctgcgggctcgcgctcgcgtggcatcggcgttccgcgattcccgccgtcctcgtcgtcggaacggtcggggccgccaccacggcaagcacggccctgaagctggtggtcgggcgcagccggccggccctcgacctgcaggaggtcctggagacggactactcgttcccgtccggacatgtcacgggtacggtggctttgctcggcatcacggccgcgatcctgctcggccgccggcgccgcctcgtccgggtgtgtggcggggccgtggtcgggtgcggggtggtgatcgttgccgcgacccgcgtgtacctcggtgtccactggctcaccgatgttgtcgccggcgcgatcctgggcgcggtgttcgtgacggtgggggccgccacgtacgagcgtgtccacccgccggtcggcacccccgctccgtcgaaaccgctcgcggccgtcgaccgagtgggaggc

SEQ ID NO: 32 - DNA, 684 bp, Artificial Sequence
  synthetic polynucleotide
atgccgcacacctcggccgcccacgccggcctgcgcatgtcggccctgaccctgatcctggccgtgctgtgcgtgcaggtgcacgacggcggcccgctgaccggcgccgacgtgccggtgacctcgtgggccgtgggcaaccgctcggccgtgctggaccacgccgccctgctggtgaccgacctgggctcgccggtggccaccgtggccctggccgtgatctgcggcctggccctggcctggcaccgccgctcggccatcccggccgtgctggtggtgggcaccgtgggcgccgccaccaccgcctcgaccgccctgaagctggtggtgggccgctcgcgcccggccctggacctgcaggaggtgctggagaccgactactcgttcccgtcgggccacgtgaccggcaccgtggccctgctgggcatcaccgccgccatcctgctgggccgccgccgccgcctggtgcgcgtgtgcggcggcgccgtggtgggctgcggcgtggtgatcgtggccgccacccgcgtgtacctgggcgtgcactggctgaccgacgtggtggccggcgccatcctgggcgccgtgttcgtgaccgtgggcgccgccacctacgagcgcgtgcacccgccggtgggcaccccggccccgtcgaagccgctggccgccgtggaccgcgtgggcggc

SEQ ID NO: 33 - DNA, 687 bp, Rhodococcus jostii
atgccccacacctccatcgccaccgccgggcttcgtgtgtccgcgctgacgctgatcctggccgtgctctgtgtgcaggtccgcgacggcggcccactgaccggtgccgacgtgcccgcgacgtcgtgggtcgtcggtcaccgctccgccacccttgaccatgtggctctcctcgtcaccgccctcggcagccccgtcgccaccgtggccctcgccgtgatctgcgggctcgccctcgcctggcgccggcgttccgcgatccccgccgccgtcgtcgtcgggacggtcggggccgccaccgcggcgagcacggccctgaagctggtcgtcgaacgcagccggccggccgtcgacctgcaggaggtcctggagacggactactcgttcccgtccgggcacgtcacgggcaccgcggccctgctcggggtcacggcggcgatcctgctcggccgccggcatctccgcgtccgggtgtgcggcgcggcggtggccgggtgcggggtggtgatcgttgccgtgacccgcgtctacctcggtgtccactggatgtccgacgtcgtcgccggcgcgatcctcggcgcggtgttcgtgacggtaggcgccgccacgtacgagcgtgtccaccgtccgctcgtcgccgtcgctccgtcgaaaccgctcgcggtcctcgaccgagtgggaggctga

SEQ ID NO: 34 - DNA, 684 bp, Artificial Sequence
  synthetic polynucleotide
atgccgcacacctcgatcgccaccgccggcctgcgcgtgtcggccctgaccctgatcctggccgtgctgtgcgtgcaggtgcgcgacggcggcccgctgaccggcgccgacgtgccggccacctcgtgggtggtgggccaccgctcggccaccctggaccacgtggccctgctggtgaccgccctgggctcgccggtggccaccgtggccctggccgtgatctgcggcctggccctggcctggcgccgccgctcggccatcccggccgccgtggtggtgggcaccgtgggcgccgccaccgccgcctcgaccgccctgaagctggtggtggagcgctcgcgcccggccgtggacctgcaggaggtgctggagaccgactactcgttcccgtcgggccacgtgaccggcaccgccgccctgctgggcgtgaccgccgccatcctgctgggccgccgccacctgcgcgtgcgcgtgtgcggcgccgccgtggccggctgcggcgtggtgatcgtggccgtgacccgcgtgtacctgggcgtgcactggatgtcggacgtggtggccggcgccatcctgggcgccgtgttcgtgaccgtgggcgccgccacctacgagcgcgtgcaccgcccgctggtggccgtggccccgtcgaagccgctggccgtgctggaccgcgtgggcggc

SEQ ID NO: 35 - protein, 228 aa, Rhodococcus opacus
MPHTSAAHAGLRMSALTLILAVLCVQVHDGGPLTGADVPVTSWAVGNRSAVLDHAALLVTDLGSPVATVALAVICGLALAWHRRSAIPAVLVVGTVGAATTASTALKLVVGRSRPALDLQEVLETDYSFPSGHVTGTVALLGITAAILLGRRRRLVRVCGGAVVGCGVVIVAATRVYLGVHWLTDVVAGAILGAVFVTVGAATYERVHPPVGTPAPSKPLAAVDRVGG

SEQ ID NO: 36 - protein, 228 aa, Rhodococcus jostii
MPHTSIATAGLRVSALTLILAVLCVQVRDGGPLTGADVPATSWVVGHRSATLDHVALLVTALGSPVATVALAVICGLALAWRRRSAIPAAVVVGTVGAATAASTALKLVVERSRPAVDLQEVLETDYSFPSGHVTGTAALLGVTAAILLGRRHLRVRVCGAAVAGCGVVIVAVTRVYLGVHWMSDVVAGAILGAVFVTVGAATYERVHRPLVAVAPSKPLAVLDRVGG

SEQ ID NO: 37 - DNA, 1506 bp, Theobroma cacao
atggccatttctgattccccagaaattttgggttctactgctactgttacctcctcttctcattccgattctgatttgaacttgttgtccatcagaagaagaacttctactactgctgctggtagagcaccagatagagatgattctggtaatggtgaagctgttgatgatagagatcaagttgaatccgctaacttgatgtctaacgttgctgaaaatgctaacgaaatgccaaactcttctgataccagattcacttacagaccaagagttccagctcacagaagaatcaaagaatctccattatcttccggtgccatcttcaaacaatctcatgctggtttgttcaacttgtgcatcgttgttttggttgccgttaactccagattgatcatcgaaaacttgatgaagtacggttggttgatcagatctggtttttggttctcttccagatctttgtctgattggccattattcatgtgttgtttgaccttgccaattttcccattggctgcttttgttgttgaaaagttggtccaaagaaactacatctccgaaccagttgttgttttcttgcatgccattatttctacaaccgctgtcttgtatccagtcatcgttaatttgagatgcgattccgcttttttgtctggtgttgctttgatgttgttcgcttgtatcgtttggttgaagttggtttcttacgctcataccaacaacgatatgagagctttggctaaatctgctgaaaagggtgatgttgatccatcctacgatgtttcttttaagtccttggcttacttcatggttgctccaactttgtgttaccaacaatcttatccaagaaccccagctgttagaaaatcttgggttgttagacaattcattaagttgatcgttttcaccggtttgatgggtttcatcatcgaacaatatatcaacccaatcgtccaaaactcccaacatccattgaaaggtaatttgttgtacgccatcgaaagagtcttgaagttgtctgttccaaacttgtatgtctggttgtgcatgttctactgtttcttccatttgtggttgaacatcttggccgaattattgagattcggtgacagagaattttacaaggattggtggaatgctaagaccgtcgaagaatattggagaatgtggaatatgccagttcacaagtggatggttagacatatctacttcccatgtttgagaaacggtattccaaaaggtgttgctatcgttattgccttcttggtttctgctgttttccacgaattgtgtattgctgttccatgccatattttcaagttgtgggctttcattggtatcatgttccaagttccattggtcttgattaccaactacttgcaagataagttcagatcctctatggtcggtaacatgattttctggttcatcttctccattttgggtcaacctatgtgtgtcttgttgtactaccatgatttgatgaacagaaagggtaaggccgattga

SEQ ID NO: 38 - DNA, 1332 bp, Artificial Sequence
  synthetic polynucleotide
atgcaagttgaatccgctaacttgatgtctaacgttgctgaaaatgctaacgaaatgccaaactcttctgataccagattcacttacagaccaagagttccagctcacagaagaatcaaagaatctccattatcttccggtgccatcttcaaacaatctcatgctggtttgttcaacttgtgcatcgttgttttggttgccgttaactccagattgatcatcgaaaacttgatgaagtacggttggttgatcagatctggtttttggttctcttccagatctttgtctgattggccattattcatgtgttgtttgaccttgccaattttcccattggctgcttttgttgttgaaaagttggtccaaagaaactacatctccgaaccagttgttgttttcttgcatgccattatttctacaaccgctgtcttgtatccagtcatcgttaatttgagatgcgattccgcttttttgtctggtgttgctttgatgttgttcgcttgtatcgtttggttgaagttggtttcttacgctcataccaacaacgatatgagagctttggctaaatctgctgaaaagggtgatgttgatccatcctacgatgtttcttttaagtccttggcttacttcatggttgctccaactttgtgttaccaacaatcttatccaagaaccccagctgttagaaaatcttgggttgttagacaattcattaagttgatcgttttcaccggtttgatgggtttcatcatcgaacaatatatcaacccaatcgtccaaaactcccaacatccattgaaaggtaatttgttgtacgccatcgaaagagtcttgaagttgtctgttccaaacttgtatgtctggttgtgcatgttctactgtttcttccatttgtggttgaacatcttggccgaattattgagattcggtgacagagaattttacaaggattggtggaatgctaagaccgtcgaagaatattggagaatgtggaatatgccagttcacaagtggatggttagacatatctacttcccatgtttgagaaacggtattccaaaaggtgttgctatcgttattgccttcttggtttctgctgttttccacgaattgtgtattgctgttccatgccatattttcaagttgtgggctttcattggtatcatgttccaagttccattggtcttgattaccaactacttgcaagataagttcagatcctctatggtcggtaacatgattttctggttcatcttctccattttgggtcaacctatgtgtgtcttgttgtactaccatgatttgatgaacagaaagggtaaggccgattga

SEQ ID NO: 39 - DNA, 984 bp, Theobroma cacao
atgatgggtgaagaaatggaagaaagaaaagctaccggttacagagaattttccggtagacatgaattcccatctaacactatgcatgctttgttggctatgggtatttggttgggtgctattcattttaacgccttgttgttgttattctccttcttgttcttgccattctccaagttcttggttgttttcggtttgttgttgttgttcatgatcttgccaatcgacccatactctaagtttggtagaagattgtctagatatatctgcaagcacgcttgttcctactttccaattacattgcacgttgaagatatccatgctttccatccagatagagcttacgtttttggtttcgaaccacattccgttttgccaattggtgttgttgctttggctgatttgactggttttatgccattgccaaagattaaggttttggcttcttctgctgttttctacactccattcttgagacatatttggacatggttgggtttgactccagctactaagaagaatttctcctctttgttggatgctggttactcctgtattttggttccaggtggtgttcaagaaacttttcatatggaaccaggttccgaaattgctttcttgagagctagaagaggtttcgttagaattgctatggaaatgggttctcctttggttcctgttttttgtttcggtcaatcccatgtttacaaatggtggaaaccaggtggtaagttctacttgcaattttccagagctattaagttcaccccaatctttttctggggtatttttggttctccattgccatatcaacatccaatgcatgttgttgtcggtaagccaattgatgtcaagaaaaatccacaacctatcgtcgaagaagttatcgaagttcacgatagatttgtcgaagccttgcaagatttgttcgaaagacataaggctcaagttggttttgccgatttgccattgaagatcttgtga

SEQ ID NO: 40 - DNA, 1419 bp, Artificial Sequence
  synthetic polynucleotide
atgaccgacgtgtcgaccaccaaccagcgctacatgacccagaccgacttcatgtcgtggcgcatggaggaggacccgatcctgcgctcgaccatcgtggccgtggccctgctggaccgctcgccggaccagtcgcgcttcgtggacatgatgcgccgcgccgtggacctggtgccgctgttccgccgcaccgccatcgaggccccgatgggcttcgccccgccgcgctgggccgacgaccacgacttcgacctgtcgtggcacctgcgccgctacaccctgccggagccgcgcacctgggacggcgtgctggacttcgcccgcaccgccgagatgaccgccttcgacaagcgccgcccgctgtgggagttcaccgtgctggacggcctgcacgacggccgctcggccctggtgatgaaggtgcaccactcgctgaccgacggcgtgtcgggcatgcagatcgcccgcgagatcgtggacttcacccgcgacggcggcccgcgcccggaccgcaccgaccaccgcaccgccgccccgaacggcgagtcgccgaccccgccgggccgcctgtcgtggtaccgcaacaccgccaccgacgtggcccgccgcgcctcgaacaccctgggccgcaactcggtgcgcctggtgcgcaccccgcgcgccacctggcgcgacgccgccgccctggccggctcgaccctgcgcctgacccgcccggtggtgtcgaccctgtcgccggtgatgaagaagcgctcgacccgccgccactgcgccgtgctggacgtgccggtggaggccctggcccaggccgccgccgccggcgccggctcgatcaacgacgccttcctggccgccgtgctgctgggcatggccaagtaccaccgcctgcacggcgccgagatctcggagctgcgcatgaccctgccgatctcgctgcgcgccgagaccgacccggtgggcggcaaccgcatcaccctggcccgcttcgccctgccggccgacatcgacgacccggccgagctgatgcaccgcgtgcacgccaccgtggacgcctggcgccacgagccggccatcccgctgtcgccgaccatcgccggcgccctgaacctgctgccggcctcgaccctgggcaacatgctgaagcacgtggacttcgtggcctcgaacgtggtgggctcgccggtgccgctgttcatcgccggctcggaggtgctgcactactacgccttctcgccgaccctgggctcggccttcaacgtgaccctgatgtcgtacaccacccgctgctgcgtgggcatcaacgccgacaccgacgccatcccggacctggccaccctgaccgactcgatcgccgacggcttccgcgccgtgctgggcctgtgcaccaagaccaccgacacccgcgtggtggtggcctcg

SEQ ID NO: 41 - DNA, 1419 bp, Rhodococcus opacus
ttgaccgacgtgagcacgacgaatcagcgctacatgacccagacggacttcatgtcgtggcggatggaggaggacccgatcctccggtcgaccatcgtggcggtcgcactgctcgaccgcagtccggatcagagccgattcgtcgacatgatgcgcagggcggtcgacctggttcccctcttccggcggacggcgatcgaggctccgatgggctttgcgccgccgaggtgggcggacgatcacgatttcgatctcagctggcacctgcgccgctacaccctccccgaaccacgaacatgggacggggttctcgacttcgcccggaccgccgagatgaccgccttcgacaagcgccgtccgctctgggagttcaccgttctcgacggactccacgacggcaggtccgccctcgtgatgaaggtgcatcactctctcaccgacggtgtcagtggaatgcagattgcccgggagatcgtggatttcacccgcgatggcgggccacggccggaccggaccgaccaccgcacggccgcgccgaacggtgaatcgcccactccgccgggccgcctctcctggtaccggaacacggccaccgacgtggcccgccgggcatcgaacacgctgggccgcaacagtgttcgactggttcggactccgagggccacctggcgcgacgcagccgcactagccggctccacactgcgcctcacgcgtcccgtggtctccacactgtccccggtcatgaaaaaacgcagcacgcggcgtcactgtgcggtgctcgacgtgccggtggaggcgctcgctcaggcggccgcggcgggtgccggttcgatcaacgacgcgtttctcgctgctgtcctgctgggaatggcgaagtaccaccgactgcacggcgccgagatcagcgaactgcggatgacgctgccgatcagcctgcgtgccgagacggacccggtgggaggcaaccgcatcaccctggcacgttttgcactacccgccgacatcgacgaccccgccgagttgatgcaccgggtgcacgccacggtggatgcctggcgccacgagcccgcgattccgctgtcaccgacgatcgccggcgccttgaacctgcttccggcctcgacactcggaaacatgctcaaacacgtcgacttcgtcgcctcgaacgtcgtcggatctccggtaccactgttcatcgccgggtcggaggttctgcactactacgcgttcagccccacactcggatcggcgttcaacgtcaccttgatgtcctacaccacgcgatgctgtgtcgggatcaacgccgacacggacgcaatcccggacctcgcaaccctgaccgattcgatagcggacgggtttcgcgccgtcctcggactgtgcacgaagacaaccgatacgagggtggtcgtggcctcg

SEQ ID NO: 42 - DNA, 1359 bp, Artificial Sequence
  synthetic polynucleotide
atgccggtgaccgactcgatcttcctgctgggcgagtcgcgcgagcacccgatgcacgtgggctcgctggagctgttcaccccgccggaggacgccggcccggactacgtgaagtcgatgcacgagaccctgctgaagcacaccgacgtggacccgaccttccgcaagaagccggccggcccggtgggctcgctgggcaacctgtggtgggccgacgagtcggacgtggacctggagtaccacgtgcgccactcggccctgccggccccgtaccgcgtgcgcgagctgctgaccctgacctcgcgcctgcacggcaccctgctggaccgccaccgcccgctgtgggagatgtacctgatcgagggcctgtcggacggccgcttcgccatctacaccaagctgcaccactcgctgatggacggcgtgtcgggcctgcgcctgctgatgcgcaccctgtcgaccgacccggacgtgcgcgacgccccgccgccgtggaacctgccgcgccgcgcctcggccaacggcgccgccccggccccggacctgtggtcggtggtgaacggcgtgcgccgcaccgtgggcgaggtggccggcctggccccggcctcgctgcgcatcgcccgcaccgccatgggccagcacgacatgcgcttcccgtacgaggccccgcgcaccatgctgaacgtgccgatcggcggcgcccgccgcttcgccgcccagtcgtggccgctggagcgcgtgcacgccgtgcgcaaggccgccggcgtgtcggtgaacgacgtggtgatggccatgtgcgccggcgccctgcgcggctacctggaggagcagaaggccctgccggacgagccgctgatcgccatggtgccggtgtcgctgcgcgacgagcagaaggccgacgccggcggcaacgccgtgggcgtgaccctgtgcaacctggccaccgacgtggacgacccggccgagcgcctgaccgccatctcggcctcgatgtcgcagggcaaggagctgttcggctcgctgacctcgatgcaggccctggcctggtcggccttcaacatgtcgccgatcgccctgaccccggtgccgggcttcgtgcgcttcaccccgccgccgttcaacgtgatcatctcgaacgtgccgggcccgcgcaagaccatgtactggaacggctcgcgcctggacggcatctacccgacctcggtggtgctggacggccaggccctgaacatcaccctgaccaccaacggcggcaacctggacttcggcgtgatcggctgccgccgctcggtgccgtcgctgcagcgcatcctgttctacctggagaccgccctgggcgagctggaggccgccctgctg

SEQ ID NO: 43 - DNA, 1362 bp, Rhodococcus opacus
atgccggttaccgattcgatattccttctcggcgaatcgcgagagcatccgatgcacgtgggatcgctcgaattgttcacaccgccggaggatgccggccccgactacgtgaagtcgatgcacgaaactctgctgaagcacacggacgtcgaccccactttccgcaagaagccagcgggccccgtgggcagtctcgggaatctgtggtgggccgacgagtcggacgtcgatctcgaataccacgtgcgtcattcggccctgccggccccgtaccgggtccgggaactgctgacgctgacgtcccggttgcacggcacgctcctggaccgtcatcgcccgctgtgggagatgtatctgatcgaggggctcagcgacggccggttcgcgatctacaccaagctgcaccattcgctgatggacggggtctccggtttgcggctgctgatgcggacgctgtcgaccgacccggacgtgcgcgacgcaccgccgccgtggaacctgccgcgcagggcgtcggccaatggtgccgcccccgctcccgacctctggtcggtggtgaacggggtccgtcgcacggtcggtgaggtggccggtctcgcgccggcgtcgctgcgcatcgcccgcaccgcgatggggcagcacgacatgaggtttccgtacgaggcgcctcgcaccatgctgaacgtcccgatcgggggcgcgcgccggttcgccgcgcagtcctggccgctcgaacgcgtccacgccgtgcggaaggcagccggggtcagtgtcaacgacgtcgtgatggccatgtgcgccggggcgttgcggggttacctcgaggaacagaaggcgctaccggacgagccgctgatcgcgatggttccggtgtccctgcgcgacgagcagaaggccgacgccggcggcaacgcggtcggggtcacgttgtgcaacctggcgaccgacgtcgacgacccggccgaacgtctgacggcgatctccgcctccatgtcccaggggaaggaactgttcggcagcctcacctcgatgcaggcgctggcgtggtccgcgttcaacatgtcgccgatcgccctgacgcccgtgcccgggttcgtccgcttcacaccgccgccgttcaacgtgatcatctccaacgtcccgggaccgcggaagaccatgtactggaacgggtcccggctggacggcatctacccgacatcggtggtgctggacgggcaggcactcaacatcacactcaccaccaacggcggcaacctcgatttcggtgtcatcgggtgccgccgctcggtgccgagcctgcaacgcatcctcttctatctcgagacggctctgggcgaactcgaggcggcattgctctga

SEQ ID NO: 44 - DNA, 1389 bp, Artificial Sequence
  synthetic polynucleotide
atgccgctgccgatgtcgccgctggactcgatgttcctgctgggcgagtcgcgcgagcacccgatgcacgtgggctgcgtggagatcttccagctgccggagggcgccgacacctacgacatgcgcgccatgctggaccgcgccctggccgacggcgacggcatcgtgaccccgcgcctggccaagcgcgcccaccgctcgttctcgaccctgggccagtggtcgtgggagaccgtggacgacatcgacctgggccaccacatccgccacgacgccctgccggccccgggcggcgaggccgagctgatggccctgtgctcgcgcctgcacggctcgctgctggaccgctcgcgcccgctgtgggagatgcacctgatcgagggcctgtcggacggccgcttcgccgtgtacaccaagatccaccacgccgtggccgacggcgtgaccgccatgaagatgctgcgcaacgccttctcggagaactcggaggaccgcgacgtgccggccccgtggcagccgcgcggcccgcgccgccagcgcaccccgtcgaaggccttctcgctgtcgggcctggccggctcgaccttccgcgccgcccgcgacaccgtgggcgaggtggccggcctggtgccggccctggccggcaccgtgtcgcgcgccttccgcgaccagggcggcccgctggccctgtcggccccgaagaccccgttcaacgtgccgatcaccggcgcctgccagttcgccgcccagtcgtggccgctggagcgcctgcgcctggtggccaagctgtcggacaccgccatcaacgacgtggtgctggccatgtcgtcgggcgccctgcgctcgtacctggaggaccagaacgccctgccggccgagccgctgatcgccatggtgccggtgtcgctgaagtcgcagcgcgaggcctcgaacggcaacaacatcggcgtgctgatgtgcaacctgggcacccacctgccggacctggccgaccgcctggacaccatccgcacctcgatgcgcgagggcaaggaggcctacgagaccctgtcggccacccagatcctggccatgtcggccctgggcgccgccccgatcggcgcctcgatgctgttcggccacaactcgcgcgtgcgcccgccgttcaacctgatcatctcgaacgtgccgggcccgtcgtcgccgctgtactggaacggcgcccgcctggacgccatctacccgctgtcggtgccggtggacggccagggcctgaacatcacctgcacctcgaacgacgacatcatctcgttcggcgtgaccggctgccgctcggccgtgccggacctgaagtcgatcccggcccgcctgggccacgagctgcgcgccctggagcgcgccgtgggcatc

SEQ ID NO: 45 - DNA, 1392 bp, Rhodococcus opacus
atgccgctccccatgtcccctctcgactcgatgttccttctcggagagtcccgtgagcacccgatgcatgttggctgcgtagagatcttccagctcccggaaggcgccgacacctacgacatgcgcgcgatgctcgaccgcgcgcttgccgacggcgacgggatcgtcacgccccggctcgccaagcgagcccaccggtccttctcgacgctcggtcagtggagctgggagaccgtcgacgacatcgacctcggtcaccacatccggcacgatgcgctgcccgcccccgggggcgaggccgagctgatggcactgtgctcgcggctgcacggatcgctgctcgaccgcagccgcccgctgtgggagatgcacctgatcgagggactgagcgacggacggttcgccgtctacaccaagatccaccacgcggtcgccgacggcgtcaccgcgatgaagatgctgcgcaacgcgttcagcgagaactccgaggaccgggacgtgccggccccgtggcagccgcggggaccgcggcgacagcggacgccgtcgaaggcgttcagcctgtcgggactggccggttccacgttccgcgccgcccgcgacaccgtcggtgaggtcgccgggctcgtgcccgcgctcgctggcaccgtgtcccgcgccttccgcgaccagggcggcccgctcgccttgtccgcacccaagaccccgttcaacgtgccgatcaccggtgcctgccagttcgcggcgcagtcgtggccgctcgaacgtctccggctcgtcgccaagctgtccgacaccgccatcaacgacgtcgtgctcgcgatgtcctcgggagcactccgcagctatctcgaggatcagaacgccctgcccgccgagccgctgatcgcgatggtgccggtgtcgctgaagagtcagcgcgaggcgtcgaacggcaacaacatcggggtgctcatgtgcaacctcggcacccacctccccgacctggcggatcgcctcgacaccatccggacgtcgatgcgcgagggcaaggaggcgtacgagacgctgagtgcgacgcagatcctcgcgatgagcgctctcggcgcggcaccgatcggcgcgagcatgctgttcgggcacaactcgcgggtgcgcccgccgttcaacctcatcatctccaatgttccgggtcccagctcgccgctgtattggaacggggcacggctcgatgccatctacccgctgtcggtgcccgtcgacggccagggcctgaacatcacctgcaccagcaacgacgacatcatctcgttcggggtcaccggctgccgcagcgcggtgcccgacctgaagtcgatccccgcccggctggggcacgaactgcgcgccctcgagcgcgcggtcggaatctga

SEQ ID NO: 46 - protein, 501 aa, Theobroma cacao
MAISDSPEILGSTATVTSSSHSDSDLNLLSIRRRTSTTAAGRAPDRDDSGNGEAVDDRDQVESANLMSNVAENANEMPNSSDTRFTYRPRVPAHRRIKESPLSSGAIFKQSHAGLFNLCIVVLVAVNSRLIIENLMKYGWLIRSGFWFSSRSLSDWPLFMCCLTLPIFPLAAFVVEKLVQRNYISEPVVVFLHAIISTTAVLYPVIVNLRCDSAFLSGVALMLFACIVWLKLVSYAHTNNDMRALAKSAEKGDVDPSYDVSFKSLAYFMVAPTLCYQQSYPRTPAVRKSWVVRQFIKLIVFTGLMGFIIEQYINPIVQNSQHPLKGNLLYAIERVLKLSVPNLYVWLCMFYCFFHLWLNILAELLRFGDREFYKDWWNAKTVEEYWRMWNMPVHKWMVRHIYFPCLRNGIPKGVAIVIAFLVSAVFHELCIAVPCHIFKLWAFIGIMFQVPLVLITNYLQDKFRSSMVGNMIFWFIFSILGQPMCVLLYYHDLMNRKGKAD

SEQ ID NO: 47 - protein, 443 aa, Artificial Sequence
  synthetic polypeptide
MQVESANLMSNVAENANEMPNSSDTRFTYRPRVPAHRRIKESPLSSGAIFKQSHAGLFNLCIVVLVAVNSRLIIENLMKYGWLIRSGFWFSSRSLSDWPLFMCCLTLPIFPLAAFVVEKLVQRNYISEPVVVFLHAIISTTAVLYPVIVNLRCDSAFLSGVALMLFACIVWLKLVSYAHTNNDMRALAKSAEKGDVDPSYDVSFKSLAYFMVAPTLCYQQSYPRTPAVRKSWVVRQFIKLIVFTGLMGFIIEQYINPIVQNSQHPLKGNLLYAIERVLKLSVPNLYVWLCMFYCFFHLWLNILAELLRFGDREFYKDWWNAKTVEEYWRMWNMPVHKWMVRHIYFPCLRNGIPKGVAIVIAFLVSAVFHELCIAVPCHIFKLWAFIGIMFQVPLVLITNYLQDKFRSSMVGNMIFWFIFSILGQPMCVLLYYHDLMNRKGKAD

SEQ ID NO: 48 - protein, 327 aa, Theobroma cacao
MMGEEMEERKATGYREFSGRHEFPSNTMHALLAMGIWLGAIHFNALLLLFSFLFLPFSKFLVVFGLLLLFMILPIDPYSKFGRRLSRYICKHACSYFPITLHVEDIHAFHPDRAYVFGFEPHSVLPIGVVALADLTGFMPLPKIKVLASSAVFYTPFLRHIWTWLGLTPATKKNFSSLLDAGYSCILVPGGVQETFHMEPGSEIAFLRARRGFVRIAMEMGSPLVPVFCFGQSHVYKWWKPGGKFYLQFSRAIKFTPIFFWGIFGSPLPYQHPMHVVVGKPIDVKKNPQPIVEEVIEVHDRFVEALQDLFERHKAQVGFADLPLKIL

SEQ ID NO: 49 - protein, 473 aa, Rhodococcus opacus
MTDVSTTNQRYMTQTDFMSWRMEEDPILRSTIVAVALLDRSPDQSRFVDMMRRAVDLVPLFRRTAIEAPMGFAPPRWADDHDFDLSWHLRRYTLPEPRTWDGVLDFARTAEMTAFDKRRPLWEFTVLDGLHDGRSALVMKVHHSLTDGVSGMQIAREIVDFTRDGGPRPDRTDHRTAAPNGESPTPPGRLSWYRNTATDVARRASNTLGRNSVRLVRTPRATWRDAAALAGSTLRLTRPVVSTLSPVMKKRSTRRHCAVLDVPVEALAQAAAAGAGSINDAFLAAVLLGMAKYHRLHGAEISELRMTLPISLRAETDPVGGNRITLARFALPADIDDPAELMHRVHATVDAWRHEPAIPLSPTIAGALNLLPASTLGNMLKHVDFVASNVVGSPVPLFIAGSEVLHYYAFSPTLGSAFNVTLMSYTTRCCVGINADTDAIPDLATLTDSIADGFRAVLGLCTKTTDTRVVVAS

SEQ ID NO: 50 - protein, 453 aa, Rhodococcus opacus
MPVTDSIFLLGESREHPMHVGSLELFTPPEDAGPDYVKSMHETLLKHTDVDPTFRKKPAGPVGSLGNLWWADESDVDLEYHVRHSALPAPYRVRELLTLTSRLHGTLLDRHRPLWEMYLIEGLSDGRFAIYTKLHHSLMDGVSGLRLLMRTLSTDPDVRDAPPPWNLPRRASANGAAPAPDLWSVVNGVRRTVGEVAGLAPASLRIARTAMGQHDMRFPYEAPRTMLNVPIGGARRFAAQSWPLERVHAVRKAAGVSVNDVVMAMCAGALRGYLEEQKALPDEPLIAMVPVSLRDEQKADAGGNAVGVTLCNLATDVDDPAERLTAISASMSQGKELFGSLTSMQALAWSAFNMSPIALTPVPGFVRFTPPPFNVIISNVPGPRKTMYWNGSRLDGIYPTSVVLDGQALNITLTTNGGNLDFGVIGCRRSVPSLQRILFYLETALGELEAALL

SEQ ID NO: 51 - protein, 463 aa, Rhodococcus opacus
MPLPMSPLDSMFLLGESREHPMHVGCVEIFQLPEGADTYDMRAMLDRALADGDGIVTPRLAKRAHRSFSTLGQWSWETVDDIDLGHHIRHDALPAPGGEAELMALCSRLHGSLLDRSRPLWEMHLIEGLSDGRFAVYTKIHHAVADGVTAMKMLRNAFSENSEDRDVPAPWQPRGPRRQRTPSKAFSLSGLAGSTFRAARDTVGEVAGLVPALAGTVSRAFRDQGGPLALSAPKTPFNVPITGACQFAAQSWPLERLRLVAKLSDTAINDVVLAMSSGALRSYLEDQNALPAEPLIAMVPVSLKSQREASNGNNIGVLMCNLGTHLPDLADRLDTIRTSMREGKEAYETLSATQILAMSALGAAPIGASMLFGHNSRVRPPFNLIISNVPGPSSPLYWNGARLDAIYPLSVPVDGQGLNITCTSNDDIISFGVTGCRSAVPDLKSIPARLGHELRALERAVGI

SEQ ID NO: 52 - DNA, 1080 bp, Theobroma cacao
atggagctctcttccctcccttccgtttcttctttctcgctatgtcacccaaagcccaggagttctgctacgttgcctttcttgcctttttcgaatcgtaaaggagcttactttggctattctctgtgtaaacgggcaactttgagaaattcatgtaactctgctcaaaataactttttgcgtatttcaaggacgcatgatggtgtatctcggtgttattttaatcaaaaggaaggtttaaatagatcgtattacaatagtaaattacataacgagaacaaattgtccagatacatagttgcgagatctgaatttgctgggactgggacccctgatgctgcctattctttatcagaaattaaaccgggttcaaaagttagaggagtatgcttttatgctgttacagcaatagcagccattttacttatttggtttatgctggttttgcatccctttgtgctcttgtttgatcgctacagaagaaaagctcagcattttattgccaaactttgggctatggcaacagttgctcctttttttaaaattgagtttgaaggattggagaatctgcctccacaggatgttcctgccgtatatgtttccaaccaccagagttttttagacatctatacacttctaactcttggaagaagcttcaagttcatcagcaagacggggatatttctttatcccattattgggtgggccatgtctatgatgggtctaattccattaaagcgcatggacagcagaagccagttggactgtcttaagagatgtatggatctcatcaggaacggtgcctctgtctttttcttcccagaaggcacacggagtaaggatgggaagctaggtgctttcaagaaaggtgcatttagtgttgcagcaaaaactggagtgcctgtcgttcctatgaccctaattggcacaggcaaaatcatgcctttaggactggagggtgtcataaattcaggatccgtaaaagttgttattcacaagccgatcaaaggaagtgatccagaaatattatgcaatgaagctagaaacacaatagcagatacgcttaagcatcaatgctga

SEQ ID NO: 53 - DNA, 933 bp, Artificial Sequence
  synthetic polynucleotide
atggagtcgtcgggctcgggctcgttcctgcgcaaccgccgcctgggctcgttcctggacaccaactcggacccgaacgtgcgcgagacccagaaggtgctgtcgaagggcggcgcccgccagcgcccgaagaccgacgacgccttcgtggacgacgacggctggatctgctcgctgatctcgtgcgtgcgcatcgtggcctgcttcctgaccatgatggtgaccaccttcatctgggccctgatcatgctgctgctgctgccgtggccgtcgcagcgcatccgccagggcaacatctacggccacgtgaccggccgcctgctgatgtggatcctgggcaacccgatcaagatcgagggcaccgagttctcgaacgagcgcgccatctacatctgcaaccacgcctcgccgatcgacatcttcctgatcatgtggctgaccccgaccggcaccgtgggcatcgccaagaaggagatcatctggtacccgctgttcggccagctgtacgtgctggccaaccacctgcgcatcgaccgctcgaacccgtcgaccgccatccagtcgatgaaggaggccgtgcaggccgtgatcaagcacaacctgtcgctgatcatcttcccggagggcacccgctcgaagaacggccgcctgctgccgttcaagaagggcttcgtgcacctggccctgcagtcgcacatcccgatcgtgccgatcgtgctgaccggcacccacctggcctggcgcaagggctcgctgcacgtgcgcccggccccgatctcggtgaagtacctgccgccgatctcgaccgactcgtggaaggacgacaagatcgacgactacatcaagatggtgcacgacatctacgtggagaacctgccggagccgcagaagccgatcgtgtcggaggacaccaccaactcgtcgcgctcgtaa

SEQ ID NO: 54 - DNA, 933 bp, Theobroma cacao
atggagagttctggaagtggttctttcttgaggaacagaagattagggagcttcctcgatacaaattctgatccaaatgtgagagaaactcagaaagttttgagcaaaggaggagcaagacagaggcctaagactgatgatgcttttgttgatgatgacggatggatttgttcattgatatcttgtgtaaggattgttgcatgtttcctgacaatgatggtcacaacattcatttgggcattgatcatgctcttgctccttccttggccttcccagcgaatcaggcaaggaaatatttatggccatgtgactggtagattgctgatgtggatcttaggaaatcctataaagattgaaggaacagagttctccaatgagagagccatttatatctgcaatcatgcatctcccatagacattttcctcattatgtggttgactccaacagggactgttggcattgcaaagaaagagatcatttggtatcccctatttggacaactatatgttctagcgaatcatctccgcattgatcgatctaaccctagtacagccattcagtccatgaaagaggcagttcaggctgtgataaaacacaacctatctttgattatttttcctgagggcacaaggtcaaaaaatggacgattgctcccctttaaaaagggctttgttcatttggccttgcagtcacacattccaatagttccaatagtcttgacaggtactcatctagcatggaggaaaggtagcttgcatgttcgaccggctcctatatctgtaaaatatctccctccgataagtaccgatagttggaaagatgacaagattgatgactacataaaaatggtgcatgacatatatgtcgaaaacctccctgagcctcaaaagcctattgtatcagaagacaccactaacagttcaagatcataa

SEQ ID NO: 55 - DNA, 798 bp, Artificial Sequence
  synthetic polynucleotide
gacgacgccttcgtggacgacgacggctggatctgctcgctgatctcgtgcgtgcgcatcgtggcctgcttcctgaccatgatggtgaccaccttcatctgggccctgatcatgctgctgctgctgccgtggccgtcgcagcgcatccgccagggcaacatctacggccacgtgaccggccgcctgctgatgtggatcctgggcaacccgatcaagatcgagggcaccgagttctcgaacgagcgcgccatctacatctgcaaccacgcctcgccgatcgacatcttcctgatcatgtggctgaccccgaccggcaccgtgggcatcgccaagaaggagatcatctggtacccgctgttcggccagctgtacgtgctggccaaccacctgcgcatcgaccgctcgaacccgtcgaccgccatccagtcgatgaaggaggccgtgcaggccgtgatcaagcacaacctgtcgctgatcatcttcccggagggcacccgctcgaagaacggccgcctgctgccgttcaagaagggcttcgtgcacctggccctgcagtcgcacatcccgatcgtgccgatcgtgctgaccggcacccacctggcctggcgcaagggctcgctgcacgtgcgcccggccccgatctcggtgaagtacctgccgccgatctcgaccgactcgtggaaggacgacaagatcgacgactacatcaagatggtgcacgacatctacgtggagaacctgccggagccgcagaagccgatcgtgtcggaggacaccaccaactcgtcgcgctcgtaa

SEQ ID NO: 56 - DNA, 798 bp, Artificial Sequence
  synthetic polynucleotide
gatgatgcttttgttgatgatgacggatggatttgttcattgatatcttgtgtaaggattgttgcatgtttcctgacaatgatggtcacaacattcatttgggcattgatcatgctcttgctccttccttggccttcccagcgaatcaggcaaggaaatatttatggccatgtgactggtagattgctgatgtggatcttaggaaatcctataaagattgaaggaacagagttctccaatgagagagccatttatatctgcaatcatgcatctcccatagacattttcctcattatgtggttgactccaacagggactgttggcattgcaaagaaagagatcatttggtatcccctatttggacaactatatgttctagcgaatcatctccgcattgatcgatctaaccctagtacagccattcagtccatgaaagaggcagttcaggctgtgataaaacacaacctatctttgattatttttcctgagggcacaaggtcaaaaaatggacgattgctcccctttaaaaagggctttgttcatttggccttgcagtcacacattccaatagttccaatagtcttgacaggtactcatctagcatggaggaaaggtagcttgcatgttcgaccggctcctatatctgtaaaatatctccctccgataagtaccgatagttggaaagatgacaagattgatgactacataaaaatggtgcatgacatatatgtcgaaaacctccctgagcctcaaaagcctattgtatcagaagacaccactaacagttcaagatcataa

SEQ ID NO: 57 - DNA, 1146 bp, Theobroma cacao
atggaagtttgcaggcccctcaaacctgatgataaattaaagcaccgccctttgactccttttaggtttttaaggggtctgatatgtttagtggtgtttctcttgactgcttttatgtttctagcgtatttaggacctggggctgtcctattgcgatttttcagcctacactactgtaggaaggcaacatccttcttctttggcctatggctagctttgtggccctttctttttgaaaaaataaacaggactaaagtggttttctctggggataatgctccacagaaggaacgtgttttacttattgtcaatcacaggactgaagttgattggatgtacctctgggatcttgcaatgcgaaagggctgcctgggctacatcaaatatattcttaagagcagcctgatgaaactacctgtccttggttggggatttcacatcttggagttcatttcagtagataggaagtgggaaactgatgaaaatgtcctgcgccaaatgctttcaacctttaagaatcctcgagatcctttatggcttgctcttttccctgaaggaaccgattttaccgaagaaaaatgcaggaacagtcagaagtttgcagctgaagttggattgcctgtgttgacaaatgtgctgctaccgagaacaagggggttttgcctttgcttagaaacacttagggactctttggatgcagtttacgatttgagtattgcatataagcaccaatgccccttctttctggacaatgtttttggtgtggatccatcagaggttcacattcatgttcgacgtatcccagttaaggagatcccaacatctaatgcagaggctgctgcttggttaattgatacattcaagctcaaggaccagttgctctcagatttcaaatctcagggacattttcctaaccaaggaactcaacaagaactttcttctttgaagtccttattaaatctaacagtgataatatccttgacagccatattcacttatcttaccttttcttccaatttgtacatgatatatgtaagcttagcttgtctataccttgcttacattactcattataaaattcgcccaatgccagttctaagctctgtaaaaccgctgtcttacccaaagggcaagagagatgaataa

SEQ ID NO: 58 - DNA, 1119 bp, Theobroma cacao
atggaagttcctagtgcaaatcatgaaatgaggcatcgttcattgaccccgctaagggtgtttaggggtctaatatgtttgctagtgctgttttcaacagcttttatgatgatagtgtattgtggctttcttaccactgttatattcaggcttttcagcatacattacagtcggaaagcaacttctttcttctttagtgcttggctgtctttatggccctttttatttgagaaaataaacaaaacaaaagtcattttttctggagatgatgttcctccaagggaacgcgttttgcttatttgcaaccacagaaccgaggttgactggatgtacttgtgggactttgcattgcggaaaggttgcctgggatacataaagtatatccttaagagcagcttgatgaaattacctgtatttggttgggctttccatattttagagttcatccctgtggaaaggaagtgggaggttgatgaatctaacatgcgcaacatgctttcaacattcaaagatcctcaagatcctctctggcttgttctctttcccgaaggaacagatttcactgagcaaaaatgcttaagaagtcaaaaatatgcagctgaaaatggcttacctatcctaaagaatttgctgcttccaaaatcaaagggttttttcgcctgcttggaagatttgaggagctctttggatgcagtttatgatgtgaccattggatataagcattgctgcccatccttcttggacaatgtcttcggggtagacccttctgaagttcatattcacatcagacgcattaccctggatgacattccaatatctgaaagggagttaaccgcttggttaatggatacatttcaacataaagatcaattgctttctaatttcaagtctgaaggttatttccctcggcaaggaccggaagtaaacctctctgcagtgaagtgcattgtagacgttgtgctggtgcttttcttgactagtgcattcatatttttcacctttttctcatccatttggtttaagatatttgtatctttatcttgtgcctatatgacttctgcaacttatttaaacacccgtccagtaccagtcttcagccttgtgaaaacttgtgtctaa

SEQ ID NO: 59 - protein, 359 aa, Theobroma cacao
MELSSLPSVSSFSLCHPKPRSSATLPFLPFSNRKGAYFGYSLCKRATLRNSCNSAQNNFLRISRTHDGVSRCYFNQKEGLNRSYYNSKLHNENKLSRYIVARSEFAGTGTPDAAYSLSEIKPGSKVRGVCFYAVTAIAAILLIWFMLVLHPFVLLFDRYRRKAQHFIAKLWAMATVAPFFKIEFEGLENLPPQDVPAVYVSNHQSFLDIYTLLTLGRSFKFISKTGIFLYPIIGWAMSMMGLIPLKRMDSRSQLDCLKRCMDLIRNGASVFFFPEGTRSKDGKLGAFKKGAFSVAAKTGVPVVPMTLIGTGKIMPLGLEGVINSGSVKVVIHKPIKGSDPEILCNEARNTIADTLKHQC

SEQ ID NO: 60 - protein, 310 aa, Theobroma cacao
MESSGSGSFLRNRRLGSFLDTNSDPNVRETQKVLSKGGARQRPKTDDAFVDDDGWICSLISCVRIVACFLTMMVTTFIWALIMLLLLPWPSQRIRQGNIYGHVTGRLLMWILGNPIKIEGTEFSNERAIYICNHASPIDIFLIMWLTPTGTVGIAKKEIIWYPLFGQLYVLANHLRIDRSNPSTAIQSMKEAVQAVIKHNLSLIIFPEGTRSKNGRLLPFKKGFVHLALQSHIPIVPIVLTGTHLAWRKGSLHVRPAPISVKYLPPISTDSWKDDKIDDYIKMVHDIYVENLPEPQKPIVSEDTTNSSRS

SEQ ID NO: 61 - protein, 266 aa, Artificial Sequence
  synthetic polypeptide
MDDAFVDDDGWICSLISCVRIVACFLTMMVTTFIWALIMLLLLPWPSQRIRQGNIYGHVTGRLLMWILGNPIKIEGTEFSNERAIYICNHASPIDIFLIMWLTPTGTVGIAKKEIIWYPLFGQLYVLANHLRIDRSNPSTAIQSMKEAVQAVIKHNLSLIIFPEGTRSKNGRLLPFKKGFVHLALQSHIPIVPIVLTGTHLAWRKGSLHVRPAPISVKYLPPISTDSWKDDKIDDYIKMVHDIYVENLPEPQKPIVSEDTTNSSRS

SEQ ID NO: 62 - protein, 381 aa, Theobroma cacao
MEVCRPLKPDDKLKHRPLTPFRFLRGLICLVVFLLTAFMFLAYLGPGAVLLRFFSLHYCRKATSFFFGLWLALWPFLFEKINRTKVVFSGDNAPQKERVLLIVNHRTEVDWMYLWDLAMRKGCLGYIKYILKSSLMKLPVLGWGFHILEFISVDRKWETDENVLRQMLSTFKNPRDPLWLALFPEGTDFTEEKCRNSQKFAAEVGLPVLTNVLLPRTRGFCLCLETLRDSLDAVYDLSIAYKHQCPFFLDNVFGVDPSEVHIHVRRIPVKEIPTSNAEAAAWLIDTFKLKDQLLSDFKSQGHFPNQGTQQELSSLKSLLNLTVIISLTAIFTYLTFSSNLYMIYVSLACLYLAYITHYKIRPMPVLSSVKPLSYPKGKRDE

SEQ ID NO: 63 - protein, 372 aa, Theobroma cacao
MEVPSANHEMRHRSLTPLRVFRGLICLLVLFSTAFMMIVYCGFLTTVIFRLFSIHYSRKATSFFFSAWLSLWPFLFEKINKTKVIFSGDDVPPRERVLLICNHRTEVDWMYLWDFALRKGCLGYIKYILKSSLMKLPVFGWAFHILEFIPVERKWEVDESNMRNMLSTFKDPQDPLWLVLFPEGTDFTEQKCLRSQKYAAENGLPILKNLLLPKSKGFFACLEDLRSSLDAVYDVTIGYKHCCPSFLDNVFGVDPSEVHIHIRRITLDDIPISERELTAWLMDTFQHKDQLLSNFKSEGYFPRQGPEVNLSAVKCIVDVVLVLFLTSAFIFFTFFSSIWFKIFVSLSCAYMTSATYLNTRPVPVFSLVKTCV

SEQ ID NO: 64 - DNA, 1503 bp, Durio zibethinus
atggctccactgaaagcggcgcaaagctttccatcgataacggaatgcgacggttctacgtacgaatcgatcgccgctgatctcgatggcacgcttttaatctctcgaagctcttttccttacttcatgctcgtcgccgtggaagcgggaagcctctttcgaggtcttatccttcttctctctctgcctctgataattatttcttatctcttcgtttccgaagctattggtatccaaatcctcatttttatctccttcgctggactcaagatccgcgacatcgagctcgtctcccgcgctgttcttcccagattttatgctgcgaatgtgaggaaggaaagtttcgaagtgtttgacagatgcaagaggaaggtggtggtgacggcgaatccgacattcatggttgagccgtttgtgaaggattttctaggtggagataaggttttgggcacggagattgaagtgaaccctaaaacaaagaaggccacggggtttgtcaagaagccaggggttttagtaggaaagttgaagagattggccattttcaaggagttcggtgatgaatcacctgatcttggaatcggagaccgtgaatctgatcacgatttcatgtcaatttgcaaggagggttacatggtgcaccctagtaagtcagcaacaccggtacaactggatcgtttaaaaagccgcatcatctttcatgatggtcgctttgttcagcgcccggacccactcaatgccttaatcacttatatttggctgccatttggcttcatcctatccatcattcgagtttacttcaatctacctttaccagagcgtattgtacgctacacatacgaaatgctgggtatccacctcgtgatccgtgggaagagacctcccccaccatctcctgggactccaggtaacctctacgtctgcaatcaccgttcagctcttgatccaattgtgatcgccatcgcactcggacgcaaagtttcgtgtgttacatacagcgtaagccgtctctcgaggttcctttccccaatcccagcgattgctttaactcgtgatcgtgcggctgatgctgcccgaatttcagaactattacaaaaaggtgatttagtggtgtgtccagaggggaccacgtgccgcgagcagttcttattacgattcagtgctttgtttgcagaaatgagcgataggatagtgcccgtggcggtcaattgcaggcaaaacatgttttatgggacgaccgtgagaggggtcaaattttgggacccttattttttcttcatgaatcctaggccaacatacgaggtcactttccttgatcgattgccggaggagatgacggtgaaggccggagggaaatcggctattgaggtggctaatcacgtgcagaaggtgctgggtgatgtcctggggtttgagtgcactggattgacaaggaaggataaatatatgttgcttgggggaaatgatggtaaggttgaatcgatttacaatgcaaagaaataa

SEQ ID NO: 65 - DNA, 1503 bp, Gossypium arboreum
atggctccaccgaaagcagggaaaacctttccatcgataacggaatgcgatggattgaagtatgaatcgatcgccgccgatctggatggcacgcttttaatctcacgaagctctttcccctacttcatgcttattgccgtcgaagcgggaagcctccttcgaggtcttatccttcttctttctctgcctctggtcatcatatcttatctcttcatttccgaagctattggtattcaaatcctcattttcatctccttcgctggactcaagatccgcgacatcgagctggtatctcgcgctgttcttcccagattctatgctgcaaatgtaaggaaggaaagtttcgaggtatttgacagatgcaagaggaaagtagtggtaacggcgaatccgacgttcatggtggagccgtttgtgaaggattttctcggcggagataaagttttaggcacagagattgaagtgaaccctaaaacaaagaaggcgacgggatttgtgaagaatcccggggttttagtagggaagtttaagagattagccattttgaaggagtttggtgatgaatcgcctgatcttggaatcggagaccgtgaatctgatcatgatttcatgtcaatttgcaaggaggggtacatggtgcaccctagcaaatcagcatcaccagtaccgcttgatcgcctaaagagccgcattatcttccacgatggtcgctttgtccaacgtccggatccactcaatgcctggctaacctacctttggctgccatttggcttcatcctctccattattcgtgtctacttcaatctacctttacccgagcgtatcgtacgctacacttacgagatgctcggcattcacctcgtgatccgcggaaagcgaccacccccaccatctgcggggaccccaggcaacctctacgtctgcaatcaccgcacagctcttgacccgattgtgatcgccatcgcacttggacgcaaagtctcgtgtgtcacatacagcgtaagccgtctctcgaggttcctatctccaatcccagccattgctttaactcgtgatcgtgcggccgatgctgcccgaatttcagaactgttgcaaaaaggtgatctagtagtttgtccggaggggaccacgtgtcgtgagcaattcttgttgaggttcagtgctttgttcgcagaaatgagcgataggatcgtccccgttgccgtcaattgcaagcagagcatgttctacgggacgaccgtgagaggggtcaaattctgggacccttatttcttcttcatgaatccaaggccaacatacgaggtcacgttccttgatcgattgccggaagagatgacagtgaaggccggagggaaatcggcgatcgaggtagcgaatcacgtgcagaaggtgttgggtgatgtcctggggtttgaatgcactggattgactaggaaagataaatacatgttgcttggaggaaacgatggtaaggtagaatcgatgtacaatggcaagaaataa

SEQ ID NO: 66 - DNA, 1503 bp, Hibiscus syriacus
atgactccactaagagctgggagaaggtttccttcaataacggaatgcaacggatcgacatacgaatcgatcgccgccgatctcgatggcacgcttttaatctcacgtagctcttttccgtatttcatgctcattgccgtcgaagcgggaagcctccttcgaggtcttatccttcttctttcactgcctcttgtcattgtatcttatctcttcatttccgaagctattggtatacaaatcctcattttcatctccttcgctggactcaagatccgcgacatcgagttggtctcccgcgctattcttcccagattttatgctgcgaatgtgaggaaggaaagtttcgaagtatttgacagatgcaagaggaaagtggtagtgacggcgaatccgacgttcatggtggagccgtttgtgaaggattttcttggtggagataaggttttaggcacagagatcgaagtgaaccctaaaacaaagaaggccacgggatttgttaagaagccaggcgttttagtaagcgaattgaagagattggccattctgaaggagtttggtgacgattcgcccgatcttggaatcggagaccgtgaatctgatcacgatttcatgtcaatttgcaaggagggctacatggtgcaccctagcaaatcagcatcaccggtaccacttgatcgcctgagaagccgcatcatcttccacgatggtcggtttgttcaacgcccggacccactcaatgccttgatcacctacatttggctgccattcggcttcatcttgtccatcattcgcgtctacttcaatctacctttacccgagcgcatcgtacgctacacatacgaaatgttgggcattcacctcgtgatccgcgggaagcgaccgtcgccgccgtctccgggtaccccaggcaacctctacgtctgtaatcaccgttcagcacttgatccaatcgtcatcgccatcgctctcggacgtaaagtttcgtgtgtcacatacagtgtaagccgtctctcgaggttcctatcaccaatcccagccatcgctttaactcgtgatcgtgcggctgatgccgcccgaatttcagagctattacaaaaaggtgatctagtagtttgtccggaggggaccacgtgtcgcgagccattcctgttgcgattcagtgctttgttcgcagaaatgagcgatagaatcgtccccgtcgccgtcaattgcaagcagagcatgttctacgggacgaccgtgagaggggtaaaattctgggacccttactttttcttcatgaacccaaggccaacatacgaggtcacgttcctcgaccggttgccggaagagatgacggtgaaggccggaggaaaatcggctatcgaggtggcgaatcacgtgcagaaggtgttgggcgatgtactgggctttgaatgcacgggattgactaggaaagacaagtatatgttgctcggtggaaatgatggtaaggtcgaatccatgtacaatggcaagaaataa

SEQ ID NO: 67 - DNA, 1503 bp, Theobroma cacao
atggctccagcgaaatcggggcgaagctttccttcgataacgaaatgcgaaggttcgacgtacgaatcgatagccgccgacctcgacggcacgcttttaatctctcgaagctcttttccttacttcatgcttgtcgccgtcgaagctggaagcctccttcgaggtcttatccttcttctctctctgcctctggtcattgtttcttatctcttcatttccgaagctattggcatccaaatcctcatatttatctccttcgctggactcaagatccgcgacatcgagctcgtctcccgtgctgttcttcccaggttttatgctgcgaatgtgaggaaggaaagtttcgaggtgtttgacagatgtaagaggaaagtggtggtgacggcgaatccgacgttcatggtggagccgtttgtgaaggattttctaggtggagacaaggttttaggcacagaaattgaagtgaaccccaaaacaaagaaggccacggggtttgtcaagaagcctggggttttagtctcggagttgaagagactggccattttgaaggagtttggagaggaatcacctgatcttggaatcggagaccgtgaatctgatcatgatttcatgtcaatttgcaaggagggctacatggtgcaccctagcaaatcagcaacaccagtacctcttgatcgcctaaagagccgcatcatcttccatgatggtcgctttgtccagcgcccggacccgctcaatgccttgatcacctatttgtggctgccatttggcttcatcctatccatcgttcgcgtttacttcaatctgcctttaccagagcgtatcgtacgctacacatacgaaatgctgggcatccacctcgtgatccgcgggaagcgtcctccccctccatcccctgggaccccaggcaacctctacgtctgcaatcaccgttcagctcttgatccaatcgtgattgccatcgcactcggtcgcaaagtttcgtgtgtcacgtacagcgtaagccgcctctcgaggttcctgtctccaatcccagccgtcgctttaactcgtgatcgtgcggctgatgctgctagaatttcagaactattgcaaaaaggtgatctagttgtgtgcccagaggggaccacgtgccgcgagcagttcctgttgcgattcagtgctttgttcgcagagttgagcgataggatcgtgcccgtggcggtcaattgcaggcagaacatgttttatggtacgaccgtgagaggggtcaaattttgggacccttattttttcttcatgaatcctaggccaacgtacgaggtcactttccttgatcgtttgccggaggagatgacggttaaggccggagggaaatcgtcgattgaggtggccaatcacgtgcagaaggtgctgggcgatgtcctggggtttgagtgcactggattgactaggaaggataaatatctgttgcttggaggaaacgacggtaaggttgaatcaatgtacaatgccaagaaataa

SEQ ID NO: 68 - protein, 420 aa, Theobroma cacao
MVATAASSAFFPVTSSPDSSDSKNKKLGSGSTNLGGIKSKPSTPSGILQVKANAQAPPKINGTTVVTTPVESFKNEDTASSPPPRTFINQLPDWSMLLAAITTIFLAAEKQWMMLDWKPRRPDMLIDPFGIGRIVQDGLVFRQNFSIRSYEIGADRTASIETLMNHLQETAINHCRSAGLLGEGFGSTPEMCKKNLIWVVTRMQVVVDRYPTWGDVVQVDTWASASGKNGMRRDWLVSDSKTGEILTRASSVWVMMNKLTRRLSKIPEEVRGEIEPYFMNSDPVVAEDSRKLVKLDDSTADYVRKGLTPRWGDLDVNQHVNNVKYIGWILESAPLPILETHELSSMTLEYRRECGRDGVLQSLTAVSDSGVGNLVNFGEIECQHLLRLEDGSEIVRGRTEWRPKYAKSFGNVGQLPAESA

SEQ ID NO: 69 - DNA, 1251 bp, Theobroma cacao
atgaagaaggaaaagctgtttggttttatcaatataaagaaaaaagaaaaagaagaaaaaaaaccaaaaccaaacaaaaatcccaacagccaaaccaaatcagaaagcgaaagggaaggcatgagtagcagaggagggaagctgagctcatccagctccgaattggacttggatggacctaacatcgaagattatctcccgtccggatcctccattaacgaacctcgcgggaagcttcgcctacgcgatttgcttgatatttctcccactttaactgaagctgctggtgccattgttgatgattcattcacacgatgttttaagtcgaatccccctgaaccatggaactggaatgtatatttgttcccactgtggtgttttggtgtggcagttcggtacttgattttattccctgcgagggttgtggtgttgacaataggatggataatatttctttcatccttcattcccgtacactttctactcaaagggcatgataagttgcggaaaaagatggagagggttttggtggagctaatgtgcagcttctttgttgcatcgtggactggagttgtgaagtaccatggaccacggcctagcattcggcccaagcaggtgtttgtggccaatcatacttctatgatcgatttcatcatattagaacagatgactgcatttgctgtcattatgcagaagcaccctggatgggttggactgttgcagagcactattttagagagtgtggggtgtatttggttcaaccgttcagaggcaaaagatcgggaaattgtagcaaagaagttaagggatcatgttcagggggttgacaataacccgcttctcatttttcctgaagggacctgcataaacaatcagtacagtgtcatgtttaagaagggtgcatttgaactcggttgcacagtttgtccgattgcaatcaagtacaataaaatttttgttgatgcgttttggaatagccggaagcagtccttcacgatgcatctgttgcagcttatgacatcctgggctgttgtttgtgatgtgtggtacctagaaccccaaaatctaaggcctggagaaacaccaattgaatttgcagagagggtcagagacataatatctgttcgagcaggtcttaaaaaggttccatgggatggatatttgaagtactctcgccctagccctaagcatagagagcgaaagcaacaaagctttgctgagtccgtgcttctgcgactggaggaaaagtga

SEQ ID NO: 70 - protein, 288 aa, Artificial Sequence
  synthetic polypeptide
MIVQDGLVFRQNFSIRSYEIGADRTASIETLMNHLQETAINHCRSAGLLGEGFGSTPEMCKKNLIWVVTRMQVVVDRYPTWGDVVQVDTWASASGKNGMRRDWLVSDSKTGEILTRASSVWVMMNKLTRRLSKIPEEVRGEIEPYFMNSDPVVAEDSRKLVKLDDSTADYVRKGLTPRWGDLDVNQHVNNVKYIGWILESAPLPILETHELSSMTLEYRRECGRDGVLQSLTAVSDSGVGNLVNFGEIECQHLLRLEDGSEIVRGRTEWRPKYAKSFGNVGQLPAESA

SEQ ID NO: 71 - DNA, 1044 bp, Artificial Sequence
  synthetic polynucleotide
atgtccattaacgaacctcgcgggaagcttcgcctacgcgatttgcttgatatttctcccactttaactgaagctgctggtgccattgttgatgattcattcacacgatgttttaagtcgaatccccctgaaccatggaactggaatgtatatttgttcccactgtggtgttttggtgtggcagttcggtacttgattttattccctgcgagggttgtggtgttgacaataggatggataatatttctttcatccttcattcccgtacactttctactcaaagggcatgataagttgcggaaaaagatggagagggttttggtggagctaatgtgcagcttctttgttgcatcgtggactggagttgtgaagtaccatggaccacggcctagcattcggcccaagcaggtgtttgtggccaatcatacttctatgatcgatttcatcatattagaacagatgactgcatttgctgtcattatgcagaagcaccctggatgggttggactgttgcagagcactattttagagagtgtggggtgtatttggttcaaccgttcagaggcaaaagatcgggaaattgtagcaaagaagttaagggatcatgttcagggggttgacaataacccgcttctcatttttcctgaagggacctgcataaacaatcagtacagtgtcatgtttaagaagggtgcatttgaactcggttgcacagtttgtccgattgcaatcaagtacaataaaatttttgttgatgcgttttggaatagccggaagcagtccttcacgatgcatctgttgcagcttatgacatcctgggctgttgtttgtgatgtgtggtacctagaaccccaaaatctaaggcctggagaaacaccaattgaatttgcagagagggtcagagacataatatctgttcgagcaggtcttaaaaaggttccatgggatggatatttgaagtactctcgccctagccctaagcatagagagcgaaagcaacaaagctttgctgagtccgtgcttctgcgactggaggaaaagtga

SEQ ID NO: 72 - DNA, 1623 bp, Artificial Sequence
  synthetic polynucleotide
atggtgttcccggtggtgttcctgaagctggccgactgggtgctgtaccagctgctggccaactcgtgctaccgcgccgcccgcaagatgcgcaactacggcttcttcctgcgcaaccagaccctgcgctcgccgccgcagcagcaggccgcctcgctgttcccgtcggtgaccaagtgcgacgtgggcaactcgcgccgcttcgacaccctggtgtgcgacatccacggcgtgctgctgggctcggacaccttcttcccgtacttcatgctggtggccttcgagggcggctcgatcgtgcgcgccttcctgctgctgctgtcgtgctcgttcctgtgggtgctggactcggagctgaagctgcgcatcatgatcttcatctcgttctgcggcctgcgcaagaaggacatcgagtcggtgggccgcgccgtgctgccgaagttctacctggagaacctgaacctgcaggtgtacgaggtgtggtcgaagacctcgtcgcgcgtggtgttcacctcgatcccgcgcgtgatggtggagggcttcctgcacgagtacatgtcggcctcgggcgtggtgggcaccgagctgcacaccgtgggcaaccgcttcaccggcctgctgtcgtcgtcgggcctgctggtgaagcacaacgccctgaaggagcacttcggcgacaagaagccggacgtgggcctgggctcgtcgtcgctgcacgaccagtacttcatctcgctgtgcaaggaggcctacgtggtgaacatggaggacggcaagtcgaacctgtcgtcgttcatgccgcgcgacaagtacccgaagccgctgatcttccacgacggccgcctggccttcctgccgaccccgttcgccaccctgtcgatgttcctgtggctgccgttcggcatcgtgctgtcgatcctgcgcatcttcgtgggcatctgcctgccgtacaagctggccgtgatctgcgccaccctgtcgggcgtgcagctgaagttccagggctgcttcccgtcgtcgaactcgcagcacaagaagggcgtgctgtacgtgtgcacccaccgcaccctgctggacccggtgttcctgtcgaccgccctgtgcaagccgctgaccgccgtgacctactcgctgtcgaagatgtcggagctgatcgccccgatcaagaccgtgcgcctgacccgcgaccgcaagcaggacggcgagaccatgcagaagctgctgtcggagggcgacctggtggtgtgcccggagggcaccacctgccgcgagccgtacctgctgcgcttctcgtcgctgttcgccgagctggccgacgagatcgtgccggtggccatcaacgcccacgtgtcgatgttctacggcaccaccgcctcgggcctgaagtgcctggacccgatcttcttcctgatgaacccgcgcccgtcgtaccacgtgcagatcctgggcaaggtgccgcaggagttcacctgcgccggcggccgctcgtcgctggaggtggccaactacatccagcgcaagctggccgacgccctgggcttcgagtgcaccaccctgacccgccgcgacaagtacctgatgctggccggcaacgagggcgtggtgcacgagaacaagcgcaactaa

SEQ ID NO: 73 - DNA, 1623 bp, Theobroma cacao
atggttttccctgtggtatttctgaagctagcagactgggtcttgtaccagctgctggccaactcatgttatagagctgcaaggaagatgagaaactacgggttctttctaaggaaccaaactcttaggtcaccaccacagcaacaagctgcttctttgttccctagtgttaccaagtgtgatgtaggcaatagtagaaggtttgatacattggtctgtgatatccatggagtcttgttaggatcagacacattttttccttacttcatgctagttgcttttgaaggtggtagcattgtgagagcctttctgttgcttttatcatgctcctttttgtgggtattggactcagagctcaagttgaggattatgatttttatttccttttgtgggcttaggaagaaggacatcgagagtgttggcagggctgttttgccaaagttttatctcgagaatctaaatctccaagtctatgaagtttggtctaaaacaagttcaagggttgtctttacaagtatacctagagtaatggtggaaggatttctccacgaatacatgagtgctagtggtgttgtaggcaccgaattgcacactgttgggaaccgattcacaggtttgttgtccagctccgggttgcttgtaaagcataatgctttaaaggaacacttcggagataaaaagcctgatgttggcctcggaagttcaagcctccatgaccaatactttatctccctttgcaaggaagcctatgtggtgaacatggaagatggcaaaagcaatctaagcagtttcatgccaagggacaagtacccgaagcctcttatatttcatgatgggaggctagctttcttgccaactccatttgcaactctttcaatgttcctgtggcttccatttggaatagttctttccattcttaggattttcgttggtatctgcctgccttacaagctagctgttatctgcgctactctgagtggtgtacagctgaaattccaagggtgcttcccttcatcaaattcacaacataaaaaaggggttctttatgtttgtacccatagaactcttctggacccagttttccttagcacagcattatgcaagcctttgactgcagttacttatagtctaagcaaaatgtctgaactgatagctcccatcaaaacagttaggttgacaagggacaggaaacaagatggagaaaccatgcaaaaattgctcagtgaaggtgatttggtagtgtgcccggaaggaaccacatgcagagagccttacttgttaaggtttagctcattgtttgctgagctagccgatgagatagtcccagtggccataaacgcacatgtaagcatgttttacgggacaactgcaagtgggttaaaatgcttggatcctatcttctttctgatgaaccccagacctagctaccatgttcaaatccttgggaaggtgcctcaagagtttacatgtgcagggggcaggtctagccttgaagtggcaaattatattcagagaaagctggctgatgctctgggatttgagtgcactacccttacaaggagagacaagtacttgatgctagcaggcaatgaaggggttgtccatgaaaataagagaaattaa

SEQ ID NO: 74 - DNA, 1566 bp, Artificial Sequence
  synthetic polynucleotide
atgaactcgtgctaccgcgccgcccgcaagatgcgcaactacggcttcttcctgcgcaaccagaccctgcgctcgccgccgcagcagcaggccgcctcgctgttcccgtcggtgaccaagtgcgacgtgggcaactcgcgccgcttcgacaccctggtgtgcgacatccacggcgtgctgctgggctcggacaccttcttcccgtacttcatgctggtggccttcgagggcggctcgatcgtgcgcgccttcctgctgctgctgtcgtgctcgttcctgtgggtgctggactcggagctgaagctgcgcatcatgatcttcatctcgttctgcggcctgcgcaagaaggacatcgagtcggtgggccgcgccgtgctgccgaagttctacctggagaacctgaacctgcaggtgtacgaggtgtggtcgaagacctcgtcgcgcgtggtgttcacctcgatcccgcgcgtgatggtggagggcttcctgcacgagtacatgtcggcctcgggcgtggtgggcaccgagctgcacaccgtgggcaaccgcttcaccggcctgctgtcgtcgtcgggcctgctggtgaagcacaacgccctgaaggagcacttcggcgacaagaagccggacgtgggcctgggctcgtcgtcgctgcacgaccagtacttcatctcgctgtgcaaggaggcctacgtggtgaacatggaggacggcaagtcgaacctgtcgtcgttcatgccgcgcgacaagtacccgaagccgctgatcttccacgacggccgcctggccttcctgccgaccccgttcgccaccctgtcgatgttcctgtggctgccgttcggcatcgtgctgtcgatcctgcgcatcttcgtgggcatctgcctgccgtacaagctggccgtgatctgcgccaccctgtcgggcgtgcagctgaagttccagggctgcttcccgtcgtcgaactcgcagcacaagaagggcgtgctgtacgtgtgcacccaccgcaccctgctggacccggtgttcctgtcgaccgccctgtgcaagccgctgaccgccgtgacctactcgctgtcgaagatgtcggagctgatcgccccgatcaagaccgtgcgcctgacccgcgaccgcaagcaggacggcgagaccatgcagaagctgctgtcggagggcgacctggtggtgtgcccggagggcaccacctgccgcgagccgtacctgctgcgcttctcgtcgctgttcgccgagctggccgacgagatcgtgccggtggccatcaacgcccacgtgtcgatgttctacggcaccaccgcctcgggcctgaagtgcctggacccgatcttcttcctgatgaacccgcgcccgtcgtaccacgtgcagatcctgggcaaggtgccgcaggagttcacctgcgccggcggccgctcgtcgctggaggtggccaactacatccagcgcaagctggccgacgccctgggcttcgagtgcaccaccctgacccgccgcgacaagtacctgatgctggccggcaacgagggcgtggtgcacgagaacaagcgcaactaa

SEQ ID NO: 75 - DNA, 1566 bp, Theobroma cacao
atgaactcatgttatagagctgcaaggaagatgagaaactacgggttctttctaaggaaccaaactcttaggtcaccaccacagcaacaagctgcttctttgttccctagtgttaccaagtgtgatgtaggcaatagtagaaggtttgatacattggtctgtgatatccatggagtcttgttaggatcagacacattttttccttacttcatgctagttgcttttgaaggtggtagcattgtgagagcctttctgttgcttttatcatgctcctttttgtgggtattggactcagagctcaagttgaggattatgatttttatttccttttgtgggcttaggaagaaggacatcgagagtgttggcagggctgttttgccaaagttttatctcgagaatctaaatctccaagtctatgaagtttggtctaaaacaagttcaagggttgtctttacaagtatacctagagtaatggtggaaggatttctccacgaatacatgagtgctagtggtgttgtaggcaccgaattgcacactgttgggaaccgattcacaggtttgttgtccagctccgggttgcttgtaaagcataatgctttaaaggaacacttcggagataaaaagcctgatgttggcctcggaagttcaagcctccatgaccaatactttatctccctttgcaaggaagcctatgtggtgaacatggaagatggcaaaagcaatctaagcagtttcatgccaagggacaagtacccgaagcctcttatatttcatgatgggaggctagctttcttgccaactccatttgcaactctttcaatgttcctgtggcttccatttggaatagttctttccattcttaggattttcgttggtatctgcctgccttacaagctagctgttatctgcgctactctgagtggtgtacagctgaaattccaagggtgcttcccttcatcaaattcacaacataaaaaaggggttctttatgtttgtacccatagaactcttctggacccagttttccttagcacagcattatgcaagcctttgactgcagttacttatagtctaagcaaaatgtctgaactgatagctcccatcaaaacagttaggttgacaagggacaggaaacaagatggagaaaccatgcaaaaattgctcagtgaaggtgatttggtagtgtgcccggaaggaaccacatgcagagagccttacttgttaaggtttagctcattgtttgctgagctagccgatgagatagtcccagtggccataaacgcacatgtaagcatgttttacgggacaactgcaagtgggttaaaatgcttggatcctatcttctttctgatgaaccccagacctagctaccatgttcaaatccttgggaaggtgcctcaagagtttacatgtgcagggggcaggtctagccttgaagtggcaaattatattcagagaaagctggctgatgctctgggatttgagtgcactacccttacaaggagagacaagtacttgatgctagcaggcaatgaaggggttgtccatgaaaataagagaaattaa

SEQ ID NO: 76 - DNA, 1614 bp, Artificial Sequence
  synthetic polynucleotide
atggccaagctgtcgatggagttctcgttcttccagaccctgttcttcctgttctgccgcgtggtgttccgccagtcgaagaaccacaagtcgctgcaccgcaacgtgtcgaacatccacgccaacgagggcaagtaccacaagtacccgtcgttcgtgcaccgctcgaacctgtcgaaccagaccctggtgttctcggtggaggaggccctgctgaagtcgtcgtcgctgttcccgtacttcatgctggtggccttcgaggccggcggcctgctgcgcgccttcatcctgttcgtgctgtacccgatcctgtgcctggtgtcggaggagatgggcctgaagatcatggtgctggtgtgcttcttcggcatcaagaagaagtcgttccgcgtgggctcggccgtgctgccgaagttcttcctggaggacgtgggcctggagccgttcgagatgctgaagaagggcggcaagaaggtggccgtgtcgaagatcccgcaggtgatgatcgagtcgttcctgaaggactacctggagatcgacttcgtggtgggccgcgagctgaaggagttctgcggctacttcctgggcgtgatggaggagaagaagcgctcgaaggccgccctggacgagatcatcggctcggagtcgatgggcttcgacgtgatcggcatctcgggcctgaagaagtcgctggactaccacttcttctcgcactgcaaggagatctaccaggtgcgcaaggccgacaagcgcaactggcgccacgtgccgcgccaggagtactcgaagccgctgatcttccacgacggccgcctggccctgcgcccgaccctggtggcctcgctgaccatgttcgtgtggttcccgttcggcctggccctgtcgatcctgcgcgccgtggtgggcctgatgctgccgtacaagatctcgatcccgctgctggcctactcgggcctgcacctgttcctgtcgaccccggagtcgtcgctgcacccgctgtcgctgccgaactcgaagaagcagaacccgaagggccgcctgtacgtgtgcaaccaccgcaccctgctggacccggtgtacctgtcgttcgccctgcagaaggacctgaccgccgtgacctactcgctgtcgcgcatctcggagctgctggccccgatcaagaccgtgcgcctggcccgcgaccgcgaccaggacggcaagatgatggagaagatgctgaacctgggcgacctggtggtgtgcccggagggcaccacctgccgcgagccgtacctgctgcgcttctcgccgctgttctcggagatgtcggacgacatcgtgccggtggccatggactcgaacgtgtcgctgttctacggcaccaccgcctcgggcctgaagtgcctggacccgctgttcttcctgatgaacccgcgcccgatctacaccgtgcagatcctggacggcgtgtcgggcctgtacacctgccacgacggccagcgctcgcgcttcaaggtggccaaccaggtgcagaacgagatcggcaaggccctgggcttcgagtgcaccaagctgacccgccgcgacaagtacctgatcatggccggcaacgagggcatcatctcgcagacctaa

SEQ ID NO: 77 - DNA, 1614 bp, Theobroma cacao
atggctaaactttctatggagttttcctttttccaaacccttttcttcttattctgtcgagttgtctttagacagtctaagaatcacaaatctctccacagaaatgtcagcaatatccatgcaaacgagggtaaatatcataagtatccttcttttgttcatagatcaaacctgtcaaaccaaactctggtcttcagtgtagaagaggctttgttgaaatcctcatcattgtttccttacttcatgcttgtagcctttgaagctggagggctcttgagggcctttattctttttgttttatacccaattctatgcttagttagcgaagagatggggttgaagataatggtcctggtttgcttctttgggattaagaaaaagagcttcagagttggaagtgctgttctgccgaagttcttcttggaggatgttggcttggaaccatttgagatgttgaagaaaggcgggaaaaaggtggctgtcagtaaaattcctcaagtgatgatcgagagtttcttgaaggattacctggaaattgattttgtagttggaagagagctgaaggagttctgtgggtactttttgggagtcatggaagagaagaagagaagtaaggctgctttggacgagataattggaagcgaaagtatgggctttgatgttattggcatcagtggcctcaagaaatctcttgactatcattttttttctcattgcaaggaaatataccaggtgagaaaagcagacaaaagaaactggcgacacgtcccaagacaggagtattccaaaccactcattttccatgacgggagactagctctcaggccaactctggtggcctccctgaccatgttcgtgtggttccccttcggcttggctctttccatactcagagctgttgtcggcttaatgcttccgtacaagatttccatccccttattagcctacagcgggttgcacctgtttctttcaaccccggaaagctccctgcatcctctttcacttccgaactcgaagaaacaaaacccaaaaggccgcctttatgtttgcaatcatagaacactgctggatcccgtctacctttcttttgcattacagaaagacctcactgctgttacttacagcttaagcaggatatcagagctgttagctccaatcaaaaccgttcgattagcaagggatcgtgatcaagatggtaaaatgatggaaaagatgctaaatttaggggacctagtcgtatgcccggaagggactacgtgtagggagccctatctcttaaggttcagccctttattttcagagatgagcgacgatatagtccctgttgcgatggacagcaatgtgagtttgttctatgggacaacagccagtggcctgaaatgtctggacccacttttctttctcatgaacccacgaccaatctacactgtccagatacttgatggtgtatctggcttgtacacttgtcatgatggtcaaagatcaaggtttaaagtggctaatcaggttcagaatgagattggtaaggctctgggttttgagtgcaccaagcttacgagaagagacaagtacctaatcatggctggcaacgaaggaataattagccaaacctaa

SEQ ID NO: 78 - DNA, 1545 bp, Artificial Sequence
  synthetic polynucleotide
atgcagtcgaagaaccacaagtcgctgcaccgcaacgtgtcgaacatccacgccaacgagggcaagtaccacaagtacccgtcgttcgtgcaccgctcgaacctgtcgaaccagaccctggtgttctcggtggaggaggccctgctgaagtcgtcgtcgctgttcccgtacttcatgctggtggccttcgaggccggcggcctgctgcgcgccttcatcctgttcgtgctgtacccgatcctgtgcctggtgtcggaggagatgggcctgaagatcatggtgctggtgtgcttcttcggcatcaagaagaagtcgttccgcgtgggctcggccgtgctgccgaagttcttcctggaggacgtgggcctggagccgttcgagatgctgaagaagggcggcaagaaggtggccgtgtcgaagatcccgcaggtgatgatcgagtcgttcctgaaggactacctggagatcgacttcgtggtgggccgcgagctgaaggagttctgcggctacttcctgggcgtgatggaggagaagaagcgctcgaaggccgccctggacgagatcatcggctcggagtcgatgggcttcgacgtgatcggcatctcgggcctgaagaagtcgctggactaccacttcttctcgcactgcaaggagatctaccaggtgcgcaaggccgacaagcgcaactggcgccacgtgccgcgccaggagtactcgaagccgctgatcttccacgacggccgcctggccctgcgcccgaccctggtggcctcgctgaccatgttcgtgtggttcccgttcggcctggccctgtcgatcctgcgcgccgtggtgggcctgatgctgccgtacaagatctcgatcccgctgctggcctactcgggcctgcacctgttcctgtcgaccccggagtcgtcgctgcacccgctgtcgctgccgaactcgaagaagcagaacccgaagggccgcctgtacgtgtgcaaccaccgcaccctgctggacccggtgtacctgtcgttcgccctgcagaaggacctgaccgccgtgacctactcgctgtcgcgcatctcggagctgctggccccgatcaagaccgtgcgcctggcccgcgaccgcgaccaggacggcaagatgatggagaagatgctgaacctgggcgacctggtggtgtgcccggagggcaccacctgccgcgagccgtacctgctgcgcttctcgccgctgttctcggagatgtcggacgacatcgtgccggtggccatggactcgaacgtgtcgctgttctacggcaccaccgcctcgggcctgaagtgcctggacccgctgttcttcctgatgaacccgcgcccgatctacaccgtgcagatcctggacggcgtgtcgggcctgtacacctgccacgacggccagcgctcgcgcttcaaggtggccaaccaggtgcagaacgagatcggcaaggccctgggcttcgagtgcaccaagctgacccgccgcgacaagtacctgatcatggccggcaacgagggcatcatctcgcagacctaa

SEQ ID NO: 79 - DNA, 1545 bp, Artificial Sequence
  synthetic polynucleotide
atgcagtctaagaatcacaaatctctccacagaaatgtcagcaatatccatgcaaacgagggtaaatatcataagtatccttcttttgttcatagatcaaacctgtcaaaccaaactctggtcttcagtgtagaagaggctttgttgaaatcctcatcattgtttccttacttcatgcttgtagcctttgaagctggagggctcttgagggcctttattctttttgttttatacccaattctatgcttagttagcgaagagatggggttgaagataatggtcctggtttgcttctttgggattaagaaaaagagcttcagagttggaagtgctgttctgccgaagttcttcttggaggatgttggcttggaaccatttgagatgttgaagaaaggcgggaaaaaggtggctgtcagtaaaattcctcaagtgatgatcgagagtttcttgaaggattacctggaaattgattttgtagttggaagagagctgaaggagttctgtgggtactttttgggagtcatggaagagaagaagagaagtaaggctgctttggacgagataattggaagcgaaagtatgggctttgatgttattggcatcagtggcctcaagaaatctcttgactatcattttttttctcattgcaaggaaatataccaggtgagaaaagcagacaaaagaaactggcgacacgtcccaagacaggagtattccaaaccactcattttccatgacgggagactagctctcaggccaactctggtggcctccctgaccatgttcgtgtggttccccttcggcttggctctttccatactcagagctgttgtcggcttaatgcttccgtacaagatttccatccccttattagcctacagcgggttgcacctgtttctttcaaccccggaaagctccctgcatcctctttcacttccgaactcgaagaaacaaaacccaaaaggccgcctttatgtttgcaatcatagaacactgctggatcccgtctacctttcttttgcattacagaaagacctcactgctgttacttacagcttaagcaggatatcagagctgttagctccaatcaaaaccgttcgattagcaagggatcgtgatcaagatggtaaaatgatggaaaagatgctaaatttaggggacctagtcgtatgcccggaagggactacgtgtagggagccctatctcttaaggttcagccctttattttcagagatgagcgacgatatagtccctgttgcgatggacagcaatgtgagtttgttctatgggacaacagccagtggcctgaaatgtctggacccacttttctttctcatgaacccacgaccaatctacactgtccagatacttgatggtgtatctggcttgtacacttgtcatgatggtcaaagatcaaggtttaaagtggctaatcaggttcagaatgagattggtaaggctctgggttttgagtgcaccaagcttacgagaagagacaagtacctaatcatggctggcaacgaaggaataattagccaaacctaa

SEQ ID NO: 80 - protein, 500 aa, Durio zibethinus
MAPLKAAQSFPSITECDGSTYESIAADLDGTLLISRSSFPYFMLVAVEAGSLFRGLILLLSLPLIIISYLFVSEAIGIQILIFISFAGLKIRDIELVSRAVLPRFYAANVRKESFEVFDRCKRKVVVTANPTFMVEPFVKDFLGGDKVLGTEIEVNPKTKKATGFVKKPGVLVGKLKRLAIFKEFGDESPDLGIGDRESDHDFMSICKEGYMVHPSKSATPVQLDRLKSRIIFHDGRFVQRPDPLNALITYIWLPFGFILSIIRVYFNLPLPERIVRYTYEMLGIHLVIRGKRPPPPSPGTPGNLYVCNHRSALDPIVIAIALGRKVSCVTYSVSRLSRFLSPIPAIALTRDRAADAARISELLQKGDLVVCPEGTTCREQFLLRFSALFAEMSDRIVPVAVNCRQNMFYGTTVRGVKFWDPYFFFMNPRPTYEVTFLDRLPEEMTVKAGGKSAIEVANHVQKVLGDVLGFECTGLTRKDKYMLLGGNDGKVESIYNAKK

SEQ ID NO: 81 - protein, 500 aa, Gossypium arboreum
MAPPKAGKTFPSITECDGLKYESIAADLDGTLLISRSSFPYFMLIAVEAGSLLRGLILLLSLPLVIISYLFISEAIGIQILIFISFAGLKIRDIELVSRAVLPRFYAANVRKESFEVFDRCKRKVVVTANPTFMVEPFVKDFLGGDKVLGTEIEVNPKTKKATGFVKNPGVLVGKFKRLAILKEFGDESPDLGIGDRESDHDFMSICKEGYMVHPSKSASPVPLDRLKSRIIFHDGRFVQRPDPLNAWLTYLWLPFGFILSIIRVYFNLPLPERIVRYTYEMLGIHLVIRGKRPPPPSAGTPGNLYVCNHRTALDPIVIAIALGRKVSCVTYSVSRLSRFLSPIPAIALTRDRAADAARISELLQKGDLVVCPEGTTCREQFLLRFSALFAEMSDRIVPVAVNCKQSMFYGTTVRGVKFWDPYFFFMNPRPTYEVTFLDRLPEEMTVKAGGKSAIEVANHVQKVLGDVLGFECTGLTRKDKYMLLGGNDGKVESMYNGKK

SEQ ID NO: 82 - protein, 500 aa, Hibiscus syriacus
MTPLRAGRRFPSITECNGSTYESIAADLDGTLLISRSSFPYFMLIAVEAGSLLRGLILLLSLPLVIVSYLFISEAIGIQILIFISFAGLKIRDIELVSRAILPRFYAANVRKESFEVFDRCKRKVVVTANPTFMVEPFVKDFLGGDKVLGTEIEVNPKTKKATGFVKKPGVLVSELKRLAILKEFGDDSPDLGIGDRESDHDFMSICKEGYMVHPSKSASPVPLDRLRSRIIFHDGRFVQRPDPLNALITYIWLPFGFILSIIRVYFNLPLPERIVRYTYEMLGIHLVIRGKRPSPPSPGTPGNLYVCNHRSALDPIVIAIALGRKVSCVTYSVSRLSRFLSPIPAIALTRDRAADAARISELLQKGDLVVCPEGTTCREPFLLRFSALFAEMSDRIVPVAVNCKQSMFYGTTVRGVKFWDPYFFFMNPRPTYEVTFLDRLPEEMTVKAGGKSAIEVANHVQKVLGDVLGFECTGLTRKDKYMLLGGNDGKVESMYNGKK

SEQ ID NO: 83 - protein, 500 aa, Theobroma cacao
MAPAKSGRSFPSITKCEGSTYESIAADLDGTLLISRSSFPYFMLVAVEAGSLLRGLILLLSLPLVIVSYLFISEAIGIQILIFISFAGLKIRDIELVSRAVLPRFYAANVRKESFEVFDRCKRKVVVTANPTFMVEPFVKDFLGGDKVLGTEIEVNPKTKKATGFVKKPGVLVSELKRLAILKEFGEESPDLGIGDRESDHDFMSICKEGYMVHPSKSATPVPLDRLKSRIIFHDGRFVQRPDPLNALITYLWLPFGFILSIVRVYFNLPLPERIVRYTYEMLGIHLVIRGKRPPPPSPGTPGNLYVCNHRSALDPIVIAIALGRKVSCVTYSVSRLSRFLSPIPAVALTRDRAADAARISELLQKGDLVVCPEGTTCREQFLLRFSALFAELSDRIVPVAVNCRQNMFYGTTVRGVKFWDPYFFFMNPRPTYEVTFLDRLPEEMTVKAGGKSSIEVANHVQKVLGDVLGFECTGLTRKDKYLLLGGNDGKVESMYNAKK

SEQ ID NO: 84 - protein, 416 aa, Theobroma cacao
MKKEKLFGFINIKKKEKEEKKPKPNKNPNSQTKSESEREGMSSRGGKLSSSSSELDLDGPNIEDYLPSGSSINEPRGKLRLRDLLDISPTLTEAAGAIVDDSFTRCFKSNPPEPWNWNVYLFPLWCFGVAVRYLILFPARVVVLTIGWIIFLSSFIPVHFLLKGHDKLRKKMERVLVELMCSFFVASWTGVVKYHGPRPSIRPKQVFVANHTSMIDFIILEQMTAFAVIMQKHPGWVGLLQSTILESVGCIWFNRSEAKDREIVAKKLRDHVQGVDNNPLLIFPEGTCINNQYSVMFKKGAFELGCTVCPIAIKYNKIFVDAFWNSRKQSFTMHLLQLMTSWAVVCDVWYLEPQNLRPGETPIEFAERVRDIISVRAGLKKVPWDGYLKYSRPSPKHRERKQQSFAESVLLRLEEK

SEQ ID NO: 85 - protein, 347 aa, Artificial Sequence
  synthetic polypeptide
MSINEPRGKLRLRDLLDISPTLTEAAGAIVDDSFTRCFKSNPPEPWNWNVYLFPLWCFGVAVRYLILFPARVVVLTIGWIIFLSSFIPVHFLLKGHDKLRKKMERVLVELMCSFFVASWTGVVKYHGPRPSIRPKQVFVANHTSMIDFIILEQMTAFAVIMQKHPGWVGLLQSTILESVGCIWFNRSEAKDREIVAKKLRDHVQGVDNNPLLIFPEGTCINNQYSVMFKKGAFELGCTVCPIAIKYNKIFVDAFWNSRKQSFTMHLLQLMTSWAVVCDVWYLEPQNLRPGETPIEFAERVRDIISVRAGLKKVPWDGYLKYSRPSPKHRERKQQSFAESVLLRLEEK

SEQ ID NO: 86 - protein, 540 aa, Theobroma cacao
MVFPVVFLKLADWVLYQLLANSCYRAARKMRNYGFFLRNQTLRSPPQQQAASLFPSVTKCDVGNSRRFDTLVCDIHGVLLGSDTFFPYFMLVAFEGGSIVRAFLLLLSCSFLWVLDSELKLRIMIFISFCGLRKKDIESVGRAVLPKFYLENLNLQVYEVWSKTSSRVVFTSIPRVMVEGFLHEYMSASGVVGTELHTVGNRFTGLLSSSGLLVKHNALKEHFGDKKPDVGLGSSSLHDQYFISLCKEAYVVNMEDGKSNLSSFMPRDKYPKPLIFHDGRLAFLPTPFATLSMFLWLPFGIVLSILRIFVGICLPYKLAVICATLSGVQLKFQGCFPSSNSQHKKGVLYVCTHRTLLDPVFLSTALCKPLTAVTYSLSKMSELIAPIKTVRLTRDRKQDGETMQKLLSEGDLVVCPEGTTCREPYLLRFSSLFAELADEIVPVAINAHVSMFYGTTASGLKCLDPIFFLMNPRPSYHVQILGKVPQEFTCAGGRSSLEVANYIQRKLADALGFECTTLTRRDKYLMLAGNEGVVHENKRN

SEQ ID NO: 87 - protein, 521 aa, Artificial Sequence
  synthetic polypeptide
MNSCYRAARKMRNYGFFLRNQTLRSPPQQQAASLFPSVTKCDVGNSRRFDTLVCDIHGVLLGSDTFFPYFMLVAFEGGSIVRAFLLLLSCSFLWVLDSELKLRIMIFISFCGLRKKDIESVGRAVLPKFYLENLNLQVYEVWSKTSSRVVFTSIPRVMVEGFLHEYMSASGVVGTELHTVGNRFTGLLSSSGLLVKHNALKEHFGDKKPDVGLGSSSLHDQYFISLCKEAYVVNMEDGKSNLSSFMPRDKYPKPLIFHDGRLAFLPTPFATLSMFLWLPFGIVLSILRIFVGICLPYKLAVICATLSGVQLKFQGCFPSSNSQHKKGVLYVCTHRTLLDPVFLSTALCKPLTAVTYSLSKMSELIAPIKTVRLTRDRKQDGETMQKLLSEGDLVVCPEGTTCREPYLLRFSSLFAELADEIVPVAINAHVSMFYGTTASGLKCLDPIFFLMNPRPSYHVQILGKVPQEFTCAGGRSSLEVANYIQRKLADALGFECTTLTRRDKYLMLAGNEGVVHENKRN

SEQ ID NO: 88 - protein, 537 aa, Theobroma cacao
MAKLSMEFSFFQTLFFLFCRVVFRQSKNHKSLHRNVSNIHANEGKYHKYPSFVHRSNLSNQTLVFSVEEALLKSSSLFPYFMLVAFEAGGLLRAFILFVLYPILCLVSEEMGLKIMVLVCFFGIKKKSFRVGSAVLPKFFLEDVGLEPFEMLKKGGKKVAVSKIPQVMIESFLKDYLEIDFVVGRELKEFCGYFLGVMEEKKRSKAALDEIIGSESMGFDVIGISGLKKSLDYHFFSHCKEIYQVRKADKRNWRHVPRQEYSKPLIFHDGRLALRPTLVASLTMFVWFPFGLALSILRAVVGLMLPYKISIPLLAYSGLHLFLSTPESSLHPLSLPNSKKQNPKGRLYVCNHRTLLDPVYLSFALQKDLTAVTYSLSRISELLAPIKTVRLARDRDQDGKMMEKMLNLGDLVVCPEGTTCREPYLLRFSPLFSEMSDDIVPVAMDSNVSLFYGTTASGLKCLDPLFFLMNPRPIYTVQILDGVSGLYTCHDGQRSRFKVANQVQNEIGKALGFECTKLTRRDKYLIMAGNEGIISQT

SEQ ID NO: 89 - protein, 514 aa, Artificial Sequence
  synthetic polypeptide
MQSKNHKSLHRNVSNIHANEGKYHKYPSFVHRSNLSNQTLVFSVEEALLKSSSLFPYFMLVAFEAGGLLRAFILFVLYPILCLVSEEMGLKIMVLVCFFGIKKKSFRVGSAVLPKFFLEDVGLEPFEMLKKGGKKVAVSKIPQVMIESFLKDYLEIDFVVGRELKEFCGYFLGVMEEKKRSKAALDEIIGSESMGFDVIGISGLKKSLDYHFFSHCKEIYQVRKADKRNWRHVPRQEYSKPLIFHDGRLALRPTLVASLTMFVWFPFGLALSILRAVVGLMLPYKISIPLLAYSGLHLFLSTPESSLHPLSLPNSKKQNPKGRLYVCNHRTLLDPVYLSFALQKDLTAVTYSLSRISELLAPIKTVRLARDRDQDGKMMEKMLNLGDLVVCPEGTTCREPYLLRFSPLFSEMSDDIVPVAMDSNVSLFYGTTASGLKCLDPLFFLMNPRPIYTVQILDGVSGLYTCHDGQRSRFKVANQVQNEIGKALGFECTKLTRRDKYLIMAGNEGIISQT

SEQ ID NO: 90 - DNA, 486 bp, Cupriavidus necator
atgcccaaaccccatccggaactgccgtccgacccgcctctgcagaggccgccgcaggcagtccagagcgctgactattcgatcgagcagaacccccacaaggccaaccgcggcctgacgcgcgcctggcatgcggccatcaattcgctgtcggggctgcgctatgcggtgctcgaggaaagcgcgttccgccaggagctgacgctggtggcaatcctggcgccgtgggcattcctgctgccggtggacgtggtcgagcgcatcctgctgctgggcacgctgctggtggtgctgatcgtcgagttgctcaattccagcgtcgaggcggcaatcgaccgcatttcgctggagcggcacagcctgtccaagcgtgccaaggatttcggcagcgccgcggtaatgctggcgctggtgctgtgcggcggcacctgggtcgccatcgccgggccgcacgtggtgcgctgggtgcggacgctggcgggctga

SEQ ID NO: 91 - protein, 161 aa, Cupriavidus necator
MPKPHPELPSDPPLQRPPQAVQSADYSIEQNPHKANRGLTRAWHAAINSLSGLRYAVLEESAFRQELTLVAILAPWAFLLPVDVVERILLLGTLLVVLIVELLNSSVEAAIDRISLERHSLSKRAKDFGSAAVMLALVLCGGTWVAIAGPHVVRWVRTLAG

SEQ ID NO: 92 - DNA, 1788 bp, Cupriavidus necator
atgggccagtacaccgcaccgttgcgcgacatgcagttcgtgctccatgaactgctcggcgccgaagccgaactcaaggcgatgccgccgcacgcggacatcgacgcggacaccatcaaccaggttatcgaggaagccggcaagttctgctcggacgtggtgttcccgctcaaccaggtgggcgaccgcgaaggctgcacctacgtcggcgacggcgtggtcaaggctcccaccggcttcaaggaagcctaccagcagtatgtcgaggccggctggccggcgctggcgtgcgatcccgagttcggcggccagggcctgccgatcgtgatcaacaatgtggtctacgagatgctgaactcggccggccaggcctggaccatgtacccgggcctgtcgcacggcgcgtacgaggcgctgcacgcgcacggcacgccagaactgcagcagacctacctgcccaagctggtctccggcgtgtggaccggcaccatgtgcctgaccgagccgcactgcggcaccgacctcggcatcctgcgttccaaggcagagccgcaggccgacggttcctacctgatctcgggcaccaagatcttcatctcggccggcgagcacgacatggccgagaacatcatccacctggtgctggcacgcctgccggacgcgccgggcggcaccaagggcatctcgctgttcgtggtgcccaagttcatccccgatgccaacggcaacccgggcgagcgcaacggcatcaagtgcggctcgatcgagcacaagatgggcatccacggcaacgccacctgcgtgatgaacctggacggcgcgcgcggctggatggtgggcgagcccaacaagggcctgaacgccatgttcgtgatgatgaacgccgcgcgcctgggcgtgggcgcgcagggcctggggctgaccgaagtggcgtaccagaactcgctggcctacgccaaggaccgcctgcagatgcgcgcgctgaccggcccgaaggcgccggacaagcccgccgacccgatcatcgtgcacccggacgtgcgccgcatgctgctgacgcagaaggcctacgccgaaggcggccgcgccttcagctactggaccgcgctgcagatcgaccgcgagttgtcgcaccctgacgaagccgtgcgcaagcaggccggcgacctggtcgcgctgctcacgccggtgatcaaggccttcctgaccgacaacgccttcacgtccaccaatgagggcatgcaggtgttcggcggccacggctatatcgccgagtggggcatggagcagtatgtgcgcgatgcgcgcatcaacatgatctacgaaggcaccaacaccatccaggcgctggacctgctgggccgcaagatcctgggcgacatgggcgccaggatgaaggccttcggcaagatcgtgcaggaattcgttgaagccgaaggcaccaacgaagccatgcaggagttcatcaacccgctcgccgacattggcgacaaggtgcagaagctcaccatggaaatcggcatgaaggcgatgggcaatgccgacgaggtgggcgctgccgcggtgccgtacctgcgcgtggtgggccacctggtgttctcatacttctgggcccgcatggccaagatcgcgctggagaaggaagcgagcggcgacaagttctacaccgccaagctggccacggcacgcttctactttgccaggctgctgccggaaaccgccgccgagatccgcaaggcgcgcgccggttcggccacgctgatggcgctggacgcagacctgttctga

SEQ ID NO: 93 - DNA, 1863 bp, Cupriavidus necator
atgtcgatgatcctatcccgccgcgacctgaatttcgtgctgtacgaatggctcaaggtcgacgagctcacgcgcatcccgcgctatgccgaccactcgcgcgagactttcgacgccgcgctggacacctgcgagaaaatcgccaccgacctgttcgcgccgcacaacaagaagaacgaccagcaagagccgcatttcgacggcgagaccgtcagcatcatccccgaggtcagcaccgcgctgaaggccttctgcgaggccggcttgatggccgccggccaggactatgaactgggcggcatgcaactgccggtggtggtcgagaaggctggctttgcctacttcaagggtgccaacgtcggcaccagctcgtacccgttcctgaccatcggcaacgccaacctgctgctgacgcacggcacgccggcgcaggtcgagaccttcgtcaagccggagatggacggtcgcttcttcggcaccatgtgcctgtccgagccgcaggcgggctcgtcgctgtcggacatcaccacgcgcgccgagtacgagggcgaatcgccgctgggcgcgcagtaccggctgcgcggcaacaagatgtggatctctgccggcgagcacgagctgtcggaaaatatcgtccacctggtgctggccaagatccccggcccggacggcaagctgatcccgggcgtgaagggcatctcgctcttcatcgtgcccaagtacctggtcaatgaagacggctcgctgggcgagcacaacgacgtggtgctggccggcctgaaccacaagatgggctaccgcggcaccaccaactgcctgctcaacttcggcgaaggcatgaagtaccggccgggcggcaaggccggcgcgatcggctacctggtgggcgagccgcacaagggcctggcctgcatgttccacatgatgaacgaggcgcgcatcggcgtgggcctgggcgcggtgatgctgggctataccggctacctgcacgcgctggactacgcgcgcaaccgcccgcaaggccgcgcggtcggccccggcggcaaggatgcggccagcccgcaggtgaagctggtcgagcacgccgatatccgccgcatgctgctggcgcagaagagctatgtcgaaggcggcctggcgctgaacctctattgcgcgcgcctggtcgacgaggaagaggccgctgcggccgccggcgaccaagccgcgcatgcgcgcctggcgctattgctcgatatcctgaccccgatcgccaagagctggccgtcgcaatggtgcctggaggccaacaacctggcgatccaggtgcatggcggctacggctatacgcgcgagtacaacgtcgagcagttctaccgcgacaaccgcctcaacccgatccacgaaggcacgcacggcatccagggcctggacctgctgggccgcaaggtggtgatgaaggacggcgccgccttcaagctgctgggcgagcgcgtgcaggacaccatcacccgcgcgctcgccgccggcaatgccgagttgtcgcagcaggcaggcgccctcggcaccgccaccaagcgcctggccgaggtcacgcaggcgctgtggagcgcgggcgaccccaacgtgacgctggccaatgcctcggtctacctggaggccttcgggcacgtggtggtggcgtggatctggctggaacaggcactgctggcgcaagccgcgctgccgcgcgcgaacggcaaggaagacgaggacttctaccgcggcaagctggccgcggcggcctacttcttccgctgggagctgcccaaggtcggcccgcagctggcgctgctcgagtcgctcgaccgcaccacgctcgacatgcaggacgcgtggttctga

SEQ ID NO: 94 - DNA, 2145 bp, Cupriavidus necator
atgcaagccccgattcagtaccacaagaccgacgacggcatcgtcacgctgacgttcgatgcgcctgagcaaagcgtcaataccatgaccgatgagatgcggcaatgtctggcggacatggtgagccggctggaagcggagaaggaagcggttagcggcgtcattcttacctcggccaaggagacgttctttgcgggaggcaatctcaatcgcctgtacaagctgcagccggcggatgcggctacgcagttcgatgcctcggagcgtgccaagtctgcgctgaggcggctcgaaacgctgggcaagccggtggtggcggcgctcaatggcacggcgctgggtggcggcttcgaaattgcgctggcctgccaccatcgcattgcgctggacaagcccaaagtgcaattcggcctgcccgaagcgacgctgggcctgatgccgggggcgggcggcgtcgtgcgtctgaatcggctgctggggcttgctgcgagccagccttatttgcaggacagcaagctcatgtcgccggcagaggcgaccaaggttgggctggtgcatgagcttgcggacacacccgcggcactgctggagaaggcacgagcatggatcgcggcccacccggaaagcaagcagccgtgggacaaggccggctacacgccgccgggaggctgggccgatgcgagtgaggcgcggcgctggatctccacggccgccgcgcaggtgcgcgccaagaccaaaggttgctaccctgcgccggaagccatcttgtgcgcttcggtcgaaggcatgcaggtggacttcgacaccgctagccgcattgagacgcgctacttcgtgaagcttgtgactggccaggttgcgaagaacatcatcagcaccttctggttccacgccaacggcatcaagtcaggcgcgcagcgtcctgcaggggtggccaagggcaagatcaagacggtgggcgtgctgggcgcagggatgatgggcaaggggattgcgtatgtggcggcctcgcgtggtatcgaggtgtgggtcaaggatgccacccttgcgcaggccgaaggggcacgtgccaatgcggaccaactgctggccaagcgtgaggagaagggggaaattgatgccgcgacccgccgacagattgtcgagcgcattcacgcgactgaccgctatgaggactttgcccatgtcgacctggtggtggaagccatcccagagaaccctgcgcttaaggcggagatcacccggcaggccgagcccgtgctcggagatggggcgatctgggcctccaacacctcgacgctgcccatcaccggcctggccaaggcatcgagccggcccgagcgcttcgtcgggctgcacttcttctcgccggtgcaccgcatgcagttggtggaagtgattaagggccagcagacctcgccggagaccctggcccatgcgctggacttcgtgatgcagcttggcaagacgccgatcgtcgtcaacgacaaccgcggcttctttaccagccgggtattcagtactttcacacgcgaagcagtggcgatgctgggtgaggggcaggacccggccgccatcgaggcggcggccatcctgtcagggttccctgccgggccgctggcggtgctggacgaggtcagcttgagcttgaactacaacaaccggctcgagacgctcagggcgcatgcggaggagggtcgtccgctgccgccacatccggccgacgcagtgatggagcgcatgctcaatgaattcggccgcaaggggcgtgccgcgggtggcggcttctacgattatccggccgacggcaagaaggtgttctggagcggtctggctaagcacttcctgcgcccggccgaacagattccacagcgtgacaaacaggatcggttgttgttctgcatggccctggagtcggtgcgtgtactgcaggatggcgtgctggacagcgcgggggacggcaacattggctcggtactggggattggcttcccgcgctggagcggcggcgtgttccagttcctgaaccagtatgggctggaaaaggccgtggcacgtgcggagtacctggccgagcattatggcgaacggttcacgccaccgcaattgctacgggaaaaggccaaacgagccgagccattctga

SEQ ID NO: 95 - protein, 595 aa, Cupriavidus necator
MGQYTAPLRDMQFVLHELLGAEAELKAMPPHADIDADTINQVIEEAGKFCSDVVFPLNQVGDREGCTYVGDGVVKAPTGFKEAYQQYVEAGWPALACDPEFGGQGLPIVINNVVYEMLNSAGQAWTMYPGLSHGAYEALHAHGTPELQQTYLPKLVSGVWTGTMCLTEPHCGTDLGILRSKAEPQADGSYLISGTKIFISAGEHDMAENIIHLVLARLPDAPGGTKGISLFVVPKFIPDANGNPGERNGIKCGSIEHKMGIHGNATCVMNLDGARGWMVGEPNKGLNAMFVMMNAARLGVGAQGLGLTEVAYQNSLAYAKDRLQMRALTGPKAPDKPADPIIVHPDVRRMLLTQKAYAEGGRAFSYWTALQIDRELSHPDEAVRKQAGDLVALLTPVIKAFLTDNAFTSTNEGMQVFGGHGYIAEWGMEQYVRDARINMIYEGTNTIQALDLLGRKILGDMGARMKAFGKIVQEFVEAEGTNEAMQEFINPLADIGDKVQKLTMEIGMKAMGNADEVGAAAVPYLRVVGHLVFSYFWARMAKIALEKEASGDKFYTAKLATARFYFARLLPETAAEIRKARAGSATLMALDADLF

SEQ ID NO: 96 - protein, 620 aa, Cupriavidus necator
MSMILSRRDLNFVLYEWLKVDELTRIPRYADHSRETFDAALDTCEKIATDLFAPHNKKNDQQEPHFDGETVSIIPEVSTALKAFCEAGLMAAGQDYELGGMQLPVVVEKAGFAYFKGANVGTSSYPFLTIGNANLLLTHGTPAQVETFVKPEMDGRFFGTMCLSEPQAGSSLSDITTRAEYEGESPLGAQYRLRGNKMWISAGEHELSENIVHLVLAKIPGPDGKLIPGVKGISLFIVPKYLVNEDGSLGEHNDVVLAGLNHKMGYRGTTNCLLNFGEGMKYRPGGKAGAIGYLVGEPHKGLACMFHMMNEARIGVGLGAVMLGYTGYLHALDYARNRPQGRAVGPGGKDAASPQVKLVEHADIRRMLLAQKSYVEGGLALNLYCARLVDEEEAAAAAGDQAAHARLALLLDILTPIAKSWPSQWCLEANNLAIQVHGGYGYTREYNVEQFYRDNRLNPIHEGTHGIQGLDLLGRKVVMKDGAAFKLLGERVQDTITRALAAGNAELSQQAGALGTATKRLAEVTQALWSAGDPNVTLANASVYLEAFGHVVVAWIWLEQALLAQAALPRANGKEDEDFYRGKLAAAAYFFRWELPKVGPQLALLESLDRTTLDMQDAWF

SEQ ID NO: 97 - protein, 714 aa, Cupriavidus necator
MQAPIQYHKTDDGIVTLTFDAPEQSVNTMTDEMRQCLADMVSRLEAEKEAVSGVILTSAKETFFAGGNLNRLYKLQPADAATQFDASERAKSALRRLETLGKPVVAALNGTALGGGFEIALACHHRIALDKPKVQFGLPEATLGLMPGAGGVVRLNRLLGLAASQPYLQDSKLMSPAEATKVGLVHELADTPAALLEKARAWIAAHPESKQPWDKAGYTPPGGWADASEARRWISTAAAQVRAKTKGCYPAPEAILCASVEGMQVDFDTASRIETRYFVKLVTGQVAKNIISTFWFHANGIKSGAQRPAGVAKGKIKTVGVLGAGMMGKGIAYVAASRGIEVWVKDATLAQAEGARANADQLLAKREEKGEIDAATRRQIVERIHATDRYEDFAHVDLVVEAIPENPALKAEITRQAEPVLGDGAIWASNTSTLPITGLAKASSRPERFVGLHFFSPVHRMQLVEVIKGQQTSPETLAHALDFVMQLGKTPIVVNDNRGFFTSRVFSTFTREAVAMLGEGQDPAAIEAAAILSGFPAGPLAVLDEVSLSLNYNNRLETLRAHAEEGRPLPPHPADAVMERMLNEFGRKGRAAGGGFYDYPADGKKVFWSGLAKHFLRPAEQIPQRDKQDRLLFCMALESVRVLQDGVLDSAGDGNIGSVLGIGFPRWSGGVFQFLNQYGLEKAVARAEYLAEHYGERFTPPQLLREKAKRAEPF

SEQ ID NO: 98 - DNA, 762 bp, Limosilactobacillus reuteri
atggagttaaagcaagaagcacaaacattcgaaatgccgcacttactaacatattatgagtgtgatgaaacaagtcatccaagcctaagcatgatattaagtatgatttccatggtatccgatgagcatagtatgtctttaggaatgggcaccaaagaaatacaatctactggcggtacatgggtagtaagcggctatgaaggacatctttccgcaaagcaaccttcttttggcgaaacagttattttaggaacaaaagctgtttcctataaccgcttttttgctgttcgtgatttttggataacgggtaaagaacaccagattgaatatgcacgaattaggtcgatttttgtgtttatgaatctaaagacgcggcgaatgcaatcaattccaccagctttaattgaaccgtttaatgctcccgttgcgaaaagaattcctcgtctaaagcgacctcaaaaattggatgaaaatgcttcggtaataaagaagaattatcaagttcgttattttgaccttgatgctaatcaccatgttaataatgctcgttactttgattggcttcttgatcctcttggccgtgattttttacgcggaaatcagataaagagatttaatttgcagtatcttcaagaggtacgcaatggagaaatggtagaaagtaaagttaataagcttcaaacaaatgatgaaaaggtaacttatcatcagattggtgttggtgagcaaattgatgcaattgctgagatagaatggtattaa

SEQ ID NO: 99 - DNA, 1245 bp, Artificial Sequence
  synthetic polynucleotide
atggtggccaccgccgccacctcgtcgttcttcccggtgacctcgcgcaccggcggcgagggcggcggcggcatcccggcctcgctgggcggcggcctgaagcagaaccaccgctcgtcgtcggtgaaggccaacgcccacgccccgtcgaagatcaacggcaccgccaccaaggtgccgaagtcgatggagtcgatgaagctggagtcgtcgtcgaccaccggcgccaacgccccgcgcaccttcatcaaccagatcccggactggtcgatgctgctggccgccatcaccaccgccttcctggccgccgagaagcagtggatgatgatcgactggaagccgaagcgctcggacgtgctgtcggacccgttcggcatcggccgcatcgtgcaggacggcctggccttccgccagaacttctcgatccgctcgtacgagatcggcgccgacaagaccgcctcgatcgagaccctgatgaaccacctgcaggagaccgccctgaaccacgtgaagaccgccggcctgctgggcgacggcttcggctcgaccccggagatgtgcaagaagaacctgatctgggtggtgacccgcatgcaggtggaggtggaccgctacccgacctggggcgacgtggtgcaggtggacacctgggtgtcggcctcgggcaagaacggcatgcgccgcgactggatcatccgcgacgccaacaccggcgagatcctgacccgcgcctcgtcgatctgggtgatgatgaacaaggtgacccgccgcctgtcgaagatcccggaggaggtgcgccaggagatcgcctcgtacttcgtggactcgccgccggtggtggaggaggacaaccgcaagctgtcgaagctggacgacaccgccgaccacatccgccgcggcctgtcgccgcgctggtcggacctggacgtgaaccagcacgtgaacaacgtgaagtacatcggctggctgctggagtcggccccgcaggccatcctggagtcgcacgagctgcgcgccatgaccctggagtaccgccgcgagtgcggcaaggactcggtgctggactcgctgaccgacgtgtcgggcgccgacatcggcaacctggccggcggcggctcgctggagtgcaagcacctgctgcgcctggaggacggcggcgagatcgtgcgcggccgcaccgagtggcgcccgaagccggtgaacaacttcggcgccatgaaccaggtgttcccggccgagaactaa

SEQ ID NO: 100 - DNA, 1245 bp, Arachis hypogaea
atggttgctactgctgctacgtcgtcgtttttccctgtgacgtcacgaaccggtggagaaggaggaggaggaatccctgccagcctcggcggagggctcaaacaaaatcacaggtcttcaagtgttaaggccaatgcgcatgctccttcaaagatcaacggaaccgccacaaaggttccaaaatccatggagagcatgaagctggaatcctcgtcgacgacgggggctaatgcgccgaggactttcattaaccagattccggattggagcatgctgctggccgccatcacgacagccttccttgcggcggagaagcagtggatgatgatcgattggaagccgaagcgatccgatgtgctatctgatccatttggtattgggaggattgtgcaggatgggcttgctttcaggcaaaatttctccattcgatcttacgagataggcgccgataagaccgcgtctatagagacgctaatgaatcatttgcaggaaactgcacttaatcatgttaagactgctgggcttcttggtgatggctttggttcgacgccggaaatgtgtaagaagaacctgatatgggttgtgactcggatgcaagttgaagttgatcgttacccaacatggggagatgtagttcaagttgacacttgggtttctgcatcagggaaaaatggtatgcgtcgtgattggatcatacgtgatgccaatacgggtgaaatcttgacaagagcctccagtatttgggtcatgatgaataaagtgacaaggagactatccaaaattccagaagaagtcaggcaagagattgcgtcgtattttgtggattctcctccagttgtcgaagaggataacagaaaactgtctaaacttgatgatactgcagatcatattcgtcgtggtctaagtcctagatggagtgatctagatgttaatcagcatgttaacaatgtgaagtacattggctggcttctggagagtgctccacaggcaatcttggagagtcatgagctgcgggccatgactctggagtacaggagggaatgtggcaaggacagtgtgctggattccctaactgatgtctctggtgctgatatcgggaacttagctggcggcggatctctcgagtgcaaacacttgcttaggcttgaagatggtggtgagattgtgaggggtaggactgaatggaggcccaagcctgtgaacaactttggtgctatgaatcaggtttttccagcagaaaactga

SEQ ID NO: 101 - DNA, 1059 bp, Artificial Sequence
  synthetic polynucleotide
atggagtcgatgaagctggagtcgtcgtcgaccaccggcgccaacgccccgcgcaccttcatcaaccagatcccggactggtcgatgctgctggccgccatcaccaccgccttcctggccgccgagaagcagtggatgatgatcgactggaagccgaagcgctcggacgtgctgtcggacccgttcggcatcggccgcatcgtgcaggacggcctggccttccgccagaacttctcgatccgctcgtacgagatcggcgccgacaagaccgcctcgatcgagaccctgatgaaccacctgcaggagaccgccctgaaccacgtgaagaccgccggcctgctgggcgacggcttcggctcgaccccggagatgtgcaagaagaacctgatctgggtggtgacccgcatgcaggtggaggtggaccgctacccgacctggggcgacgtggtgcaggtggacacctgggtgtcggcctcgggcaagaacggcatgcgccgcgactggatcatccgcgacgccaacaccggcgagatcctgacccgcgcctcgtcgatctgggtgatgatgaacaaggtgacccgccgcctgtcgaagatcccggaggaggtgcgccaggagatcgcctcgtacttcgtggactcgccgccggtggtggaggaggacaaccgcaagctgtcgaagctggacgacaccgccgaccacatccgccgcggcctgtcgccgcgctggtcggacctggacgtgaaccagcacgtgaacaacgtgaagtacatcggctggctgctggagtcggccccgcaggccatcctggagtcgcacgagctgcgcgccatgaccctggagtaccgccgcgagtgcggcaaggactcggtgctggactcgctgaccgacgtgtcgggcgccgacatcggcaacctggccggcggcggctcgctggagtgcaagcacctgctgcgcctggaggacggcggcgagatcgtgcgcggccgcaccgagtggcgcccgaagccggtgaacaacttcggcgccatgaaccaggtgttcccggccgagaactaa

SEQ ID NO: 102 - DNA, 1059 bp, Artificial Sequence
  synthetic polynucleotide
atggagagcatgaagctggaatcctcgtcgacgacgggggctaatgcgccgaggactttcattaaccagattccggattggagcatgctgctggccgccatcacgacagccttccttgcggcggagaagcagtggatgatgatcgattggaagccgaagcgatccgatgtgctatctgatccatttggtattgggaggattgtgcaggatgggcttgctttcaggcaaaatttctccattcgatcttacgagataggcgccgataagaccgcgtctatagagacgctaatgaatcatttgcaggaaactgcacttaatcatgttaagactgctgggcttcttggtgatggctttggttcgacgccggaaatgtgtaagaagaacctgatatgggttgtgactcggatgcaagttgaagttgatcgttacccaacatggggagatgtagttcaagttgacacttgggtttctgcatcagggaaaaatggtatgcgtcgtgattggatcatacgtgatgccaatacgggtgaaatcttgacaagagcctccagtatttgggtcatgatgaataaagtgacaaggagactatccaaaattccagaagaagtcaggcaagagattgcgtcgtattttgtggattctcctccagttgtcgaagaggataacagaaaactgtctaaacttgatgatactgcagatcatattcgtcgtggtctaagtcctagatggagtgatctagatgttaatcagcatgttaacaatgtgaagtacattggctggcttctggagagtgctccacaggcaatcttggagagtcatgagctgcgggccatgactctggagtacaggagggaatgtggcaaggacagtgtgctggattccctaactgatgtctctggtgctgatatcgggaacttagctggcggcggatctctcgagtgcaaacacttgcttaggcttgaagatggtggtgagattgtgaggggtaggactgaatggaggcccaagcctgtgaacaactttggtgctatgaatcaggtttttccagcagaaaactga

SEQ ID NO: 103 - DNA, 1149 bp, Mangifera indica
atgacatccgtggcatgtaagatcatcctttccagagaattattcaaggaagagaagaagattaagcccatggcgacggccaaagtggggctttgttcatcagggaatttgataagacggaaacatggaaggcatttgttgatagcaagtgccagtaatccaaatggtctagacatgatgaaaggaaaaaaggtgaacggaattcaccataacgaagagactcatcatcagctgctacttaaacaaagggtttctaaggcaccccttcatgcatgtttgcttggaaggtttgtaggtgataggtttatgtatagacaaaccttcattataagatcatatgaaattggaccagataaaactgccactatggaaacactcttgaatctccttcaggagactgctctgaatcatgtaacgggctcgggactagctgggaacgggtttggcgctacccgagagatgagtcttcgaaaactcatctgggtcgtcactcgcatcaacattcaagtgcagagatatagctgctggggagatgttgtggagatagatacttgggttgattcttcaggaaagaatgccatgcgcagggactggattatccgagattatcatacccaggaaataataacaagggcaacaagcacctgggtgaccatgaacagagaaacaaggaggttatcaaagattcctgaacaagtaaagcaagaagtgtttccgttttacctagacagagttgcaattgcaaaagaacaaaatgatgttgggaaaattgacaagctaactgatgaaactgcagagagaattcgatctggtttagctccaagatggaatgacatggatgccaatcagcatgtaaacaatgtcaaatacattggatggattttagagagtgttccaatacatgtattaaaagattacaatatgacaagcatgaccctggagtatcgacgtgagtgtcgccaatcaaatttgttggagtccttgacaagctcaacagccagcgtcactggagaccccaacaataattccaacaatcgcattgcagacttgaaatacacacatctacttcgaatgcaagctgataaggctgagatagtccgtgccagatcagaatggcaatccaaacaaataacacaagtcatcacatag

SEQ ID NO: 104 - DNA, 1302 bp, Morella rubra
atgttggaaacttttattttttgtctcctcatcaggcaacgtgagttcgaagctacatcggccaccttatattttcaaacctatattttgctcggttctacaatttcccgcggtatagtatatctctctgttcaatcagcgatgacagtgatggctgcactgagtaacgctcgactgtattttggaggggatttttgcaggggagataagaagaatatggccatggcgaggttgggttattatccttcattgaattgtgcaatcaagccgaagcagccaagtttattagtgatagcaagtgctagtactcctccgagcatatataccatcaacggaaagaaggtgaatggaataaacttcggggaggctccatttaggagtaacaagtatagcgattcagcaaaagaaagctgtgttgatgcacctcttcatgcagttttgcttggaaggttcgtggaggaccagtttgtttatagacagacattcattatcaggtcttacgagattggaccagacaaaactgctaccatggaaacactgatgaatctccttcaggagacagctctcaatcatgtgacaagctctggccttgctgggaatggctttggtgccacccgcgagatgagcctacggaaacttatttgggttgtcacacgggtccacattcaagtacagagatatagctgctggggagatgttgttgagattgatacttgggtggatgccgaggggaaaaatgggatgcgcagggactggataatccgggattaccacacacaagagatcattaccagggccacaagtacttgggttatcatgaatcaagaaacacgacggttgtctaagatcccagaacaagtgagagaggaagtggtaccgttctacctggacagacttgcagtttccgcagaaatgaatgatagtgaaaaaattgacaagctcactgacgaaaccgcagaaagaattcgatcaggattagccccaagatggagcgacatggatgccaatcagcacgtgaacaacgtgaaatatattggatggatcctggagagcgtgcccataaacgtgcttggaaattatgatcttacgagcatgactctggagtaccgccgtgagtgccggcaatcaaatttgctggagtctttgacaagttcaacagcaaatgcaactgaagcctcgtataactccttacatcgatctaaaccagacttggaattcacacacctgcttcgcatgctagcagacaaggcagagatagtacgggcaagaacacagtggcattccaaacaaaaacgaaactga

SEQ ID NO: 105 - DNA, 1161 bp, Pistacia vera
atgatatccgtggcacgaacaagttatgtaagattatcctttccagagaatttattcaaggaagagaaggagatcgtacccatggccatggccaaggtcgggttttgttgctcgttgaatttgatccgaccaaaacatggaaggcttttggtgatagcaagtgctagtaatgcgaagagcctggacatcatgaatggaaaaaaggtgaatggaattcacgttaatgaagagactcgtcatcagcggctacttaatcaaagagttgctgacgcacccctccatgcatgtttgcttggaaaatttgtagaggataggtttttgtatagacaaaccttcattgtcaggtcatatgaaattggaccagataaaactgccactatggaaacactcttgaatctccttcaggagacagctctgaatcatgtaacgagctcgggacttgctgggaacgggtttggtgctacccgagagatgagtgttagaaaactcatctgggtcgtcactcgcatcaacattcaagtacagagatatagctgctggggagatgttgttgagatagatacttgggttgatgcagcaggaaagaatgcgatgcgccgggactggattatccgagattatcgtacccaagagataataacaagagcaacaagcacctgggtgatcatgaacagagaaacaagaagattatcaaagattcctgaacaagtaaggcaagaagtgttaccattttacctaggtagagttgcaattgcaaaagaacaaaatgatgttgggaaaattgacaagcttactgatgaaactgcagagagaattcggtctggtttagctccaagatggaatgacatggatgccaatcagcatgtaaataatgtcaaatacataggatggattttggagagtgtgccaatacacgtcttaaaagattacaatctgacaagcatgaccctggagtatcgacgtgaatgtcgccaatcaaatttgctagagtccttgacaagttcaacagccagtgtcactggagaccccaacaataattccaataatcgcattgcagacttggaatacacacatctacttcgtatgcaagctgataaagctgagatagtccgagccagatcagaatggcagtccaaacaaataacacaagccatcacttga

SEQ ID NO: 106 - DNA, 1128 bp, Artificial Sequence
  synthetic polynucleotide
atgctgaagctgtcgtcgtgcaacgtgaccgaccagcgccaggccctggcccagtgccgcttcctggccccgccggccccgttctcgttccgctggcgcaccccggtggtggtgtcgtgctcgccgtcgtcgcgcccgaacctgtcgccgctgcaggtggtgctgtcgggccagcagcaggccggcatggagctggtggagtcgggctcgggctcgctggccgaccgcctgcgcctgggctcgctgaccgaggacggcctgtcgtacaaggagaagttcatcgtgcgctgctacgaggtgggcatcaacaagaccgccaccgtggagaccatcgccaacctgctgcaggaggtgggctgcaaccacgcccagtcggtgggctactcgaccgacggcttcgccaccacccgcaccatgcgcaagctgcacctgatctgggtgaccgcccgcatgcacatcgagatctacaagtacccggcctggtcggacgtgatcgagatcgagacctggtgccagtcggagggccgcatcggcacccgccgcgactggatcctgaaggacttcggcaccggcgaggtgatcggccgcgccacctcgaagtgggtgatgatgaaccaggacacccgccgcctgcagaaggtgtcggacgacgtgcgcgaggagtacctggtgttctgcccgcgcgagctgcgcctggccttcccggaggagaacaacaactcgctgaagaagatcgccaagctggacgactcgttccagtactcgcgcctgggcctgatgccgcgccgcgccgacctggacatgaaccagcacgtgaacaacgtgacctacatcggctgggtgctggagtcgatgccgcaggagatcatcgacacccacgagctgcagaccatcaccctggactaccgccgcgagtgccagcaggacgacgtggtggactcgctgacctcgccggagcaggtggagggcaccgagaaggtgtcggccatccacggcaccaacggctcggccgccgcccgcgaggacaagcaggactgccgccagttcctgcacctgctgcgcctgtcgtcggacggccaggagatcaaccgcggccgcaccgagtggcgcaagaagccggcccgctaa

SEQ ID NO: 107 - DNA, 1128 bp, Theobroma cacao
atgttgaagctttcttcctgcaatgtgacggaccaaagacaggccttggcccaatgcagattcctcgctccgcctgctccgttctcctttcgctggcgtaccccagtcgtcgtctcctgctctccttccagcagacctaacctgtcgcctcttcaagttgtcctgtctggccagcagcaagctgggatggagctggtcgaatccgggtcggggagtttggctgaccggctccggttgggtagcttgacggaagatggtttgtcctataaggagaagtttattgtgaggtgttatgaggtgggaattaacaaaactgccactgttgaaaccattgccaatctcttgcaggaggttggatgtaaccatgctcaaagtgttggatattccacagatgggtttgctaccactcgcaccatgagaaaattgcatctcatttgggtaactgcacgcatgcacattgaaatatacaaataccctgcttggagtgatgtgattgaaatagagacatggtgccaaagtgagggaagaattggaaccagaagggactggattcttaaggactttggaactggtgaagttattggaagagctactagcaagtgggtgatgatgaaccaggacactaggcggctacagaaagtcagtgatgatgtcagggaagaatatttagtcttctgtccacgagaactcagattagcatttccagaggagaacaataatagtttgaagaaaattgccaaattagatgactcttttcagtattccaggctagggcttatgccaagaagagctgatctggacatgaaccagcatgtcaataatgtcacctacattggatgggttctggagagcatgcctcaagagatcattgacacccatgaactgcaaactatcacgttagattacagacgggaatgccaacaggatgacgtggtggattcacttaccagtccagaacaagtggagggtaccgaaaaagtttcagcgattcacggaacaaatgggtctgcagctgcaagagaagataagcaggactgccgtcagtttttgcatctgttgagattgtctagtgatggacaggaaataaatcgaggccgtactgagtggagaaagaaacctgcgagatga

SEQ ID NO: 108 - DNA, 888 bp, Artificial Sequence
  synthetic polynucleotide
atgctgaccgaggacggcctgtcgtacaaggagaagttcatcgtgcgctgctacgaggtgggcatcaacaagaccgccaccgtggagaccatcgccaacctgctgcaggaggtgggctgcaaccacgcccagtcggtgggctactcgaccgacggcttcgccaccacccgcaccatgcgcaagctgcacctgatctgggtgaccgcccgcatgcacatcgagatctacaagtacccggcctggtcggacgtgatcgagatcgagacctggtgccagtcggagggccgcatcggcacccgccgcgactggatcctgaaggacttcggcaccggcgaggtgatcggccgcgccacctcgaagtgggtgatgatgaaccaggacacccgccgcctgcagaaggtgtcggacgacgtgcgcgaggagtacctggtgttctgcccgcgcgagctgcgcctggccttcccggaggagaacaacaactcgctgaagaagatcgccaagctggacgactcgttccagtactcgcgcctgggcctgatgccgcgccgcgccgacctggacatgaaccagcacgtgaacaacgtgacctacatcggctgggtgctggagtcgatgccgcaggagatcatcgacacccacgagctgcagaccatcaccctggactaccgccgcgagtgccagcaggacgacgtggtggactcgctgacctcgccggagcaggtggagggcaccgagaaggtgtcggccatccacggcaccaacggctcggccgccgcccgcgaggacaagcaggactgccgccagttcctgcacctgctgcgcctgtcgtcggacggccaggagatcaaccgcggccgcaccgagtggcgcaagaagccggcccgctaa

SEQ ID NO: 109 - DNA, 888 bp, Artificial Sequence
  synthetic polynucleotide
atgttgacggaagatggtttgtcctataaggagaagtttattgtgaggtgttatgaggtgggaattaacaaaactgccactgttgaaaccattgccaatctcttgcaggaggttggatgtaaccatgctcaaagtgttggatattccacagatgggtttgctaccactcgcaccatgagaaaattgcatctcatttgggtaactgcacgcatgcacattgaaatatacaaataccctgcttggagtgatgtgattgaaatagagacatggtgccaaagtgagggaagaattggaaccagaagggactggattcttaaggactttggaactggtgaagttattggaagagctactagcaagtgggtgatgatgaaccaggacactaggcggctacagaaagtcagtgatgatgtcagggaagaatatttagtcttctgtccacgagaactcagattagcatttccagaggagaacaataatagtttgaagaaaattgccaaattagatgactcttttcagtattccaggctagggcttatgccaagaagagctgatctggacatgaaccagcatgtcaataatgtcacctacattggatgggttctggagagcatgcctcaagagatcattgacacccatgaactgcaaactatcacgttagattacagacgggaatgccaacaggatgacgtggtggattcacttaccagtccagaacaagtggagggtaccgaaaaagtttcagcgattcacggaacaaatgggtctgcagctgcaagagaagataagcaggactgccgtcagtttttgcatctgttgagattgtctagtgatggacaggaaataaatcgaggccgtactgagtggagaaagaaacctgcgagatga

SEQ ID NO: 110 - DNA, 1188 bp, Theobroma cacao
atggctactttctcttgtccaatctccttccctttcagatgttcttttaacggtaatcataaccataaccacagagtcgatatcaaaatcaatggaactcatacagggcccattaagctcgacacattgaatgaaatagctgcagttgtaaaagctgattccactcctcttgctaatgtccacgaaaacgggtacatatgccaagagaagattcggcagaggattccaacgcagaagcagctggttgatccttaccgtcaagggcttatcattgaaaggggagttggctatagacagactgttgtcatccgctcctatgaagttggccctgataaaactgctaccctggagagcctccttaatcttttccaggaaacagcactaaatcatgtctggatgtctggacttctgagcaatggatttggagccacacatggaatgatgaggaacaatctcatatgggtcgtctcaagaatgcacgtccaagtgcatcactatcccatatggggagaggtagtggaaatcgacacatgggttggagcatcagggaagaatgggatgaggcgagactggctaattcggagtcaagccaccggcatcacctacgcacgtgcaaccagcacttgggtaatgatgaacgagcaaacaaggcgcctctcaaagatgccggaggaagtgaggggtgaaatctctccttggtttattgagaagcaagcaatcaaagaagatgctcccgagaaaattgtcaagttggacgacaaagcaaaatatgtgaactctgacttgaagccaaagaggagtgatttggacatgaaccaccatgtaaacaatgtcaagtatgtacgatggatgcttgagacaattcctgacaaatttttggagtctcaccagctatctagtattgtactagaatatagaagggaatgtgggagttcggataaagttcaatcactttgccaaccagatgaagatagaattttagcaaatggactggaacaaagtctacttgaaaatatagttttggcatcgggaatcatgctaggaaatggacacctaggctccctgggtatgaagacatatggatttactcatctcctccaaatcaaaggggacagtcaaaatgacgagatagtcagagggaggaccagatggaagaaaaagcaatctaccacgccgtattccacttaa

SEQ ID NO: 111 - DNA, 912 bp, Artificial Sequence
  synthetic polynucleotide
atgggagttggctatagacagactgttgtcatccgctcctatgaagttggccctgataaaactgctaccctggagagcctccttaatcttttccaggaaacagcactaaatcatgtctggatgtctggacttctgagcaatggatttggagccacacatggaatgatgaggaacaatctcatatgggtcgtctcaagaatgcacgtccaagtgcatcactatcccatatggggagaggtagtggaaatcgacacatgggttggagcatcagggaagaatgggatgaggcgagactggctaattcggagtcaagccaccggcatcacctacgcacgtgcaaccagcacttgggtaatgatgaacgagcaaacaaggcgcctctcaaagatgccggaggaagtgaggggtgaaatctctccttggtttattgagaagcaagcaatcaaagaagatgctcccgagaaaattgtcaagttggacgacaaagcaaaatatgtgaactctgacttgaagccaaagaggagtgatttggacatgaaccaccatgtaaacaatgtcaagtatgtacgatggatgcttgagacaattcctgacaaatttttggagtctcaccagctatctagtattgtactagaatatagaagggaatgtgggagttcggataaagttcaatcactttgccaaccagatgaagatagaattttagcaaatggactggaacaaagtctacttgaaaatatagttttggcatcgggaatcatgctaggaaatggacacctaggctccctgggtatgaagacatatggatttactcatctcctccaaatcaaaggggacagtcaaaatgacgagatagtcagagggaggaccagatggaagaaaaagcaatctaccacgccgtattccacttaa

SEQ ID NO: 112 - DNA, 1398 bp, Theobroma cacao
atgcaccaatgccactcgaatgctcccctatttgcgtgcaagctaacgactgaggtttctcgatttctcccccacccattggaagtggttgagctgcaaattgcatctgctcctctagatgctggagctagggagctagggactgccatctgtttctttttccgaactcgaaagtttgctagcttggctctttttttttgttcgcgggtgagcaaatgcgaggcacttaccatggcatcaatggccaaagcaagcaatgtaacttcattatttctagggggtgtatgcaaggaagagaaaacgaagaatgtagccatggcgaagttgggtttttattcttcatggaacttgatcaaaccgaaacggaaaggccttttgctaattgcaagtgctaaaaatcctcataatctggacatgatcaacgggaaaaaagtaaatggaatttttgttggtgaagctccatatacgggaaagaagagcactgtgttgataaaagaacacgtcccttataaacaagcccacgcggcgagtttggttggaaggtttgtggaggataggcatgtctacaggcagaccttcatcatcaggtcttatgaaactggaccggacaaaactgccaccatggaaacggttatgaatctccttcaggaaacagctttgaatcatgtaaggagctctggtcttgctgggaatggctttggggctacccgtgagatgagccttaggaaactcatatgggtcgtcacccgcatccacgttcaagtggagagatatagctgctggggagatgttgtggagattgatacttgggttgatgcagcaggaaagaatgcaatgcgtagggactggataatcagagactacaatacacaggagatcataacaagagcaacaagcacatgggtgattatgaaccacgaaacacgaagattaaccaagattcctgaacaagttaggcaagaagtgattccattctacctaaacaggattgcaatcgctgaagagaagaacgatatcggaaagattgataagcttactgatgaaaacgcagaaagaatacggtctggcttagctccaagatggagcgacatggatgccaatcagcatgtaaacaatgttaaatacatcggatggattttggagagtgtgccaatggacgtcctggaagagtatcgtctgacgagcatgaccctggagtatcgacgtgaatgccggaaatccaatttgctagagtccttgacgagttcaacagcaaatgtgacagaagattccaacaacaattctaataatcgcaaggcagacttggaatacacacatctgcttcgcatgcaagacgacgtggcagagatagtccgagctagatcagaatggcaatccaaggacaaacacagctggtga

SEQ ID NO: 113 - DNA, 855 bp, Artificial Sequence
  synthetic polynucleotide
atggtggaggataggcatgtctacaggcagaccttcatcatcaggtcttatgaaactggaccggacaaaactgccaccatggaaacggttatgaatctccttcaggaaacagctttgaatcatgtaaggagctctggtcttgctgggaatggctttggggctacccgtgagatgagccttaggaaactcatatgggtcgtcacccgcatccacgttcaagtggagagatatagctgctggggagatgttgtggagattgatacttgggttgatgcagcaggaaagaatgcaatgcgtagggactggataatcagagactacaatacacaggagatcataacaagagcaacaagcacatgggtgattatgaaccacgaaacacgaagattaaccaagattcctgaacaagttaggcaagaagtgattccattctacctaaacaggattgcaatcgctgaagagaagaacgatatcggaaagattgataagcttactgatgaaaacgcagaaagaatacggtctggcttagctccaagatggagcgacatggatgccaatcagcatgtaaacaatgttaaatacatcggatggattttggagagtgtgccaatggacgtcctggaagagtatcgtctgacgagcatgaccctggagtatcgacgtgaatgccggaaatccaatttgctagagtccttgacgagttcaacagcaaatgtgacagaagattccaacaacaattctaataatcgcaaggcagacttggaatacacacatctgcttcgcatgcaagacgacgtggcagagatagtccgagctagatcagaatggcaatccaaggacaaacacagctggtga

SEQ ID NO: 114 - DNA, 1167 bp, Theobroma cacao
atggcatcaatggccaaagcaagcaatgtaacttcattatttctagggggtgtatgcaaggaagagaaaacgaagaatgtagccatggcgaagttgggtttttattcttcatggaacttgatcaaaccgaaacggaaaggccttttgctaattgcaagtgctaaaaatcctcataatctggacatgatcaacgggaaaaaagtaaatggaatttttgttggtgaagctccatatacgggaaagaagagcactgtgttgataaaagaacacgtcccttataaacaagcccacgcggcgagtttggttggaaggtttgtggaggataggcatgtctacaggcagaccttcatcatcaggtcttatgaaactggaccggacaaaactgccaccatggaaacggttatgaatctccttcaggaaacagctttgaatcatgtaaggagctctggtcttgctgggaatggctttggggctacccgtgagatgagccttaggaaactcatatgggtcgtcacccgcatccacgttcaagtggagagatatagctgctggggagatgttgtggagattgatacttgggttgatgcagcaggaaagaatgcaatgcgtagggactggataatcagagactacaatacacaggagatcataacaagagcaacaagcacatgggtgattatgaaccacgaaacacgaagattaaccaagattcctgaacaagttaggcaagaagtgattccattctacctaaacaggattgcaatcgctgaagagaagaacgatatcggaaagattgataagcttactgatgaaaacgcagaaagaatacggtctggcttagctccaagatggagcgacatggatgccaatcagcatgtaaacaatgttaaatacatcggatggattttggagagtgtgccaatggacgtcctggaagagtatcgtctgacgagcatgaccctggagtatcgacgtgaatgccggaaatccaatttgctagagtccttgacgagttcaacagcaaatgtgacagaagattccaacaacaattctaataatcgcaaggcagacttggaatacacacatctgcttcgcatgcaagacgacgtggcagagatagtccgagctagatcagaatggcaatccaaggacaaacacagctggtga

SEQ ID NO: 115 - DNA, 855 bp, Artificial Sequence; identical to SEQ ID NO: 113
  synthetic polynucleotide

SEQ ID NO: 116 - DNA, 1158 bp, Theobroma cacao
atgcaccaatgccactcgaatgctcccctatttgcgtgcaagctaacgactgaggtttctcgatttctcccccacccattggaagtggttgagctgcaaattgcatctgctcctctagatgctggagctagggagctagggactgccatctgtttctttttccgaactcgaaagtttgctagcttggctctttttttttgttcgcgggtgagcaaatgcgaggcacttaccatggcatcaatggccaaagcaagcaatgtaacttcattatttctagggggtgtatgcaaggaagagaaaacgaagaatgtagccatggcgaagttgggtttttattcttcatggaacttgatcaaaccgaaacggaaaggccttttgctaattgcaagtgctaaaaatcctcataatctggacatgatcaacgggaaaaaagtaaatggaatttttgttggtgaagctccatatacgggaaagaagagcactgtgttgataaaagaacacgtcccttataaacaagcccacgcggcgagtttggttggaaggtttgtggaggataggcatgtctacaggcagaccttcatcatcaggtcttatgaaactggaccggacaaaactgccaccatggaaacggttatgaatctccttcaggaaacagctttgaatcatgtaaggagctctggtcttgctgggaatggctttggggctacccgtgagatgagccttaggaaactcatatgggtcgtcacccgcatccacgttcaagtggagagatatagctgctggggagatgttgtggagattgatacttgggttgatgcagcaggaaagaatgcaatgcgtagggactggataatcagagactacaatacacaggagatcataacaagagcaacaagcacatgggtgattatgaaccacgaaacacgaagattaaccaagattcctgaacaagttaggcaagaagtgattccattctacctaaacaggattgcaatcgctgaagagaagaacgatatcggaaagattgataagcttactgatgaaaacgcagaaagaatacggtctggcttagctccaagatggagcgacatggatgccaatcagcatgtaaacaatgttaaatacatcggatggattttggaggcattcaccaactaa

SEQ ID NO: 117 - DNA, 615 bp, Artificial Sequence
  synthetic polynucleotide
atggtggaggataggcatgtctacaggcagaccttcatcatcaggtcttatgaaactggaccggacaaaactgccaccatggaaacggttatgaatctccttcaggaaacagctttgaatcatgtaaggagctctggtcttgctgggaatggctttggggctacccgtgagatgagccttaggaaactcatatgggtcgtcacccgcatccacgttcaagtggagagatatagctgctggggagatgttgtggagattgatacttgggttgatgcagcaggaaagaatgcaatgcgtagggactggataatcagagactacaatacacaggagatcataacaagagcaacaagcacatgggtgattatgaaccacgaaacacgaagattaaccaagattcctgaacaagttaggcaagaagtgattccattctacctaaacaggattgcaatcgctgaagagaagaacgatatcggaaagattgataagcttactgatgaaaacgcagaaagaatacggtctggcttagctccaagatggagcgacatggatgccaatcagcatgtaaacaatgttaaatacatcggatggattttggaggcattcaccaactaa

SEQ ID NO: 118 - DNA, 1131 bp, Theobroma cacao
atggcagcatcttcaaacattatgacatcgaagttcttcatggcgacctctccctcatcctggaattcaacgaataaatcaaagatttgcttgcaacaaatcgatagaagttcaaatacgaatggaaagatggttaagtttactcgagacagtagtttgaaggtcaaattgcaggctcaagcactgcttactaatgacagtagagcaacatcaatgatagaaagcctgaaggatgaggaaatgatgacttcaccacccgcaacaatggaacacctgacaacggaaggaagattgataaatgacggactggttttccagcagaatttttccattaggtcattcgagatagactctgagtacaaagtttcagcaagggctataatgaattatttgcaggagtcatcacttaaccatagaaagaagatggggatgtcaagcgattcccttgtaggtgtaacgccagagatgattaaaagggacttgctatggatattccgtggcatgtgtattgaggtggatcgctatccttcttgggctgatgttgtccagatataccaccggatctatacatcaggaaggactggtttgcgtttggaatggattgtcaatgacagcaagacaggcgaaactctagttcgagcatcatgcttagctgtgatgatgaataagaaaacaagaaaaacatgcaagtttccggaggaagtcaaacaggagctaaagccttatcttacgacagacgctgagcctctcttcgaagctgacaaaattttatgtccccaagttgggaaaatggataacatccgaaccggattgacttcctgttggcatgacctggatttcaattaccatgtaaacaatgcaaagtatcttgactggattttggagggtactcctacctctcttatacatagccatgagctttctagagtaagtctccagtaccgaaaggagtgcttgaaagatgatgtgattcaatctttatccagagttgttaccaaggaaactggtctctcaacgaacaatcaagaaattgaattagaacacgtcctccgtcttgagagtggaccggaacttgcaggggcaaggaccgcttggaggccaaaatctatctgccgacaaactataaattaa

SEQ ID NO: 119 - DNA, 843 bp, Artificial Sequence
  synthetic polynucleotide
atgttgataaatgacggactggttttccagcagaatttttccattaggtcattcgagatagactctgagtacaaagtttcagcaagggctataatgaattatttgcaggagtcatcacttaaccatagaaagaagatggggatgtcaagcgattcccttgtaggtgtaacgccagagatgattaaaagggacttgctatggatattccgtggcatgtgtattgaggtggatcgctatccttcttgggctgatgttgtccagatataccaccggatctatacatcaggaaggactggtttgcgtttggaatggattgtcaatgacagcaagacaggcgaaactctagttcgagcatcatgcttagctgtgatgatgaataagaaaacaagaaaaacatgcaagtttccggaggaagtcaaacaggagctaaagccttatcttacgacagacgctgagcctctcttcgaagctgacaaaattttatgtccccaagttgggaaaatggataacatccgaaccggattgacttcctgttggcatgacctggatttcaattaccatgtaaacaatgcaaagtatcttgactggattttggagggtactcctacctctcttatacatagccatgagctttctagagtaagtctccagtaccgaaaggagtgcttgaaagatgatgtgattcaatctttatccagagttgttaccaaggaaactggtctctcaacgaacaatcaagaaattgaattagaacacgtcctccgtcttgagagtggaccggaacttgcaggggcaaggaccgcttggaggccaaaatctatctgccgacaaactataaattaa

SEQ ID NO: 120 - DNA, 1263 bp, Theobroma cacao
atggttgctactgctgcatcatctgcattctttccggtcacttcatccccggactcctctgactcaaaaaacaagaagcttggaagtggatctactaaccttggaggtatcaagtcgaaaccatctactccttctggaattttgcaagtcaaggcaaatgctcaagctcctccaaaaataaatggtaccacggtcgtgacaactccagtggaaagtttcaagaatgaggacactgctagttcccctcctcccaggacatttataaaccagttacctgattggagcatgcttcttgctgctatcacgacaattttcttggctgctgagaagcagtggatgatgcttgattggaaacccaggcggcctgacatgctcattgatccatttggtatagggaggattgttcaggatggtcttgttttccgccagaatttctctataaggtcttacgagataggtgctgatcggacagcatccatagagacgctaatgaatcatttacaggaaacggctattaatcattgtagaagcgctggactgcttggagaaggttttggttcaacccctgagatgtgcaagaaaaacctaatatgggtagtcactcgcatgcaagttgtggttgatcgctatcctacatggggtgatgttgttcaagtagacacttgggccagtgcatcgggaaagaatggtatgcgacgggattggcttgtcagtgatagtaaaactggtgaaattttaacaagagcctcaagtgtatgggtgatgatgaataagcttactagaaggttatctaaaattccagaagaggtccgaggagaaatagaaccttattttatgaattctgatcctgttgtggctgaggatagtaggaaattagtgaaacttgatgacagcacagcagattatgtccgtaaaggtttaactcccagatggggtgatttggatgtcaaccagcatgtcaataatgtgaagtacattggctggatccttgagagtgctccactgccaatcttggagactcacgagctttcttcaatgacactggaatataggagggagtgtgggagagacggtgtgctgcagtccctaactgctgtctctgactctggtgtgggcaacttggtgaactttggtgaaatcgagtgccagcacttgctccgactcgaggatgggtctgagattgtgagagggaggactgagtggaggccgaagtatgcgaaaagttttggtaatgtgggccaacttcctgctgaaagtgcatag

SEQ ID NO: 121 - DNA, 867 bp, Artificial Sequence
  synthetic polynucleotide
atgattgttcaggatggtcttgttttccgccagaatttctctataaggtcttacgagataggtgctgatcggacagcatccatagagacgctaatgaatcatttacaggaaacggctattaatcattgtagaagcgctggactgcttggagaaggttttggttcaacccctgagatgtgcaagaaaaacctaatatgggtagtcactcgcatgcaagttgtggttgatcgctatcctacatggggtgatgttgttcaagtagacacttgggccagtgcatcgggaaagaatggtatgcgacgggattggcttgtcagtgatagtaaaactggtgaaattttaacaagagcctcaagtgtatgggtgatgatgaataagcttactagaaggttatctaaaattccagaagaggtccgaggagaaatagaaccttattttatgaattctgatcctgttgtggctgaggatagtaggaaattagtgaaacttgatgacagcacagcagattatgtccgtaaaggtttaactcccagatggggtgatttggatgtcaaccagcatgtcaataatgtgaagtacattggctggatccttgagagtgctccactgccaatcttggagactcacgagctttcttcaatgacactggaatataggagggagtgtgggagagacggtgtgctgcagtccctaactgctgtctctgactctggtgtgggcaacttggtgaactttggtgaaatcgagtgccagcacttgctccgactcgaggatgggtctgagattgtgagagggaggactgagtggaggccgaagtatgcgaaaagttttggtaatgtgggccaacttcctgctgaaagtgcatag

SEQ ID NO: 122 - protein, 253 aa, Limosilactobacillus reuteri
MELKQEAQTFEMPHLLTYYECDETSHPSLSMILSMISMVSDEHSMSLGMGTKEIQSTGGTWVVSGYEGHLSAKQPSFGETVILGTKAVSYNRFFAVRDFWITGKEHQIEYARIRSIFVFMNLKTRRMQSIPPALIEPFNAPVAKRIPRLKRPQKLDENASVIKKNYQVRYFDLDANHHVNNARYFDWLLDPLGRDFLRGNQIKRFNLQYLQEVRNGEMVESKVNKLQTNDEKVTYHQIGVGEQIDAIAEIEWY

SEQ ID NO: 123 - protein, 414 aa, Arachis hypogaea
MVATAATSSFFPVTSRTGGEGGGGIPASLGGGLKQNHRSSSVKANAHAPSKINGTATKVPKSMESMKLESSSTTGANAPRTFINQIPDWSMLLAAITTAFLAAEKQWMMIDWKPKRSDVLSDPFGIGRIVQDGLAFRQNFSIRSYEIGADKTASIETLMNHLQETALNHVKTAGLLGDGFGSTPEMCKKNLIWVVTRMQVEVDRYPTWGDVVQVDTWVSASGKNGMRRDWIIRDANTGEILTRASSIWVMMNKVTRRLSKIPEEVRQEIASYFVDSPPVVEEDNRKLSKLDDTADHIRRGLSPRWSDLDVNQHVNNVKYIGWLLESAPQAILESHELRAMTLEYRRECGKDSVLDSLTDVSGADIGNLAGGGSLECKHLLRLEDGGEIVRGRTEWRPKPVNNFGAMNQVFPAEN

SEQ ID NO: 124 - protein, 352 aa, Artificial Sequence
  synthetic polypeptide
MESMKLESSSTTGANAPRTFINQIPDWSMLLAAITTAFLAAEKQWMMIDWKPKRSDVLSDPFGIGRIVQDGLAFRQNFSIRSYEIGADKTASIETLMNHLQETALNHVKTAGLLGDGFGSTPEMCKKNLIWVVTRMQVEVDRYPTWGDVVQVDTWVSASGKNGMRRDWIIRDANTGEILTRASSIWVMMNKVTRRLSKIPEEVRQEIASYFVDSPPVVEEDNRKLSKLDDTADHIRRGLSPRWSDLDVNQHVNNVKYIGWLLESAPQAILESHELRAMTLEYRRECGKDSVLDSLTDVSGADIGNLAGGGSLECKHLLRLEDGGEIVRGRTEWRPKPVNNFGAMNQVFPAEN

SEQ ID NO: 125 - protein, 382 aa, Mangifera indica
MTSVACKIILSRELFKEEKKIKPMATAKVGLCSSGNLIRRKHGRHLLIASASNPNGLDMMKGKKVNGIHHNEETHHQLLLKQRVSKAPLHACLLGRFVGDRFMYRQTFIIRSYEIGPDKTATMETLLNLLQETALNHVTGSGLAGNGFGATREMSLRKLIWVVTRINIQVQRYSCWGDVVEIDTWVDSSGKNAMRRDWIIRDYHTQEIITRATSTWVTMNRETRRLSKIPEQVKQEVFPFYLDRVAIAKEQNDVGKIDKLTDETAERIRSGLAPRWNDMDANQHVNNVKYIGWILESVPIHVLKDYNMTSMTLEYRRECRQSNLLESLTSSTASVTGDPNNNSNNRIADLKYTHLLRMQADKAEIVRARSEWQSKQITQVIT

SEQ ID NO: 126 - protein, 433 aa, Morella rubra
MLETFIFCLLIRQREFEATSATLYFQTYILLGSTISRGIVYLSVQSAMTVMAALSNARLYFGGDFCRGDKKNMAMARLGYYPSLNCAIKPKQPSLLVIASASTPPSIYTINGKKVNGINFGEAPFRSNKYSDSAKESCVDAPLHAVLLGRFVEDQFVYRQTFIIRSYEIGPDKTATMETLMNLLQETALNHVTSSGLAGNGFGATREMSLRKLIWVVTRVHIQVQRYSCWGDVVEIDTWVDAEGKNGMRRDWIIRDYHTQEIITRATSTWVIMNQETRRLSKIPEQVREEVVPFYLDRLAVSAEMNDSEKIDKLTDETAERIRSGLAPRWSDMDANQHVNNVKYIGWILESVPINVLGNYDLTSMTLEYRRECRQSNLLESLTSSTANATEASYNSLHRSKPDLEFTHLLRMLADKAEIVRARTQWHSKQKRN

SEQ ID NO: 127 - protein, 386 aa, Pistacia vera
MISVARTSYVRLSFPENLFKEEKEIVPMAMAKVGFCCSLNLIRPKHGRLLVIASASNAKSLDIMNGKKVNGIHVNEETRHQRLLNQRVADAPLHACLLGKFVEDRFLYRQTFIVRSYEIGPDKTATMETLLNLLQETALNHVTSSGLAGNGFGATREMSVRKLIWVVTRINIQVQRYSCWGDVVEIDTWVDAAGKNAMRRDWIIRDYRTQEIITRATSTWVIMNRETRRLSKIPEQVRQEVLPFYLGRVAIAKEQNDVGKIDKLTDETAERIRSGLAPRWNDMDANQHVNNVKYIGWILESVPIHVLKDYNLTSMTLEYRRECRQSNLLESLTSSTASVTGDPNNNSNNRIADLEYTHLLRMQADKAEIVRARSEWQSKQITQAIT

SEQ ID NO: 128 - protein, 375 aa, Theobroma cacao
MLKLSSCNVTDQRQALAQCRFLAPPAPFSFRWRTPVVVSCSPSSRPNLSPLQVVLSGQQQAGMELVESGSGSLADRLRLGSLTEDGLSYKEKFIVRCYEVGINKTATVETIANLLQEVGCNHAQSVGYSTDGFATTRTMRKLHLIWVTARMHIEIYKYPAWSDVIEIETWCQSEGRIGTRRDWILKDFGTGEVIGRATSKWVMMNQDTRRLQKVSDDVREEYLVFCPRELRLAFPEENNNSLKKIAKLDDSFQYSRLGLMPRRADLDMNQHVNNVTYIGWVLESMPQEIIDTHELQTITLDYRRECQQDDVVDSLTSPEQVEGTEKVSAIHGTNGSAAAREDKQDCRQFLHLLRLSSDGQEINRGRTEWRKKPAR

SEQ ID NO: 129 - protein, 295 aa, Artificial Sequence
  synthetic polypeptide
MLTEDGLSYKEKFIVRCYEVGINKTATVETIANLLQEVGCNHAQSVGYSTDGFATTRTMRKLHLIWVTARMHIEIYKYPAWSDVIEIETWCQSEGRIGTRRDWILKDFGTGEVIGRATSKWVMMNQDTRRLQKVSDDVREEYLVFCPRELRLAFPEENNNSLKKIAKLDDSFQYSRLGLMPRRADLDMNQHVNNVTYIGWVLESMPQEIIDTHELQTITLDYRRECQQDDVVDSLTSPEQVEGTEKVSAIHGTNGSAAAREDKQDCRQFLHLLRLSSDGQEINRGRTEWRKKPAR

SEQ ID NO: 130 - protein, 395 aa, Theobroma cacao
MATFSCPISFPFRCSFNGNHNHNHRVDIKINGTHTGPIKLDTLNEIAAVVKADSTPLANVHENGYICQEKIRQRIPTQKQLVDPYRQGLIIERGVGYRQTVVIRSYEVGPDKTATLESLLNLFQETALNHVWMSGLLSNGFGATHGMMRNNLIWVVSRMHVQVHHYPIWGEVVEIDTWVGASGKNGMRRDWLIRSQATGITYARATSTWVMMNEQTRRLSKMPEEVRGEISPWFIEKQAIKEDAPEKIVKLDDKAKYVNSDLKPKRSDLDMNHHVNNVKYVRWMLETIPDKFLESHQLSSIVLEYRRECGSSDKVQSLCQPDEDRILANGLEQSLLENIVLASGIMLGNGHLGSLGMKTYGFTHLLQIKGDSQNDEIVRGRTRWKKKQSTTPYST

SEQ ID NO: 131 - protein, 303 aa, Artificial Sequence
  synthetic polypeptide
MGVGYRQTVVIRSYEVGPDKTATLESLLNLFQETALNHVWMSGLLSNGFGATHGMMRNNLIWVVSRMHVQVHHYPIWGEVVEIDTWVGASGKNGMRRDWLIRSQATGITYARATSTWVMMNEQTRRLSKMPEEVRGEISPWFIEKQAIKEDAPEKIVKLDDKAKYVNSDLKPKRSDLDMNHHVNNVKYVRWMLETIPDKFLESHQLSSIVLEYRRECGSSDKVQSLCQPDEDRILANGLEQSLLENIVLASGIMLGNGHLGSLGMKTYGFTHLLQIKGDSQNDEIVRGRTRWKKKQSTTPYST

SEQ ID NO: 132 - protein, 465 aa, Theobroma cacao
MHQCHSNAPLFACKLTTEVSRFLPHPLEVVELQIASAPLDAGARELGTAICFFFRTRKFASLALFFCSRVSKCEALTMASMAKASNVTSLFLGGVCKEEKTKNVAMAKLGFYSSWNLIKPKRKGLLLIASAKNPHNLDMINGKKVNGIFVGEAPYTGKKSTVLIKEHVPYKQAHAASLVGRFVEDRHVYRQTFIIRSYETGPDKTATMETVMNLLQETALNHVRSSGLAGNGFGATREMSLRKLIWVVTRIHVQVERYSCWGDVVEIDTWVDAAGKNAMRRDWIIRDYNTQEIITRATSTWVIMNHETRRLTKIPEQVRQEVIPFYLNRIAIAEEKNDIGKIDKLTDENAERIRSGLAPRWSDMDANQHVNNVKYIGWILESVPMDVLEEYRLTSMTLEYRRECRKSNLLESLTSSTANVTEDSNNNSNNRKADLEYTHLLRMQDDVAEIVRARSEWQSKDKHSW

SEQ ID NO: 133 - protein, 284 aa, Artificial Sequence
  synthetic polypeptide
MVEDRHVYRQTFIIRSYETGPDKTATMETVMNLLQETALNHVRSSGLAGNGFGATREMSLRKLIWVVTRIHVQVERYSCWGDVVEIDTWVDAAGKNAMRRDWIIRDYNTQEIITRATSTWVIMNHETRRLTKIPEQVRQEVIPFYLNRIAIAEEKNDIGKIDKLTDENAERIRSGLAPRWSDMDANQHVNNVKYIGWILESVPMDVLEEYRLTSMTLEYRRECRKSNLLESLTSSTANVTEDSNNNSNNRKADLEYTHLLRMQDDVAEIVRARSEWQSKDKHSW

SEQ ID NO: 134 - protein, 388 aa, Theobroma cacao
MASMAKASNVTSLFLGGVCKEEKTKNVAMAKLGFYSSWNLIKPKRKGLLLIASAKNPHNLDMINGKKVNGIFVGEAPYTGKKSTVLIKEHVPYKQAHAASLVGRFVEDRHVYRQTFIIRSYETGPDKTATMETVMNLLQETALNHVRSSGLAGNGFGATREMSLRKLIWVVTRIHVQVERYSCWGDVVEIDTWVDAAGKNAMRRDWIIRDYNTQEIITRATSTWVIMNHETRRLTKIPEQVRQEVIPFYLNRIAIAEEKNDIGKIDKLTDENAERIRSGLAPRWSDMDANQHVNNVKYIGWILESVPMDVLEEYRLTSMTLEYRRECRKSNLLESLTSSTANVTEDSNNNSNNRKADLEYTHLLRMQDDVAEIVRARSEWQSKDKHSW

SEQ ID NO: 135 - protein, 284 aa, Artificial Sequence; identical to SEQ ID NO: 133
  synthetic polypeptide

SEQ ID NO: 136 - protein, 385 aa, Theobroma cacao
MHQCHSNAPLFACKLTTEVSRFLPHPLEVVELQIASAPLDAGARELGTAICFFFRTRKFASLALFFCSRVSKCEALTMASMAKASNVTSLFLGGVCKEEKTKNVAMAKLGFYSSWNLIKPKRKGLLLIASAKNPHNLDMINGKKVNGIFVGEAPYTGKKSTVLIKEHVPYKQAHAASLVGRFVEDRHVYRQTFIIRSYETGPDKTATMETVMNLLQETALNHVRSSGLAGNGFGATREMSLRKLIWVVTRIHVQVERYSCWGDVVEIDTWVDAAGKNAMRRDWIIRDYNTQEIITRATSTWVIMNHETRRLTKIPEQVRQEVIPFYLNRIAIAEEKNDIGKIDKLTDENAERIRSGLAPRWSDMDANQHVNNVKYIGWILEAFTN

SEQ ID NO: 137 - protein, 204 aa, Artificial Sequence
  synthetic polypeptide
MVEDRHVYRQTFIIRSYETGPDKTATMETVMNLLQETALNHVRSSGLAGNGFGATREMSLRKLIWVVTRIHVQVERYSCWGDVVEIDTWVDAAGKNAMRRDWIIRDYNTQEIITRATSTWVIMNHETRRLTKIPEQVRQEVIPFYLNRIAIAEEKNDIGKIDKLTDENAERIRSGLAPRWSDMDANQHVNNVKYIGWILEAFTN

SEQ ID NO: 138 - protein, 376 aa, Theobroma cacao
MAASSNIMTSKFFMATSPSSWNSTNKSKICLQQIDRSSNTNGKMVKFTRDSSLKVKLQAQALLTNDSRATSMIESLKDEEMMTSPPATMEHLTTEGRLINDGLVFQQNFSIRSFEIDSEYKVSARAIMNYLQESSLNHRKKMGMSSDSLVGVTPEMIKRDLLWIFRGMCIEVDRYPSWADVVQIYHRIYTSGRTGLRLEWIVNDSKTGETLVRASCLAVMMNKKTRKTCKFPEEVKQELKPYLTTDAEPLFEADKILCPQVGKMDNIRTGLTSCWHDLDFNYHVNNAKYLDWILEGTPTSLIHSHELSRVSLQYRKECLKDDVIQSLSRVVTKETGLSTNNQEIELEHVLRLESGPELAGARTAWRPKSICRQTIN

SEQ ID NO: 139 - protein, 280 aa, Artificial Sequence
  synthetic polypeptide
MLINDGLVFQQNFSIRSFEIDSEYKVSARAIMNYLQESSLNHRKKMGMSSDSLVGVTPEMIKRDLLWIFRGMCIEVDRYPSWADVVQIYHRIYTSGRTGLRLEWIVNDSKTGETLVRASCLAVMMNKKTRKTCKFPEEVKQELKPYLTTDAEPLFEADKILCPQVGKMDNIRTGLTSCWHDLDFNYHVNNAKYLDWILEGTPTSLIHSHELSRVSLQYRKECLKDDVIQSLSRVVTKETGLSTNNQEIELEHVLRLESGPELAGARTAWRPKSICRQTIN